(19) United States
(12) Patent Application Publication
(10) Pub. No.: US 2006/0063828 A1
(43) Pub. Date: Mar. 23, 2006

(54) 1,2-Bis-(substituted-phenyl)-2-propen-1-ones and pharmaceutical compositions thereof

(76) Inventors: M. David Weingarten, Cumming, GA (US); Jason Skudlarek, Suwanee, GA (US); Lee K. Hoong, Swanee, GA (US); James A. Sikorski, Atlanta, GA (US)

Correspondence Address: Sherry M. Knowles, Esq.;King & Spalding LLP, 191 Peachtree Street, Atlanta, GA, 30303-1763, US

(21) Appl. No.: 11/170,647

(22) Filed: Jun. 28, 2005

Related U.S. Application Data
(60) Provisional application No. 60/583,806, filed on Jun. 28, 2004.

(51) Int. Cl. C07D 333/72; C07D 333/16; A61K 31/381

(52) U.S. Cl. 514/438; 514/443; 549/49; 549/78

(57) ABSTRACT

The invention relates to compounds, pharmaceutical compositions and methods of using compounds of the general formula 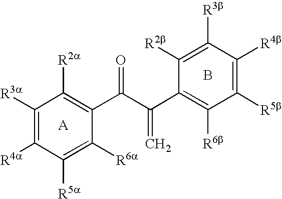 or its pharmaceutically acceptable salt or ester, wherein the substituents are defined in the application.

### CROSS-REFERENCE TO RELATED APPLICATIONS

This application claims priority to U.S. Provisional Application No. 60/583,806, filed Jun. 28, 2004.

### FIELD OF THE INVENTION

The present invention is in the field of novel 1,2-bis-(substituted-phenyl)-2-propen-1-one derivatives, pharmaceutical compositions and methods for treating a variety of diseases and disorders, including inflammation and cardiovascular disease.

### DESCRIPTION OF RELATED ART

Adhesion of leukocytes to the endothelium represents a fundamental, early event in a wide variety of inflammatory conditions, autoimmune disorders and bacterial and viral infections. Leukocyte recruitment to endothelium is mediated in part by the inducible expression of adhesion molecules on the surface of endothelial cells that interact with counterreceptors on immune cells. Endothelial cells determine which types of leukocytes are recruited by selectively expressing specific adhesion molecules, such as vascular cell adhesion molecule-1 (VCAM-1), intercellular adhesion molecule-1 (ICAM-1), and E-selectin. VCAM-1 binds to the integrin VLA-4 expressed on lymphocytes, monocytes, macrophages, eosinophils, and basophils but not neutrophils. This interaction facilitates the firm adhesion of these leukocytes to the endothelium. VCAM-1 is an inducible gene that is not expressed, or expressed at very low levels, in normal tissues. VCAM-1 is upregulated in a number of inflammatory diseases, including arthritis (including rheumatoid arthritis), asthma, dermatitis, psoriasis, cystic fibrosis, post transplantation late and chronic solid organ rejection, multiple sclerosis, systemic lupus erythematosis, inflammatory bowel diseases, autoimmune diabetes, diabetic retinopathy, rhinitis, ischemia-reperfusion injury, post-angioplasty restenosis, chronic obstructive pulmonary disease (COPD), glomerulonephritis, Graves disease, gastrointestinal allergies, conjunctivitis, atherosclerosis, coronary artery disease, angina and small artery disease.

WO 02/074307 discloses isoflavone compounds, metabolites and derivatives which are useful for inhibiting or down-regulating the expression or activity of adhesion molecules in endothelial cells. The WO 02/074307 compounds are used to treat vascular disease and for the treatment of procedural vascular trauma.

WO 03/051864 and WO 03/035635 disclose the use of certain isoflavones and derivatives for the preparation of pharmaceutical compositions for the treatment, amerlioration and prevention of diseases, conditions and disorders including i) all forms of cancer, ii) diseases and disorders associated with inflammatory reactions, iii) autoimmune diseases, and iv) conditions in men or women associated with abnormal estrogen/androgen balance.

WO 02/066034 and WO 02/066035 disclose the use of a group of azole containing compounds for the treatment of certain metabolic diseases. In particular, the invention relates to the prophylaxis, management or treatment of cardiovascular diseases, diabetes, cancers, and obesity through the inhibition of malonyl CoA decarboxylase.

WO 02/055072 discloses that the combined administration of isoflavones and lipid regulating drugs aids in the regulation of circulating lipid levels, the regulation of bone density, and has application in the treatment and/or prevention of osteoporosis.

U.S. Pat. Nos. 6,462,072; 6,384,056; and 6,429,215 disclose small molecule cyclic ester and amide derivatives of the formula: 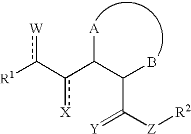
with an affinity for FKBP-type immunophilins. The compounds are described as being effective in stimulating neurite growth, alopecia, promoting hair growth, treating vision disorders, improving vision, treating memory impairment, and enhancing memory performance.

U.S. Pat. No. 6,297,270 discloses the preparation of particular 2,3-dihydroindoles from halostyrene derivatives.

U.S. Pat. No. 5,650,633 discloses compositions which can be used to prepare water-soluble polymers that are useful in oil field applications and processes for producing the compositions.

Given that VCAM-1 is involved in chronic inflammatory disorders, it is a goal of the present work to identify new compounds, compositions and methods that inhibit the expression of VCAM-1. An even more general goal is to identify selective compounds, pharmaceutical compositions and methods of using the compounds for the treatment of inflammatory diseases.

It is therefore an object of the present invention to provide new compounds, compositions and methods for the treatment of disorders involving VCAM-1, including cardiovascular and inflammatory diseases.

### SUMMARY OF THE INVENTION

It has been discovered that particular 1,2-bis-(substituted-phenyl)-2-propen-1-ones inhibit the expression of VCAM-1, and thus can be used to treat a patient with a disorder involving VCAM-1. Compounds of the present invention are of the formula 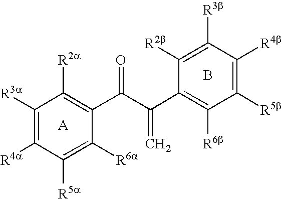
or its pharmaceutically acceptable salt or ester, wherein the substituents are defined herein.

Examples of inflammatory disorders that are involve VCAM-1 include, but are not limited to arthritis, asthma, dermatitis, cystic fibrosis, post transplantation late and chronic solid organ rejection, multiple sclerosis, systemic lupus erythematosis, inflammatory bowel diseases, autoimmune diabetes, diabetic retinopathy, diabetic nephropathy, diabetic vasculopathy, rhinitis, ischemia-reperfusion injury, post-angioplasty restenosis, chronic obstructive pulmonary disease (COPD), glomerulonephritis, Graves disease, gastrointestinal allergies, conjunctivitis, atherosclerosis, coronary artery disease, angina and small artery disease.

The compounds disclosed herein can also be used in the treatment of inflammatory skin diseases that involve VCAM-1, as well as human endothelial disorders that involve VCAM-1, which include, but are not limited to psoriasis, dermatitis, including eczematous dermatitis, Kaposi's sarcoma, multiple sclerosis, as well as proliferative disorders of smooth muscle cells.

In yet another embodiment, the compounds disclosed herein can be selected to treat anti-inflammatory conditions that are mediated by mononuclear leucocytes.

In one embodiment, the compounds of the present invention are selected for the prevention or treatment of tissue or organ transplant rejection. Treatment and prevention of organ or tissue transplant rejection includes, but is not limited to treatment of recipients of heart, lung, combined heart-lung, liver, kidney, pancreatic, skin, spleen, small bowel, or corneal transplants. The compounds can also be used in the prevention or treatment of graft-versus-host disease, such as sometimes occurs following bone marrow transplantation.

In an alternative embodiment, the compounds described herein are useful in both the primary and adjunctive medical treatment of cardiovascular disease. The compounds are used in primary treatment of, for example, coronary disease states including atherosclerosis, post-angioplasty restenosis, coronary artery diseases and angina. The compounds can be administered to treat small vessel disease that is not treatable by surgery or angioplasty, or other vessel disease in which surgery is not an option. The compounds can also be used to stabilize patients prior to revascularization therapy.

### Definitions

The following definitions are provided in order to aid those skilled in the art in understanding the detailed description of the present invention.

When not used as a bond, the wavy line indicates the point of attachment of the particular substituent.

The terms “alkyl” or “alk”, alone or in combination, unless otherwise specified, means a saturated straight or branched primary, secondary, or tertiary hydrocarbon having for example from 1 to 16 carbon atoms, including, but not limited to methyl, ethyl, propyl, isopropyl, butyl, isobutyl, t-butyl, and sec-butyl. The term “lower alkyl” alone or in combination refers to an alkyl having from 1 to 6 carbon atoms. The alkyl group may be optionally substituted where possible with any moiety that does not otherwise interfere with the reaction or that provides an improvement in the process, including but not limited to halo, haloalkyl, hydroxyl, carboxyl, acyl, aryl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrozine, carbamate, phosphonic acid, phosphonate, either unprotected, or protected as necessary, as known to those skilled in the art, for example, as taught in Greene et al., Protective Groups in Organic Synthesis, John Wiley & Sons, Third Edition, 1998, hereby incorporated by reference.

The term “alkenyl”, alone or in combination, means a non-cyclic alkyl of e.g., 2 to 10 carbon atoms having one or more unsaturated carbon-carbon bonds. The alkenyl group may be optionally substituted where possible with any moiety that does not otherwise interfere with the reaction or that provides an improvement in the process, including but not limited to halo, haloalkyl, hydroxyl, carboxyl, acyl, aryl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrozine, carbamate, phosphonic acid, phosphonate, either unprotected, or protected as necessary, as known to those skilled in the art.

The term “alkynyl”, alone or in combination, means a non-cyclic alkyl of e.g. 2 to 10 carbon atoms having one or more triple carbon-carbon bonds, including but not limited to ethynyl and propynyl. The alkynyl group may be optionally substituted where possible with any moiety that does not otherwise interfere with the reaction or that provides an improvement in the process, including but not limited to halo, haloalkyl, hydroxyl, carboxyl, acyl, aryl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrozine, carbamate, phosphonic acid, phosphonate, either unprotected, or protected as necessary, as known to those skilled in the art.

The terms “carboxy”, “COOH” and “C(O)OH” are used interchangeably.

The terms “alkoxycarbonyl” and “carboalkoxy” are used interchangeably. Used alone or in combination, the terms mean refer to the radical —C(O)OR, wherein R is alkyl as defined herein.

The term “thio”, alone or in combination, means the radical —S—.

The term “thiol”, alone or in combination, means the radical —SH.

The term “hydroxy”, alone or in combination means the radical —OH.

The term “sulfonyl”, alone or in combination means the radical —S(O)₂—.

The term “oxo” refers to an oxygen attached by a double bond (═O).

The term “carbocycle”, alone or in combination, means any stable 3- to 7-membered monocyclic or bicyclic or 7- to 14-membered bicyclic or tricyclic or an up to 26-membered polycyclic carbon ring, any of which may be saturated, partially unsaturated, or aromatic. Examples of such carbocyles include, but are not limited to, cyclopropyl, cyclopentyl, cyclohexyl, phenyl, biphenyl, naphthyl, indanyl, adamantyl, or tetrahydronaphthyl (tetralin).

The term “cycloalkyl”, alone or in combination, means a saturated or partially unsaturated cyclic alkyl, having from 1 to 10 carbon atoms, including but not limited to mono- or bi-cyclic ring systems such as cyclopropyl, cyclobutyl, cyclopentyl, cyclohexenyl, and cyclohexyl.

The term “aryl”, alone or in combination, means a carbocyclic aromatic system containing one, two or three rings wherein such rings may be attached together in a pendent manner or may be fused. The “aryl” group can be optionally substituted where possible with one or more of the moieties selected from the group consisting of alkyl, alkenyl, alkynyl, heteroaryl, heterocyclic, carbocycle, alkoxy, oxo, aryloxy, arylalkoxy, cycloalkyl, tetrazolyl, heteroaryloxy; heteroarylalkoxy, carbohydrate, amino acid, amino acid esters, amino acid amides, alditol, halogen, haloalkylthi, haloalkoxy, haloalkyl, hydroxyl, carboxyl, acyl, acyloxy, amino, aminoalkyl, aminoacyl, amido, alkylamino, dialkylamino, arylamino, nitro, cyano, thiol, imide, sulfonic acid, sulfate, sulfonate, sulfonyl, alkylsulfonyl, aminosulfonyl, alkylsulfonylamino, haloalkylsulfonyl, sulfanyl, sulfinyl, sulfamoyl, carboxylic ester, carboxylic acid, amide, phosphonyl, phosphinyl, phosphoryl, thioester, thioether, oxime, hydrazine, carbamate, phosphonic acid, phosphate, phosphonate, phosphinate, sulfonamido, carboxamido, hydroxamic acid, sulfonylimide or any other desired functional group that does not inhibit the pharmacological activity of this compound, either unprotected, or protected as necessary, as known to those skilled in the art. In addition, adjacent groups on an “aryl” ring may combine to form a 5- to 7-membered saturated or partially unsaturated carbocyclic, aryl, heteroaryl or heterocyclic ring, which in turn may be substituted as above.

The term “heterocyclic”, alone or in combination, refers to a nonaromatic cyclic group that may be partially (containing at least one double bond) or fully saturated and wherein the ring contains at least one heteroatom selected from oxygen, sulfur, nitrogen, or phosphorus. The terms “heteroaryl” or “heteroaromatic”, alone or in combination, refer to an aromatic ring containing at least one heteroatom selected from sulfur, oxygen, nitrogen or phosphorus. The heteroaryl or heterocyclic ring may optionally be substituted where possible by one or more substituent listed as optional substituents for aryl. In addition, adjacent groups on the heteroaryl or heterocyclic ring may combine to form a 5- to 7-membered carbocyclic, aryl, heteroaryl or heterocyclic ring, which in turn may be substituted as above. Nonlimiting examples of heterocylics and heteroaromatics are pyrrolidinyl, tetrahydrofuryl, tetrahydrofuranyl, pyranyl, purinyl, tetrahydropyranyl, piperazinyl, piperidinyl, morpholino, thiomorpholino, tetrahydropyranyl, imidazolyl, pyrolinyl, pyrazolinyl, indolinyl, dioxolanyl, or 1,4-dioxanyl. aziridinyl, furyl, furanyl, pyridyl, pyridinyl, pyridazinyl, pyrimidinyl, benzoxazolyl, 1,2,4-oxadiazolyl, 1,3,4-oxadiazolyl, 1,3,4-thiadiazole, indazolyl, triazinayl, 1,3,5-triazinyl, thienyl, isothiazolyl, imidazolyl, tetrazolyl, pyrazinyl, benzofuranyl, quinolyl, isoquinolyl, benzothienyl, isobenzofuryl, pyrazolyl, indolyl, isoindolyl, benzimidazolyl, purinyl, carbazolyl, oxazolyl, thiazolyl, benzothiazolyl, isothiazolyl, 1,2,4-thiadiazolyl, isooxazolyl, 1,2,4-oxadiazolyl, 1,3,4-oxadiazolyl, pyrrolyl, quinazolinyl, quinoxalinyl, benzoxazolyl, quinolinyl, isoquinolinyl, cinnolinyl, phthalazinyl, xanthinyl, hypoxanthinyl, pyrazole, imidazole, 1,2,3-triazole, 1,2,4-triazole, 1,2,3-oxadiazole, thiazine, pyridazine, triazolopyridinyl or pteridinyl wherein said heteroaryl or heterocyclic group can be optionally substituted with one or more substituent selected from the same substituents as set out above for aryl groups. Functional oxygen and nitrogen groups on the heteroaryl group can be protected as necessary or desired. Suitable protecting groups can include trimethylsilyl, dimethylhexylsilyl, t-butyldimethylsilyl, and t-butyldiphenylsilyl, trityl or substituted trityl, alkyl groups, acyl groups (such as acetyl and propionyl), methanesulfonyl, and p-toluenesulfonyl.

The term “thienyl”, alone or in combination, refers to a five member cyclic group wherein the ring contains one sulfur atom and two double bonds.

The term “benzothienyl”, alone or in combination, refers to a five member cyclic group wherein the ring contains one sulfur atom and two double bonds fused to a phenyl ring.

The term “aryloxy”, alone or in combination, refers to an aryl group bound to the molecule through an oxygen atom.

The term “heteroaryloxy”, alone or in combination, refers to a heteroaryl group bound to the molecule through an oxygen atom.

The term “aralkoxy”, alone or in combination, refers to an aryl group attached to an alkyl group which is attached to the molecule through an oxygen atom.

The term “heterocyclearalkoxy” refers to a heterocyclic group attached to an aryl group attached to an alkyl-O— group. The heterocyclic, aryl and alkyl groups can be optionally substituted as described above.

The terms “halo” and “halogen”, alone or in combination, refer to chloro, bromo, iodo and fluoro.

The terms “alkoxy” or “alkylthio”, alone or in combination, refers to an alkyl group as defined above bonded through an oxygen linkage (—O—) or a sulfur linkage (—S—), respectively. The terms “lower alkoxy” or “lower alkylthio”, alone or in combination, refers to a lower alkyl group as defined above bonded through an oxygen linkage (—O—) or a sulfur linkage (—S—), respectively.

The term “acyl”, alone or in combination, refers to a group of the formula C(O)R′, wherein R′ is an alkyl, aryl, alkaryl or aralkyl group, or substituted alkyl, aryl, aralkyl or alkaryl, wherein these groups are as defined above.

The term “acetyl”, alone or in combination, refers to the radical —C(O)CH₃.

The term “amino”, alone or in combination, refers to a group of the formula NR′R″, wherein R′ and R″ are independently selected from a group consisting of a bond, hydrogen, alkyl, aryl, alkaryl, and aralkyl, wherein said alkyl, aryl, alkaryl and aralkyl may be optionally substituted where possible as defined above.

The term “nitro”, alone or in combination, denotes the radical —NO₂.

The term “substituted”, means that one or more hydrogen on the designated atom or substituent is replaced with a selection from the indicated group, provided that the designated atom's normal valency is not exceeded, and the that the substitution results in a stable compound. When a subsitutent is “oxo” (keto) (i.e., ═O), then 2 hydrogens on the atom are replaced.

The term “alditol”, as referred to herein, and unless otherwise specified, refers to a carbohydrate in which the aldehyde or ketone group has been reduced to an alcohol moiety. The alditols of the present invention can also be optionally substituted where possible or deoxygenated at one or more positions. Exemplary substituents include hydrogen, halo, haloalkyl, carboxyl, acyl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamonyl, ester, carboxylic acid, amide, amino acid, amino acid esters and amides, phosphonyl, phosphinyl, phosphoryl, thioester, thioether, oxime, hydrazine, carbamate, phosphonic acid, and phosphonate. Particular exemplary substituents include amine and halo, particularly fluorine. The substituent or alditol can be either unprotected, or protected as necessary, as known to those skilled in the art. The alditol may have 3, 4, 5, 6 or 7 carbons. Examples of useful alditols are those derived from reduction of monosaccharides, including specifically those derived from the reduction of pyranose and furanose sugars.

The term “carbohydrate”, as referred to herein, and unless otherwise specified, refers to a compound of carbon, hydrogen and oxygen that contains an aldehyde or ketone group in combination with at least two hydroxyl groups. The carbohydrates of the present invention can also be optionally substituted where possible or deoxygenated at one or more positions. Carbohydrates thus include substituted and unsubstituted monosaccharides, disaccharides, oligosaccharides, and polysaccharides. The saccharide can be an aldose or ketose, and may comprise 3, 4, 5, 6, or 7 carbons. In one embodiment the carbohydrates are monosaccharides. In another embodiment the carbohydrates are pyranose and furanose sugars.

As used herein, the term “patient” refers to warm-blooded animals or mammals, and in particular humans, who are in need of the therapy described herein. The term “host”, as used herein, refers to a unicellular or multicellular organism, including cell lines and animals, and preferably a human.

### DETAILED DESCRIPTION OF THE INVENTION

While compositions and methods are described in terms of “comprising” various components or steps, the compositions and methods can also “consist essentially of” or “consist of” the various components and steps.

It has been discovered that compounds of the invention inhibit the expression of VCAM-1, and thus can be used to treat a patient with a disorder involving VCAM-1. These compounds can be administered to a host as monotherapy, or if desired, in combination with another compound of the invention or another biologically active agent, as described in more detail below.

In one embodiment, the invention is represented by Formula I 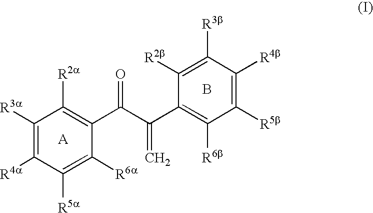
or its pharmaceutically acceptable salt or ester, wherein:

R^{2α}, R^{3α}, R^{4α}, R^{5α}, R^{6α}, R^{2β}, R^{3β}, R^{4β}, R^{5β} and R^{6β} are independently selected from the group consisting of hydrogen, halogen, nitro, alkyl, lower alkyl, alkenyl, alkynyl, carbocycle, cycloalkyl, cycloalkylalkyl, haloalkyl, aryl, arylalkyl, heteroaryl, heteroaryl lower alkyl, heterocyclic, heterocyclic lower alkyl, alkylthioalkyl, cycloalkylthioalkyl, arylthio lower alkyl, aralkyl lower thioalkyl, heteroarylthio lower alkyl, heteroaralkyl lower thioalkyl, heterocyclicthio lower alkyl, heterocyclicalkyl lower thioalkyl, lower alkyl S(O)-lower alkyl, lower alkyl-S(O)₂-lower alkyl, arylsulfinyl lower alkyl, arylsulfonyl lower alkyl, —C(O)R², R²C(O)alkyl, aminoalkyl, cycloalkylaminoalkyl, arylamino lower alkyl, heteroarylamino lower alkyl, heterocyclicamino lower alkyl, hydroxyl, hydroxyalkyl, alditol, carbohydrate, polyol alkyl, alkoxy, lower alkoxy, —(O(CH₂)₂)₁₋₃—O-lower alkyl, polyoxyalkylene, cycloalkyloxy, cycloalkylalkoxy, haloalkoxy, aryloxy, arylalkoxy, heteroaryloxy, heteroarylalkoxy, heteroaryl lower alkoxy, heterocyclicoxy, heterocyclicalkoxy, heterocyclic lower alkoxy, —OC(R¹)₂C(O)OH, —OC(R¹)₂C(O)OR², —OC(R¹)₂C(O)NH₂, —OC(R¹)₂C(O)NHR², —OC(R¹)₂C(O)N(R²)₂, —OC(R¹)₂C(O)NR⁷R⁸, amino, alkylamino, acylamino, dialkylamino, cycloalkylamino, arylamino, aralkylamino, heteroarylamino, heteroaralkylamino, heterocyclicamino, heterocyclicalkylamino, —NHR², N(R²)₂, —NR⁷R⁸, —NHC(R¹)₂C(O)OH, —NHC(R¹)₂C(O)OR², —NHC(O)R², —N(R²)C(O)R², —NHC(O)OR², —NHC(O)SR², —NHSO₂NHR², —NHSO₂R², —NHSO₂NR⁷R⁸, —N(C(O)NHR²)₂, —NR²SO₂R², —NHC(O)NHR², —NHC(O)NR⁷R⁸, —NHC(O)N(R²)₂, thiol, alkylthio, cycloalkylthio, cycloalkylalkylthio, haloalkylthio, arylthio, aralkylthio, heteroarylthio, heteroaralkylthio, heterocyclicthio, heterocyclicalkylthio, alkylsulfonyl, arylsulfonyl, haloalkylsulfonyl, —SC(R¹)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR⁷R⁸, —SO₂NHC(O)R², —SR², —SO₂NHC(O)NHR², —SO₂NHC(O)N(R²)₂, —SO₂NHC(O)NR⁷R⁸, sulfonic acid, sulfonate, sulfate, sulfinic acid, sulfenic acid, cyano, tetrazol-5-yl, carboxy, —C(O)OR², —C(O)NH₂, —C(O)NHR², —C(O)N(R²)₂, —C(O)NR⁷R⁸, —C(O)NHC(O)R², —C(O)NHC(O)NHR², —C(O)NHC(O)N(R²)₂, —C(O)NHC(O)NR⁷R⁸, —C(O)NHSO₂R², —C(O)NHSO₂NHR², —C(O)NHSO₂N(R²), —C(O)NHSO₂NR⁷R⁸, —C(CH₃)₂C(O)OH, —(CH₂)_{y}C(O)OH, wherein y is 1, 2, 3, 4, 5, or 6, —PO₂H₂, —PO₃H₂, —P(R²)O₂H, and phosphate, all of which can be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R¹ is independently selected from the group consisting of hydrogen, lower alkyl, carbocycle, cycloalkyl, aryl, heteroaryl, heterocyclic, arylalkyl, heteroarylalkyl, and heterocyclicalkyl, wherein all may be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R² is independently selected from the group consisting of alkyl, lower alkyl, alkenyl, alkynyl, carbocycle, cycloalkyl, aryl, heteroaryl, heterocyclic, arylalkyl, heteroarylalkyl, and heterocyclicalkyl, wherein all may be substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R⁷ and R⁸ are independently selected from the group consisting of alkyl, alkenyl, heteroaryl and aryl and linked together forming a 4- to 12-membered monocyclic, bicylic, tricyclic or benzofused ring;
wherein one of R^{2β}, R^{3β}, R^{4β}, R^{5β}, or R^{6β}, or one of R^{2α}, R^{3α}, R^{4α}, R^{5α} or R^{6α} must be a carbon-carbon linked heterocyclic or heteroaryl; or
R^{2α} and R^{3α} taken together or R^{3α} and R^{4α} taken together or R^{4α} and R^{5α} taken together, or R^{2β} and R^{3β} taken together or R^{3β} and R^{4β} taken together or R^{4β} and R^{5β} taken together form a heterocyclic or heteroaryl optionally substituted by one or more alkoxycarbonylalkyl, carboxyalkyl, hydroxyalkyl or aminoalkyl and optionally substituted where possible with one or more selected from the group consisting of hydroxy, alkyl, carboxy, hydroxyalkyl, carboxyalkyl, amino, cyano, alkoxy, alkoxycarbonyl, acyl, oxo, —NR⁷R⁸, and halo; or
R^{2α} and R^{3α} taken together or R^{3α} and R^{4α} taken together or R^{4α} and R^{5α} taken together or R^{2β} and R^{3β} taken together or R^{3β} and R^{4β} taken together or R^{4β} and R^{5β} taken together form a 5- or 6-membered ring containing one nitrogen, which may optionally be substituted where possible with one or more selected from the group consisting of halo, alkyl, lower alkyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
wherein all R¹, R², R⁷ and R⁸ substituents can be optionally substituted where possible with one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂.

In a further embodiment, the invention is represented by Formula I or its pharmaceutically acceptable salt or ester, wherein:

R^{2α}, R^{3α}, R^{4α}, R^{5α}, R^{6α}, R^{2β}, R^{3β}, R^{4β}, R^{5β} and R^{6β} are independently selected from the group consisting of hydrogen, halogen, nitro, alkyl, lower alkyl, alkenyl, alkynyl, carbocycle, cycloalkyl, cycloalkylalkyl, haloalkyl, aryl, arylalkyl, heteroaryl, heteroaryl lower alkyl, heterocyclic, heterocyclic lower alkyl, alkylthioalkyl, cycloalkylthioalkyl, arylthio lower alkyl, aralkyl lower thioalkyl, heteroarylthio lower alkyl, heteroaralkyl lower thioalkyl, heterocyclicthio lower alkyl, heterocyclicalkyl lower thioalkyl, lower alkyl S(O)-lower alkyl, lower alkyl-S(O)₂-lower alkyl, arylsulfinyl lower alkyl, arylsulfonyl lower alkyl, —C(O)R², R²C(O)alkyl, aminoalkyl, cycloalkylaminoalkyl, arylamino lower alkyl, heteroarylamino lower alkyl, heterocyclicamino lower alkyl, hydroxyl, hydroxyalkyl, alkoxy, lower alkoxy, —(O(CH₂)₂)₁₋₃—O-lower alkyl, cycloalkyloxy, cycloalkylalkoxy, haloalkoxy, aryloxy, arylalkoxy, heteroaryloxy, heteroarylalkoxy, heteroaryl lower alkoxy, heterocyclicoxy, heterocyclicalkoxy, heterocyclic lower alkoxy, —OC(R¹)₂C(O)OH, —OC(R¹)₂C(O)OR², —OC(R¹)₂C(O)NH₂, —OC(R¹)₂C(O)NHR², —OC(R¹)₂C(O)N(R²)₂, —OC(R¹)₂C(O)NR⁷R⁸, amino, alkylamino, acylamino, dialkylamino, cycloalkylamino, arylamino, aralkylamino, heteroarylamino, heteroaralkylamino, heterocyclicamino, heterocyclicalkylamino, —NHR², N(R²)₂, —NR⁷R⁸, —NHC(R¹)₂C(O)OH, —NHC(R¹)₂C(O)OR, —NHC(O)R², —N(R²)C(O)R², —NHC(O)OR², —NHC(O)SR², —NHSO₂NHR², —NHSO₂R², —NHSO₂NR⁷R⁸, —N(C(O)NHR²)₂, —NR²SO₂R², —NHC(O)NHR², —NHC(O)NR⁷R⁸, —NHC(O)N(R²)₂, thiol, alkylthio, cycloalkylthio, cycloalkylalkylthio, haloalkylthio, arylthio, aralkylthio, heteroarylthio, heteroaralkylthio, heterocyclicthio, heterocyclicalkylthio, alkylsulfonyl, arylsulfonyl, haloalkylsulfonyl, —SC(R¹)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR⁷R⁸, —SO₂NHC(O)R², —SR², —SO₂NHC(O)NHR², —SO₂NHC(O)N(R²)₂, —SO₂NHC(O)NR⁷R⁸, sulfonic acid, sulfonate, sulfate, sulfinic acid, sulfenic acid, cyano, tetrazol-5-yl, carboxy, —C(O)OR², —C(O)NH₂, —C(O)NHR², —C(O)N(R²)₂, —C(O)NR⁷R⁸, —C(O)NHC(O)R², —C(O)NHC(O)NHR², —C(O)NHC(O)N(R²)₂, —C(O)NHC(O)NR⁷R⁸, —C(O)NHSO₂R², —C(O)NHSO₂NHR², —C(O)NHSO₂N(R²), —C(O)NHSO₂NR⁷R⁸, —C(CH₃)₂C(O)OH, —(CH₂)_{y}C(O)OH, wherein y is 1, 2, 3, 4, 5, or 6, —PO₂H₂, —PO₃H₂, —P(R²)O₂H, and phosphate, all of which can be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R¹ is independently selected from the group consisting of hydrogen, lower alkyl, carbocycle, cycloalkyl, aryl, heteroaryl, heterocyclic, arylalkyl, heteroarylalkyl, and heterocyclicalkyl, wherein all may be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R² is independently selected from the group consisting of alkyl, lower alkyl, alkenyl, alkynyl, carbocycle, cycloalkyl, aryl, heteroaryl, heterocyclic, arylalkyl, heteroarylalkyl, and heterocyclicalkyl, wherein all may be substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R⁷ and R⁸ are independently selected from the group consisting of alkyl, alkenyl, heteroaryl and aryl and linked together forming a 4- to 12-membered monocyclic, bicylic, tricyclic or benzofused ring;
wherein one of R^{2β}, R^{3β}, R^{4β}, R^{5β} or R^{6β}, or one of R^{2α}, R^{3α}, R^{4α}, R^{5α} or R^{6α} must be a carbon-carbon linked heterocyclic or heteroaryl;
wherein all R¹, R², R⁷ and R⁸ substituents can be optionally substituted where possible with one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂.

In a further embodiment, the invention is represented by Formula I or its pharmaceutically acceptable salt or ester, wherein:

R^{2α}, R^{3α}, R^{4α}, R^{5α}, R^{6α}, R^{2β}, R^{3β}, R^{4β}, R^{5β} and R^{6β} are independently selected from the group consisting of hydrogen, halogen, nitro, alkyl, lower alkyl, alkenyl, alkynyl, carbocycle, cycloalkyl, cycloalkylalkyl, haloalkyl, aryl, arylalkyl, heteroaryl, heteroaryl lower alkyl, heterocyclic, heterocyclic lower alkyl, alkylthioalkyl, cycloalkylthioalkyl, arylthio lower alkyl, aralkyl lower thioalkyl, heteroarylthio lower alkyl, heteroaralkyl lower thioalkyl, heterocyclicthio lower alkyl, heterocyclicalkyl lower thioalkyl, —C(O)R², R²C(O)alkyl, aminoalkyl, cycloalkylaminoalkyl, arylamino lower alkyl, heteroarylamino lower alkyl, heterocyclicamino lower alkyl, hydroxyl, hydroxyalkyl, alkoxy, lower alkoxy, —(O(CH₂)₂)₁₋₃—O-lower alkyl, cycloalkyloxy, cycloalkylalkoxy, haloalkoxy, aryloxy, arylalkoxy, heteroaryloxy, heteroarylalkoxy, heteroaryl lower alkoxy, heterocyclicoxy, heterocyclicalkoxy, heterocyclic lower alkoxy, —OC(R¹)₂C(O)OH, —OC(R¹)₂C(O)OR², —OC(R¹)₂C(O)NH₂, —OC(R¹)₂C(O)NHR², —OC(R¹)₂C(O)N(R²)₂, —OC(R¹)₂C(O)NR⁷R⁸, amino, alkylamino, acylamino, dialkylamino, cycloalkylamino, arylamino, aralkylamino, heteroarylamino, heteroaralkylamino, heterocyclicamino, heterocyclicalkylamino, —NHR², N(R²)₂, —NR⁷R⁸, —NHC(R¹)₂C(O)OH, —NHC(R¹)₂C(O)OR², —NHC(O)R², —N(R²)C(O)R², —NHC(O)OR², —NHC(O)SR², —NHSO₂NHR², —NHSO₂R², —NHSO₂NR⁷R⁸, —N(C(O)NHR²)₂, —NR²SO₂R², —NHC(O)NHR², —NHC(O)NR⁷R⁸, —NHC(O)N(R²)₂, thiol, alkylthio, cycloalkylthio, cycloalkylalkylthio, haloalkylthio, arylthio, aralkylthio, heteroarylthio, heteroaralkylthio, heterocyclicthio, heterocyclicalkylthio, alkylsulfonyl, arylsulfonyl, haloalkylsulfonyl, —SC(R¹)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR⁷R⁸, —SO₂NHC(O)R², —SR², —SO₂NHC(O)NHR², —SO₂NHC(O)N(R²)₂, —SO₂NHC(O)NR⁷R⁸, sulfonic acid, sulfonate, sulfate, sulfinic acid, sulfenic acid, cyano, tetrazol-5-yl, carboxy, —C(O)OR², —C(O)NH₂, —C(O)NHR², —C(O)N(R²)₂, —C(O)NR⁷R⁸, —C(O)NHC(O)R², —C(O)NHC(O)NHR², —C(O)NHC(O)N(R²)₂, —C(O)NHC(O)NR⁷R⁸, —C(O)NHSO₂R², —C(O)NHSO₂NHR², —C(O)NHSO₂N(R²), —C(O)NHSO₂NR⁷R⁸, —C(CH₃)₂C(O)OH, —(CH₂)_{y}C(O)OH, wherein y is 1, 2, 3, 4, 5, or 6, —PO₂H₂, —PO₃H₂, —P(R²)O₂H, and phosphate, all of which can be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R¹ is independently selected from the group consisting of hydrogen, lower alkyl, carbocycle, cycloalkyl, aryl, heteroaryl, heterocyclic, arylalkyl, heteroarylalkyl, and heterocyclicalkyl, wherein all may be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R² is independently selected from the group consisting of alkyl, lower alkyl, alkenyl, alkynyl, carbocycle, cycloalkyl, aryl, heteroaryl, heterocyclic, arylalkyl, heteroarylalkyl, and heterocyclicalkyl, wherein all may be substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R⁷ and R⁸ are independently selected from the group consisting of alkyl, alkenyl, heteroaryl and aryl and linked together forming a 4- to 12-membered monocyclic, bicylic, tricyclic or benzofused ring;
wherein one of R^{2β}, R^{3β}, R^{4β}, R^{5β} or R^{6β}, or one of R^{2α}, R^{3α}, R^{4α}, R^{5α} or R^{6α} must be a carbon-carbon linked heteroaryl;
wherein all R¹, R², R⁷ and R⁸ substituents can be optionally substituted where possible with one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂.

In yet another embodiment, the invention is represented by Formula I or its pharmaceutically acceptable salt or ester, wherein:

R^{3α}, R^{4α}, R^{5α}, R^{2β}, R^{3β}, R^{4β}, R^{5β} and R^{6β} are independently selected from the group consisting of hydrogen, halogen, nitro, alkyl, lower alkyl, alkenyl, alkynyl, carbocycle, cycloalkyl, cycloalkylalkyl, haloalkyl, aryl, arylalkyl, heteroaryl, heteroaryl lower alkyl, heterocyclic, heterocyclic lower alkyl, alkylthioalkyl, cycloalkylthioalkyl, arylthio lower alkyl, aralkyl lower thioalkyl, heteroarylthio lower alkyl, heteroaralkyl lower thioalkyl, heterocyclicthio lower alkyl, heterocyclicalkyl lower thioalkyl, —C(O)R², R²C(O)alkyl, aminoalkyl, cycloalkylaminoalkyl, arylamino lower alkyl, heteroarylamino lower alkyl, heterocyclicamino lower alkyl, hydroxyl, hydroxyalkyl, alkoxy, lower alkoxy, —(O(CH₂)₂)₁₋₃—O-lower alkyl, cycloalkyloxy, cycloalkylalkoxy, haloalkoxy, aryloxy, arylalkoxy, heteroaryloxy, heteroarylalkoxy, heteroaryl lower alkoxy, heterocyclicoxy, heterocyclicalkoxy, heterocyclic lower alkoxy, —OC(R¹)₂C(O)OH, —OC(R¹)₂C(O)OR², —OC(R¹)₂C(O)NH₂, —OC(R¹)₂C(O)NHR², —OC(R¹)₂C(O)N(R²)₂, —OC(R¹)₂C(O)NR⁷R⁸, amino, alkylamino, acylamino, dialkylamino, cycloalkylamino, arylamino, aralkylamino, heteroarylamino, heteroaralkylamino, heterocyclicamino, heterocyclicalkylamino, —NHR², N(R²)₂, —N⁷R⁸, —NHC(R¹)₂C(O)OH, —NHC(R¹)₂C(O)OR², —NHC(O)R², —N(R²)C(O)R₂, —NHC(O)OR², —NHC(O)SR², —NHSO₂NHR², —NHSO₂R, —NHSO₂NR⁷R⁸, —N(C(O)NHR²)₂, —NR²SO₂R², —NHC(O)NHR², —NHC(O)NR⁷R⁸, —NHC(O)N(R²)₂, thiol, alkylthio, cycloalkylthio, cycloalkylalkylthio, haloalkylthio, arylthio, aralkylthio, heteroarylthio, heteroaralkylthio, heterocyclicthio, heterocyclicalkylthio, alkylsulfonyl, arylsulfonyl, haloalkylsulfonyl, —SC(R¹)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR⁷R⁸, —SO₂NHC(O)R², —SR², —SO₂NHC(O)NHR², —SO₂NHC(O)N(R²)₂, —SO₂NHC(O)NR⁷R⁸, sulfonic acid, sulfonate, sulfate, sulfinic acid, sulfenic acid, cyano, tetrazol-5-yl, carboxy, —C(O)OR², —C(O)NH₂, —C(O)NHR², —C(O)N(R²)₂, —C(O)NR⁷R⁸, —C(O)NHC(O)R², —C(O)NHC(O)NHR², —C(O)NHC(O)N(R²)₂, —C(O)NHC(O)NR⁷R⁸, —C(O)NHSO₂R², —C(O)NHSO₂NHR², —C(O)NHSO₂N(R²), —C(O)NHSO₂N⁷R⁸, —C(CH₃)₂C(O)OH, —(CH₂)_{y}C(O)OH, wherein y is 1, 2, 3, 4, 5, or 6, —PO₂H₂, —PO₃H₂, —P(R²)O₂H, and phosphate, all of which can be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R^{2α} and R^{6α} are independently selected from the group consisting of halogen, nitro, alkyl, lower alkyl, alkenyl, alkynyl, carbocycle, cycloalkyl, cycloalkylalkyl, haloalkyl, aryl, arylalkyl, heteroaryl, heteroaryl lower alkyl, heterocyclic, heterocyclic lower alkyl, alkylthioalkyl, cycloalkylthioalkyl, arylthio lower alkyl, aralkyl lower thioalkyl, heteroarylthio lower alkyl, heteroaralkyl lower thioalkyl, heterocyclicthio lower alkyl, heterocyclicalkyl lower thioalkyl, R²C(O)alkyl, aminoalkyl, cycloalkylaminoalkyl, arylamino lower alkyl, heteroarylamino lower alkyl, heterocyclicamino lower alkyl, hydroxyalkyl, alkoxy, lower alkoxy, —(O(CH₂)₂)₁₋₃—O-lower alkyl, cycloalkyloxy, cycloalkylalkoxy, haloalkoxy, aryloxy, arylalkoxy, heteroaryloxy, heteroarylalkoxy, heteroaryl lower alkoxy, heterocyclicoxy, heterocyclicalkoxy, heterocyclic lower alkoxy, —OC(R¹)₂C(O)OH, —OC(R¹)₂C(O)OR², —OC(R¹)₂C(O)NH₂, —OC(R¹)₂C(O)NHR², —OC(R¹)₂C(O)N(R¹)₂, —OC(R¹)₂C(O)NR⁷R⁸, —NHSO₂NHR², —NHSO₂R², —NHSO₂NR⁷R⁸, —NR²SO₂R², alkylthio, cycloalkylthio, cycloalkylalkylthio, haloalkylthio, arylthio, aralkylthio, heteroarylthio, heteroaralkylthio, heterocyclicthio, heterocyclicalkylthio, alkylsulfonyl, arylsulfonyl, haloalkylsulfonyl, —SC(R¹)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR⁷R⁸, —SO₂NHC(O)R², —SR², —SO₂NHC(O)NHR², —SO₂NHC(O)N(R²)₂, —SO₂NHC(O)NR⁷R⁸, sulfonic acid, sulfonate, sulfate, sulfinic acid, sulfenic acid, cyano, tetrazol-5-yl, carboxy, —C(O)OR², —C(O)NH₂, —C(O)NHR², —C(O)N(R²)₂, —C(O)NR⁷R⁸, —C(O)NHC(O)R², —C(O)NHC(O)NHR², —C(O)NHC(O)N(R²)₂, —C(O)NHC(O)NR⁷R⁸, —C(O)NHSO₂R², —C(O)NHSO₂NHR², —C(O)NHSO₂N(R²), —C(O)NHSO₂NR⁷R⁸, —C(CH₃)₂C(O)OH, —(CH₂)_{y}C(O)OH, wherein y is 1, 2, 3, 4, 5, or 6, —PO₂H₂, —PO₃H₂, —P(R²)O₂H, and phosphate, all of which can be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R¹ is independently selected from the group consisting of hydrogen, lower alkyl, carbocycle, cycloalkyl, aryl, heteroaryl, heterocyclic, arylalkyl, heteroarylalkyl, and heterocyclicalkyl, wherein all may be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R² is independently selected from the group consisting of alkyl, lower alkyl, alkenyl, alkynyl, carbocycle, cycloalkyl, aryl, heteroaryl, heterocyclic, arylalkyl, heteroarylalkyl, and heterocyclicalkyl, wherein all may be substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R⁷ and R⁸ are independently selected from the group consisting of alkyl, alkenyl, heteroaryl and aryl and linked together forming a 4- to 12-membered monocyclic, bicylic, tricyclic or benzofused ring;
wherein one of R^{2β}, R^{3β}, R^{4β}, R^{5β} or R^{6β}, or one of R^{2α}, R^{3α}, R^{4α}, R^{5α} or R^{6α} must be a carbon-carbon linked heteroaryl;
wherein all R¹, R², R⁷ and R⁸ substituents can be optionally substituted where possible with one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂.

In another embodiment, the invention is represented by Formula I or its pharmaceutically acceptable salt or ester, wherein:

R^{3α}, R^{4α}, R^{5α}, R^{2β}, R^{3β}, R^{4β}, R^{5β} and R^{6β} are independently selected from the group consisting of hydrogen, halogen, nitro, alkyl, lower alkyl, alkenyl, alkynyl, carbocycle, cycloalkyl, cycloalkylalkyl, haloalkyl, aryl, arylalkyl, heteroaryl, heteroaryl lower alkyl, heterocyclic, heterocyclic lower alkyl, alkylthioalkyl, cycloalkylthioalkyl, arylthio lower alkyl, aralkyl lower thioalkyl, heteroarylthio lower alkyl, heteroaralkyl lower thioalkyl, heterocyclicthio lower alkyl, heterocyclicalkyl lower thioalkyl, —C(O)R², R²C(O)alkyl, aminoalkyl, cycloalkylaminoalkyl, arylamino lower alkyl, heteroarylamino lower alkyl, heterocyclicamino lower alkyl, hydroxyl, hydroxyalkyl, alkoxy, lower alkoxy, —(O(CH₂)₂)₁₋₃—O-lower alkyl, cycloalkyloxy, cycloalkylalkoxy, haloalkoxy, aryloxy, arylalkoxy, heteroaryloxy, heteroarylalkoxy, heteroaryl lower alkoxy, heterocyclicoxy, heterocyclicalkoxy, heterocyclic lower alkoxy, —OC(R¹)₂C(O)OH, —OC(R¹)₂C(O)OR², —OC(R¹)₂C(O)NH₂, —OC(R¹)₂C(O)NHR², —OC(R¹)₂C(O)N(R²)₂, —OC(R¹)₂C(O)NR⁷R⁸, amino, alkylamino, acylamino, dialkylamino, cycloalkylamino, arylamino, aralkylamino, heteroarylamino, heteroaralkylamino, heterocyclicamino, heterocyclicalkylamino, —NHR², N(R²)₂, —NR⁷R⁸, —NHC(R¹)₂C(O)OH, —NHC(R¹)₂C(O)OR², —NHC(O)R², —N(R²)C(O)R², —NHC(O)OR², —NHC(O)SR², —NHSO₂NHR², —NHSO₂R², —NHSO₂NR⁷R⁸, —N(C(O)NHR²)₂, —NR²SO₂R², —NHC(O)NHR², —NHC(O)NR⁷R⁸, —NHC(O)N(R²)₂, thiol, alkylthio, cycloalkylthio, cycloalkylalkylthio, haloalkylthio, arylthio, aralkylthio, heteroarylthio, heteroaralkylthio, heterocyclicthio, heterocyclicalkylthio, alkylsulfonyl, arylsulfonyl, haloalkylsulfonyl, —SC(R¹)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR R, —SO₂NHC(O)R², —SR², —SO₂NHC(O)NHR², —SO₂NHC(O)N(R²)₂, —SO₂NHC(O)NR⁷R⁸, sulfonic acid, sulfonate, sulfate, sulfinic acid, sulfenic acid, cyano, tetrazol-5-yl, carboxy, —C(O)OR², —C(O)NH₂, —C(O)NHR², —C(O)N(R²)₂, —C(O)NR⁷R⁸, —C(O)NHC(O)R², —C(O)NHC(O)NHR², —C(O)NHC(O)N(R²)₂, —C(O)NHC(O)NR⁷R⁸, —C(O)NHSO₂R², —C(O)NHSO₂NHR², C(O)NHSO₂N(R²), —C(O)NHSO₂NR⁷R⁸, —C(CH₃)₂C(O)OH, —(CH₂)_{y}C(O)OH, wherein y is 1, 2, 3, 4, 5, or 6, —PO₂H₂, —PO₃H₂, —P(R²)O₂H, and phosphate, all of which can be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R^{2α} and R^{6α} are independently selected from the group consisting of halogen, nitro, alkyl, lower alkyl, alkenyl, alkynyl, carbocycle, cycloalkyl, haloalkyl, aryl, arylalkyl, heteroaryl, heteroaryl lower alkyl, heterocyclic, heterocyclic lower alkyl, R²C(O)alkyl, aminoalkyl, cycloalkylaminoalkyl, arylamino lower alkyl, heteroarylamino lower alkyl, heterocyclicamino lower alkyl, hydroxyalkyl, alkoxy, lower alkoxy, cycloalkyloxy, cycloalkylalkoxy, haloalkoxy, aryloxy, arylalkoxy, heteroaryloxy, heteroarylalkoxy, heteroaryl lower alkoxy, heterocyclicoxy, heterocyclicalkoxy, heterocyclic lower alkoxy, —OC(R¹)₂C(O)OH, —OC(R¹)₂C(O)OR², —OC(R¹)₂C(O)NH₂, —OC(R¹)₂C(O)NHR², —OC(R¹)₂C(O)N(R²)₂, —OC(R¹)₂C(O)NR⁷R⁸, —NHSO₂NHR², —NHSO₂R², —NHSO₂NR⁷R⁸, —NR²SO₂R², alkylthio, cycloalkylthio, —SC(R¹)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR⁷R⁸, —SO₂NHC(O)R², —SR², —SO₂NHC(O)NHR², —SO₂NHC(O)N(R²)₂, —SO₂NHC(O)NR⁷R⁸, cyano, tetrazol-5-yl, carboxy, —C(O)OR², —C(O)NH₂, —C(O)NHR², —C(O)N(R²)₂, —C(O)NR⁷R⁸, —C(O)NHC(O)R², —C(O)NHC(O)NHR², —C(O)NHC(O)N(R²)₂, —C(O)NHC(O)NR⁷R⁸, —C(O)NHSO₂R², —C(O)NHSO₂NHR², —C(O)NHSO₂N(R²), —C(O)NHSO₂NR⁷R⁸, —C(CH₃)₂C(O)OH, —(CH₂)_{y}C(O)OH, wherein y is 1, 2, 3, 4, 5, or 6, all of which can be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R¹ is independently selected from the group consisting of hydrogen, lower alkyl, carbocycle, cycloalkyl, aryl, heteroaryl, heterocyclic, arylalkyl, heteroarylalkyl, and heterocyclicalkyl, wherein all may be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R² is independently selected from the group consisting of alkyl, lower alkyl, alkenyl, alkynyl, carbocycle, cycloalkyl, aryl, heteroaryl, heterocyclic, arylalkyl, heteroarylalkyl, and heterocyclicalkyl, wherein all may be substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R⁷ and R⁸ are independently selected from the group consisting of alkyl, alkenyl, heteroaryl and aryl and linked together forming a 4- to 12-membered monocyclic, bicylic, tricyclic or benzofused ring;
wherein one of R^{2α}, R^{3α}, R^{4α}, R^{5α} or R^{6α} must be a carbon-carbon linked heteroaryl;
wherein all R¹, R², R⁷ and R⁸ substituents can be optionally substituted where possible with one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂.

In a further embodiment, the invention is represented by Formula I or its pharmaceutically acceptable salt or ester, wherein:
R^{3α}, R^{4α}, R^{5α}, R^{2β}, R^{3β}, R^{4β}, R^{5β} and R^{6β} are independently selected from the group consisting of hydrogen, halogen, nitro, alkyl, lower alkyl, alkenyl, alkynyl, carbocycle, cycloalkyl, cycloalkylalkyl, haloalkyl, aryl, arylalkyl, heteroaryl, heteroaryl lower alkyl, heterocyclic, heterocyclic lower alkyl, R²C(O)alkyl, aminoalkyl, hydroxyl, hydroxyalkyl, alkoxy, lower alkoxy, cycloalkyloxy, haloalkoxy, aryloxy, arylalkoxy, heteroaryloxy, heteroarylalkoxy, —OC(R¹)₂C(O)OH, —OC(R¹)₂C(O)OR², —OC(R¹)₂C(O)NH₂, —OC(R¹)₂C(O)NHR², —OC(R¹)₂C(O)N(R²)₂, —OC(R¹)₂C(O)NR⁷R⁸, amino, alkylamino, acylamino, dialkylamino, cycloalkylamino, arylamino, aralkylamino, heteroarylamino, heteroaralkylamino, heterocyclicamino, heterocyclicalkylamino, —NHR², N(R²)₂, —NR⁷R⁸, —NHC(R¹)₂C(O)OH, —NHC(R¹)₂C(O)OR², —NHC(O)R², —N(R²)C(O)R², —NHC(O)OR², —NHC(O)SR², —NHSO₂NHR², —NHSO₂R², —NHSO₂N⁷R⁸, —N(C(O)NHR²)₂, —NR²SO₂R², —NHC(O)NHR², —NHC(O)NR⁷R⁸, —NHC(O)N(R²)₂, alkylsulfonyl, arylsulfonyl, haloalkylsulfonyl, —SC(R¹)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR⁷R⁸, —SO₂NHC(O)R², —SR², —SO₂NHC(O)NHR², —SO₂NHC(O)N(R²)₂, —SO₂NHC(O)NR⁷R⁸, cyano, tetrazol-5-yl, carboxy, —C(O)OR², —C(O)NH₂, —C(O)NHR², —C(O)N(R²)₂, —C(O)NR⁷R⁸, —C(O)NHC(O)R², —C(O)NHC(O)NHR², —C(O)NHC(O)N(R²)₂, —C(O)NHC(O)NR⁷R⁸, —C(O)NHSO₂R², —C(O)NHSO₂NHR², —C(O)NHSO₂N(R²), —C(O)NHSO₂NR⁷R⁸, —C(CH₃)₂C(O)OH, all of which can be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)N⁷R⁸, and —C(O)N(R²)₂;
R^{2α} and R^{6α} are independently selected from the group consisting of halogen, nitro, alkyl, lower alkyl, alkenyl, alkynyl, carbocycle, cycloalkyl, haloalkyl, aryl, arylalkyl, heteroaryl, heteroaryl lower alkyl, heterocyclic, heterocyclic lower alkyl, R²C(O)alkyl, aminoalkyl, hydroxyalkyl, alkoxy, lower alkoxy, cycloalkyloxy, cycloalkylalkoxy, haloalkoxy, aryloxy, arylalkoxy, heteroaryloxy, heteroarylalkoxy, heteroaryl lower alkoxy, heterocyclicoxy, heterocyclicalkoxy, heterocyclic lower alkoxy, —OC(R¹)₂C(O)OH, —OC(R¹)₂C(O)OR², —OC(R¹)₂C(O)NH₂, —OC(R¹)₂C(O)NHR², —OC(R¹)₂C(O)N(R²)₂, —OC(R¹)₂C(O)NR⁷R⁸, —SC(R¹)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR⁷R⁸, —SO₂NHC(O)R², —SR², —SO₂NHC(O)NHR², —SO₂NHC(O)N(R²)₂, —SO₂NHC(O)NR⁷R⁸, all of which can be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R¹ is independently selected from the group consisting of hydrogen, lower alkyl, carbocycle, cycloalkyl, aryl, heteroaryl, heterocyclic, arylalkyl, heteroarylalkyl, and heterocyclicalkyl, wherein all may be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R² is independently selected from the group consisting of alkyl, lower alkyl, alkenyl, alkynyl, carbocycle, cycloalkyl, aryl, heteroaryl, heterocyclic, arylalkyl, heteroarylalkyl, and heterocyclicalkyl, wherein all may be substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R⁷ and R⁸ are independently selected from the group consisting of alkyl, alkenyl, heteroaryl and aryl and linked together forming a 4- to 12-membered monocyclic, bicylic, tricyclic or benzofused ring;
wherein one of R^{2α}, R^{3α}, R^{4α}, R^{5α} or R^{6α} must be a carbon-carbon linked heteroaryl;
wherein all R¹, R², R⁷ and R⁸ substituents can be optionally substituted where possible with one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂.

In a further embodiment, the invention is represented by Formula I or its pharmaceutically acceptable salt or ester, wherein:

R^{3α}, R^{4α}, R^{5α}, R^{2β}, R^{3β}, R^{4β}, R^{5β} and R^{6β} are independently selected from the group consisting of hydrogen, halogen, nitro, alkyl, lower alkyl, alkenyl, alkynyl, carbocycle, cycloalkyl, cycloalkylalkyl, haloalkyl, aryl, arylalkyl, heteroaryl, heteroaryl lower alkyl, heterocyclic, heterocyclic lower alkyl, R²C(O)alkyl, aminoalkyl, hydroxyl, hydroxyalkyl, alkoxy, lower alkoxy, cycloalkyloxy, haloalkoxy, aryloxy, arylalkoxy, heteroaryloxy, heteroarylalkoxy, —OC(R¹)₂C(O)OH, —OC(R¹)₂C(O)OR², —OC(R¹)₂C(O)NH₂, —OC(R¹)₂C(O)NHR², —OC(R¹)₂C(O)N(R²)₂, —OC(R¹)₂C(O)NR⁷R⁸, amino, alkylamino, acylamino, dialkylamino, cycloalkylamino, arylamino, aralkylamino, heteroarylamino, heteroaralkylamino, heterocyclicamino, heterocyclicalkylamino, —NHR², N(R²)₂, —NR⁷R⁸, —NHC(R¹)₂C(O)OH, —NHC(R¹)₂C(O)OR², —NHC(O)R², —N(R²)C(O)R², —NHC(O)OR², —NHC(O)SR², —NHSO₂NHR², —NHSO₂R², —NHSO₂NR⁷R⁸, —N(C(O)NHR²)₂, —NR²SO₂R², —NHC(O)NHR², —NHC(O)NR⁷R⁸, —NHC(O)N(R²)₂, alkylsulfonyl, arylsulfonyl, haloalkylsulfonyl, —SC(R¹)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR⁷R⁸, —SO₂NHC(O)R², —SR², —SO₂NHC(O)NHR², —SO₂NHC(O)N(R²)₂, —SO₂NHC(O)NR⁷R⁸, cyano, tetrazol-5-yl, carboxy, —C(O)OR², —C(O)NH₂, —C(O)NHR², —C(O)N(R²)₂, —C(O)NR⁷R⁸, —C(O)NHC(O)R², —C(O)NHC(O)NHR², —C(O)NHC(O)N(R²)₂, —C(O)NHC(O)NR⁷R⁸, —C(O)NHSO₂R², —C(O)NHSO₂NHR², —C(O)NHSO₂N(R²), —C(O)NHSO₂NR⁷R⁸, —C(CH₃)₂C(O)OH, all of which can be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R^{2α} and R^{6α} are independently selected from the group consisting of halogen, nitro, lower alkyl, haloalkyl, aryl, heteroaryl, aminoalkyl, hydroxyalkyl, alkoxy, lower alkoxy, haloalkoxy, aryloxy, arylalkoxy, heteroaryloxy, heteroarylalkoxy, —OC(R¹)₂C(O)OH, —OC(R¹)₂C(O)OR², —OC(R¹)₂C(O)NH₂, —OC(R¹)₂C(O)NHR², —OC(R¹)₂C(O)N(R²)₂, —OC(R¹)₂C(O)NR⁷R⁸, —SC(R¹)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR⁷R⁸, —SO₂NHC(O)R², —SR², —SO₂NHC(O)NHR², —SO₂NHC(O)N(R²)₂, —SO₂NHC(O)NR⁷R⁸, all of which can be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R¹ is independently selected from the group consisting of hydrogen, lower alkyl, carbocycle, cycloalkyl, aryl, heteroaryl, heterocyclic, arylalkyl, heteroarylalkyl, and heterocyclicalkyl, wherein all may be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R² is independently selected from the group consisting of alkyl, lower alkyl, alkenyl, alkynyl, carbocycle, cycloalkyl, aryl, heteroaryl, heterocyclic, arylalkyl, heteroarylalkyl, and heterocyclicalkyl, wherein all may be substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R⁷ and R⁸ are independently selected from the group consisting of alkyl, alkenyl, heteroaryl and aryl and linked together forming a 4- to 12-membered monocyclic, bicylic, tricyclic or benzofused ring;
wherein one of R^{2α}, R^{3α}, R^{4α}, R^{5α} or R^{6α} must be a carbon-carbon linked heteroaryl;
wherein all R¹, R², R⁷ and R⁸ substituents can be optionally substituted where possible with one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂.

In another embodiment, the invention is represented by Formula I or its pharmaceutically acceptable salt or ester, wherein:
R^{3α}, R^{4α}, R^{5α}, R^{2β}, R^{3β}, R^{4β}, R^{5β} and R^{6β} are independently selected from the group consisting of hydrogen, halogen, nitro, alkyl, lower alkyl, haloalkyl, aryl, arylalkyl, heteroaryl, heterocyclic, aminoalkyl, hydroxyl, hydroxyalkyl, alkoxy, lower alkoxy, haloalkoxy, aryloxy, arylalkoxy, heteroaryloxy, heteroarylalkoxy, —OC(R¹)₂C(O)OH, —OC(R¹)₂C(O)OR², amino, alkylamino, acylamino, dialkylamino, cycloalkylamino, arylamino, aralkylamino, heteroarylamino, heteroaralkylamino, heterocyclicamino, heterocyclicalkylamino, —NHR², N(R²)₂, —NR⁷R⁸, —NHC(R¹)₂C(O)OH, —NHC(R¹)₂C(O)OR², —NHC(O)R², —N(R²)C(O)R², —NHC(O)OR², —NHC(O)SR², NHSO₂NHR², —NHSO₂R², —NHSO₂NR⁷R⁸, —N(C(O)NHR²)₂, —NR²SO₂R², —NHC(O)NHR², —NHC(O)NR⁷R⁸, —NHC(O)N(R²)₂, —SC(R¹)₂C(O)OH, —SC(R¹)₂C(O)OR², —SO₂NH₂, —SO₂NHR², —SO₂N(R²)₂, SO₂NR⁷R⁸, —SO₂NHC(O)R², —SR², —SO₂NHC(O)NHR², —SO₂NHC(O)N(R²)₂, —SO₂NHC(O)NR⁷R⁸, cyano, tetrazol-5-yl, carboxy, —C(O)OR², —C(O)NH₂, —C(O)NHR², —C(O)N(R²)₂, —C(O)NR⁷R⁸, —C(O)NHC(O)R², —C(O)NHC(O)NHR², —C(O)NHC(O)N(R²)₂, —C(O)NHC(O)NR⁷R⁸, —C(O)NHSO₂R², —C(O)NHSO₂NHR², —C(O)NHSO₂N(R²), —C(O)NHSO₂NR⁷R⁸, —C(CH₃)₂C(O)OH, all of which can be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R^{2α} and R^{6α} are independently selected from the group consisting of halogen, lower alkyl, lower alkoxy, aryl, and heteroaryl;
R¹ is independently selected from the group consisting of hydrogen, lower alkyl, carbocycle, cycloalkyl, aryl, heteroaryl, heterocyclic, arylalkyl, heteroarylalkyl, and heterocyclicalkyl, wherein all may be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R² is independently selected from the group consisting of alkyl, lower alkyl, alkenyl, alkynyl, carbocycle, cycloalkyl, aryl, heteroaryl, heterocyclic, arylalkyl, heteroarylalkyl, and heterocyclicalkyl, wherein all may be substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R⁷ and R⁸ are independently selected from the group consisting of alkyl, alkenyl, heteroaryl and aryl and linked together forming a 4- to 12-membered monocyclic, bicylic, tricyclic or benzofused ring;
wherein one of R^{2α}, R^{3α}, R^{4α}, R^{5α} or R^{6α} must be a carbon-carbon linked heteroaryl;
wherein all R¹, R², R⁷ and R⁸ substituents can be optionally substituted where possible with one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂.

In a further embodiment, the invention is represented by Formula I or its pharmaceutically acceptable salt or ester, wherein:
R^{2β}, R^{3β}, R^{4β}, R^{5β} and R^{6β} are independently selected from the group consisting of hydrogen, halogen, nitro, lower alkyl, haloalkyl, heteroaryl, heterocyclic, hydroxyl, lower alkoxy, —OC(R¹)₂C(O)OH, —OC(R¹)₂C(O)OR², —SO₂NHR², —SO₂N(R²)₂, carboxy, and —C(O)OR²;
R^{2α} and R^{6α} are independently selected from the group consisting of halogen, heteroaryl, aryl, lower alkyl, and lower alkoxy;
R¹ is independently selected from the group consisting of hydrogen and lower alkyl;
R² is lower alkyl, optionally substituted by one or more carboxy groups;
wherein one of R^{2α}, R^{3α}, R^{4α}, R^{5α}, or R^{6α} must be a carbon-carbon linked heteroaryl.

In another embodiment, the invention is represented by Formula I or its pharmaceutically acceptable salt or ester, wherein:
R^{2β}, R^{3β}, R^{5β} and R^{6β} are independently selected from the group consisting of hydrogen, nitro, lower alkyl, haloalkyl, heteroaryl, heterocyclic, hydroxyl, lower alkoxy, —OC(R¹)₂C(O)OH, —OC(R¹)₂C(O)OR², —SO₂NHR², —SO₂N(R²)₂, carboxy, and C(O)OR²;
R^{4β} is independently selected from the group consisting of hydrogen, halogen, nitro, lower alkyl, haloalkyl, heteroaryl, heterocyclic, hydroxyl, lower alkoxy, —OC(R¹)₂C(O)OH, —OC(R¹)₂C(O)OR², —SO₂NHR², —SO₂N(R²)₂, carboxy, and C(O)OR²;
R^{2α} and R^{6α} are independently selected from the group consisting of halogen, heteroaryl, aryl, lower alkyl, and lower alkoxy;
R¹ is independently selected from the group consisting of hydrogen and lower alkyl;
R² is lower alkyl, optionally substituted by one or more carboxy groups;
wherein one of R^{2α}, R^{3α}, R^{4α}, R^{5α}, or R^{6α} must be a carbon-carbon linked heteroaryl.

In a further embodiment, the invention is represented by Formula I or its pharmaceutically acceptable salt or ester, wherein:
R^{2β}, R^{3β}, R^{4β}, R^{5β} and R^{6β} are independently selected from the group consisting of hydrogen, chloro, fluoro, bromo, nitro, methyl, tert-butyl, trifluoromethyl, thienyl, benzothienyl, methoxypyridyl, pyridyl, hydroxyl, methoxy, carboxy, —OCH₂C(O)OH, —OC(CH₃)₂C(O)OH, and —SO₂N(CH₃)CH₂C(O)OH;
R^{2α} and R^{6α} are independently selected from the group consisting of chloro, fluoro, bromo, nitro, methyl, and methoxy;
wherein one of R^{2α}, R^{3α}, R^{4α}, R^{5α}, or R^{6α} must be thienyl, benzothienyl, methoxypyridyl, or pyridyl.

In another embodiment, the invention is represented by Formula I or its pharmaceutically acceptable salt or ester, wherein the compound is selected from the group consisting of:
4-[1-(2,6-Dimethoxy-3-thien-2-yl-benzoyl)-vinyl]-benzoic acid;
1-(2,6-Dimethoxy-3-thien-2-yl-phenyl)-2-(2-methoxy-phenyl)-propenone;
2-(3,5-Di-tert-butyl-4-hydroxy-phenyl)-1-(2,6-dimethoxy-3-thien-2-yl-phenyl) propenone;
2-{4-[1-(2,6-Dimethoxy-3-thien-2-yl-benzoyl)-vinyl]-phenoxy}-2-methyl-propionic acid;
1-(2,6-Dimethoxy-3-thien-2-yl-phenyl)-2-(4-trifluoromethyl-phenyl)-propenone;
1-(2,6-Dimethoxy-3-thien-2-yl-phenyl)-2-(4-nitro-phenyl)-propenone;
2-(2,6-Dichloro-phenyl)-1-(2,6-dimethoxy-3-thien-2-yl-phenyl)-propenone;
2-{3-[1-(2,6-Dimethoxy-3-thien-2-yl-benzoyl)-vinyl]-phenoxy}-2-methyl-propionic acid;
2-{3-[1-(3-Benzo[b]thien-2-yl-2,6-dimethoxy-benzoyl)-vinyl]-phenoxy}-2-methyl propionic acid;
({4-[1-(2,6-Dimethoxy-3-thien-2-yl-benzoyl)-vinyl]-benzenesulfonyl}-methyl-amino)-acetic acid; and
1-(2,6-Dimethoxy-3-thien-2-yl-phenyl)-2-(2-fluoro-6-trifluoromethyl-phenyl)-propenone.

In yet another embodiment, the invention is represented by Formula I or its pharmaceutically acceptable salt or ester, wherein:

R^{3α}, R^{4α}, R^{5α}, R^{2β}, R^{3β}, R^{4β}, R^{5β} and R^{6β} are independently selected from the group consisting of hydrogen, halogen, nitro, alkyl, lower alkyl, alkenyl, alkynyl, carbocycle, cycloalkyl, cycloalkylalkyl, haloalkyl, aryl, arylalkyl, heteroaryl, heteroaryl lower alkyl, heterocyclic, heterocyclic lower alkyl, alkylthioalkyl, cycloalkylthioalkyl, arylthio lower alkyl, aralkyl lower thioalkyl, heteroarylthio lower alkyl, heteroaralkyl lower thioalkyl, heterocyclicthio lower alkyl, heterocyclicalkyl lower thioalkyl, —C(O)R², R²C(O)alkyl, aminoalkyl, cycloalkylaminoalkyl, arylamino lower alkyl, heteroarylamino lower alkyl, heterocyclicamino lower alkyl, hydroxyl, hydroxyalkyl, alkoxy, lower alkoxy, —(O(CH₂)₂)₁₋₃—O-lower alkyl, cycloalkyloxy, cycloalkylalkoxy, haloalkoxy, aryloxy, arylalkoxy, heteroaryloxy, heteroarylalkoxy, heteroaryl lower alkoxy, heterocyclicoxy, heterocyclicalkoxy, heterocyclic lower alkoxy, —OC(R¹)₂C(O)OH, —OC(R¹)₂C(O)OR², —OC(R¹)₂C(O)NH₂, —OC(R¹)₂C(O)NHR², —OC(R¹)₂C(O)N(R²)₂, —OC(R¹)₂C(O)NR⁷R⁸, amino, alkylamino, acylamino, dialkylamino, cycloalkylamino, arylamino, aralkylamino, heteroarylamino, heteroaralkylamino, heterocyclicamino, heterocyclicalkylamino, —NHR², N(R²)₂, —NR⁷R⁸, —NHC(R¹)₂C(O)OH, —NHC(R¹)₂C(O)OR², —NHC(O)R², —N(R²)C(O)R², —NHC(O)OR², —NHC(O)SR², NHSO₂NHR², —NHSO₂R², —NHSO₂NR⁷R⁸, —N(C(O)NHR²)₂, —NR²SO₂R², —NHC(O)NHR², —NHC(O)NR⁷R⁸, —NHC(O)N(R²)₂, thiol, alkylthio, cycloalkylthio, cycloalkylalkylthio, haloalkylthio, arylthio, aralkylthio, heteroarylthio, heteroaralkylthio, heterocyclicthio, heterocyclicalkylthio, alkylsulfonyl, arylsulfonyl, haloalkylsulfonyl, —SC(R¹)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR⁷R⁸, —SO₂NHC(O)R², —SR², —SO₂NHC(O)NHR², —SO₂NHC(O)N(R²) 2, —SO₂NHC(O)NR⁷R⁸, sulfonic acid, sulfonate, sulfate, sulfinic acid, sulfenic acid, cyano, tetrazol-5-yl, carboxy, —C(O)OR², —C(O)NH₂, —C(O)NHR², —C(O)N(R²)₂, —C(O)NR⁷R⁸, —C(O)NHC(O)R², —C(O)NHC(O)NHR², —C(O)NHC(O)N(R²)₂, —C(O)NHC(O)NR⁷R⁸, —C(O)NHSO₂R², —C(O)NHSO₂NHR², —C(O)NHSO₂N(R²), —C(O)NHSO₂NR⁷R⁸, —C(CH₃)₂C(O)OH, —(CH₂)_{y}C(O)OH, wherein y is 1, 2, 3, 4, 5, or 6, —PO₂H₂, —PO₃H₂, —P(R²)O₂H, and phosphate, all of which can be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R^{2α} and R^{6α} are independently selected from the group consisting of halogen, nitro, alkyl, lower alkyl, alkenyl, alkynyl, carbocycle, cycloalkyl, haloalkyl, aryl, arylalkyl, heteroaryl, heteroaryl lower alkyl, heterocyclic, heterocyclic lower alkyl, R²C(O)alkyl, aminoalkyl, cycloalkylaminoalkyl, arylamino lower alkyl, heteroarylamino lower alkyl, heterocyclicamino lower alkyl, hydroxyalkyl, alkoxy, lower alkoxy, cycloalkyloxy, cycloalkylalkoxy, haloalkoxy, aryloxy, arylalkoxy, heteroaryloxy, heteroarylalkoxy, heteroaryl lower alkoxy, heterocyclicoxy, heterocyclicalkoxy, heterocyclic lower alkoxy, —OC(R¹)₂C(O)OH, —OC(R¹)₂C(O)OR², —OC(R¹)₂C(O)NH₂, —OC(R¹)₂C(O)NHR², —OC(R¹)₂C(O)N(R²)₂, —OC(R¹)₂C(O)NR⁷R⁸, —NHSO₂NHR², —NHSO₂R², —NHSO₂NR⁷R⁸, —NR²SO₂R², alkylthio, cycloalkylthio, —SC(R¹)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR⁷R⁸, —SO₂NHC(O)R², —SR², —SO₂NHC(O)NHR², —SO₂NHC(O)N(R²)₂, —SO₂NHC(O)NR⁷R⁸, cyano, tetrazol-5-yl, carboxy, —C(O)OR², —C(O)NH₂, —C(O)NHR², —C(O)N(R²)₂, —C(O)NR⁷R⁸, —C(O)NHC(O)R², —C(O)NHC(O)NHR², —C(O)NHC(O)N(R²)₂, —C(O)NHC(O)NR⁷R⁸, —C(O)NHSO₂R², —C(O)NHSO₂NHR², —C(O)NHSO₂N(R²), —C(O)NHSO₂NR⁷R⁸, —C(CH₃)₂C(O)OH, —(CH₂)_{y}C(O)OH, wherein y is 1, 2, 3, 4, 5, or 6, all of which can be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R¹ is independently selected from the group consisting of hydrogen, lower alkyl, carbocycle, cycloalkyl, aryl, heteroaryl, heterocyclic, arylalkyl, heteroarylalkyl, and heterocyclicalkyl, wherein all may be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R² is independently selected from the group consisting of alkyl, lower alkyl, alkenyl, alkynyl, carbocycle, cycloalkyl, aryl, heteroaryl, heterocyclic, arylalkyl, heteroarylalkyl, and heterocyclicalkyl, wherein all may be substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R⁷ and R⁸ are independently selected from the group consisting of alkyl, alkenyl, heteroaryl and aryl and linked together forming a 4- to 12-membered monocyclic, bicylic, tricyclic or benzofused ring;
wherein one of R²′, R^{3β}, R^{4β}, R^{5β} or R^{6β} must be a carbon-carbon linked heteroaryl;
wherein all R¹, R², R⁷ and R⁸ substituents can be optionally substituted where possible with one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂.

In a further embodiment, the invention is represented by Formula I or its pharmaceutically acceptable salt or ester, wherein:
R^{3α}, R^{4α}, R^{5α}, R^{2β}, R^{3β}, R^{4β}, R^{5β} and R^{6β} are independently selected from the group consisting of hydrogen, halogen, nitro, alkyl, lower alkyl, alkenyl, alkynyl, carbocycle, cycloalkyl, cycloalkylalkyl, haloalkyl, aryl, arylalkyl, heteroaryl, heteroaryl lower alkyl, heterocyclic, heterocyclic lower alkyl, R²C(O)alkyl, aminoalkyl, hydroxyl, hydroxyalkyl, alkoxy, lower alkoxy, cycloalkyloxy, haloalkoxy, aryloxy, arylalkoxy, heteroaryloxy, heteroarylalkoxy, —OC(R¹)₂C(O)OH, —OC(R¹)₂C(O)OR², —OC(R¹)₂C(O)NH₂, —OC(R¹)₂C(O)NHR², —OC(R¹)₂C(O)N(R²)₂, —OC(R¹)₂C(O)NR⁷R⁸, amino, alkylamino, acylamino, dialkylamino, cycloalkylamino, arylamino, aralkylamino, heteroarylamino, heteroaralkylamino, heterocyclicamino, heterocyclicalkylamino, —NHR², N(R²)₂, —NR⁷R⁸, —NHC(R¹)₂C(O)OH, —NHC(R¹)₂C(O)OR², —NHC(O)R², —N(R²)C(O)R², —NHC(O)OR², —NHC(O)SR², —NHSO₂NHR², —NHSO₂R², —NHSO₂NR⁷R⁸, —N(C(O)NHR²)₂, —NR²SO₂R², —NHC(O)NHR², —NHC(O)NR⁷R⁸, —NHC(O)N(R²)₂, alkylsulfonyl, arylsulfonyl, haloalkylsulfonyl, —SC(R¹)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR⁷R⁸, —SO₂NHC(O)R², —SR², —SO₂NHC(O)NHR², —SO₂NHC(O)N(R²) 2, —SO₂NHC(O)NR⁷R⁸, cyano, tetrazol-5-yl, carboxy, —C(O)OR², —C(O)NH₂, —C(O)NHR², —C(O)N(R²)₂, —C(O)NR⁷R⁸, —C(O)NHC(O)R², —C(O)NHC(O)NHR², —C(O)NHC(O)N(R²)₂, —C(O)NHC(O)NR⁷R⁸, —C(O)NHSO₂R², —C(O)NHSO₂NHR², —C(O)NHSO₂N(R²), —C(O)NHSO₂NR⁷R⁸, —C(CH₃)₂C(O)OH, all of which can be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R^{2α} and R^{6α} are independently selected from the group consisting of halogen, nitro, alkyl, lower alkyl, alkenyl, alkynyl, carbocycle, cycloalkyl, haloalkyl, aryl, arylalkyl, heteroaryl, heteroaryl lower alkyl, heterocyclic, heterocyclic lower alkyl, R²C(O)alkyl, aminoalkyl, hydroxyalkyl, alkoxy, lower alkoxy, cycloalkyloxy, cycloalkylalkoxy, haloalkoxy, aryloxy, arylalkoxy, heteroaryloxy, heteroarylalkoxy, heteroaryl lower alkoxy, heterocyclicoxy, heterocyclicalkoxy, heterocyclic lower alkoxy, —OC(R¹)₂C(O)OH, —OC(R¹)₂C(O)OR², —OC(R¹)₂C(O)NH₂, —OC(R¹)₂C(O)NHR², —OC(R¹)₂C(O)N(R²)₂, —OC(R¹)₂C(O)NR⁷R⁸, —SC(R¹)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR⁷R⁸, —SO₂NHC(O)R², —SR², —SO₂NHC(O)NHR², —SO₂NHC(O)N(R²)₂, —SO₂NHC(O)NR⁷R⁸, all of which can be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R¹ is independently selected from the group consisting of hydrogen, lower alkyl, carbocycle, cycloalkyl, aryl, heteroaryl, heterocyclic, arylalkyl, heteroarylalkyl, and heterocyclicalkyl, wherein all may be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R² is independently selected from the group consisting of alkyl, lower alkyl, alkenyl, alkynyl, carbocycle, cycloalkyl, aryl, heteroaryl, heterocyclic, arylalkyl, heteroarylalkyl, and heterocyclicalkyl, wherein all may be substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and C(O)N(R²)₂;
R⁷ and R⁸ are independently selected from the group consisting of alkyl, alkenyl, heteroaryl and aryl and linked together forming a 4- to 12-membered monocyclic, bicylic, tricyclic or benzofused ring;
wherein one of R^{2β}, R^{3β}, R^{4β}, R^{5β} or R^{6β} must be a carbon-carbon linked heteroaryl;
wherein all R¹, R², R⁷ and R⁸ substituents can be optionally substituted where possible with one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂.

In another embodiment, the invention is represented by Formula I or its pharmaceutically acceptable salt or ester, wherein:

R^{3α}, R^{4α}, R^{5α}, R^{2β}, R^{3β}, R^{4β}, R^{5β} and R^{6β} are independently selected from the group consisting of hydrogen, halogen, nitro, alkyl, lower alkyl, alkenyl, alkynyl, carbocycle, cycloalkyl, cycloalkylalkyl, haloalkyl, aryl, arylalkyl, heteroaryl, heteroaryl lower alkyl, heterocyclic, heterocyclic lower alkyl, R²C(O)alkyl, aminoalkyl, hydroxyl, hydroxyalkyl, alkoxy, lower alkoxy, cycloalkyloxy, haloalkoxy, aryloxy, arylalkoxy, heteroaryloxy, heteroarylalkoxy, —OC(R¹)₂C(O)OH, —OC(R¹)₂C(O)OR², —OC(R¹)₂C(O)NH₂, —OC(R¹)₂C(O)NHR², —OC(R¹)₂C(O)N(R²)₂, —OC(R¹)₂C(O)NR⁷R⁸, amino, alkylamino, acylamino, dialkylamino, cycloalkylamino, arylamino, aralkylamino, heteroarylamino, heteroaralkylamino, heterocyclicamino, heterocyclicalkylamino, NHR², N(R²)₂, —NR⁷R⁸, —NHC(R¹)₂C(O)OH, —NHC(R¹)₂C(O)OR², —NHC(O)R², —N(R²)C(O)R², —NHC(O)OR², —NHC(O)SR², —NHSO₂NHR², —NHSO₂R², —NHSO₂NR⁷R⁸, —N(C(O)NHR²)₂, —NR²SO₂R², —NHC(O)NHR², —NHC(O)NR⁷R⁸, —NHC(O)N(R²)₂, alkylsulfonyl, arylsulfonyl, haloalkylsulfonyl, —SC(R¹)₂C(O)OH, —SC(R²)₂C(O)OR², —SCH₂C(O)OH, —SCF₂C(O)OH, —SO₂NH₂, —SO₂NHR², —SO₂N(R²)₂, SO₂NR⁷R⁸, —SO₂NHC(O)R², —SR², —SO₂NHC(O)NHR², —SO₂NHC(O)N(R²)₂, —SO₂NHC(O)NR⁷R⁸, cyano, tetrazol-5-yl, carboxy, —C(O)OR², —C(O)NH₂, —C(O)NHR², —C(O)N(R²)₂, —C(O)NR⁷R⁸, —C(O)NHC(O)R², —C(O)NHC(O)NHR², —C(O)NHC(O)N(R²)₂, —C(O)NHC(O)NR⁷R⁸, —C(O)NHSO₂R², —C(O)NHSO₂NHR², —C(O)NHSO₂N(R²), —C(O)NHSO₂NR⁷R⁸, —C(CH₃)₂C(O)OH, all of which can be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R^{2α} and R^{6α} are independently selected from the group consisting of halogen, nitro, lower alkyl, haloalkyl, aryl, heteroaryl, aminoalkyl, hydroxyalkyl, alkoxy, lower alkoxy, haloalkoxy, aryloxy, arylalkoxy, heteroaryloxy, heteroarylalkoxy, OC(R¹)₂C(O)OH, —OC(R¹)₂C(O)OR², —OC(R¹)₂C(O)NH₂, —OC(R¹)₂C(O)NHR², —OC(R¹)₂C(O)N(R²)₂, —OC(R¹)₂C(O)NR⁷R⁸, —SC(R¹)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR⁷R⁸, —SO₂NHC(O)R², —SR², —SO₂NHC(O)NHR², —SO₂NHC(O)N(R²)₂, —SO₂NHC(O)NR⁷R⁸, all of which can be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R¹ is independently selected from the group consisting of hydrogen, lower alkyl, carbocycle, cycloalkyl, aryl, heteroaryl, heterocyclic, arylalkyl, heteroarylalkyl, and heterocyclicalkyl, wherein all may be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R² is independently selected from the group consisting of alkyl, lower alkyl, alkenyl, alkynyl, carbocycle, cycloalkyl, aryl, heteroaryl, heterocyclic, arylalkyl, heteroarylalkyl, and heterocyclicalkyl, wherein all may be substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R⁷ and R⁸ are independently selected from the group consisting of alkyl, alkenyl, heteroaryl and aryl and linked together forming a 4- to 12-membered monocyclic, bicylic, tricyclic or benzofused ring;
wherein one of R^{2β}, R^{3β}, R^{4β}, R^{5β} or R^{6β} must be a carbon-carbon linked heteroaryl;
wherein all R¹, R², R⁷ and R⁸ substituents can be optionally substituted where possible with one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂.

In another embodiment, the invention is represented by Formula I or its pharmaceutically acceptable salt or ester, wherein:

R^{3α}, R^{4α}, R^{5α}, R^{2β}, R^{3β}, R^{4β}, R^{5β} and R^{6β} are independently selected from the group consisting of hydrogen, halogen, nitro, alkyl, haloalkyl, aryl, arylalkyl, heteroaryl, heterocyclic, aminoalkyl, hydroxyl, hydroxyalkyl, alkoxy, haloalkoxy, aryloxy, arylalkoxy, heteroaryloxy, heteroarylalkoxy, —OC(R¹)₂C(O)OH, —OC(R¹)₂C(O)OR², amino, alkylamino, acylamino, dialkylamino, cycloalkylamino, arylamino, aralkylamino, heteroarylamino, heteroaralkylamino, heterocyclicamino, heterocyclicalkylamino, NHR², N(R²)₂, —NR⁷R⁸, —NHC(R¹)₂C(O)OH, —NHC(R¹)₂C(O)OR², —NHC(O)R², —N(R²)C(O)R², —NHC(O)OR², —NHC(O)SR², —NHSO₂NHR², —NHSO₂R², —NHSO₂NR⁷R⁸, —N(C(O)NHR²)₂, —NR²SO₂R², —NHC(O)NHR², —NHC(O)NR⁷R⁸, —NHC(O)N(R²)₂, —SC(R¹)₂C(O)OH, —SC(R¹)₂C(O)OR², —SO₂NH₂, —SO₂NHR², —SO₂N(R²)₂, SO₂NR⁷R⁸, —SO₂NHC(O)R², —SR², —SO₂NHC(O)NHR², —SO₂NHC(O)N(R²)₂, —SO₂NHC(O)NR⁷R⁸, cyano, tetrazol-5-yl, carboxy, —C(O)OR², —C(O)NH₂, —C(O)NHR², —C(O)N(R²)₂, —C(O)NR⁷R⁸, —C(O)NHC(O)R², —C(O)NHC(O)NHR², —C(O)NHC(O)N(R²)₂, —C(O)NHC(O)NR⁷R⁸, —C(O)NHSO₂R², —C(O)NHSO₂NHR², —C(O)NHSO₂N(R²), —C(O)NHSO₂NR⁷R⁸, —C(CH₃)₂C(O)OH, all of which can be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R^{2α} and R^{6α} are independently selected from the group consisting of F, Cl, Br, —CH₃, —OCH₃, aryl, and heteroaryl;
R¹ is independently selected from the group consisting of hydrogen, lower alkyl, carbocycle, cycloalkyl, aryl, heteroaryl, heterocyclic, arylalkyl, heteroarylalkyl, and heterocyclicalkyl, wherein all may be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R² is independently selected from the group consisting of alkyl, lower alkyl, alkenyl, alkynyl, carbocycle, cycloalkyl, aryl, heteroaryl, heterocyclic, arylalkyl, heteroarylalkyl, and heterocyclicalkyl, wherein all may be substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R⁷ and R⁸ are independently selected from the group consisting of alkyl, alkenyl, heteroaryl and aryl and linked together forming a 4- to 12-membered monocyclic, bicylic, tricyclic or benzofused ring;
wherein one of R^{2β}, R^{3β}, R^{4β}, R^{5β} or R^{6β} must be a carbon-carbon linked heteroaryl;
wherein all R¹, R², R⁷ and R⁸ substituents can be optionally substituted where possible with one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂.

In a further embodiment, the invention is represented by Formula I or its pharmaceutically acceptable salt or ester, wherein:
R^{2β}, R^{3β}, R^{4β}, R^{5β} and R^{6β} are independently selected from the group consisting of hydrogen, halogen, lower alkyl, haloalkyl, heteroaryl, heterocyclic, lower alkoxy, —OC(R¹)₂C(O)OH, —OC(R¹)₂C(O)OR², —SO₂NHR², —SO₂N(R²)₂, carboxy, and —C(O)OR²;
R^{2α} and R^{6α} are independently selected from the group consisting of halogen, aryl, heteroaryl, lower alkyl, and lower alkoxy;
R¹ is independently selected from the group consisting of hydrogen and lower alkyl;
R² is lower alkyl, optionally substituted by one or more carboxy groups;
wherein one of R^{2β}, R^{3β}, R^{4β}, R^{5β} or R^{6β} must be a carbon-carbon linked heteroaryl.

In yet another embodiment, the invention is represented by Formula I or its pharmaceutically acceptable salt or ester, wherein:
R^{2β}, R^{3β}, R^{4β}, R^{5β} and R^{6β} are independently selected from the group consisting of hydrogen, lower alkyl, haloalkyl, heteroaryl, heterocyclic, lower alkoxy, —OC(R¹)₂C(O)OH, —OC(R¹)₂C(O)OR², —SO₂NHR², —SO₂N(R²)₂, carboxy, and —C(O)OR²;
R^{4β} is selected from the group consisting of hydrogen, halogen, lower alkyl, haloalkyl, heteroaryl, heterocyclic, lower alkoxy, —OC(R¹)₂C(O)OH, —OC(R¹)₂C(O)OR², —SO₂NHR², —SO₂N(R²)₂, carboxy, and —C(O)OR²;
R^{2α} and R^{6α} are independently selected from the group consisting of halogen, aryl, heteroaryl, lower alkyl, and lower alkoxy;
R¹ is independently selected from the group consisting of hydrogen and lower alkyl;
R² is lower alkyl, optionally substituted by one or more carboxy groups;
wherein one of R^{2β}, R^{3β}, R^{4β}, R^{5β} or R^{6β} must be a carbon-carbon linked heteroaryl.

In another embodiment, the invention is represented by Formula I or its pharmaceutically acceptable salt or ester, wherein:
R^{2β}, R^{3β}, R^{4β}, R^{5β} and R^{6β} are independently selected from the group consisting of hydrogen, chloro, fluoro, bromo, nitro, methyl, tert-butyl, trifluoromethyl, thienyl, benzothienyl, methoxypyridyl, pyridyl, hydroxyl, methoxy, carboxy, —OCH₂C(O)OH, —OC(CH₃)₂C(O)OH, and —SO₂N(CH₃)CH₂C(O)OH;
R^{2α} and R^{6α} are independently selected from the group consisting of chloro, fluoro, bromo, nitro, methyl, and methoxy;
wherein one of R^{2β}, R^{3β}, R^{4β}, R^{5β} or R^{6β} must be thienyl, benzothienyl, methoxypyridyl, or pyridyl.

In a further embodiment, the invention is represented by Formula I or its pharmaceutically acceptable salt or ester, wherein the compound is selected from the group consisting of:
1-(2,6-Dichloro-phenyl)-2-(2-methoxy-5-thien-2-yl-phenyl)-propenone;
1-(2,6-Dimethyl-phenyl)-2-(2-methoxy-5-thien-2-yl-phenyl)-propenone;
4-[1-(2-Chloro-6-methyl-benzoyl)-vinyl]-3-thien-2-yl-benzoic acid;
4-[1-(2,6-Dimethyl-benzoyl)-vinyl]-3-thien-2-yl-benzoic acid;
4-[1-(2,6-Dimethoxy-benzoyl)-vinyl]-3-thien-2-yl-benzoic acid;
2-{4-[1-(2,6-Dimethoxy-benzoyl)-vinyl]-2-thien-2-yl-phenoxy}-2-methyl-propionic acid;
1-(2,6-Dimethyl-phenyl)-2-(2-thien-2-yl-phenyl)-propenone;
1-(2,6-Dimethyl-phenyl)-2-[2-(2-methoxy-pyridin-3-yl)-phenyl]-propenone;
1-(2,6-Dimethoxy-phenyl)-2-(2-thien-2-yl-phenyl)-propenone;
1-(2,6-Dimethoxy-phenyl)-2-(2-methoxy-5-thien-2-yl-phenyl)-propenone; and
2-{2-[1-(2,6-Dimethoxy-benzoyl)-vinyl]-4-thien-2-yl-phenoxy}-2-methyl-propionic acid.
In another embodiment, the invention is represented by Formula I or its pharmaceutically acceptable salt or ester, wherein:

R^{2α}, R^{3α}, R^{4α}, R^{5α}, R^{6α}, and R^{4β} are independently selected from the group consisting of hydrogen, halogen, nitro, alkyl, lower alkyl, alkenyl, alkynyl, carbocycle, cycloalkyl, cycloalkylalkyl, haloalkyl, aryl, arylalkyl, heteroaryl, heteroaryl lower alkyl, heterocyclic, heterocyclic lower alkyl, alkylthioalkyl, cycloalkylthioalkyl, arylthio lower alkyl, aralkyl lower thioalkyl, heteroarylthio lower alkyl, heteroaralkyl lower thioalkyl, heterocyclicthio lower alkyl, heterocyclicalkyl lower thioalkyl, —C(O)R², R²C(O)alkyl, aminoalkyl, cycloalkylaminoalkyl, arylamino lower alkyl, heteroarylamino lower alkyl, heterocyclicamino lower alkyl, hydroxyl, hydroxyalkyl, alkoxy, lower alkoxy, —(O(CH₂)₂)₁₋₃—O-lower alkyl, cycloalkyloxy, cycloalkylalkoxy, haloalkoxy, aryloxy, arylalkoxy, heteroaryloxy, heteroarylalkoxy, heteroaryl lower alkoxy, heterocyclicoxy, heterocyclicalkoxy, heterocyclic lower alkoxy, —OC(R¹)₂C(O)OH, —OC(R¹)₂C(O)OR², —OC(R¹)₂C(O)NH₂, —OC(R¹)₂C(O)NHR², —OC(R¹)₂C(O)N(R²)₂, —OC(R¹)₂C(O)NR⁷R⁸, amino, alkylamino, acylamino, dialkylamino, cycloalkylamino, arylamino, aralkylamino, heteroarylamino, heteroaralkylamino, heterocyclicamino, heterocyclicalkylamino, —NHR², N(R²)₂, —NR⁷R⁸, —NHC(R¹)₂C(O)OH, —NHC(R¹)₂C(O)OR², —NHC(O)R², —N(R²)C(O)R², —NHC(O)OR², —NHC(O)SR², —NHSO₂NHR², —NHSO₂R², —NHSO₂NR⁷R⁸, —N(C(O)NHR²)₂, —NR²SO₂R², —NHC(O)NHR², —NHC(O)NR⁷R⁸, —NHC(O)N(R²)₂, thiol, alkylthio, cycloalkylthio, cycloalkylalkylthio, haloalkylthio, arylthio, aralkylthio, heteroarylthio, heteroaralkylthio, heterocyclicthio, heterocyclicalkylthio, alkylsulfonyl, arylsulfonyl, haloalkylsulfonyl, —SC(R¹)₂C(O)OH, —SC(R¹)₂C(O)OR², —SCH₂C(O)OH, SCF₂C(O)OH, —SO₂NH₂, —SO₂NHR², —SO₂N(R²)₂, SO₂NR⁷R⁸, —SO₂NHC(O)R², —SR², —SO₂NHC(O)NHR², —SO₂NHC(O)N(R²) 2, —SO₂NHC(O)NR⁷R⁸, sulfonic acid, sulfonate, sulfate, sulfinic acid, sulfenic acid, cyano, tetrazol-5-yl, carboxy, —C(O)OR², —C(O)NH₂, —C(O)NHR², —C(O)N(R²)₂, —C(O)NR⁷R⁸, —C(O)NHC(O)R², —C(O)NHC(O)NHR², —C(O)NHC(O)N(R²)₂, —C(O)NHC(O)NR⁷R⁸, —C(O)NHSO₂R², —C(O)NHSO₂NHR², —C(O)NHSO₂N(R²), —C(O)NHSO₂NR⁷R⁸, —C(CH₃)₂C(O)OH, —(CH₂)_{y}C(O)OH, wherein y is 1, 2, 3, 4, 5, or 6, —PO₂H₂, —PO₃H₂, —P(R²)O₂H, and phosphate, all of which can be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R^{2β}, R^{3β}, R^{5β} and R^{6β} are independently selected from the group consisting of hydrogen, nitro, alkyl, lower alkyl, alkenyl, alkynyl, carbocycle, cycloalkyl, cycloalkylalkyl, haloalkyl, aryl, arylalkyl, heteroaryl, heteroaryl lower alkyl, heterocyclic, heterocyclic lower alkyl, alkylthioalkyl, cycloalkylthioalkyl, arylthio lower alkyl, aralkyl lower thioalkyl, heteroarylthio lower alkyl, heteroaralkyl lower thioalkyl, heterocyclicthio lower alkyl, heterocyclicalkyl lower thioalkyl, —C(O)R², R²C(O)alkyl, aminoalkyl, cycloalkylaminoalkyl, arylamino lower alkyl, heteroarylamino lower alkyl, heterocyclicamino lower alkyl, hydroxyl, hydroxyalkyl, alkoxy, lower alkoxy, —(O(CH₂)₂)₁₋₃—O-lower alkyl, cycloalkyloxy, cycloalkylalkoxy, haloalkoxy, aryloxy, arylalkoxy, heteroaryloxy, heteroarylalkoxy, heteroaryl lower alkoxy, heterocyclicoxy, heterocyclicalkoxy, heterocyclic lower alkoxy, —OC(R¹)₂C(O)OH, —OC(R¹)₂C(O)OR², —OC(R¹)₂C(O)NH₂, —OC(R¹)₂C(O)NHR², —OC(R¹)₂C(O)N(R²)₂, —OC(R¹)₂C(O)NR⁷R⁸, amino, alkylamino, acylamino, dialkylamino, cycloalkylamino, arylamino, aralkylamino, heteroarylamino, heteroaralkylamino, heterocyclicamino, heterocyclicalkylamino, —NHR², N(R²)₂, —NR R, —NHC(R¹)₂C(O)OH, —NHC(R¹)₂C(O)OR², —NHC(O)R², —N(R²)C(O)R², —NHC(O)OR², —NHC(O)SR², —NHSO₂NHR², —NHSO₂R, —NHSO₂NR⁷R⁸, —N(C(O)NHR²)₂, —NR²SO₂R², —NHC(O)NHR², —NHC(O)NR⁷R⁸, —NHC(O)N(R²)₂, thiol, alkylthio, cycloalkylthio, cycloalkylalkylthio, haloalkylthio, arylthio, aralkylthio, heteroarylthio, heteroaralkylthio, heterocyclicthio, heterocyclicalkylthio, alkylsulfonyl, arylsulfonyl, haloalkylsulfonyl, —SC(R¹)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR⁷R⁸, —SO₂NHC(O)R, —SR², —SO₂NHC(O)NHR², —SO₂NHC(O)N(R²) 2, —SO₂NHC(O)NR⁷R⁸, sulfonic acid, sulfonate, sulfate, sulfinic acid, sulfenic acid, cyano, tetrazol-5-yl, carboxy, —C(O)OR², —C(O)NH₂, —C(O)NHR², —C(O)N(R²)₂, —C(O)NR⁷R⁸, —C(O)NHC(O)R², —C(O)NHC(O)NHR², —C(O)NHC(O)N(R²)₂, —C(O)NHC(O)NR⁷R⁸, —C(O)NHSO₂R², —C(O)NHSO₂NHR², —C(O)NHSO₂N(R²), —C(O)NHSO₂NR⁷R⁸, —C(CH₃)₂C(O)OH, —(CH₂)_{y}C(O)OH, wherein y is 1, 2, 3, 4, 5, or 6, —PO₂H₂, —PO₃H₂, —P(R²)O₂H, and phosphate, all of which can be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R¹ is independently selected from the group consisting of hydrogen, lower alkyl, carbocycle, cycloalkyl, aryl, heteroaryl, heterocyclic, arylalkyl, heteroarylalkyl, and heterocyclicalkyl, wherein all may be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R² is independently selected from the group consisting of alkyl, lower alkyl, alkenyl, alkynyl, carbocycle, cycloalkyl, aryl, heteroaryl, heterocyclic, arylalkyl, heteroarylalkyl, and heterocyclicalkyl, wherein all may be substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R⁷ and R⁸ are independently selected from the group consisting of alkyl, alkenyl, heteroaryl and aryl and linked together forming a 4- to 12-membered monocyclic, bicylic, tricyclic or benzofused ring;
wherein one of R^{2α}, R^{3α}, R^{4α}, R^{5α} or R^{6α} must be a carbon-carbon linked heteroaryl;
wherein all R¹, R², R⁷ and R⁸ substituents can be optionally substituted where possible with one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂.

In a further embodiment, the invention is represented by Formula I or its pharmaceutically acceptable salt or ester, wherein:
R^{2α}, R^{3α}, R^{4α}, R^{5α}, R^{6α} and R^{4β}, are independently selected from the group consisting of hydrogen, halogen, nitro, alkyl, haloalkyl, heteroaryl, heterocyclic, hydroxyl, alkoxy, haloalkoxy, heteroaryloxy, heteroarylalkoxy, heterocyclicoxy, heterocyclicalkoxy, —OC(R¹)₂C(O)OH, —OC(R¹)₂C(O)OR², —OC(R¹)₂C(O)NH₂, OC(R¹)₂C(O)NHR², —OC(R¹)₂C(O)N(R²)₂, —OC(R¹)₂C(O)NR⁷R⁸, heteroarylamino, heterocyclicamino, —NHR², N(R²)₂, —NR⁷R⁸, —NHC(O)R², —N(R²)C(O)R², —NHC(O)OR², —NHSO₂R², —NR²SO₂R², cyano, tetrazol-5-yl, carboxy, —C(O)OR², —C(O)NH₂, —C(O)NHR², —C(O)N(R²)₂, —C(O)NR⁷R⁸, all of which can be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R^{2β}, R^{3β}, R^{5β} and R^{6β} are independently selected from the group consisting of hydrogen, heteroaryl, heterocyclic, alkyl, alkoxy, and lower alkoxy, all of which can be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R¹ is independently selected from the group consisting of hydrogen, and lower alkyl, optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R² is independently selected from the group consisting of alkyl and lower alkyl, optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R⁷ and R⁸ are independently selected from the group consisting of alkyl, alkenyl, heteroaryl and aryl and linked together forming a 4- to 12-membered monocyclic, bicylic, tricyclic or benzofused ring, optionally substituted where possible with one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
wherein one of R^{2α}, R^{3α}, R^{4α}, R^{5α} or R^{6α} must be a carbon-carbon linked heteroaryl.

In a further embodiment, the invention is represented by Formula I or its pharmaceutically acceptable salt or ester, wherein the compound is selected from the group consisting of:
4-[1-(5-Benzo[b]thien-2-yl-2,4-dimethoxy-benzoyl)-vinyl]-benzoic acid methyl ester;
4-[1-(4-Methoxy-3-thien-2-yl-benzoyl)-vinyl]-benzoic acid;
4-[1-(3,4-Dimethoxy-5-thien-2-yl-benzoyl)-vinyl]-benzoic acid;
1-(5-Benzo[b]thien-2-yl-2,4-dimethoxy-phenyl)-2-(4-methoxy-phenyl)-propenone;
1-(5-Benzo[b]thien-2-yl-2,4-dimethoxy-phenyl)-2-(4-fluoro-phenyl)-propenone;
1-(2-Methoxy-5-thien-2-yl-phenyl)-2-(4-nitro-phenyl)-propenone;
4-[1-(2-Methoxy-5-thien-2-yl-benzoyl)-vinyl]-benzonitrile; and
4-[1-(2-Methoxy-5-thien-2-yl-benzoyl)-vinyl]-benzoic acid.

In another embodiment, the invention is represented by Formula I or its pharmaceutically acceptable salt or ester, wherein:

R^{2α}, R^{3α}, R^{4α}, R^{5α}, R^{6α}, and R^{4β} are independently selected from the group consisting of hydrogen, halogen, nitro, alkyl, lower alkyl, alkenyl, alkynyl, carbocycle, cycloalkyl, cycloalkylalkyl, haloalkyl, aryl, arylalkyl, heteroaryl, heteroaryl lower alkyl, heterocyclic, heterocyclic lower alkyl, alkylthioalkyl, cycloalkylthioalkyl, arylthio lower alkyl, aralkyl lower thioalkyl, heteroarylthio lower alkyl, heteroaralkyl lower thioalkyl, heterocyclicthio lower alkyl, heterocyclicalkyl lower thioalkyl, —C(O)R², R²C(O)alkyl, aminoalkyl, cycloalkylaminoalkyl, arylamino lower alkyl, heteroarylamino lower alkyl, heterocyclicamino lower alkyl, hydroxyl, hydroxyalkyl, alkoxy, lower alkoxy, —(O(CH₂)₂)₁₋₃—O-lower alkyl, cycloalkyloxy, cycloalkylalkoxy, haloalkoxy, aryloxy, arylalkoxy, heteroaryloxy, heteroarylalkoxy, heteroaryl lower alkoxy, heterocyclicoxy, heterocyclicalkoxy, heterocyclic lower alkoxy, —OC(R¹)₂C(O)OH, —OC(R¹)₂C(O)OR², —OC(R¹)₂C(O)NH₂, —OC(R¹)₂C(O)NHR², —OC(R¹)₂C(O)N(R²)₂, —OC(R¹)₂C(O)NR⁷R⁸, amino, alkylamino, acylamino, dialkylamino, cycloalkylamino, arylamino, aralkylamino, heteroarylamino, heteroaralkylamino, heterocyclicamino, heterocyclicalkylamino, —NHR², N(R²)₂, —NR⁷R⁸, —NHC(R¹)₂C(O)OH, —NHC(R¹)₂C(O)OR², —NHC(O)R², —N(R²)C(O)R², —NHC(O)OR², —NHC(O)SR², —NHSO₂NHR², —NHSO₂R², —NHSO₂NR⁷R⁸, —N(C(O)NHR²)₂, —NR²SO₂R², —NHC(O)NHR², —NHC(O)NR⁷R⁸, —NHC(O)N(R²)₂, thiol, alkylthio, cycloalkylthio, cycloalkylalkylthio, haloalkylthio, arylthio, aralkylthio, heteroarylthio, heteroaralkylthio, heterocyclicthio, heterocyclicalkylthio, alkylsulfonyl, arylsulfonyl, haloalkylsulfonyl, —SC(R¹)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR⁷R⁸, —SO₂NHC(O)R², —SR², —SO₂NHC(O)NHR², —SO₂NHC(O)N(R²) 2, —SO₂NHC(O)NR⁷R⁸, sulfonic acid, sulfonate, sulfate, sulfinic acid, sulfenic acid, cyano, tetrazol-5-yl, carboxy, —C(O)OR², —C(O)NH₂, —C(O)NHR², —C(O)N(R²)₂, —C(O)NR⁷R⁸, —C(O)NHC(O)R², —C(O)NHC(O)NHR², —C(O)NHC(O)N(R²)₂, —C(O)NHC(O)NR⁷R⁸, —C(O)NHSO₂R², —C(O)NHSO₂NHR², —C(O)NHSO₂N(R²), —C(O)NHSO₂NR⁷R⁸, —C(CH₃)₂C(O)OH, —(CH₂)_{y}C(O)OH, wherein y is 1, 2, 3, 4, 5, or 6, —PO₂H₂, —PO₃H₂, —P(R²)O₂H, and phosphate, all of which can be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R^{2β}, R^{3β}, R^{5β} and R^{6β} are independently selected from the group consisting of hydrogen, nitro, alkyl, lower alkyl, alkenyl, alkynyl, carbocycle, cycloalkyl, cycloalkylalkyl, haloalkyl, aryl, arylalkyl, heteroaryl, heteroaryl lower alkyl, heterocyclic, heterocyclic lower alkyl, alkylthioalkyl, cycloalkylthioalkyl, arylthio lower alkyl, aralkyl lower thioalkyl, heteroarylthio lower alkyl, heteroaralkyl lower thioalkyl, heterocyclicthio lower alkyl, heterocyclicalkyl lower thioalkyl, —C(O)R², R²C(O)alkyl, aminoalkyl, cycloalkylaminoalkyl, arylamino lower alkyl, heteroarylamino lower alkyl, heterocyclicamino lower alkyl, hydroxyl, hydroxyalkyl, alkoxy, lower alkoxy, —(O(CH₂)₂)₁₋₃—O-lower alkyl, cycloalkyloxy, cycloalkylalkoxy, haloalkoxy, aryloxy, arylalkoxy, heteroaryloxy, heteroarylalkoxy, heteroaryl lower alkoxy, heterocyclicoxy, heterocyclicalkoxy, heterocyclic lower alkoxy, —OC(R¹)₂C(O)OH, —OC(R¹)₂C(O)OR², —OC(R¹)₂C(O)NH₂, —OC(R¹)₂C(O)NHR², —OC(R¹)₂C(O)N(R²)₂, —OC(R¹)₂C(O)NR⁷R⁸, amino, alkylamino, acylamino, dialkylamino, cycloalkylamino, arylamino, aralkylamino, heteroarylamino, heteroaralkylamino, heterocyclicamino, heterocyclicalkylamino, —NHR², N(R²)₂, —NR⁷R⁸, —NHC(R¹)₂C(O)OH, —NHC(R¹)₂C(O)OR², —NHC(O)R², —N(R²)C(O)R², —NHC(O)OR², —NHC(O)SR², —NHSO₂NHR², —NHSO₂R, —NHSO₂NR⁷R⁸, —N(C(O)NHR²)₂, —NR²SO₂R², —NHC(O)NHR², —NHC(O)NR⁷R⁸, —NHC(O)N(R²)₂, thiol, alkylthio, cycloalkylthio, cycloalkylalkylthio, haloalkylthio, arylthio, aralkylthio, heteroarylthio, heteroaralkylthio, heterocyclicthio, heterocyclicalkylthio, alkylsulfonyl, arylsulfonyl, haloalkylsulfonyl, —SC(R¹)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR⁷R⁸, —SO₂NHC(O)R², —SR², —SO₂NHC(O)NHR², —SO₂NHC(O)N(R²)₂, —SO₂NHC(O)NR⁷R⁸, sulfonic acid, sulfonate, sulfate, sulfinic acid, sulfenic acid, cyano, tetrazol-5-yl, carboxy, —C(O)OR², —C(O)NH₂, —C(O)NHR², —C(O)N(R²)₂, —C(O)NR⁷R⁸, —C(O)NHC(O)R², —C(O)NHC(O)NHR², —C(O)NHC(O)N(R²)₂, —C(O)NHC(O)NR⁷R⁸, —C(O)NHSO₂R², —C(O)NHSO₂NHR², —C(O)NHSO₂N(R²), —C(O)NHSO₂N⁷R⁸, —C(CH₃)₂C(O)OH, —(CH₂)_{y}C(O)OH, wherein y is 1, 2, 3, 4, 5, or 6, —PO₂H₂, —PO₃H₂, —P(R²)O₂H, and phosphate, all of which can be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R¹ is independently selected from the group consisting of hydrogen, lower alkyl, carbocycle, cycloalkyl, aryl, heteroaryl, heterocyclic, arylalkyl, heteroarylalkyl, and heterocyclicalkyl, wherein all may be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R² is independently selected from the group consisting of alkyl, lower alkyl, alkenyl, alkynyl, carbocycle, cycloalkyl, aryl, heteroaryl, heterocyclic, arylalkyl, heteroarylalkyl, and heterocyclicalkyl, wherein all may be substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R⁷ and R⁸ are independently selected from the group consisting of alkyl, alkenyl, heteroaryl and aryl and linked together forming a 4- to 12-membered monocyclic, bicylic, tricyclic or benzofused ring;
wherein one of R^{2β}, R^{3β}, R^{4β}, R^{5β} or R^{6β} must be a carbon-carbon linked heteroaryl;
wherein all R¹, R², R⁷ and R⁸ substituents can be optionally substituted where possible with one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)N⁷R⁸, and —C(O)N(R²)₂.

In a further embodiment, the invention is represented by Formula I or its pharmaceutically acceptable salt or ester, wherein:

R^{2α}, R^{3α}, R^{4α}, R^{5α}, R^{6α} and R^{4β}, are independently selected from the group consisting of hydrogen, halogen, nitro, alkyl, haloalkyl, heteroaryl, heterocyclic, hydroxyl, alkoxy, haloalkoxy, heteroaryloxy, heteroarylalkoxy, heterocyclicoxy, heterocyclicalkoxy, —OC(R¹)₂C(O)OH, —OC(R¹)₂C(O)OR², —OC(R¹)₂C(O)NH₂, —OC(R¹)₂C(O)NHR², —OC(R¹)₂C(O)N(R²)₂, —OC(R¹)₂C(O)NR⁷R⁸, heteroarylamino, heterocyclicamino, —NHR², N(R²)₂, —NR⁷R⁸, —NHC(O)R², —N(R²)C(O)R², —NHC(O)OR², —NHSO₂R², —NR²SO₂R², cyano, tetrazol-5-yl, carboxy, —C(O)OR², —C(O)NH₂, —C(O)NHR², —C(O)N(R²)₂, —C(O)NR⁷R⁸, all of which can be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R^{2β}, R^{3β}, R^{5β} and R^{6β} are independently selected from the group consisting of hydrogen, heteroaryl, heterocyclic, alkyl, alkoxy, and lower alkoxy, all of which can be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R¹ is independently selected from the group consisting of hydrogen, and lower alkyl, optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R² is independently selected from the group consisting of alkyl and lower alkyl, optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R⁷ and R⁸ are independently selected from the group consisting of alkyl, alkenyl, heteroaryl and aryl and linked together forming a 4- to 12-membered monocyclic, bicylic, tricyclic or benzofused ring, optionally substituted where possible with one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
wherein one of R^{2β}, R^{3β}, R^{4β}, R^{5β} or R^{6β} must be a carbon-carbon linked heteroaryl.

In another embodiment, the invention is represented by Formula I or its pharmaceutically acceptable salt or ester, wherein the compound is selected from the group consisting of:
4-[2-(4-Methoxy-3-thien-2-yl-phenyl)-acryloyl]-benzoic acid;
4-[2-(2-Methoxy-5-quinolin-3-yl-phenyl)-acryloyl]-benzoic acid;
2-{3-[1-(4-Carboxy-benzoyl)-vinyl]-4-methoxy-phenyl}-pyrrole-1-carboxylic acid tert-butyl ester;
4-[2-(2-Methoxy-5-pyridin-3-yl-phenyl)-acryloyl]-benzoic acid;
4-[2-(5-Benzo[b]thien-2-yl-2-methoxy-phenyl)-acryloyl]-benzoic acid;
4-[2-(2-Methoxy-5-thien-2-yl-phenyl)-acryloyl]-benzoic acid;
4-[2-(2-Methoxy-5-thien-2-yl-phenyl)-acryloyl]-benzoic acid;
4-[2-(2-Methoxy-5-thien-2-yl-phenyl)-acryloyl]-benzoic acid methyl ester;
N-(2-Hydroxy-1,1-bis-hydroxymethyl-ethyl)-4-[2-(2-methoxy-5-thien-2-yl-phenyl)-acryloyl]-benzamide;
1-[4-(4-Hydroxy-piperidine-1-carbonyl)-phenyl]-2-(2-methoxy-5-thien-2-yl-phenyl)-propenone;
1-(4-Fluoro-phenyl)-2-(2-methoxy-5-thien-2-yl-phenyl)-propenone;
2-(2-Methoxy-5-thien-2-yl-phenyl)-1-(4-pyrrolidin-1-yl-phenyl)-propenone;
1-(4-Hydroxy-phenyl)-2-(2-methoxy-5-thien-2-yl-phenyl)-propenone;
2-(2-Methoxy-5-thien-2-yl-phenyl)-1-(4-nitro-phenyl)-propenone;
N-{4-[2-(2-Methoxy-5-thien-2-yl-phenyl)-acryloyl]-phenyl}-methanesulfonamide;
N-{4-[2-(2-Methoxy-5-thien-2-yl-phenyl)-acryloyl]-phenyl}-N-methyl-Methane-sulfonamide;
N-{4-[2-(2-Methoxy-5-thien-2-yl-phenyl)-acryloyl]-phenyl}-isobutyramide;
2-(2-Methoxy-5-thien-2-yl-phenyl)-1-[4-(pyrimidin-2-ylamino)-phenyl]-propenone;
2-{4-[2-(2-Methoxy-5-thien-2-yl-phenyl)-acryloyl]-phenylamino}-nicotinic acid ethyl ester;
2-{4-[2-(2-Methoxy-5-thien-2-yl-phenyl)-acryloyl]-phenylamino}-nicotinic acid;
2-(2-Methoxy-5-thien-2-yl-phenyl)-1-[4-(pyrazin-2-ylamino)-phenyl]-propenone;
3,5-Dimethyl-isoxazole-4-sulfonic acid {4-[2-(2-methoxy-5-thien-2-yl-phenyl)-acryloyl]-phenyl}-amide;
Isoxazole-5-carboxylic acid {4-[2-(2-methoxy-5-thien-2-yl-phenyl)-acryloyl]-phenyl}-amide;
1-Methyl-1H-pyrrole-2-carboxylic acid {4-[2-(2-methoxy-5-thien-2-yl-phenyl)-acryloyl]-phenyl}-amide;
4-[2-(3,4-Dimethoxy-5-thien-2-yl-phenyl)-acryloyl]-benzoic acid methyl ester;
4-[2-(3,4-Dimethoxy-5-thien-2-yl-phenyl)-acryloyl]-benzoic acid;
4-[2-(3,4-Dimethoxy-5-thien-2-yl-phenyl)-acryloyl]-N-(2-morpholin-4-yl-ethyl) benzamide;
2-(5-Benzo[b]thien-2-yl-2,4-dimethoxy-phenyl)-1-(4-fluoro-phenyl)-propenone;
4-[2-(2,4-Dimethoxy-5-thien-2-yl-phenyl)-acryloyl]-benzoic acid;
3-[2-(2-Methoxy-5-thien-2-yl-phenyl)-acryloyl]-benzoic acid;
1-(4-tert-Butyl-phenyl)-2-(2-methoxy-5-thien-2-yl-phenyl)-propenone;
2-(2-Methoxy-5-thien-2-yl-phenyl)-1-(4-trifluoromethyl-phenyl)-propenone;
4-[2-(2-Isopropoxy-5-thien-2-yl-phenyl)-acryloyl]-benzoic acid;
4-{2-[2-(2-Oxo-2-piperidin-1-yl-ethoxy)-5-thien-2-yl-phenyl]-acryloyl}-benzoic acid;
4-{2-[2-(2-Piperidin-1-yl-ethoxy)-5-thien-2-yl-phenyl]-acryloyl}-benzoic acid hydrochloride salt; and
4-[2-(2-Methoxy-5-thien-3-yl-phenyl)-acryloyl]-benzoic acid.

In another embodiment the present invention provides a pharmaceutical composition for the treatment and/or prophylaxis of a disease or disorder involving VCAM-1, or a pharmaceutically acceptable salt or prodrug thereof, optionally with a pharmaceutically acceptable carrier or diluent; and optionally with one or more other effective therapeutic agents.

In a further embodiment of the present invention, a method for the treatment of a disease or disorder involving VCAM-1 in a host is provided, the method comprising administering an effective amount of a compound disclosed herein, or a pharmaceutically acceptable salt or prodrug thereof, optionally with a pharmaceutically acceptable carrier, excipient or diluent, and optionally in combination and/or alternation with one or more other effective therapeutic agents.

In another embodiment of the present invention, the use of a compound or compounds as disclosed herein, or a pharmaceutically acceptable salt or prodrug thereof, optionally with a pharmaceutically acceptable carrier or diluent, for the treatment of a disease or disorder involving VCAM-1 in a host, and optionally in combination and/or alternation with one or more other effective therapeutic agents, is provided.

A further embodiment of the present invention is the use of a compound or compounds as disclosed herein, or a pharmaceutically acceptable salt or prodrug thereof, optionally in combination and/or alternation with one or more other effective therapeutic agents, and optionally with a pharmaceutically acceptable carrier or diluent, in the manufacture of a medicament for the treatment of a diseases or disorders involving VCAM-1 in a host.

Schemes

The compounds of the present invention can be readily prepared by those skilled in the art of organic synthesis using commonly known methods, many of which are described by Smith, M. B., and March, J. in Advanced Organic Chemistry: Reactions, Mechanisms, and Structure, 5^{th} Edition (Wiley Interscience, New York, 2000), incorporated herein by reference. The following schemes are non-limiting embodiments that describe the invention. For the purposes of the schemes, R, and R′ are considered independent for each scheme and can be any substituent including hydrogen. R, and R′ can be suitably functionalized and can represent multiple substitutions. In addition two adjacent R and R′ can form a ring. X, independently for each scheme, represents Cl, Br, or I. HetAr represents a suitably substituted heteroaryl.

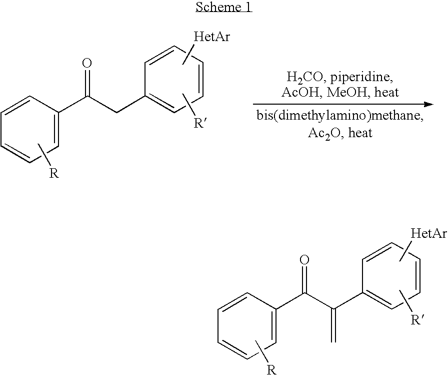

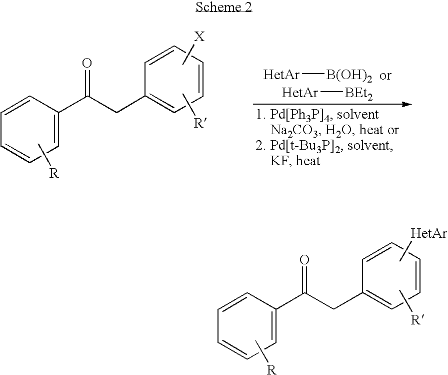

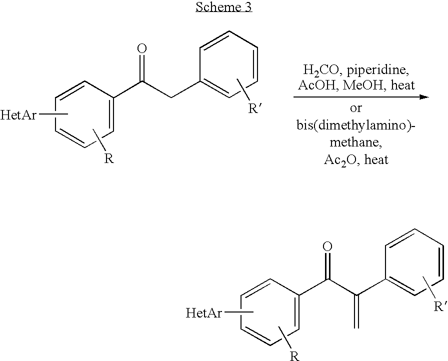

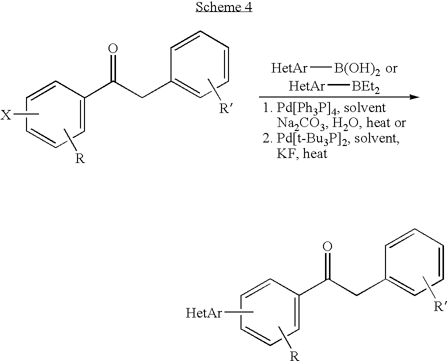

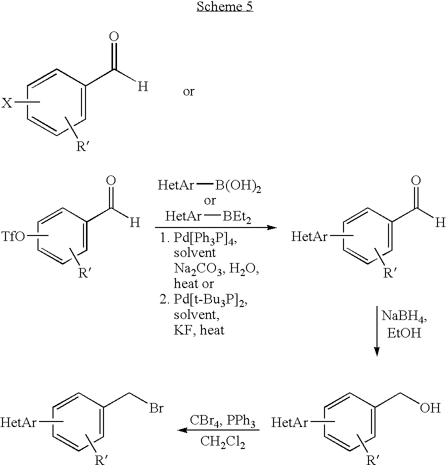

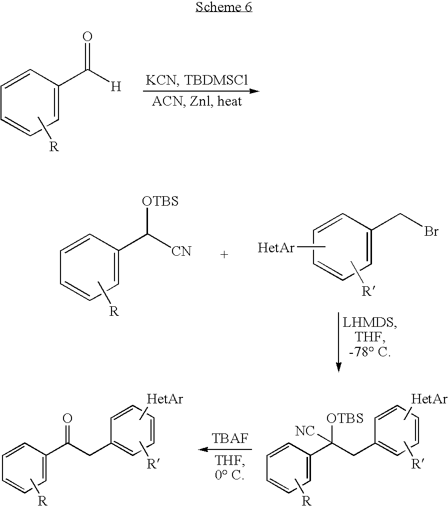

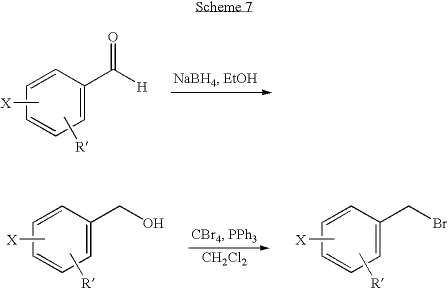

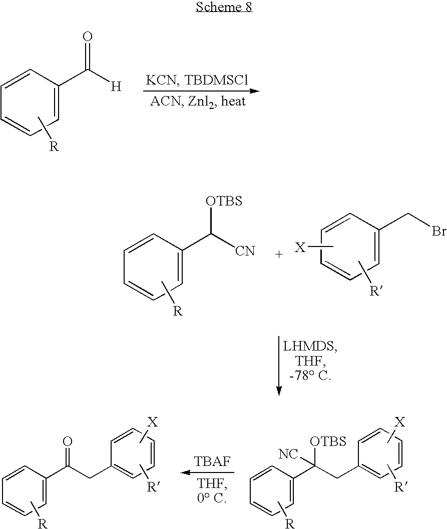

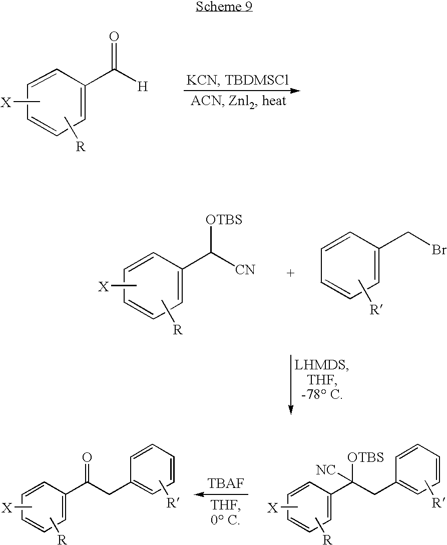

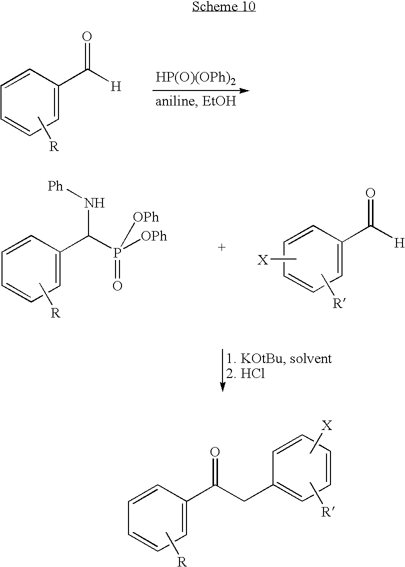

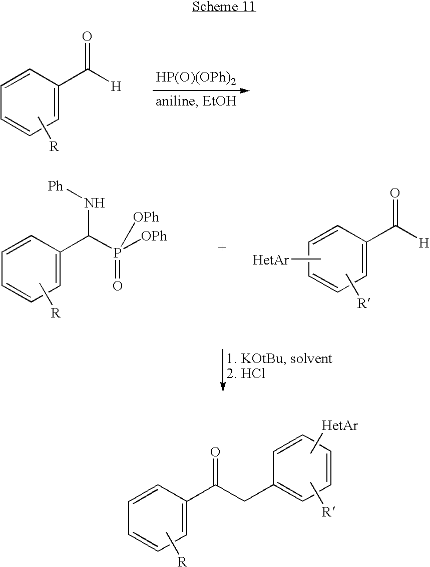

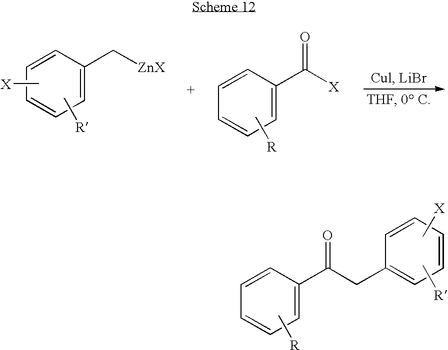

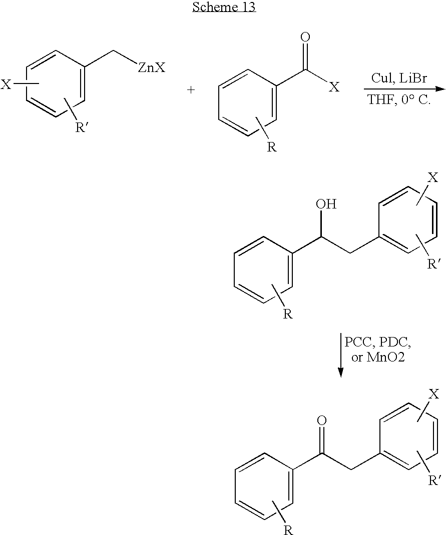

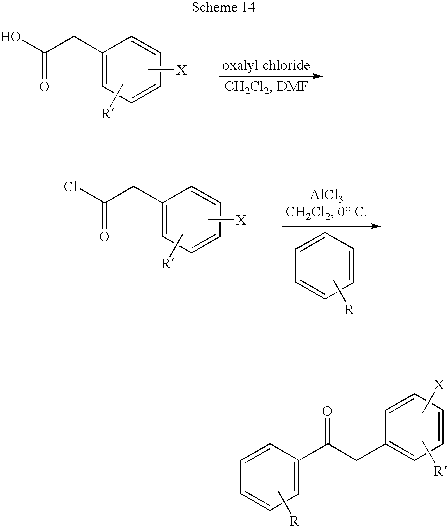

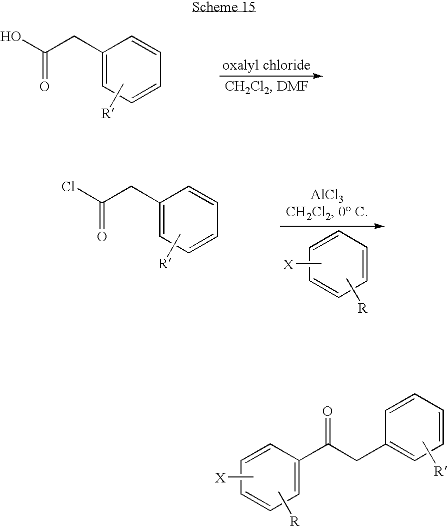

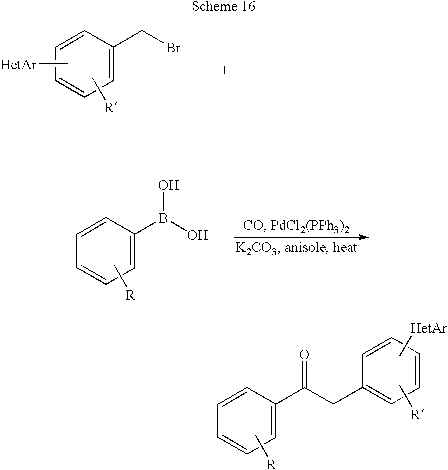

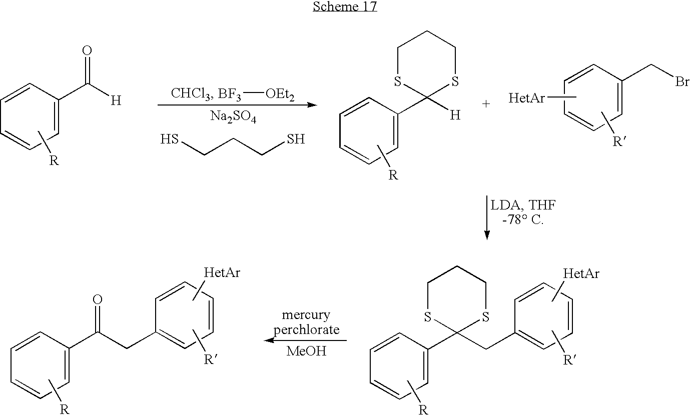

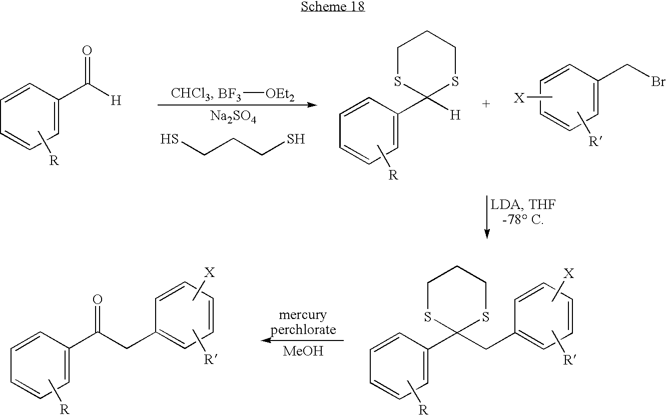

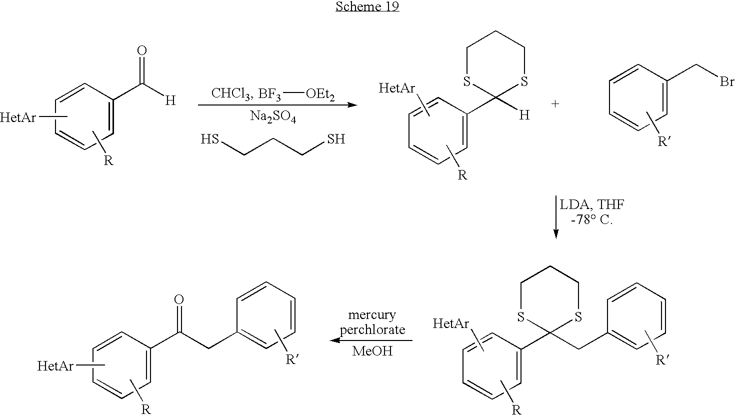

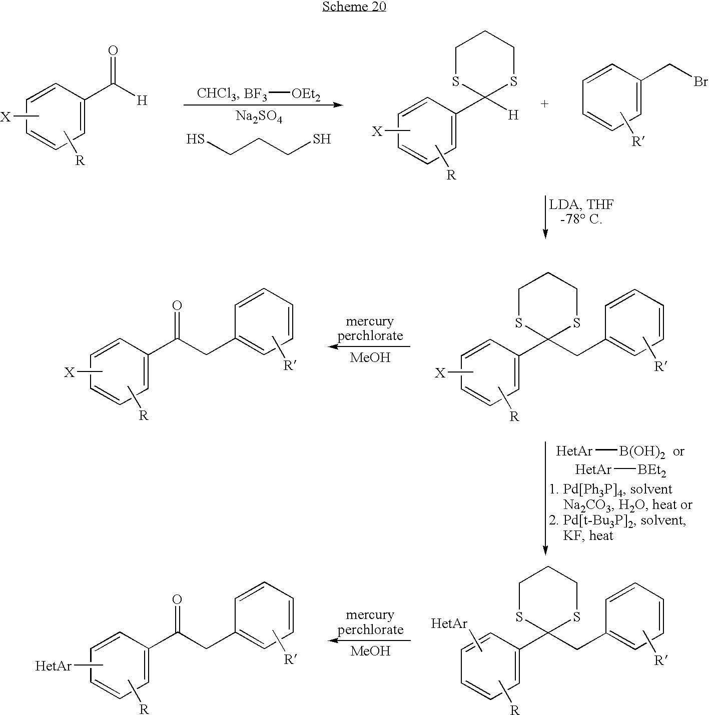

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the scope of the invention.

### EXAMPLES

### Example 1

### 4-[2-(4-Methoxy-3-thien-2-yl-phenyl)-acryloyl]-benzoic acid

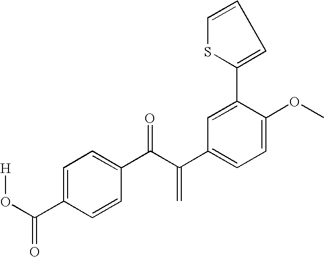

Ex-1A: In a 500 mL round-bottom flask 4-formyl methylbenzoate (6.80 g, 41.42 mmol) was combined with KCN (10.8 g, 165.8 mmol), MeCN (100 mL), ZnI₂ (2.6 g, 8.15 mmol), and TBSCl (7.5 g, 49.8 mmol) and the resulting mixture was stirred rapidly and heated to 65° C.; HPLC indicated the reaction was complete after 22 h. The mix was diluted with ether and filtered. The filtrate was diluted with EtOAc, washed with ½-saturated brine, dried over Na₂SO₄, filtered, concentrated, and dried. The crude material was purified by silica gel chromatography (33% CH₂Cl₂ in hexanes) to provide 5.19 g (41%) of 4-[(tert-butyl-dimethyl-silanyloxy)-cyano-methyl]-benzoic acid methyl ester as a colorless oil. ¹H-NMR (CDCl₃) δ 8.10 (d, J=8.3 Hz, 2H), 7.55 (d, J=8.3 Hz, 2H), 5.56 (s, 1H), 3.94 (s, 3H), 0.95 (s, 9H), 0.25 (s, 3H), 0.17 (s, 3H).

Ex-1B: In a 1 L round-bottom flask 3-bromo-4-methoxy-benzaldehyde (12.23 g, 56.87 mmol) was combined with thiophene 2-boronic acid (8.38 g, 65.5 mmol) and dimethoxyethane (310 mL). The resulting solution was purged subsurface with nitrogen while adding 2 M Na₂CO₃ (120 mL, 240 mmol) and tetrakis (triphenylphospine) palladium (0) (6.58 g, 5.69 mol). The solution was purged another 10 min, then heated to reflux; HPLC indicated the reaction was complete after 5 h. The cooled reaction mixture was diluted with H₂O and extracted with EtOAc. The organic phase was washed with brine, dried over Na₂SO₄, filtered, concentrated, and dried. The crude material was purified by silica gel chromatography (20-25% EtOAc in hexanes) to provide 11.31 g (91%) of 4-methoxy-3-thien-2-yl-benzaldehyde as an orange oil. ¹H-NMR (CDCl₃) δ 9.94 (s, 1H), 8.16 (d, J=2.4 Hz, 1H), 7.80 (dd, J=8.8, 2.4 Hz, 1H), 7.57 (dd, J=3.7, 1.5 Hz, 1H), 7.38 (d, J=5.4 Hz, 1H), 7.12 (dd, J=5.4, 3.7 Hz, 1H), 7.09 (d, J=8.8 Hz, 1H), 4.03 (s, 3H).

Ex-1C: In a 100 mL round-bottom flask 4-methoxy-3-thien-2-yl-benzaldehyde obtained from Ex-1B (1.32 g, 6.05 mmol) was combined with THF (21 mL) and EtOH (17 mL). NaBH₄ (0.25 g, 6.61 mmol) was added in portions. After 3 h, HPLC indicated the reaction was complete. The mixture was poured into H₂O and extracted with CH₂Cl₂. The organic phase was washed with brine, dried over Na₂SO₄, filtered, concentrated, and dried to an off-white solid. The resulting (4-methoxy-3-thien-2-yl-phenyl)-methanol was used without purification (1.27 g, 95%). ¹H-NMR (CDCl₃) δ 7.65 (d, J=2.2 Hz, 1H), 7.52 (d, J=3.7 Hz, 1H), 7.33 (d, J=5.2 Hz, 1H), 7.27 (dd, J=8.4, 2.2 Hz, 1H), 7.09 (dd, J=5.2, 3.7 Hz, 1H), 6.97 (d, J=8.4 Hz, 1H), 4.67 (d, J=4.2 Hz, 2H), 3.94 (s, 3H).

Ex-1D: In a 200 mL round-bottom flask (4-methoxy-3-thien-2-yl-phenyl)-methanol obtained from Ex-1C (1.27 g, 5.77 mmol) was combined with CH₂Cl₂ (30 mL) and carbon tetrabromide (2.09 g, 6.30 mmol). The solution was cooled in a 0° C. bath and triphenylphosphine (1.67 g, 6.37 mmol) was added in portions. The reaction was stirred for 1 h 20 min, then warmed to room temperature. After 1 h 25 min, TLC indicated the reaction was complete and the reaction mix was concentrated in vacuo and stored in the freezer overnight. The crude material was purified by silica gel chromatography (15-20% EtOAc in hexanes) to provide 1.33 g (82%) of 2-(5-bromomethyl-2-methoxy-phenyl)-thiophene as a yellow oil which was used immediately in the following reaction.

¹H-NMR (CDCl₃) δ 7.66 (d, J=2.1 Hz, 1H), 7.50 (dd, J=3.9, 1.8 Hz, 1H), 7.34 (d, J=4.6 Hz, 1H), 7.29 (dd, J=8.8, 2.1 Hz, 1H), 7.09 (dd, J=4.6, 3.9 Hz, 1H), 6.94 (d, J=8.8 Hz, 1H), 4.53 (s, 2H), 3.93 (s, 3H).

Ex-1E: In a 50 mL round-bottom flask 4-[(tert-butyl-dimethyl-silanyloxy)-cyano-methyl]-benzoic acid methyl ester obtained from Ex-1A (1.31 g, 4.29 mmol) was combined with THF (12 mL). The solution was cooled in a −78° C. bath and LHMDS (1 M in THF, 4.7 mL) was added dropwise. The resulting solution was stirred at −78° C. for 15 min. In a second 50 mL round-bottom flask freshly-prepared 2-(5-bromomethyl-2-methoxy-phenyl)-thiophene obtained from Ex-1D (˜1.33 g, ˜4.7 mmol) was combined with THF (10 mL) and the solution was cooled in a −78° C. bath. Via cannula, the anion solution was transferred to the second 50 mL flask in a slow stream. Approximately halfway through the addition, the cold baths were removed. After 1 h, HPLC indicated very little cyanohydrin remained. The solution was left stirring overnight, then diluted with aqueous NH₄Cl and extracted with EtOAc. The organic phase was washed with brine, dried over Na₂SO₄, filtered, concentrated, and dried. The crude material was purified by silica gel chromatography (10% EtOAc in hexanes) to provide 1.14 g (52%) of 4-[1-(tert-butyl-dimethyl-silanyloxy)-1-cyano-2-(4-methoxy-3-thien-2-yl-phenyl)-ethyl]-benzoic acid methyl ester as a faint yellow solid. ¹H-NMR (CDCl₃) δ 8.05 (d, J=8.7 Hz, 2H), 7.56 (d, J=8.7 Hz, 2H), 7.36 (d, J=3.7 Hz, 1H), 7.29-7.31 (m, 2H), 7.05 (dd, J=5.4, 3.7 Hz, 1H), 6.99 (dd, J=8.1, 2.4 Hz, 1H), 6.85 (d, J=8.1 Hz, 1H), 3.94 (s, 3H), 3.91 (s, 3H), 3.25 (d, J=13.8 Hz, 1H), 3.13 (d, J=13.8 Hz, 1H), 0.90 (s, 9H), 0.05 (s, 3H), −0.09 (s, 3H).

Ex-1F: In a 25 mL round-bottom flask 4-[1-(tert-butyl-dimethyl-silanyloxy)-1-cyano-2-(4-methoxy-3-thien-2-yl-phenyl)-ethyl]-benzoic acid methyl ester obtained from Ex-1E (1.14 g, 2.25 mmol) was combined with THF (14 mL). The solution was cooled in a 0° C. bath and TBAF (1 M in THF, 2.3 mL) was added dropwise. After 10 min HPLC indicated the reaction was complete. The reaction mixture was diluted with aqueous NaHCO₃ and extracted with EtOAc. The organic phase was washed with brine, dried over Na₂SO₄, filtered, concentrated, and dried. The crude material was purified by silica gel chromatography (20-25% EtOAc in hexanes) to provide 712 mg (86%) of 4-[2-(4-methoxy-3-thien-2-yl-phenyl)-acetyl]-benzoic acid methyl ester as a yellow oil. ¹H-NMR (CDCl₃) δ 8.12 (d, J=8.5 Hz, 2H), 8.06 (d, J=8.5 Hz, 2H), 7.51 (d, J=2.1 Hz, 1H), 7.47 (dd, J=3.6, 1.3 Hz, 1H), 7.32 (dd, J=5.2, 1.3 Hz, 1H), 7.14 (dd, J=8.4, 2.1 Hz, 1H), 7.07 (dd, J=5.2, 3.6 Hz, 1H), 6.94 (d, J=8.4 Hz, 1H), 4.29 (s, 2H), 3.94 (s, 3H), 3.91 (s, 3H).

Ex-1G: In a 200 mL round-bottom flask 4-[2-(4-methoxy-3-thien-2-yl-phenyl)-acetyl]-benzoic acid methyl ester obtained from Ex-1F (481 mg, 1.31 mmol) was combined with THF (8 mL), H₂O (2 mL), and MeOH (2 mL). 5 N NaOH (3 mL) was added dropwise and the solution was stirred at room temperature overnight, at which point HPLC indicated conversion of starting material to multiple species. The solution was acidified to pH˜2 with 1 N HCl, then extracted with CH₂Cl₂ (with a small amount of MeOH added). The organic phase was washed with ½-saturated brine, dried over Na₂SO₄, filtered, concentrated, and dried. The crude material was purified by silica gel chromatography (5% MeOH in CH₂Cl₂) to provide 96 mg (21%) of 4-[2-(4-methoxy-3-thien-2-yl-phenyl)-acetyl]-benzoic acid as a yellow solid [in addition, 47 mg of 4-methoxy-3-thien-2-yl-benzoic acid was isolated as a reaction byproduct]. ¹H-NMR (CDCl₃) δ 8.18 (d, J=8.3 Hz, 2H), 8.09 (d, J=8.3 Hz, 2H), 7.52 (d, J=2.1 Hz, 1H), 7.47 (d, J=3.7 Hz, 1H), 7.32 (d, J=5.5 Hz, 1H), 7.14 (dd, J=8.8, 2.1 Hz, 1H), 7.07 (dd, J=5.5, 3.7 Hz, 1H), 6.95 (d, J=8.8 Hz, 1H), 4.30 (s, 2H), 3.91 (s, 3H).

Ex-1H: In a 10 mL round-bottom flask 4-[2-(4-methoxy-3-thien-2-yl-phenyl)-acetyl]-benzoic acid obtained from Ex-1G (103 mg, 0.273 mmol) was combined with THF (4 mL), formaldehyde (37 wt % in H₂O, 220 μL, 2.95 mmol), piperidine (3 μL, 0.03 mmol), and AcOH (3 μL, 0.05 mmol). The solution was heated at 65° C.; HPLC indicated the reaction was complete after 4 h 15 min. The solution was cooled to room temperature, diluted with H₂O, and extracted with EtOAc. The organic phase was washed with brine, dried over Na₂SO₄, filtered, concentrated, and dried. The crude material was purified by slurrying (EtOH/hexanes) and filtration to provide 54 mg (55%) of the title compound as an off-white solid, mp 163-164° C. ¹H-NMR (CDCl₃) δ 8.16 (d, J=8.0 Hz, 2H), 7.97 (d, J=8.0 Hz, 2H), 7.70 (d, J=2.4 Hz, 1H), 7.46 (d, J=3.6 Hz, 1H), 7.34 (d, J=5.1 Hz, 1H), 7.27 (dd, J=8.7, 2.4 Hz, 1H), 7.08 (dd, J=5.1, 3.6 Hz, 1H), 6.96 (d, J=8.7 Hz, 1H), 6.12 (s, 1H), 5.68 (s, 1H), 3.94 (s, 3H). HRMS (EI⁺) m/z: calc. 364.0769, found 364.0765.

### Example 2

### 4-[2-(2-Methoxy-5-quinolin-3-yl-phenyl)-acryloyl]-benzoic acid

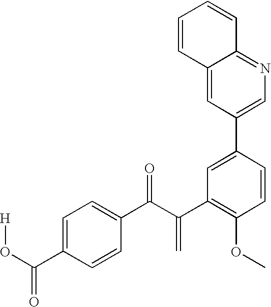

Ex-2A: In a 200 mL round-bottom flask 4-formyl methylbenzoate (11.65 g, 70.97 mmol) was combined with EtOH (50 mL). The solution was cooled in a 0° C. bath and a solution of aniline (6.5 mL, 71.3 mmol) in EtOH (12 mL) was added. Additional EtOH (25 mL) was added over 1 h as the resulting mixture became thick. After 2.5 h, TLC indicated the starting material had been consumed. A solution of diphenyl phosphite (16.5 mL, 85.7 mmol) in EtOH (15 mL) was added and the mixture was allowed to warm to room temperature. The reaction was left stirring overnight, at which point HPLC indicated the reaction was complete. Approximately ⅓ of the solution was removed in vacuo and the resulting mixture was diluted with MTBE. The mixture was mixed thoroughly and filtered. The white solid was dried to provide 25.00 g (74%) of 4-[(diphenoxy-phosphoryl)-phenylamino-methyl]-benzoic acid methyl ester. ¹H-NMR (CDCl₃) δ 8.02 (d, J=7.9 Hz, 2H), 7.63 (dd, J=8.7, 3.3 Hz, 2H), 7.06-7.31 (m, 10H), 6.89-6.92 (m, 2H), 6.76 (t, J=7.2 Hz, 1H), 6.60 (d, J=7.9 Hz, 2H), 5.18 (dd, J=25.2, 8.4 Hz, 1H), 4.91 (t, J=9.6 Hz, 1H), 3.90 (s, 3H).

Ex-2B: 2-Methoxy-5-quinolin-3-yl-benzaldehyde was prepared from 3-quinoline boronic acid in a similar manner as described in Ex-1B. The crude material was purified by silica gel chromatography (4% MeOH in CH₂Cl₂) to provide 0.80 g (74%) of pure product as an off-white solid. ¹H-NMR (CDCl₃) δ 10.57 (s, 1H), 9.17 (d, J=2.1 Hz, 1H), 8.31 (d, J=2.1 Hz, 1H), 8.21 (d, J=2.5 Hz, 1H), 8.13 (d, J=8.4 Hz, 1H), 7.93 (dd, J=8.5, 2.5 Hz, 1H), 7.87 (d, J=8.4 Hz, 1H), 7.73 (m, 1H), 7.59 (dt, J=8.5, 1.2 Hz, 1H), 7.17 (d, J=8.5 Hz, 1H), 4.03 (s, 3H).

Ex-2C: In a 200 mL round-bottom flask 4-[(diphenoxy-phosphoryl)-phenylamino-methyl]-benzoic acid methyl ester obtained from Ex-2A (1.44 g, 3.04 mmol) was combined with 2-methoxy-5-quinolin-3-yl-benzaldehyde obtained from Ex-2B (0.80 g, 3.04 mmol) and THF (16 mL). Potassium tert-butoxide (1 M in THF, 3.1 mL) was added dropwise over 10 min. After 4.5 h, HPLC indicated the starting material had been consumed. 1 N HCl (6.5 mL) was added dropwise and the solution stirred for 10 min. HPLC indicated the reaction intermediate had been consumed. The reaction was diluted with H₂O and saturated, aqueous NaHCO₃ (the pH was adjusted to ˜7), then extracted with EtOAc. The organic phase was washed with brine, dried over Na₂SO₄, filtered, concentrated, and dried. The crude material was purified by silica gel chromatography (4% MeOH in CH₂Cl₂) to provide 1.20 g of impure 4-[2-(2-methoxy-5-quinolin-3-yl-phenyl)-acetyl]-benzoic acid methyl ester as a yellow foam. ¹H-NMR (CDCl₃) δ 9.14 (d, J=2.2 Hz, 1H), 8.25 (d, J=2.2 Hz, 1H), 8.11-8.18 (m, 4H), 7.85-7.88 (m, 2H), 7.55-7.73 (m, 4H), 7.05 (d, J=7.8 Hz, 1H), 4.41 (s, 2H), 3.96 (s, 3H), 3.86 (s, 3H).

Ex-2D: In a 200 mL round-bottom flask 4-[2-(2-methoxy-5-quinolin-3-yl-phenyl)-acetyl]-benzoic acid methyl ester obtained from Ex-2C (impure, 1.20 g, ˜2.9 mmol) was combined with THF (15 mL) and MeOH (8 mL). The solution was purged subsurface with nitrogen and 1 N NaOH (6 mL) was added dropwise. The solution was purged with nitrogen for several more min and then stirred at room temperature; HPLC indicated the reaction was complete after 1 h. The solution was acidified to pH ˜2 with 1 N HCl. The pH of the solution was adjusted to ˜6 by addition of saturated, aqueous NaHCO₃; the mixture was then diluted with H₂O and filtered. The filter cake was washed with H₂O and a small amount of CH₂Cl₂. The isolated yellow solid was purified by silica gel chromatography (10% MeOH in CH₂Cl₂) to provide 0.24 g (2 steps, 20%) of 4-[2-(2-methoxy-5-quinolin-3-yl-phenyl)-acetyl]-benzoic acid as a faint yellow solid. ¹H-NMR (DMSO-d₆) δ 9.23 (d, J=2.2 Hz, 1H), 8.57 (d, J=2.2 Hz, 1H), 8.15 (d, J=8.9 Hz, 2H), 8.08 (d, J=8.9 Hz, 2H), 8.03 (d, J=8.4 Hz, 2H), 7.80-7.85 (m, 2H), 7.75 (t, J=7.3 Hz, 1H), 7.64 (t, J=7.3 Hz, 1H), 7.17 (d, J=7.5 Hz, 1H), 4.48 (s, 2H), 3.78 (s, 3H).

Ex-2E: In a 10 mL round-bottom flask 4-[2-(2-methoxy-5-quinolin-3-yl-phenyl)-acetyl]-benzoic acid obtained from Ex-2D (0.24 g, 0.60 mmol) was combined with THF (8 mL), MeOH (4 mL), formaldehyde (37 wt % in H₂O, 500 μL, 6.71 mmol), piperidine (4.5 μL, 0.046 mmol), and AcOH (4.5 μL, 0.079 mmol). The solution was heated at 65° C. for 2 h 40 min, at which point NMR indicated ˜40% conversion. Additional formaldehyde (37 wt % in H₂O, 350 μL, 4.70 mmol) was added and the solution left heating overnight, at which point NMR indicated the reaction was complete. The solution was cooled to room temperature, diluted with H₂O, acidified to pH˜2 with 1 N HCl, and then basified to pH˜6 with saturated, aqueous NaHCO₃. The solution was extracted with CH₂Cl₂ (with a small amount of MeOH added). The organic phase was washed with ½-saturated brine, dried over Na₂SO₄, filtered, concentrated, and dried. The crude material was purified by slurrying (EtOH/hexanes) and filtration to provide 185 mg (75%) of the title compound as a white solid, mp 232° C. (dec.). ¹H-NMR (DMSO-d₆) δ 9.33 (d, J=2.4 Hz, 1H), 8.70 (d, J=2.4 Hz, 1H), 8.06 (d, J=8.2 Hz, 2H), 8.03 (d, J=8.2 Hz, 2H), 7.99 (d, J=2.4 Hz, 1H), 7.88-7.92 (m, 3H), 7.77 (dt, J=6.6, 1.2 Hz, 1H), 7.65 (t, J=6.6 Hz, 1H), 7.13 (d, J=8.4 Hz, 1H), 6.32 (s, 1H), 5.82 (s, 1H), 3.56 (s, 3H). HRMS (EI⁺) m/z: calc. 409.1314, found 409.1309.

### Example 3

### 2-{3-[1-(4-Carboxy-benzoyl)-vinyl]-4-methoxy-phenyl}-pyrrole-1-carboxylic acid tert-butyl ester

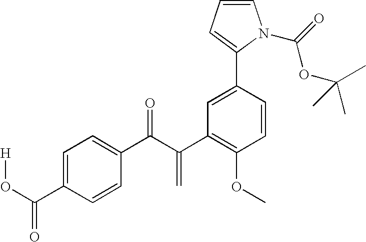

Ex-3A: 2-(3-Formyl-4-methoxy-phenyl)-pyrrole-1-carboxylic acid tert-butyl ester was prepared from 1-N-(boc)-pyrrole-2-boronic acid in a similar manner as described in Ex-1B. The crude material was purified by silica gel chromatography (20-30% EtOAc in hexanes) to provide 0.86 g (77%) of pure product as a yellow-orange, crystalline solid. ¹H-NMR (CDCl₃) δ 10.49 (s, 1H), 7.82 (d, J=2.7 Hz, 1H), 7.56 (dd, J=8.2, 2.7 Hz, 1H), 7.34 (dd, J=2.7, 0.9 Hz, 1H), 6.99 (d, J=8.7 Hz, 1H), 6.22 (t, J=2.7 Hz, 1H), 6.16-6.18 (m, 1H), 3.96 (s, 3H), 1.41 (s, 9H).

Ex-3B: 2-{4-methoxy-3-[2-(4-methoxycarbonyl-phenyl)-2-oxo-ethyl]-phenyl}-pyrrole-1-carboxylic acid tert-butyl ester was prepared by condensing 2-(3-formyl-4-methoxy-phenyl)-pyrrole-1-carboxylic acid tert-butyl ester obtained from Ex-3A with 4-[(diphenoxy-phosphoryl)-phenylamino-methyl]-benzoic acid methyl ester obtained from Ex-2A in a similar manner as described in Ex-2C. The crude material was purified by silica gel chromatography (25% EtOAc in hexanes) to provide 0.56 g of semi-pure product as a yellow-orange oil. ¹H-NMR (CDCl₃) δ 8.11 (d, J=8.8 Hz, 2H), 8.06 (d, J=8.8 Hz, 2H), 7.16-7.31 (m, 3H), 6.86 (d, J=8.4 Hz, 1H), 6.19 (t, J=3.0 Hz, 1H), 6.11-6.13 (m, 1H), 4.28 (s, 2H), 3.95 (s, 3H), 3.80 (s, 3H), 1.36 (s, 9H).

Ex-3C: In a 200 mL round-bottom flask 2-{4-Methoxy-3-[2-(4-methoxycarbonyl-phenyl)-2-oxo-ethyl]-phenyl}-pyrrole-1-carboxylic acid tert-butyl ester obtained from Ex-3B (impure, 0.56 g, ˜1.3 mmol) was combined with THF (7 mL) and MeOH (2 mL). The solution was purged subsurface with nitrogen and 1 N NaOH (2.5 mL) was added dropwise. The solution was purged with nitrogen for several more min and then stirred at room temperature; HPLC indicated the reaction was complete after 1 h. The solution was acidified to pH˜2 with 1 N HCl. The pH of the solution was adjusted to ˜6 by addition of saturated, aqueous NaHCO₃; the mixture was diluted with H₂O and extracted with CH₂Cl₂ (with a small amount of MeOH added). The organic phase was washed with ½-saturated brine, dried over Na₂SO₄, filtered, and concentrated. The crude material was purified by silica gel chromatography (5% MeOH in CH₂Cl₂) to provide 0.23 g (2 steps, 19%) of 2-{3-[2-(4-carboxy-phenyl)-2-oxo-ethyl]-4-methoxy-phenyl}-pyrrole-1-carboxylic acid tert-butyl ester as a green foam. ¹H-NMR (DMSO-d₆) δ 8.11 (d, J=8.2 Hz, 2H), 8.06 (d, J=8.2 Hz, 2H), 7.29 (d, J=1.5 Hz, 1H), 7.21 (dd, J=8.7, 1.5 Hz, 1H), 7.16 (d, J=2.4 Hz, 1H), 6.99 (d, J=8.7 Hz, 1H), 6.24 (t, J=2.4 Hz, 1H), 6.12-6.13 (m, 1H), 4.35 (s, 2H), 3.72 (s, 3H), 1.31 (s, 9H).

Ex-3D: In a 25 mL round-bottom flask 2-{3-[2-(4-carboxy-phenyl)-2-oxo-ethyl]-4-methoxy-phenyl}-pyrrole-1-carboxylic acid tert-butyl ester obtained from Ex-3C (0.23 g, 0.53 mmol) was combined with THF (7.5 mL), formaldehyde (37 wt % in H₂O, 475 μL, 6.38 mmol), piperidine (3.5 μL, 0.035 mmol), and AcOH (3.5 μL, 0.061 mmol). The solution was heated at 65° C. overnight, at which point NMR indicated the reaction was complete. The solution was cooled to room temperature, diluted with H₂O, acidified to pH˜2 with 1 N HCl, and extracted with CH₂Cl₂. The organic phase was washed with ½-saturated brine, dried over Na₂SO₄, filtered, concentrated, and dried. The crude material was purified by silica gel chromatography (5% MeOH in CH₂Cl₂) to provide 193 mg (82%) of the title compound as a light green foam, mp 90° C. (dec.). ¹H-NMR (DMSO-d₆) δ 8.00 (d, J=8.1 Hz, 2H), 7.85 (d, J=8.1 Hz, 2H), 7.40 (d, J=2.5 Hz, 1H), 7.33-7.35 (m, 1H), 7.30 (dd, J=8.5, 2.5 Hz, 1H), 6.93 (d, J=8.5 Hz, 1H), 6.26-6.28 (m, 2H), 6.08 (s, 1H), 5.71 (s, 1H), 3.49 (s, 3H), 1.37 (s, 9H). HRMS (EI⁺) m/z: calc. 447.1682, found 447.1676.

### Example 4

### 4-[2-(2-Methoxy-5-pyridin-3-yl-phenyl)-acryloyl]-benzoic acid

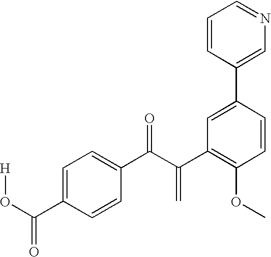

Ex-4A: 2-Methoxy-5-pyridin-3-yl-benzaldehyde was prepared from pyridine-3-boronic acid in a similar manner as described in Ex-1B. The crude material was purified by silica gel chromatography (60-90% EtOAc in hexanes) to provide 1.30 g of semi-pure product (>100%) as a yellow solid. ¹H-NMR (CDCl₃) δ 10.53 (s, 1H), 8.84 (s, 1H), 8.59 (d, J=4.2 Hz, 1H), 8.07 (d, J=2.1 Hz, 1H), 7.85-7.92 (m, 1H), 7.78-7.82 (m, 1H), 7.36 (dd, J=8.4, 5.7 Hz, 1H), 7.12 (d, J=9.0 Hz, 1H), 4.00 (s, 3H).

Ex-4B: 4-[2-(2-Methoxy-5-pyridin-3-yl-phenyl)-acetyl]-benzoic acid methyl ester was prepared by condensing 2-methoxy-5-pyridin-3-yl-benzaldehyde obtained from Ex-4A with 4-[(diphenoxy-phosphoryl)-phenylamino-methyl]-benzoic acid methyl ester obtained from Ex-2A in a similar manner as described in Ex-2C. The crude material was purified by silica gel chromatography (4-5% MeOH in CH₂Cl₂) to provide 0.68 g of semi-pure product as a yellow solid. ¹H-NMR (CDCl₃) δ 8.80 (d, J=2.7 Hz, 1H), 8.53 (dd, J=3.3, 1.8 Hz, 1H), 8.14 (d, J=8.5 Hz, 2H), 8.10 (d, J=8.5 Hz, 2H), 7.80-7.84 (m, 1H), 7.64-7.71 (m, 2H), 7.32 (dd, J=7.2, 4.2 Hz, 1H), 6.99 (d, J=8.4 Hz, 1H), 4.37 (s, 2H), 3.96 (s, 3H), 3.84 (s, 3H).

Ex-4C: In a 200 mL round-bottom flask 4-[2-(2-methoxy-5-pyridin-3-yl-phenyl)-acetyl]-benzoic acid methyl ester obtained from Ex-4B (impure, 0.68 g, ˜1.9 mmol) was combined with THF (10 mL) and MeOH (3 mL). The solution was purged subsurface with nitrogen and 1 N NaOH (3.8 mL) was added dropwise. The solution was purged with nitrogen for several more min and then stirred at room temperature; HPLC indicated the reaction was complete after 1 h. The solution was acidified to pH ˜6 with 1 N HCl. The pH of the solution was adjusted to ˜7 by addition of saturated, aqueous NaHCO₃; the mixture was then extracted with EtOAc. The organic phase was washed with brine, dried over Na₂SO₄, filtered, and concentrated. The aqueous phase was re-acidified to pH˜1 with 1 N HCl. The pH of the solution was adjusted to ˜6 by addition of saturated, aqueous NaHCO₃; the mixture was then extracted with CH₂Cl₂ (with a small amount of MeOH added). The organic phase was washed with 12-saturated brine, dried over Na₂SO₄, filtered, and concentrated. The two portions of isolated crude material were combined and purified by silica gel chromatography (10% MeOH in CH₂Cl₂) to provide 203 mg (2 steps, 20%) of 4-[2-(2-methoxy-5-pyridin-3-yl-phenyl)-acetyl]-benzoic acid as an off-white solid. ¹H-NMR (DMSO-d₆) 68.85 (d, J=1.5 Hz, 1H), 8.51 (d, J=4.5 Hz, 1H), 8.12 (d, J=9.0 Hz, 2H), 8.07 (d, J=9.0 Hz, 2H), 7.99-8.04 (m, 1H), 7.62-7.67 (m, 2H), 7.46 (dd, J=7.5, 4.5 Hz, 1H), 7.12 (d, J=8.4 Hz, 1H), 4.44 (s, 2H), 3.75 (s, 3H).

Ex-4D: The title compound was prepared from 4-[2-(2-methoxy-5-pyridin-3-yl-phenyl)-acetyl]-benzoic acid obtained from Ex-4C in a similar manner as described in Ex-2E. The crude material was purified by silica gel chromatography (10% MeOH in CH₂Cl₂) to provide 63 mg (30%) of pure product as a white solid, mp 215° C. (dec.). ¹H-NMR (DMSO-d₆) δ 8.96 (d, J=2.4 Hz, 1H), 8.55 (d, J=4.5 Hz, 1H), 8.13 (d, 7.8 Hz, 1H), 8.01 (d, J=8.2 Hz, 2H), 7.86 (d, J=8.2 Hz, 2H), 7.81 (d, J=2.7 Hz, 1H), 7.23 (dd, J=8.1, 2.7 Hz, 1H), 7.48 (dd, J=7.8, 4.5 Hz, 1H), 7.07 (d, J=8.1 Hz, 1H), 6.27 (s, 1H), 5.78 (s, 1H), 3.53 (s, 3H). HRMS (EI⁺) m/z: calc. 359.1158, found 359.1153.

### Example 5

### 4-[2-(5-Benzo[b]thien-2-yl-2-methoxy-phenyl)-acryloyl]-benzoic acid

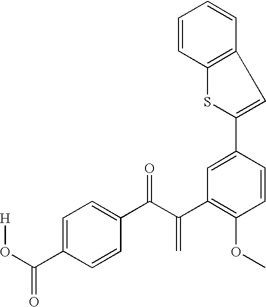

Ex-5A: 5-Benzo[b]thien-2-yl-2-methoxy-benzaldehyde was prepared from benzo[b]thiophene-2-boronic acid in a similar manner as described in Ex-1B. The crude material was purified by silica gel chromatography (20-50% EtOAc in hexanes) to provide 3.29 g (88%) of pure product as a yellow-brown crystalline solid. ¹H-NMR (CDCl₃) δ 10.51 (s, 1H), 8.18 (d, J=2.7 Hz, 1H), 7.88 (dd, J=8.2, 2.7 Hz, 1H), 7.82 (d, J=8.2 Hz, 1H), 7.76 (dd, J=8.2, 1.8 Hz, 1H), 7.52 (s, 1H), 7.28-7.38 (m, 2H), 7.06 (d, J=8.2 Hz, 1H), 3.99 (s, 3H).

Ex-5B: In a 100 mL round-bottom flask 4-[(diphenoxy-phosphoryl)-phenylamino-methyl]-benzoic acid methyl ester obtained from Ex-2A (2.33 g, 4.92 mmol) was combined with 5-benzo[b]thien-2-yl-2-methoxy-benzaldehyde obtained from Ex-5A (1.32 g, 4.92 mmol) and THF (38 mL). Potassium tert-butoxide (1 M in THF, 5.4 mL) was added dropwise over 10 min. After 2 h 15 min, HPLC indicated some aldehyde starting material remained. Additional 4-[(diphenoxy-phosphoryl)-phenylamino-methyl]-benzoic acid methyl ester (0.20 g, 0.09 mmol) was added and stirring continued for 2 h, at which point HPLC indicated less aldehyde remained. 1 N HCl (10 mL) was added dropwise and the solution stirred for 1 h. The reaction was diluted with H₂O and extracted with EtOAc. The organic phase was washed with brine, dried over Na₂SO₄, filtered, concentrated, and dried. The crude material was purified by silica gel chromatography (20-35% EtOAc in hexanes) to provide 0.86 g (42%) of 4-[2-(5-benzo[b]thien-2-yl-2-methoxy-phenyl)-acetyl]-benzoic acid methyl ester as a yellow solid. ¹H-NMR (CDCl₃) δ 8.15 (d, J=8.8 Hz, 2H), 8.09 (d, J=8.8 Hz, 2H), 7.79 (d, J=7.3 Hz, 1H), 7.72 (d, J=7.3 Hz, 1H), 7.61 (dd, J=8.8, 2.2 Hz, 1H), 7.54 (d, J=2.2 Hz, 1H), 7.42 (s, 1H), 7.26-7.33 (m, 2H), 6.94 (d, J=8.8 Hz, 1H), 4.35 (s, 2H), 3.95 (s, 3H), 3.82 (s, 3H).

Ex-5C: In a 200 mL round-bottom flask 4-[2-(5-benzo[b]thien-2-yl-2-methoxy-phenyl)-acetyl]-benzoic acid methyl ester obtained from Ex-5B (0.86 g, 2.06 mmol) was combined with THF (20 mL) and MeOH (5 mL). The solution was purged subsurface with nitrogen and 1 N NaOH (5.5 mL) was added dropwise. The solution was purged with nitrogen for several more min and then stirred at room temperature; HPLC indicated the reaction was complete after 40 min. The solution was acidified to pH˜2 with 1 N HCl, diluted with H₂O, and extracted with EtOAc. The organic phase was washed with brine, dried over Na₂SO₄, filtered, and concentrated. The crude material was purified by slurrying (5% MeOH in CH₂Cl₂) and filtration to provide 0.50 g (60%) of 4-[2-(5-benzo[b]thien-2-yl-2-methoxy-phenyl)-acetyl]-benzoic acid as a beige solid. ¹H-NMR (DMSO-d₆) δ 13.35 (bs, 1H), 8.16 (d, J=8.8 Hz, 2H), 8.10 (d, J=8.8 Hz, 2H), 7.95 (d, J=7.5 Hz, 1H), 7.81 (d, J=7.5 Hz, 1H), 7.66-7.71 (m, 3H), 7.30-7.39 (m, 2H), 7.1 (d, J=7.8 Hz, 1H), 4.46 (s, 2H), 3.76 (s, 3H).

Ex-5D: The title compound was prepared from 4-[2-(5-benzo[b]thien-2-yl-2-methoxy-phenyl)-acetyl]-benzoic acid obtained from Ex-5C in a similar manner as described in Ex-3D. The crude material was purified by slurrying (EtOH/hexanes) to provide 183 mg (71%) of pure product as a beige solid, mp 240-242° C. (dec.). ¹H-NMR (DMSO-d₆) δ 13.30 (bs, 1H), 8.03 (d, J=8.4 Hz, 2H), 7.97 (d, J=6.9 Hz, 1H), 7.80-7.90 (m, 5H), 7.74 (dd, J=8.4, 2.1 Hz, 1H), 7.32-7.42 (m, 2H), 7.06 (d, J=9.3 Hz, 1H), 6.26 (s, 1H), 5.82 (s, 1H), 3.55 (s, 3H). HRMS (EI⁺) m/z: calc. 414.0926, found 414.0927.

### Example 6

### 4-[2-(2-Methoxy-5-thien-2-yl-phenyl)-acryloyl]-benzoic acid

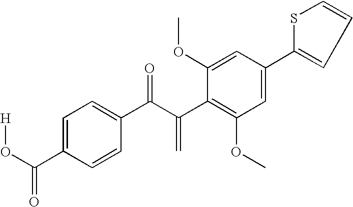

Ex-6A: A solution of 4-hydroxy-2,6-dimethoxy-benzaldehyde (2.3 g, 12.62 mmol) in dichloromethane (25 mL) was cooled to 0° C. and then dimethylamino pyridine (5.6 g, 45.84 mmol) was added in 1 portion. Triflic anhydride (2.5 mL, 14.86 mmol) was then added over 15 min while maintaining an internal temperature below 5° C. The resulting solution was aged for 1 h and then was slowly poured into cold 1 N HCl. The organic phase was dried over magnesium sulfate and concentrated under reduced pressure affording 3.76 g (73%) of methanesulfonic acid 4-formyl-3,5-dimethoxy-phenyl ester.

Ex-6B: A solution of methanesulfonic acid 4-formyl-3,5-dimethoxy-phenyl ester obtained from Ex-6A (2.71 g, 8.63 mmol) in 1,4-dioxane (35 mL) was stirred at room temperature under nitrogen for 15 min. Thiophene-2-boronic acid (1.64 g, 12.82 mmol), tetrakis(triphenylphosphine) palladium (0) (1.02 g, 0.88 mmol) and potassium phosphate (4.59 g, 21.62 mmol) were then added and the resulting mixture was heated to 95° C. under nitrogen overnight. Upon cooling to room temperature the reaction was diluted with EtOAc and water and the layers were cut. The organic phase was concentrated under reduced pressure. Silica gel chromatography (hexane/ethyl acetate, 4:1) gave 2.14 g (75%) of 2,6-dimethoxy-4-thien-2-yl-benzaldehyde, mp 168-170° C. ¹H-NMR (300 MHz, CDCl₃): 10.48 (s, 1H), 7.43 (dd, J=3.6, 1.3 Hz, 1H), 7.41 (d, J=5.3 Hz, 1H), 7.13 (dd, J=5.3, 3.6 Hz, 1H), 6.79 (s, 2H), 3.96 (s, 6H).

Ex-6C: 4-[2-(2,6-Dimethoxy-4-thien-2-yl-phenyl)-acetyl]-benzoic acid methyl ester was prepared from 4-[(diphenoxy-phosphoryl)-phenylamino-methyl]-benzoic acid methyl ester obtained from Ex-2A and 2,6-dimethoxy-4-thien-2-yl-benzaldehyde obtained from Ex-6B in a similar manner as described in Ex-5B. The crude material was purified by silica gel chromatography (20-25% EtOAc in hexanes) to provide 104 mg (50%) of semi-pure product as a light yellow solid. ¹H-NMR (CDCl₃) δ 8.10 (d, J=8.5 Hz, 2H), 8.05 (d, J=8.5 Hz, 2H), 7.27 (m, 2H), 7.07 (dd, J=4.5, 3.0 Hz, 1H), 6.78 (s, 2H), 4.33 (s, 2H), 3.94 (s, 3H), 3.81 (s, 6H).

Ex-6D: 4-[2-(2,6-Dimethoxy-4-thien-2-yl-phenyl)-acetyl]-benzoic acid was prepared from 4-[2-(2,6-dimethoxy-4-thien-2-yl-phenyl)-acetyl]-benzoic acid methyl ester obtained from Ex-6C in a similar manner as described in Ex-5C. The crude material was purified by silica gel chromatography (5-8% MeOH in CH₂Cl₂) to provide 75 mg (75%) of pure product as an off-white solid. ¹H-NMR (DMSO-d₆) δ 8.12 (d, J=8.4 Hz, 2H), 8.06 (d, J=8.4 Hz, 2H), 7.59 (dd, J=3.6, 1.3 Hz, 1H), 7.56 (dd, J=5.2, 1.3 Hz, 1H), 7.16 (dd, J=5.2, 3.6 Hz, 1H), 6.90 (s, 2H), 4.29 (s, 2H), 3.79 (s, 6H).

Ex-6E: In a 200 mL round-bottom flask 4-[2-(2,6-dimethoxy-4-thien-2-yl-phenyl)-acetyl]-benzoic obtained from Ex-6D (73 mg, 19 mmol) was combined with N,N,N′,N′-tetramethyl diaminomethane (2.2 mL, 16 mmol). Acetic anhydride (0.36 mL, 3.8 mmol) was added at 0° C. and the resulting solution was stirred at room temperature overnight, at which point NMR indicated very little conversion. Additional diamine (2 mL) and acetic anhydride (0.15 mL) were added and the solution transferred to a smaller flask and heated at 40° C. for 5 h 20 min, at which point NMR indicated the reaction almost complete. Additional acetic anhydride (0.1 mL) was added and heating continued; NMR indicated the reaction was complete after 12 h. The solution was cooled in an ice bath and 1 N HCl was added until the pH was ˜2. The whitish mix was extracted with CH₂Cl₂ (with a small amount of MeOH added). The organic phase was washed with ½-saturated brine, dried over Na₂SO₄, filtered, concentrated, and dried. The crude material was purified by silica gel chromatography (5-50% MeOH in CH₂Cl₂) to provide 25 mg (33%) of the title compound as a white solid, mp>250° C. ¹H-NMR (DMSO-d₆) δ 7.88 (d, J=8.1 Hz, 2H), 7.57-7.64 (m, 4H), 7.15 (dd, J=5.4, 3.9 Hz, 1H), 6.89 (s, 2H), 5.98 (s, 1H), 5.93 (s, 1H), 3.73 (s, 6H). HRMS (EI⁺) m/z: calc. 395.0953, found 395.0953.

### Example 7

### 1-(2,6-Dichloro-phenyl)-2-(2-methoxy-5-thien-2-yl-phenyl)-propenone

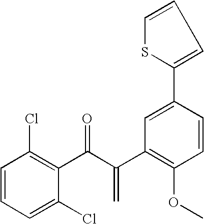

Ex-7A: In a 50 mL round-bottom flask 2,6-dichlorobenzaldehyde (3.01 g, 17.2 mmol) was combined with KCN (5.58 g, 85.7 mmol), MeCN (20 mL), ZnI₂ (1.44 g, 4.51 mmol), and TBSCl (3.10 g, 20.6 mmol) and the resulting mixture was stirred rapidly and heated to 65° C. for 17 h, at which point TLC indicated the reaction was complete. Approximately half of the solvent was removed in vacuo and the resulting mix was diluted with ether and filtered. The filtrate was diluted with EtOAc, washed with ½saturated brine, dried over Na₂SO₄, filtered, concentrated, and dried. The crude material was purified by silica gel chromatography (5% EtOAc in hexanes) to provide 4.36 g (80%) of (tert-butyl-dimethyl-silanyloxy)-(2,6-dichloro-phenyl)-acetonitrile as a faint yellow oil. ¹H-NMR (CDCl₃) δ 7.36-7.39 (m, 2H), 7.25-7.30 (m, 1H), 6.29 (s, 1H), 0.90 (s, 9H), 0.26 (s, 3H), 0.05 (s, 3H).

Ex-7B: In a 1 L round-bottom flask 5-bromo-2-methoxybenzaldehyde (15.06 g, 70.00 mmol) was combined with thiophene 2-boronic acid (10.3 g, 80.5 mmol) and dimethoxyethane (385 mL). The resulting solution was purged subsurface with nitrogen while adding 2 M Na₂CO₃ (120 mL, 240 mmol) and tetrakis(triphenylphosphine) palladium (0) (8.08 g, 6.99 mmol). The solution was purged another 10 min, then heated to reflux for 2 h, at which point HPLC indicated the reaction was complete. The cooled reaction mixture was diluted with H₂O and extracted with CH₂Cl₂. The organic phase was washed with brine, dried over Na₂SO₄, filtered, concentrated, and dried. The crude material was purified by silica gel chromatography (10-35% EtOAc in hexanes) to provide 13.66 g (89%) of 2-methoxy-5-thien-2-yl-benzaldehyde as a yellow, crystalline solid. ¹H-NMR (CDCl₃) δ 10.50 (s, 1H), 8.06 (d, J=2.8 Hz, 1H), 7.79 (dd, J=8.4, 2.8 Hz, 1H), 7.26-7.29 (m, 2H), 7.07 (dd, J=5.4, 3.9 Hz, 1H), 7.02 (d, J=8.4 Hz, 1H), 3.97 (s, 3H).

Ex-7C: In a 200 mL round-bottom flask 2-methoxy-5-thien-2-yl-benzaldehyde obtained from Ex-7B (3.52 g, 16.1 mmol) was combined with THF (55 mL) and EtOH (45 mL). NaBH₄ (0.67 g, 17.7 mmol) was added in portions. After 2.5 h, HPLC indicated the reaction was complete. The mixture was poured into H₂O and extracted with CH₂Cl₂. The organic phase was washed with brine, dried over Na₂SO₄, filtered, concentrated, and dried to an off-white solid. The resulting (2-methoxy-5-thien-2-yl-phenyl)-methanol was used without purification (3.63 g, quantitative). ¹H-NMR (CDCl₃) δ 7.50-7.55 (m, 2H), 7.21-7.23 (m, 2H), 7.06 (dd, J=5.1, 3.6 Hz, 1H), 6.89 (d, J=7.8 Hz, 1H), 4.72 (d, J=6.6 Hz, 2H), 3.90 (s, 3H).

Ex-7D: In a 200 mL round-bottom flask (2-methoxy-5-thien-2-yl-phenyl)-methanol obtained from Ex-7C (3.63 g, 16.5 mmol) was combined with CH₂Cl₂ (75 mL) and carbon tetrabromide (5.59 g, 16.9 mmol). The solution was cooled in an ice/salt bath (−5° C.) and triphenylphosphine (4.55 g, 17.4 mmol) was added in portions. When addition was complete, the reaction was warmed to room temperature. After 2 h, additional carbon tetrabromide (0.50 g, 1.51 mmol) and triphenylphosphine (0.43 g, 0.16 mmol) were added to drive the reaction. After another 2 h, the reaction mix was concentrated in vacuo and stored in the freezer overnight. The crude material was purified by silica gel chromatography (15% EtOAc in hexanes) to provide 4.30 g (92%) of 2-(3-bromomethyl-4-methoxy-phenyl)-thiophene as an off-white solid. ¹H-NMR (CDCl₃) δ 7.57 (d, J=2.5 Hz, 1H), 7.53 (dd, J=9.0, 2.5 Hz, 1H), 7.20-7.24 (m, 2H), 7.05 (dd, J=5.4, 3.9 Hz, 1H), 6.90 (d, J=9.0 Hz, 1H), 4.59 (s, 2H), 3.93 (s, 3H).

Ex-7E: In a 50 mL round-bottom flask (tert-butyl-dimethyl-silanyloxy)-(2,6-dichloro-phenyl)-acetonitrile obtained from Ex-7A (1.00 g, 3.16 mmol) was combined with THF (10 mL). The solution was cooled in a −78° C. bath and LHMDS (1 M in THF, 3.48 mL) was added dropwise. After 3 min, the cold bath was removed. After stirring an additional 3 min, the solution was placed in a 0° C. bath and stirred for 1¾ hr.

In a 25 mL round-bottom flask 2-(3-bromomethyl-4-methoxy-phenyl)-thiophene obtained from Ex-7D (821 mg, 2.90 mmol) was combined with THF (9 mL) and the solution was cooled in a 0° C. bath. Via cannula, the solution was transferred to the 50 mL flask in a slow stream. The resulting solution was allowed to warm to room temperature immediately and left stirring overnight. The solution was diluted with aqueous NH₄Cl and extracted with EtOAc. The organic phase was washed with brine, dried over Na₂SO₄, filtered, concentrated, and dried. The crude material was purified by silica gel chromatography (10% EtOAc in hexanes) to provide 0.78 g (52%) of 2-(tert-butyl-dimethyl-silanyloxy)-2-(2,6-dichloro-phenyl)-3-(2-methoxy-5-thien-2-yl-phenyl)-propionitrile as a faint yellow solid. ¹H-NMR (CDCl₃) δ 7.48 (dd, J=8.5, 2.2 Hz, 1H), 7.43 (d, J=2.2 Hz, 1H), 7.31 (d, J=7.8 Hz, 2H), 7.19 (t, J=4.8 Hz, 1H), 7.12-7.14 (m, 2H), 7.02 (dd, J=5.7, 3.9 Hz, 1H), 6.79 (d, J=8.5 Hz, 1H), 3.98 (d, J=13.5 Hz, 1H), 3.65 (s, 3H), 3.64 (d, J=13.5 Hz, 1H), 0.90 (s, 9H), 0.22 (s, 3H), 0.12 (s, 3H).

Ex-7F: In a 25 mL round-bottom flask 2-(tert-butyl-dimethyl-silanyloxy)-2-(2,6-dichloro-phenyl)-3-(2-methoxy-5-thien-2-yl-phenyl)-propionitrile obtained from Ex-7E (402 mg, 0.775 mmol) was combined with THF (7.5 mL). The solution was cooled in a 0° C. bath and TBAF (1 M in THF, 0.79 mL) was added dropwise. After 15 min HPLC indicated the reaction was complete. The reaction mixture was diluted with aqueous NaHCO₃ and extracted with EtOAc. The organic phase was washed with brine, dried over Na₂SO₄, filtered, concentrated, and dried. The crude material was purified by silica gel chromatography (15% EtOAc in hexanes) to provide 282 mg (97%) of 1-(2,6-dichloro-phenyl)-2-(2-methoxy-5-thien-2-yl-phenyl)-ethanone as a white solid. ¹H-NMR (CDCl₃) δ 7.50 (dd, J=8.5, 2.0 Hz, 1H), 7.44 (d, J=2.0 Hz, 1H), 7.18-7.32 (m, 5H), 7.04 (dd, J=5.1, 3.6 Hz, 1H), 6.87 (d, J=8.5 Hz, 1H), 4.22 (s, 2H), 3.78 (s, 3H).

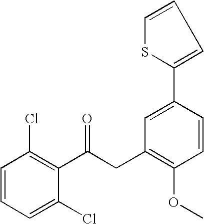

### 1-(2,6-Dichloro-phenyl)-2-(2-methoxy-5-thien-2-yl-phenyl)-ethanone

Ex-7G: In a 10 mL round-bottom flask 1-(2,6-dichloro-phenyl)-2-(2-methoxy-5-thien-2-yl-phenyl)-ethanone obtained from Ex-7F (103 mg, 0.273 mmol) was combined with THF (3.5 mL), formaldehyde (37 wt % in H₂O, 300 μL, 4.03 mmol), piperidine (3.6 μL, 0.036 mmol), and AcOH (3.6 μL, 0.063 mmol). The solution was heated at 65° C. for 4.5 h, at which point NMR indicated the reaction was complete. The solution was cooled to room temperature, diluted with H₂O, and extracted with EtOAc. The organic phase was washed with brine, dried over Na₂SO₄, filtered, concentrated, and dried. The crude material was purified by silica gel chromatography (15% EtOAc in hexanes) to provide 90 mg (85%) of the title compound as a colorless syrup. ¹H-NMR (CDCl₃) δ 7.58 (dd, J=8.1, 2.5 Hz, 1H), 7.47 (d, J=2.5 Hz, 1H), 7.22-7.37 (m, 5H), 7.06 (dd, J=5.7, 3.9 Hz, 1H), 6.95 (d, J=8.1 Hz, 1H), 6.29 (s, 1H), 6.09 (s, 1H), 3.83 (s, 3H). HRMS (EI⁺) m/z: calc. 388.0092, found 388.0081. Anal. calculated for C₂₀H₁₄Cl₂O₂S: C, 61.71; H, 3.62; S, 8.24. Found: C, 62.14; H, 3.70; S, 8.03%.

### Example 8

### 4-[2-(2-Methoxy-5-thien-2-yl-phenyl)-acryloyl]-benzoic acid

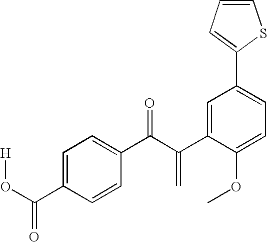

Ex-8A: In a 2-neck, 2 L round-bottom flask CuI (30.66 g, 161 mmol) was combined with LiBr (27.97 g, 322 mmol). The flask was placed in a 0° C. bath and THF (360 mL) was added. The mixture exothermed briefly to 55° C. The solution was cooled in an acetone/CO₂ bath and the temperature maintained at −30 to −40° C. while 5-bromo-2-methoxy-benzylzinc chloride (0.46 M in THF, 350 mL, 161 mmol) was added via cannula over 25 min. After addition, the cold bath was removed to allow the solution to warm to −5° C., then immediately recooled to −30° C.

In a 500 mL round-bottom flask terephthalic acid monomethyl ester chloride (95%, 35.33 g, 169 mmol) was combined with THF (115 mL). This solution was transferred to the 2 L flask via cannula while maintaining a reaction temperature of −25 to −35° C. When the transfer was complete, the resulting solution was allowed to warm slowly to room temperature. After 2.5 h, HPLC indicated the reaction was complete. The solution was cooled in a 0° C. bath and 1 N HCl (100 mL) was added slowly in portions. The solution was diluted with H₂O (250 mL) and extracted with EtOAc (1×250 mL, 2×100 mL). The organic phase was washed with H₂O (4×250 mL) and brine (1×25 mL), dried over Na₂SO₄, filtered, and concentrated to ˜400-500 mL. The resulting mix was stirred rapidly for 25 min, then filtered. The solids were washed with EtOAc (3×40 mL) and dried to provide 20.91 g pure product as a beige solid. Additional product was obtained by concentrating the filtrate and subsequently slurrying the resulting solids in EtOAc (2×). A total of 40.1 g (69%) of 4-[2-(5-bromo-2-methoxy-phenyl)-acetyl]-benzoic acid methyl ester was obtained in 3 crops; Crop 3 (4.51 g) contained an impurity. ¹H-NMR (CDCl₃) δ 8.13 (d, J=7.8 Hz, 2H), 8.06 (d, J=7.8 Hz, 2H), 7.36 (dd, J=7.9, 1.8 Hz, 1H), 7.29 (d, J=1.8 Hz, 1H), 6.75 (d, J=7.9 Hz, 1H), 4.25 (s, 2H), 3.96 (s, 3H), 3.75 (s, 3H).

Ex-8B: In a 500 mL round-bottom flask 4-[2-(5-bromo-2-methoxy-phenyl)-acetyl]-benzoic acid methyl ester obtained from Ex-8A (30.04 g, 82.71 mmol) was combined with thiophene 2-boronic acid (11.65 g, 91.05 mmol) and THF (200 mL). The resulting solution was purged subsurface with nitrogen while adding KF (14.44 g, 248.5 mmol) and bis-(tri-t-butylphosphine) palladium (0) (0.42 g, 0.82 mmol). The solution was purged another 5 min, then heated to reflux for 1.5 h, at which point HPLC indicated starting material remained. Additional thiophene 2-boronic acid (3.03 g, 23.7 mmol) was added and heating continued for 2 h. HPLC indicated starting material remained; additional thiophene 2-boronic acid (4.20 g, 32.97 mmol) and bis-(tri-t-butylphosphine) palladium (0) (0.20 g, 0.39 mmol) were added and heating continued for 35 min. HPLC indicated the reaction was nearly complete. Additional thiophene 2-boronic acid (2.10 g, 16.5 mmol) was added and heating continued for 3 h. The cooled reaction mixture was diluted with H₂O and EtOAc. The mixture was filtered over celite and the layers separated. The organic phase was dried over Na₂SO₄, filtered, concentrated, and dried. The crude material was purified by slurrying (EtOAc/hexanes) and filtration to provide 5.77 g pure product and by silica gel chromatography (20-25% EtOAc in hexanes) to provide 6.58 g (12.35 g, 41% total yield) of 4-[2-(2-methoxy-5-thien-2-yl-phenyl)-acetyl]-benzoic acid methyl ester as a light yellow crystalline solid. ¹H-NMR (CDCl₃) δ 8.12 (d, J=8.4 Hz, 2H), 8.08 (d, J=8.4 Hz, 2H), 7.50 (dd, J=8.8, 2.1 Hz, 1H), 7.42 (d, J=2.1 Hz, 1H), 7.20 (d, J=5.1 Hz, 1H), 7.18 (d, J=3.7 Hz, 1H), 7.03 (dd, J=5.1, 3.7 Hz, 1H), 6.88 (d, J=8.8 Hz, 1H), 4.32 (s, 2H), 3.95 (s, 3H), 3.80 (s, 3H).

Ex-8BB: (alternative procedure) 4-[2-(2-Methoxy-5-thien-2-yl-phenyl)-acetyl]-benzoic acid methyl ester was prepared from 4-[(diphenoxy-phosphoryl)-phenylamino-methyl]-benzoic acid methyl ester obtained from Ex-2A and 2-methoxy-5-thien-2-yl-benzaldehyde obtained from Ex-7B in a similar manner as described in Ex-5B. The crude material was purified by slurrying (20 mL of 30% EtOAc in hexanes) and filtration. The filtrate was concentrated and further purified by silica gel chromatography (20-25% EtOAc in hexanes) to provide 0.92 g (62%) of semi-pure product as a yellow solid.

Ex-8C: 4-[2-(2-Methoxy-5-thien-2-yl-phenyl)-acetyl]-benzoic acid was prepared from 4-[2-(2-methoxy-5-thien-2-yl-phenyl)-acetyl]-benzoic acid methyl ester obtained from Ex-8B or Ex-8BB in a similar manner as described in Ex-5C. The crude material was purified by repeated slurrying (MeOH/CH₂Cl₂) and filtration and by silica gel chromatography (2.5% MeOH in CH₂Cl₂) to provide a total of 10.00 g (84%) of pure product as a yellow solid. ¹H-NMR (DMSO-d₆) δ 13.34 (bs, 1H), 8.14 (d, J=8.1 Hz, 2H), 8.08 (d, J=8.1 Hz, 2H), 7.55 (dd, J=8.4, 2.7 Hz, 1H), 7.53 (s, 1H), 7.45 (d, J=5.2 Hz, 1H), 7.36 (d, J=3.6 Hz, 1H), 7.10 (dd, J=5.2, 3.6 Hz, 1H), 7.02 (d, J=8.4 Hz, 1H), 4.41 (s, 2H), 3.72 (s, 3H).

Ex-8D: The title compound was prepared from 4-[2-(2-methoxy-5-thien-2-yl-phenyl)-acetyl]-benzoic acid obtained from Ex-8C in a similar manner as described in Ex-3D. The crude material was purified by silica gel chromatography (4% MeOH in CH₂Cl₂) followed by repeated slurrying (EtOAc and EtOAc/hexanes) and filtration to provide 6.21 g (63%) of pure product as a light yellow solid. ¹H-NMR (DMSO-d₆) δ 13.30 (bs, 1H), 8.02 (d, J=9.0 Hz, 2H), 7.86 (d, J=9.0 Hz, 2H), 7.71 (d, J=2.1 Hz, 1H), 7.62 (dd, J=8.5, 2.1 Hz, 1H), 7.49-7.52 (m, 2H), 7.13 (dd, J=5.7, 3.9 Hz, 1H), 6.98 (d, J=8.5 Hz, 1H), 6.21 (s, 1H), 5.78 (s, 1H), 3.51 (s, 3H). HRMS (EI⁺) m/z: calc. 364.0769, found 364.0766. Anal. calculated for C₂₁H₁₆O₄S: C, 69.21; H, 4.43; S, 8.80. Found: C, 69.17; H, 4.53; S, 8.78%.

### Example 9

### 4-[2-(2-Methoxy-5-thien-2-yl-phenyl)-acryloyl]-benzoic acid methyl ester

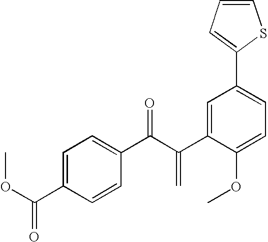

Ex-9: The title compound was prepared from 4-[2-(2-methoxy-5-thien-2-yl-phenyl)-acetyl]-benzoic acid methyl ester obtained from Ex-8B or Ex-8BB in a similar manner as described in Ex-6E. The crude material was purified by crystallization from EtOAc and hexane to provide 35 mg (36%) of desired product, mp 126-128° C. ¹H-NMR (DMSO-d₆) δ 8.02 (d, J=8.1 Hz, 2H), 7.85 (d, J=8.1 Hz, 2H), 7.68 (d, J=2.2 Hz, 1H), 7.59 (dd, J=8.3, 2.6 Hz, 1H), 7.46-7.49 (m, 2H), 7.10 (dd, J=5.4, 3.7 Hz, 1H), 6.96 (d, J=8.3 Hz, 1H), 6.18 (s, 1H), 5.76 (s, 1H), 3.85 (s, 3H), 3.47 (s, 3H). HRMS (EI⁺) m/z: calc. 378.0926, found 378.0914.

### Example 10

### N-(2-Hydroxy-1,1-bis-hydroxymethyl-ethyl)-4-[2-(2-methoxy-5-thien-2-yl-phenyl)-acryloyl]-benzamide

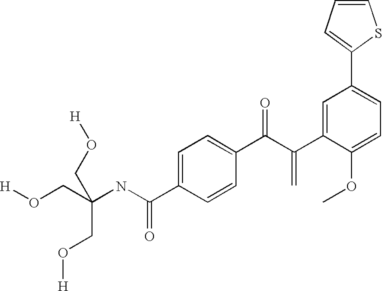

Ex-10A: In a 10 mL round-bottom flask 4-[2-(2-methoxy-5-thien-2-yl-phenyl)-acetyl]-benzoic acid obtained from Ex-8C (300 mg, 0.85 mmol) was combined with THF (2.5 mL) and H₂O (0.1 mL). 2-Amino-2-hydroxymethyl-propane-1,3-diol (110 mg, 0.91 mmol), and pyridine N-oxide (10 mg, 0.09 mmol) were added followed by 1-(3-dimethylaminopropyl)-3-ethylcarbodimide hydrochloride (170 mg, 0.89 mmol) and the solution was stirred at room temperature overnight. The reaction was diluted with EtOAc and washed sequentially with 1 N HCl (2×) and NaHCO₃. The organic layer was dried over MgSO₄, filtered, and concentrated onto silica gel. The crude material was purified by silica gel chromatography (100% EtOAc) to afford 110 mg (28%) of N-(2-hydroxy-1,1-bis-hydroxymethyl-ethyl)-4-[2-(2-methoxy-5-thien-2-yl-phenyl)-acetyl]-benzamide. ¹H-NMR (DMSO-d₆) δ 8.07 (d, J=7.9 Hz, 2H), 7.90 (d, J=7.9 Hz, 2H), 7.50-7.54 (m, 2H), 7.41-7.43 (m, 2H), 7.33 (d, J=3.4 Hz, 1H), 7.07 (dd, J=5.5, 3.9 Hz, 1H), 7.00 (d, J=7.7 Hz, 1H), 4.69 (t, J=5.7 Hz, 3H), 4.37 (s, 2H), 3.67-3.70 (m, 9H).

Ex-10B: The title compound was prepared from N-(2-hydroxy-1,1-bis-hydroxymethyl-ethyl)-4-[2-(2-methoxy-5-thien-2-yl-phenyl)-acetyl]-benzamide obtained from Ex-10A in a similar manner as described in Ex-3D. The crude material was purified by silica gel chromatography (5% MeOH in CH₂Cl₂) followed by crystallization from EtOAc and hexanes to provide 45 mg (44%) of pure product, mp 144-146° C. ¹H-NMR (DMSO-d₆) δ 7.83 (d, J=8.4 Hz, 2H), 7.78 (d, J=8.4 Hz, 2H), 7.69 (d, J=1.9 Hz, 1H), 7.59 (dd, J=8.2, 2.9 Hz, 1H), 7.46-7.49 (m, 2H), 7.36 (s, 1H), 7.11 (dd, J=5.5, 4.0 Hz, 1H), 6.96 (d, J=8.4 Hz, 1H), 6.15 (s, 1H), 5.71 (s, 1H), 4.68 (t, J=5.9 Hz, 3H), 3.66 (d, J=5.9 Hz, 6H), 3.48 (s, 3H). HRMS (EI⁺) m/z: calc. 467.1403, found 467.1394.

### Example 11

### 1-[4-(4-Hydroxy-piperidine-1-carbonyl)-phenyl]-2-(2-methoxy-5-thien-2-yl-phenyl)-propenone

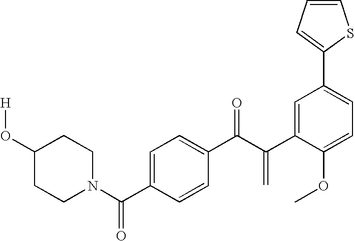

Ex-11A: 1-[4-(4-Hydroxy-piperidine-1-carbonyl)-phenyl]-2-(2-methoxy-5-thien-2-yl-phenyl)-ethanone was prepared from 4-[2-(2-methoxy-5-thien-2-yl-phenyl)-acetyl]-benzoic acid obtained from Ex-8C in a similar manner as described in Ex-10A. The crude material was purified by slurrying in EtOAc and filtering to provide 295 mg (80%) of desired product. ¹H-NMR (DMSO-d₆) δ 8.05 (d, J=8.1 Hz, 2H), 7.47-7.53 (m, 4H), 7.41 (d, J=5.5 Hz, 1H), 7.31 (d, J=2.9 Hz, 1H), 7.05 (dd, J=5.5, 4.0 Hz, 1H), 6.99 (d, J=8.3 Hz, 1H), 4.77 (d, J=4.3 Hz, 1H), 4.35 (s, 2H), 3.95 (m, 1H), 3.69-3.75 (m, 4H), 3.07-3.24 (m, 3H), 1.75 (m, 1H), 1.65 (m, 1H), 1.30 (m, 2H).

Ex-11B: The title compound was prepared from 1-[4-(4-hydroxy-piperidine-1-carbonyl)-phenyl]-2-(2-methoxy-5-thien-2-yl-phenyl)-ethanone obtained from Ex-11A in a similar manner as described in Ex-3D. The crude material was purified by slurrying in EtOAc and filtering to provide 295 mg (80%) of product, mp 197-199° C. ¹H-NMR (DMSO-d₆) δ 7.79 (d, J=8.2 Hz, 2H), 7.64-7.65 (m, 1H), 7.56-7.60 (m, 1H), 7.41-7.47 (m, 4H), 7.07-7.10 (m, 1H), 6.95 (d, J=8.1 Hz, 1H), 6.15 (s, 1H), 5.73 (s, 1H), 4.76 (d, J=3.5 Hz, 1H), 3.95 (m, 1H), 3.66-3.72 (m, 2H), 3.49 (s, 3H), 3.38 (m, 1H), 3.21 (m, 1H), 3.10 (m, 1H), 1.71 (m, 2H), 1.33 (m, 2H). HRMS (EI⁺) m/z: calc. 447.1504, found 447.1509.

### Example 12

### 4-[1-(5-Benzo[b]thien-2-yl-2,4-dimethoxy-benzoyl)-vinyl]-benzoic acid methyl ester

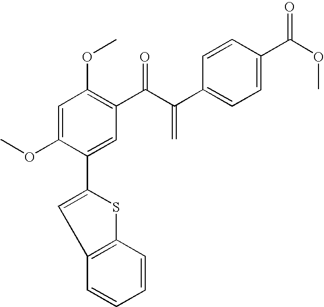

Ex-12A: 4-Bromomethyl-benzoic acid methyl ester (2.5 g, 10.91 mmol), potassium iodide (91 mg, 0.55 mmol) and chloro (1,5-cyclooctadiene) rhodium (I) dimer (540 mg, 1.1 mmol) were suspended in formic acid (20 mL). The reaction was flushed with nitrogen and then heated to 75° C. under an atmosphere of carbon monoxide. After aging overnight, the reaction was concentrated to dryness, diluted with EtOAc and washed with 1 N HCl. The organic layer was extracted with saturated NaHCO₃ and the resulting aqueous was acidified with HCl and then extracted with EtOAC. The organic layer was dried with MgSO₄ and concentrated onto silica gel. The crude was purified by silica gel chromatography (10% MeOH in CH₂Cl₂) to afford 0.53 g (25% yield) of desired 4-carboxymethyl-benzoic acid methyl ester. ¹H-NMR (CDCl₃) δ 8.01 (d, J=7.9 Hz, 2H), 7.36 (d, J=7.9 Hz, 2H), 3.91 (s, 3H), 3.71 (s, 2H).

Ex-12B: 4-Carboxymethyl-benzoic acid methyl ester obtained from Ex-12A (500 mg, 2.57 mmol) and CH₂Cl₂ (10 mL) were sequentially charged into a clean reaction vessel and the resulting solution was treated with oxalyl chloride (250 μL, 2.87 mmol). A catalytic amount of DMF (1 drop) was then added and the reaction was aged at room temperature overnight. The reaction was concentrated to dryness under reduced pressure and used without further purification.

Ex-12C: 1-Bromo-2,4-dimethoxybenzene (0.375 μL, 2.60 mmol), 4-chlorocarbonylmethyl-benzoic acid methyl ester obtained from Ex-12B (1.11 mL, 2.57 mmol) and CH₂Cl₂ (10 mL) were sequentially charged into a clean reaction vessel and the resulting solution was cooled to −40° C. AlCl₃ (375 mg, 2.81 mmol) was then added and the resulting solution was aged at −40° C. After 1 h, additional AlCl₃ (375 mg, 2.81 mmol) was added and the reaction was aged. HPLC indicated incomplete reaction after an 1.5 h therefore additional AlCl₃ (75 mg, 0.56 mmol) was added. After 30 min the reaction was poured into 1 N HCl and extracted with EtOAc. The organic cut was washed with saturated NaHCO₃, dried with MgSO₄ and concentracted to dryness. The crude product was purified by crystallization from EtOAc and heptane affording 0.51 g (50% yield) of desired 4-[2-(5-bromo-2,4-dimethoxy-phenyl)-2-oxo-ethyl]-benzoic acid methyl ester. ¹H-NMR (DMSO-d₆) δ 7.85 (d, J=8.1 Hz, 2H), 7.78 (s, 1H), 7.30 (d, J=8.1 Hz, 2H), 6.78 (s, 1H), 4.29 (s, 2H), 3.94 (s, 6H), 3.80 (s, 3H).

Ex-12D: 4-[2-(5-Benzo[b]thien-2-yl-2,4-dimethoxy-phenyl)-2-oxo-ethyl]-benzoic acid methyl ester was prepared from 4-[2-(5-bromo-2,4-dimethoxy-phenyl)-2-oxo-ethyl]-benzoic acid methyl ester obtained from Ex-12C in a similar manner as described in Ex-8B. The crude material was purified by silica gel chromatography (35% EtOAc in hexanes) to provide 302 mg (74%) of pure product as an orange-beige solid. ¹H-NMR (CDCl₃) δ 8.22 (s, 1H), 8.00 (d, J=7.8 Hz, 2H), 7.80 (d, J=6.9 Hz, 1H), 7.75 (dd, J=6.9, 1.5 Hz, 1H), 7.66 (s, 1H), 7.31 (d, J=7.8 Hz, 2H), 7.28-7.35 (m, 1H), 6.53 (s, 1H), 4.37 (s, 2H), 4.04 (s, 3H), 3.98 (s, 3H), 3.90 (s, 3H).

Ex-12E: The title compound was prepared from 4-[2-(5-benzo[b]thien-2-yl-2,4-dimethoxy-phenyl)-2-oxo-ethyl]-benzoic acid methyl ester obtained from Ex-12D in a similar manner as described in Ex-6E. The crude material was purified by silica gel chromatography (100% CH₂Cl₂) to provide 123 mg (40%) of pure product as a light yellow foam, mp 184-186° C. ¹H-NMR (CDCl₃) δ 8.05 (s, 1H), 8.01 (d, J=8.7 Hz, 2H), 7.81 (d, J=7.6 Hz, 1H), 7.77 (dd, J=7.6, 1.2 Hz, 1H), 7.67 (s, 1H), 7.47 (d, J=8.7 Hz, 2H), 7.27-7.34 (m, 2H), 6.43 (s, 1H), 5.97 (s, 1H), 5.86 (s, 1H), 4.01 (s, 3H), 3.92 (s, 3H), 3.70 (s, 3H). HRMS (EI+) m/z: calc. 458.1188, found 458.1195.

### Example 13

### 1-(4-Fluoro-phenyl)-2-(2-methoxy-5-thien-2-yl-phenyl)-propen one

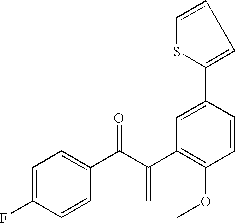

Ex-13A: In a 2-neck, 1 L round-bottom flask (methoxymethyl) triphenylphosphonium chloride (37.7 g, 110 mmol) was combined with THF (300 mL). The reaction was cooled to 2° C. and nBuLi (1.6 M in hexanes, 72.2 mL, 116 mmol) was added while maintaining the reaction temperature at <10° C. The resulting solution was stirred for 35 min. In a separate flask 2-methoxy-5-thien-2-yl-benzaldehyde obtained from Ex-7B (12.03 g, 55.11 mmol) was combined with THF (80 mL). The resulting solution (and a 20 mL THF rinse) was transferred to the ylide solution via cannula over ˜10 min while maintaining the reaction temperature <10° C. The resulting solution was allowed to warm to room temperature; HPLC indicated the reaction was complete in 2 h 15 min. The solution was diluted with brine and H₂O and then extracted with EtOAc. The organic phase was washed with brine, dried over Na₂SO₄, filtered, concentrated, and dried. The crude material was purified by silica gel chromatography (10-15% EtOAc in hexanes) to provide 11.73 g of 2-[4-methoxy-3-(2-methoxy-vinyl)-phenyl]-thiophene (mixture of cis- and trans-isomers) as a yellow oil. ¹H-NMR (CDCl₃) δ 8.28 (d, J=2.2 Hz, 1H), 7.47 (d, J=2.2 Hz, 2H), 7.36 (dd, J=8.1, 2.2 Hz, 2H), 7.17-7.22 (m, 4H), 7.04 (dd, J=5.1, 3.9 Hz, 2H), 6.85 (d, J=10.8 Hz, 1H), 6.83 (d, J=8.1 Hz, 1H), 6.22 (d, J=7.3 Hz, 1H), 6.00 (d, J=10.8 Hz, 1H), 5.63 (d, J=7.3 Hz, 1H), 3.87 (s, 3H), 3.85 (s, 3H), 3.79 (s, 3H), 3.72 (s, 3H).

Ex-13B: In a 200 mL round-bottom flask 2-[4-methoxy-3-(2-methoxy-vinyl)-phenyl]-thiophene obtained from Ex-13A (11.73 g, 47.62 mmol) was combined with THF (80 mL), H₂O (40 mL), and concentrated HCl (2.5 mL). The resulting solution was heated at 65° C.; HPLC indicated the reaction was incomplete after 1 h 10 min. Additional concentrated HCl was added (10 mL) and the solution was heated for another 3.5 h; HPLC indicated the reaction was complete. The reaction was cooled and extracted with EtOAc. The organic phase was washed with brine, dried over Na₂SO₄, filtered, concentrated, and dried. The crude material was purified by silica gel chromatography (10-20% EtOAc in hexanes) to provide 6.5 g (2 steps, 51%) of (2-methoxy-5-thien-2-yl-phenyl)-acetaldehyde as a yellow oil. ¹H-NMR (CDCl₃) δ 9.70-9.72 (m, 1H), 7.53 (dd, J=8.4, 3.1 Hz, 1H), 7.38 (d, J=3.1 Hz, 1H), 7.22 (d, J=5.1 Hz, 1H), 7.19 (dd, J=3.7, 1.5 Hz, 1H), 7.05 (dd, J=5.1, 3.7 Hz, 1H), 6.91 (d, J=8.4 Hz, 1H), 3.85 (s, 3H), 3.68 (d, J=1.8 Hz, 2H).

Ex-13C: In a 200 mL round-bottom flask (2-methoxy-5-thien-2-yl-phenyl)-acetaldehyde obtained from Ex-13B (6.5 g, 28 mmol) was combined with THF (100 mL). 4-Fluoro-phenylmagnesium bromide (2 M in Et₂O, 14.3 mL, 28.6 mmol) was added dropwise over 15 min. An exotherm was observed and the solution was cooled in a 0° C. bath during addition. After stirring for 1 h, HPLC indicated some aldehyde remained. Additional 4-fluoro-phenylmagnesium bromide (2 M in Et₂O, 0.5 mL, 1 mmol) was added dropwise. After 1 h, HPLC indicated most of the starting material had been consumed. The reaction was quenched with saturated, aqueous NH⁴Cl and H₂O, then extracted with EtOAc. The organic phase was washed with brine, dried over Na₂SO₄, filtered, concentrated, and dried. The crude material was purified by silica gel chromatography (25% EtOAc in hexanes) to provide 2.22 g of 1-(4-fluoro-phenyl)-2-(2-methoxy-5-thien-2-yl-phenyl)-ethanol (24%) as a yellow oil. An additional 1.00 g of semi-pure product was also isolated. ¹H-NMR (CDCl₃) δ 7.47 (dd, J=8.5, 2.8 Hz, 1H), 7.35 (dd, J=8.1, 5.1 Hz, 2H), 7.27 (d, J=2.8 Hz, 1H), 7.21 (d, J=5.4 Hz, 1H), 7.14 (d, J=3.6 Hz, 1H), 6.99-7.06 (m, 3H), 6.89 (d, J=8.5 Hz, 1H), 4.98 (p, J=4.9 Hz, 1H), 3.87 (s, 3H), 3.11 (dd, J=13.9, 4.9 Hz, 1H), 2.98 (dd, J=13.9, 4.9 Hz, 1H), 2.47 (d, J=3.0 Hz, 1H).

Ex-13D: In a 200 mL round-bottom flask 1-(4-fluoro-phenyl)-2-(2-methoxy-5-thien-2-yl-phenyl)-ethanol obtained from Ex-13C (2.22 g, 6.76 mmol) was combined with 4A molecular sieves (6.8 g) and CH₂Cl₂ (30 mL). Pyridinium chlorochromate (2.48 g, 11.50 mmol) was added and the reaction was stirred at room temperature. After 35 min, HPLC indicated starting material remained. Additional PCC (0.45 g, 2.09 mmol) and CH₂Cl₂ (10 mL) were added over 2 h 20 min. After 4 h total reaction time, HPLC indicated the reaction was complete. Celite was added to the crude reaction mixture and it was filtered through a 1 inch silica bed, washing with CH₂Cl₂. The filtrate was concentrated to give crude product, which was purified by silica gel chromatography (15% EtOAc in hexanes) to provide 1.37 g (62%) of semi-pure 1-(4-fluoro-phenyl)-2-(2-methoxy-5-thien-2-yl-phenyl)-ethanone as a light orange solid. ¹H-NMR (CDCl₃) δ 8.05-8.10 (m, 2H), 7.50 (dd, J=8.1, 2.2 Hz, 1H), 7.41 (d, J=2.2 Hz, 1H), 7.15-7.21 (m, 2H), 7.10-7.14 (m, 2H), 7.03 (dd, J=5.1, 3.6 Hz, 1H), 6.89 (d, J=8.1 Hz, 1H), 4.27 (s, 2H), 3.81 (s, 3H).

Ex-13E: The title compound was prepared from 1-(4-fluoro-phenyl)-2-(2-methoxy-5-thien-2-yl-phenyl)-ethanone obtained from Ex-13D in a similar manner as described in Ex-1H. The crude material was purified by silica gel chromatography (15% EtOAc in hexanes) to provide 27 mg (38%) of product (93.5% pure by HPLC) as a white solid, mp 116-121° C. ¹H-NMR (CDCl₃) δ 7.90-7.95 (m, 2H), 7.64 (d, J=2.4 Hz, 1H), 7.56 (dd, J=8.4, 2.5 Hz, 1H), 7.24-7.26 (m, 2H), 7.05-7.11 (m, 3H), 6.81 (d, J=8.4 Hz, 1H), 6.03 (s, 1H), 5.77 (s, 1H), 3.58 (s, 3H). HRMS (EI⁺) m/z: calc. 338.0777, found 338.0782.

### Example 14

### 2-(2-Methoxy-5-thien-2-yl-phenyl)-1-(4-pyrrolidin-1-yl-phenyl)-propenone

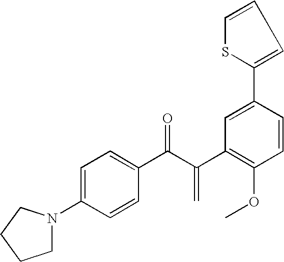

Ex-14A: In a 50 mL round-bottom flask 1-(4-fluoro-phenyl)-2-(2-methoxy-5-thien-2-yl-phenyl)-ethanone obtained from Ex-13D (502 mg, 1.53 mmol) was combined with DMF (9 mL) and pyrrolidine (0.26 mL, 3.1 mmol). Sodium carbonate (193 mg, 1.82 mmol) was added and the reaction was heated at 85° C. After heating overnight, HPLC indicated the starting material had been consumed. The reaction was diluted with H₂O and extracted with EtOAc. The organic phase was washed with brine, dried over Na₂SO₄, filtered, concentrated, and dried. The crude material was purified by silica gel chromatography (2 columns at 25% EtOAc in hexanes) to provide 77 mg (13%) of 2-(2-methoxy-5-thien-2-yl-phenyl)-1-(4-pyrrolidin-1-yl-phenyl)-ethanone as a yellow solid. ¹H-NMR (CDCl₃) δ 7.96 (d, J=9.0 Hz, 2H), 7.45-7.48 (m, 2H), 7.16-7.19 (m, 2H), 7.01 (t, J=4.8 Hz, 1H), 6.87 (d, J=8.1 Hz, 1H), 6.51 (d, J=9.0 Hz, 2H), 4.22 (s, 2H), 3.82 (s, 3H), 3.36 (t, J=6.6 Hz, 4H), 2.01-2.05 (m, 4H).

Ex-14B: The title compound was prepared from 2-(2-methoxy-5-thien-2-yl-phenyl)-1-(4-pyrrolidin-1-yl-phenyl)-ethanone obtained from Ex-14A in a similar manner as described in Ex-3D. The crude material was purified by silica gel chromatography (25% EtOAc in hexanes) to provide 53 mg (67%) of pure product as an off-white solid, mp 187-190° C. ¹H-NMR (CDCl₃) δ 8.70 (d, J=8.7 Hz, 2H), 7.65 (d, J=2.4 Hz, 1H), 7.52 (dd, J=9.0, 2.4 Hz, 1H), 7.22-7.25 (m, 2H), 7.06 (dd, J=5.8, 3.7 Hz, 1H), 6.82 (d, J=9.0 Hz, 1H), 6.49 (d, J=8.7 Hz, 2H), 5.93 (s, 1H), 5.68 (s, 1H), 3.61 (s, 3H), 3.36 (t, J=6.7 Hz, 4H), 2.00-2.05 (m, 4H). HRMS (EI⁺) m/z: calc. 389.1450, found 389.1453.

### Example 15

### 4-[1-(4-Methoxy-3-thien-2-yl-benzoyl)-vinyl]-benzoic acid

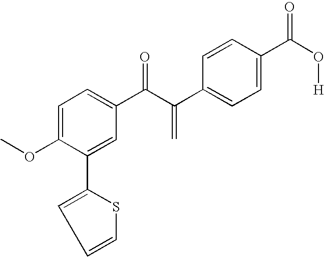

Ex-15A: In a 100 mL round-bottom flask 3-bromo-4-methoxy-benzaldehyde (2.09 g, 9.72 mmol) was combined with CHCl₃ (10 mL), 1,3-propane dithiol (1.02 mL, 10.16 mmol), and Na₂SO₄ (0.90 g). The mixture was cooled in a 0° C. bath and boron trifluoride diethyletherate (0.62 mL, 4.89 mmol) was added dropwise. After 5 min, the reaction was allowed to warm to room temperature. HPLC indicated the reaction was complete in 3 h 15 min. The reaction was diluted with CH₂Cl₂ and washed with H₂O. The organic phase was dried over Na₂SO₄, filtered, concentrated, and dried. The crude material was purified by silica gel chromatography (10-25% EtOAc in hexanes) to provide 2.15 g (73%) of 2-(3-bromo-4-methoxy-phenyl)-[1,3]dithiane as a crystalline, white solid. ¹H-NMR (CDCl₃) δ 7.67 (d, J=2.1 Hz, 1H), 7.38 (dd, J=8.1, 2.1 Hz, 1H), 6.85 (d, J=8.1 Hz, 1H), 5.08 (s, 1H), 3.89 (s, 3H), 3.04 (td, J=14.7, 3.0 Hz, 2H), 2.89 (dt, J=14.7, 3.0 Hz, 2H), 2.17 (dp, J=12.3, 2.7 Hz, 1H), 1.93 (qt, J=12.3, 2.76 Hz, 1H).

Ex-15B: In a 50 mL round-bottom flask 2-(3-bromo-4-methoxy-phenyl)-[1,3]dithiane obtained from Ex-15A (1.04 g, 3.41 mmol) was combined with THF (10 mL). The solution was cooled in a −78° C. bath and LDA (2 M in heptane/THF/ethylbenzene, 1.87 mL, 3.74 mmol) was added dropwise. The reaction was allowed to warm to 0° C. and transferred to a 50 mL round-bottom flask (via cannula) which contained a 0° C. solution of 4-bromomethyl-benzoic acid methyl ester (781 mg, 3.41 mmol) dissolved in THF (10 mL). The resulting solution was allowed to warm to room temperature. After stirring overnight, HPLC indicated some starting material remained. The reaction was quenched with 1 N HCl and diluted with H₂O, then extracted with CH₂Cl₂. The organic phase was washed with brine, dried over Na₂SO₄, filtered, concentrated, and dried. The crude material was purified by silica gel chromatography (10% EtOAc in hexanes) to provide 501 mg (30%) of semi-pure 4-[2-(3-bromo-4-methoxy-phenyl)-[1,3]dithian-2-ylmethyl]-benzoic acid methyl ester as a white solid.

¹H-NMR (CDCl₃) δ 7.94 (d, J=2.4 Hz, 1H), 7.80 (d, J=8.7 Hz, 2H), 7.50 (dd, J=7.9, 2.4 Hz, 1H), 6.83 (d, J=7.9 Hz, 2H), 6.78 (d, J=8.7 Hz, 1H), 3.91 (s, 3H), 3.88 (s, 3H), 3.27 (s, 2H), 2.61-2.67 (m, 4H), 1.88-1.96 (m, 2H).

Ex-15C: 4-[2-(4-Methoxy-3-thien-2-yl-phenyl)-[1,3]dithian-2-ylmethyl]-benzoic acid methyl ester was prepared from 4-[2-(3-bromo-4-methoxy-phenyl)-[1,3]dithian-2-ylmethyl]-benzoic acid methyl ester obtained from Ex-15B in a similar manner as described in Ex-1B. The crude material was purified by silica gel chromatography (15% EtOAc in hexanes) to provide 442 mg (88%) of semi-pure product as a yellow foam. ¹H-NMR (CDCl₃) δ 8.00 (d, J=2.5 Hz, 1H), 7.79 (d, J=8.4 Hz, 2H), 7.52 (dd, J=9.0, 2.5 Hz, 1H), 7.37 (d, J=3.6 Hz, 1H), 7.32 (d, J=4.8 Hz, 1H), 7.07 (dd, J=4.8, 3.6 Hz, 1H), 6.86-6.90 (m, 3H), 3.95 (s, 3H), 3.87 (s, 3H), 3.33 (s, 2H), 2.63-2.73 (m, 4H), 1.89-1.99 (m, 2H).

Ex-15D: In a 200 mL round-bottom flask 4-[2-(4-methoxy-3-thien-2-yl-phenyl)-[1,3]dithian-2-ylmethyl]-benzoic acid methyl ester obtained from Ex-15C (442 mg, 0.968 mmol) was combined with CH₂Cl₂ (12 mL). A solution of mercury (II) perchlorate hydrate (860 mg, 2.15 mmol) in MeOH (20 mL) was added. TLC indicated the reaction was complete in 20 min. The reaction was filtered into ice water and the filter cake was washed with CH₂Cl₂. The filtrate was extracted with CH₂Cl₂ and the organic phase was washed with 12-saturated brine, dried over Na₂SO₄, filtered, concentrated, and dried. The crude material was purified by silica gel chromatography (20% EtOAc in hexanes) to provide 133 mg (37%) of 4-[2-(4-methoxy-3-thien-2-yl-phenyl)-2-oxo-ethyl]-benzoic acid methyl ester as a white solid. ¹H-NMR (CDCl₃) δ 8.30 (d, J=2.2 Hz, 1H), 8.01 (d, J=8.1 Hz, 2H), 7.93 (dd, J=9.3, 2.2 Hz, 1H), 7.52 (d, J=3.4 Hz, 1H), 7.35-7.38 (m, 3H), 7.10 (dd, J=5.4, 3.4 Hz, 1H), 7.02 (d, J=8.1 Hz, 1H), 4.39 (s, 2H), 4.00 (s, 3H), 3.90 (s, 3H).

Ex-15E: 4-[2-(4-Methoxy-3-thien-2-yl-phenyl)-2-oxo-ethyl]-benzoic acid was prepared from 4-[2-(4-methoxy-3-thien-2-yl-phenyl)-2-oxo-ethyl]-benzoic acid methyl ester obtained from Ex-15D in a similar manner as described in Ex-5C. The crude material was purified by silica gel chromatography (5% MeOH in CH₂Cl₂) to provide 53 mg (41%) of pure product as a white solid. An additional 47 mg of impure material was also isolated. ¹H-NMR (CDCl₃) δ 8.30 (d, J=2.1 Hz, 1H), 8.05 (d, J=8.7 Hz, 2H), 7.94 (dd, J=8.7, 2.1 Hz, 1H), 7.52 (d, J=3.9 Hz, 1H), 7.37 (m, 3H), 7.11 (dd, J=5.1, 3.6 Hz, 1H), 7.02 (d, J=8.7 Hz, 1H), 4.35 (s, 2H), 4.00 (s, 3H).

Ex-15F: The title compound was prepared from 4-[2-(4-methoxy-3-thien-2-yl-phenyl)-2-oxo-ethyl]-benzoic acid obtained from Ex-15E in a similar manner as described in Ex-3D. The crude material was purified by silica gel chromatography (3% MeOH in CH₂Cl₂) to provide 39 mg (71%) of pure product as a white solid, mp 204-205° C. ¹H-NMR (CDCl₃) δ 8.24 (d, J=2.4 Hz, 1H), 8.09 (d, J=8.7 Hz, 2H), 7.84 (dd, J=8.7, 2.4 Hz, 1H), 7.56 (d, J=8.7 Hz, 2H), 7.48 (d, J=3.6 Hz, 1H), 7.36 (d, J=5.3 Hz, 1H), 7.09 (dd, J=5.3, 3.6 Hz, 1H), 6.99 (d, J=8.7 Hz, 1H), 6.16 (s, 1H), 5.77 (s, 1H), 4.00 (s, 3H). HRMS (ES⁺) m/z: calc. 365.0848, found 365.0846.

### Example 16

### 4-[1-(3,4-Dimethoxy-5-thien-2-yl-benzoyl)-vinyl]-benzoic acid

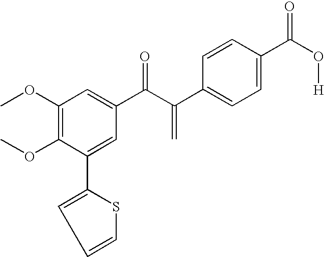

Ex-16A: (3-Bromo-4,5-dimethoxy-phenyl)-(tert-butyl-dimethyl-silanyloxy)-acetonitrile was prepared from 3-bromo-4,5-dimethoxy-benzaldehdye in a similar manner as described in Ex-1A. The crude material was purified by silica gel chromatography (10% EtOAc in hexanes) to provide 4.50 g (50%) of pure product as a faint yellow oil. An additional 2.99 g semi-pure product was also obtained. ¹H-NMR (CDCl₃) δ 7.21 (d, J=1.8 Hz, 1H), 6.98 (d, J=1.8 Hz, 1H), 5.43 (s, 1H), 3.89 (s, 3H), 3.78 (s, 3H), 0.95 (s, 9H), 0.24 (s, 3H), 0.17 (s, 3H).

Ex-16B: 4-[2-(3-Bromo-4,5-dimethoxy-phenyl)-2-(tert-butyl-dimethyl-silanyloxy)-2-cyano-ethyl]-benzoic acid methyl ester was prepared from (3-Bromo-4,5-dimethoxy-phenyl)-(tert-butyl-dimethyl-silanyloxy)-acetonitrile obtained from Ex-16A and 4-bromomethyl-benzoic acid methyl ester in a similar manner as described in Ex-1E. The crude material was purified by silica gel chromatography (15% EtOAc in hexanes) to provide 881 mg (66%) of pure product as a faint yellow oil. ¹H-NMR (CDCl₃) δ 7.96 (d, J=7.8 Hz, 2H), 7.32 (d, J=2.2 Hz, 1H), 7.23-7.26 (m, 2H), 6.86 (d, J=2.2 Hz, 1H), 3.92 (s, 3H), 3.88 (s, 3H), 3.79 (s, 3H), 3.24 (d, J=13.2 Hz, 1H), 3.16 (d, J=13.2 Hz, 1H), 0.92 (s, 9H), −0.02 (s, 3H), −0.04 (s, 3H).

Ex-16C: 4-[2-(3-Bromo-4,5-dimethoxy-phenyl)-2-oxo-ethyl]-benzoic acid methyl ester was prepared from 4-[2-(3-bromo-4,5-dimethoxy-phenyl)-2-(tert-butyl-dimethyl-silanyloxy)-2-cyano-ethyl]-benzoic acid methyl ester obtained from Ex-16B in a similar manner as described in Ex-1F. The crude material was purified by silica gel chromatography (25% EtOAc in hexanes) to provide 580 mg (85%) of pure product as a faint yellow oil which slowly crystallized upon standing. ¹H-NMR (CDCl₃) δ 8.01 (d, J=8.5 Hz, 2H), 7.80 (d, J=2.1 Hz, 1H), 7.49 (d, J=2.1 Hz, 1H), 7.32 (d, J=8.5 Hz, 2H), 4.29 (s, 3H), 3.93 (s, 3H), 3.91 (s, 3H), 3.90 (s, 3H).

Ex-16D: 4-[2-(3,4-Dimethoxy-5-thien-2-yl-phenyl)-2-oxo-ethyl]-benzoic acid methyl ester was prepared from 4-[2-(3-bromo-4,5-dimethoxy-phenyl)-2-oxo-ethyl]-benzoic acid methyl ester obtained from Ex-16C in a similar manner as described in Ex-8B. The crude material was purified by silica gel chromatography (25% EtOAc in hexanes) to provide 554 mg (94%) of pure product as a faint yellow oil which slowly solidified upon standing. ¹H-NMR (CDCl₃) δ 8.01 (d, J=8.3 Hz, 2H), 7.93 (d, J=2.7 Hz, 1H), 7.49 (d, J=2.7 Hz, 1H), 7.48 (d, J=1.5 Hz, 1H), 7.40 (dd, J=5.1, 1.5 Hz, 1H), 7.36 (d, J=8.3 Hz, 2H), 7.12 (dd, J=5.1, 3.6 Hz, 1H), 4.35 (s, 2H), 3.93 (s, 3H), 3.90 (s, 3H), 3.89 (s, 3H).

Ex-16E: 4-[2-(3,4-Dimethoxy-5-thien-2-yl-phenyl)-2-oxo-ethyl]-benzoic acid was prepared from 4-[2-(3,4-dimethoxy-5-thien-2-yl-phenyl)-2-oxo-ethyl]-benzoic acid methyl ester obtained from Ex-16D in a similar manner as described in Ex-5C. The crude material was purified by silica gel chromatography (5% MeOH in CH₂Cl₂) to provide 338 mg of semi-pure product as a white foam. The product contained a 15 mol % impurity. ¹H-NMR (CDCl₃) δ 8.09 (d, J=8.5 Hz, 2H), 7.94 (d, J=1.6 Hz, 1H), 7.51 (dd, J=3.4, 1.2 Hz, 1H), 7.49 (d, J=1.6 Hz, 1H), 7.40 (d, J=8.5 Hz, 2H), 7.39-7.42 (m, 1H), 8.13 (dd, J=5.1, 3.4 Hz, 1H), 4.36 (s, 2H), 3.93 (s, 3H), 3.90 (s, 3H).

Ex-16F: The title compound was prepared from 4-[2-(3,4-dimethoxy-5-thien-2-yl-phenyl)-2-oxo-ethyl]-benzoic acid obtained from Ex-16E in a similar manner as described in Ex-3D. The crude material was purified by slurrying (EtOH/hexanes) and filtration to provide 163 mg (2 steps, 30%) of pure product as a white, semi-crystalline solid, mp 159-160° C. ¹H-NMR (CDCl₃) δ 8.11 (d, J=9.0 Hz, 2H), 7.80 (d, J=1.9 Hz, 1H), 7.56 (d, J=9.0 Hz, 2H), 7.45 (d, J=1.9 Hz, 1H), 7.41 (d, J=4.2 Hz, 1H), 7.37 (d, J=5.2 Hz, 1H), 7.08 (dd, J=5.2, 4.2 Hz, 1H), 6.19 (s, 1H), 5.81 (s, 1H), 3.94 (s, 3H), 3.91 (s, 3H). HRMS (ES⁺) m/z: calc. 395.0953, found 395.0957.

### Example 17

### 1-(4-Hydroxy-phenyl)-2-(2-methoxy-5-thien-2-yl-phenyl)-propenone

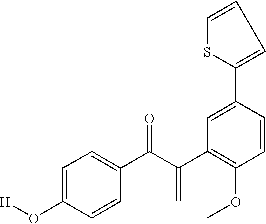

Ex-17A: 4-[1,3]Dithian-2-yl-phenol was prepared from 4-hydroxy benzaldehdye in a similar manner as described in Ex-15A. The crude material was purified by slurrying (hot CH₂Cl₂/hexanes) and filtration to provide 4.17 g (79%) of pure product as a crystalline, faint yellow solid. ¹H-NMR (CDCl₃) δ 7.35 (d, J=9.0 Hz, 2H), 6.78 (d, J=9.0 Hz, 2H), 5.12 (s, 1H), 4.73 (bs, 1H), 3.04 (td, J=14.3, 3.3 Hz, 2H), 2.89 (dt, J=14.3, 3.3 Hz, 2H), 2.11-2.21 (m, 1H), 1.84-1.99 (m, 1H).

Ex-17B: In a 100 mL round-bottom flask 4-[1,3]dithian-2-yl-phenol (1.96 g, 9.23 mmol) was combined with THF (35 mL). The solution was cooled in a −40° C. bath and nBuLi (1.6 M in hexanes, 12.0 mL, 19.2 mmol) was added dropwise. The reaction was allowed to warm to 0° C. and stirred for 20 min. In a 10 mL round-bottom flask 2-(3-bromomethyl-4-methoxy-phenyl)-thiophene obtained from Ex-7D (2.59 g, 9.15 mmol) was dissolved in THF (10 mL). The resulting solution was transferred to the 100 mL round-bottom flask quickly. The resulting solution was allowed to warm to room temperature immediately; HPLC indicated starting material remained after 5.5 h. The reaction was quenched with 1 N HCl and diluted with H₂O, then extracted with EtOAc. The organic phase was washed with brine, dried over Na₂SO₄, filtered, concentrated, and dried. The crude material was purified by silica gel chromatography (20-25% EtOAc in hexanes) to provide 0.66 g of semi-pure 4-[2-(2-methoxy-5-thien-2-yl-benzyl)-[1,3]dithian-2-yl]-phenol as an orange foam. ¹H-NMR (CDCl₃) δ 7.58 (d, J=9.3 Hz, 2H), 7.36 (dd, J=8.9, 3.0 Hz, 1H), 7.13 (dd, J=3.9, 0.9 Hz, 1H), 6.96-7.00 (m, 2H), 6.85 (d, J=2.4 Hz, 1H), 6.73 (d, J=9.3 Hz, 2H), 6.67 (d, J=8.9 Hz, 1H), 5.80 (bs, 1H), 3.55 (s, 3H), 3.34 (s, 2H), 2.62-2.68 (m, 4H), 1.88-1.98 (m, 2H).

Ex-17C: 1-(4-Hydroxy-phenyl)-2-(2-methoxy-5-thien-2-yl-phenyl)-ethanone was prepared from 4-[2-(2-methoxy-5-thien-2-yl-benzyl)-[1,3]dithian-2-yl]-phenol obtained from Ex-17B in a similar manner as described in Ex-15D. The crude material was purified by silica gel chromatography (35% EtOAc in hexanes) to provide 121 mg (2 steps, 4%) of pure product as an off-white solid. ¹H-NMR (DMSO-d₆) δ 7.91 (d, J=8.5 Hz, 2H), 7.53 (dd, J=9.0, 2.7 Hz, 1H), 7.44-7.47 (m, 2H), 7.34 (d, J=2.7 Hz, 1H), 7.09 (dd, J=5.4, 3.9 Hz, 1H), 7.01 (d, J=9.0 Hz, 1H), 6.86 (d, J=8.5 Hz, 2H), 4.26 (s, 2H), 3.73 (s, 3H), 3.30 (s, 3H).

Ex-17D: The title compound was prepared from 1-(4-hydroxy-phenyl)-2-(2-methoxy-5-thien-2-yl-phenyl)-ethanone obtained from Ex-17C in a similar manner as described in Ex-3D. MeOH added to the reaction solution to increase the solubility of the substrate. The crude material was purified by silica gel chromatography (5% MeOH in CH₂Cl₂) followed by slurrying (CH₂Cl₂/hexanes) and filtration to provide 71 mg (57%) of semi-pure product as an off-white solid. The product contained a piperidine-adduct impurity which could not be removed. ¹H-NMR (DMSO-d₆) δ 7.71 (d, J=8.5 Hz, 2H), 7.59-7.63 (m, 2H), 7.49 (d, J=5.4 Hz, 1H), 7.47 (d, J=3.3 Hz, 1H), 7.12 (dd, J=5.4, 3.3 Hz, 1H), 7.00 (d, J=9.0 Hz, 1H), 6.82 (d, J=8.5 Hz, 2H), 6.05 (s, 1H), 5.62 (s, 1H), 3.52 (s, 3H). HRMS (ES⁺) m/z: calc. 337.0898, found 337.0914.

### Example 18

### 2-(2-Methoxy-5-thien-2-yl-phenyl)-1-(4-nitro-phenyl)-propenone

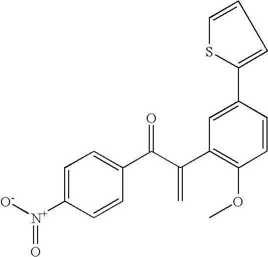

Ex-18A: [(4-Nitro-phenyl)-phenylamino-methyl]-phosphonic acid diphenyl ester was prepared from 4-nitro benzaldehyde in a similar manner as described in Ex-2A. EtOAc was added to the reaction mix in order to solubilize the substrate. The crude reaction mixture was diluted with MTBE and filtered to provide 2.10 g of pure product. A second crop was obtained by concentrating the filtrate, slurrying the residue (MTBE/EtOAc) and filtration to provide 1.34 g semi-pure product (3.44 g total, 55%). ¹H-NMR (DMSO-d₆) δ 8.26 (d, J=7.8 Hz, 2H), 7.99 (dd, J=5.4, 1.5 Hz, 2H), 6.98 (d, J=7.8 Hz, 2H), 6.92-7.38 (bm, 12H), 6.61 (t, J=7.8 Hz, 1H), 5.93 (dd, J=26.7, 10.5 Hz, 1H).

Ex-18B: 2-(2-Methoxy-5-thien-2-yl-phenyl)-1-(4-nitro-phenyl)-ethanone was prepared from [(4-Nitro-phenyl)-phenylamino-methyl]-phosphonic acid diphenyl ester obtained from Ex-18A and 2-methoxy-5-thien-2-yl-benzaldehyde obtained from Ex-7B in a similar manner as described in Ex-2C. The crude material was purified by slurrying (EtOAc/hexanes) and filtration to provide 1.02 g (63%) of pure product as an orange solid. ¹H-NMR (CDCl₃) δ 8.30 (d, J=9.0 Hz, 2H), 8.19 (d, J=9.0 Hz, 2H), 7.51 (dd, J=8.1, 2.1 Hz, 1H), 7.42 (d, J=2.1 Hz, 1H), 7.22 (d, J=5.2 Hz, 1H), 7.18 (d, J=3.3 Hz, 1H), 7.04 (dd, J=5.2, 3.3 Hz, 1H), 6.90 (d, J=8.1 Hz, 1H), 4.32 (s, 2H), 3.81 (s, 3H).

Ex-18C: The title compound was prepared from 2-(2-methoxy-5-thien-2-yl-phenyl)-1-(4-nitro-phenyl)-ethanone obtained from Ex-18B in a similar manner as described in Ex-3D. The crude material was purified by slurrying (EtOH/hexanes) and filtration to provide 179 mg (70%) of pure product as a faint yellow solid, mp 160-162° C. ¹H-NMR (CDCl₃) δ 8.25 (d, J=9.0 Hz, 2H), 8.00 (d, J=9.0 Hz, 2H), 7.60 (d, J=2.1 Hz, 1H), 7.57 (dd, J=8.9, 2.1 Hz, 1H), 7.26-7.28 (m, 2H), 7.08 (dd, J=5.1, 3.9 Hz, 1H), 6.80 (d, J=8.9 Hz, 1H), 6.10 (s, 1H), 5.86 (s, 1H), 3.57 (s, 3H). HRMS (EI⁺) m/z: calc. 365.0722, found 365.0729.

### Example 19

### N-{4-[2-(2-Methoxy-5-thien-2-yl-phenyl)-acryloyl]-phenyl}-methane-sulfonamide

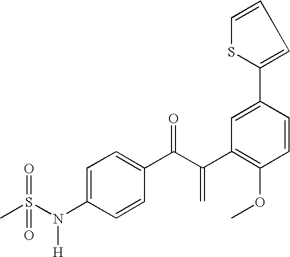

Ex-19A: In a 100 mL round-bottom flask 2-(2-methoxy-5-thien-2-yl-phenyl)-1-(4-nitro-phenyl)-ethanone obtained from Ex-18B (0.76 g, 2.15 mmol) was combined with acetic acid (30 mL) and zinc dust (0.83 g, 12.7 mmol). The resulting mix was heated at 70° C.; HPLC indicated the reaction was complete in 15 min. The reaction was cooled and filtered. The filtrate was cooled in a 0° C. bath and neutralized with 5 N NaOH, then extracted with EtOAc. The organic phase was washed with 1 N NaOH, then brine, then dried over Na₂SO₄, filtered, concentrated, and dried to a yellow solid. The crude 1-(4-amino-phenyl)-2-(2-methoxy-5-thien-2-yl-phenyl)-ethanone was used without further purification (0.65 g, 93%). ¹H-NMR (CDCl₃) δ 7.91 (d, J=8.5 Hz, 2H), 7.47 (dd, J=7.6, 1.9 Hz, 1H), 7.42 (d, J=1.9 Hz, 1H), 7.16-7.20 (m, 2H), 7.02 (t, J=3.6 Hz, 1H), 6.87 (d, J=7.6 Hz, 1H), 6.65 (d, J=8.5 Hz, 2H), 4.22 (s, 2H), 4.10 (bs, 2H), 3.18 (s, 3H).

Ex-19B: In a 25 mL round-bottom flask 1-(4-amino-phenyl)-2-(2-methoxy-5-thien-2-yl-phenyl)-ethanone obtained from Ex-19A (212 mg, 0.655 mmol) was combined with CH₂Cl₂ (10 mL). The solution was cooled in a 0° C. bath and pyridine (56 μL, 0.69 mmol) was added. Mesyl chloride (53 μL, 0.68 mmol) was added dropwise. The reaction was allowed to warm to room temperature and was stirred for 48 h. HPLC indicated starting material remained. Additional pyridine (26 μL, 0.32 mmol) and mesyl chloride (5 μL, 0.06 mmol) were added and the reaction stirred another 48 h. HPLC indicated a small amount of starting material remained. The reaction was diluted with H₂O and extracted with CH₂Cl₂. The organic phase was washed with 0.5 N HCl, followed by ½-saturated brine, then dried over Na₂SO₄, filtered, concentrated, and dried. The crude material was purified by silica gel chromatography (45-50% EtOAc in hexanes) to provide 186 mg (71%) of N-{4-[2-(2-methoxy-5-thien-2-yl-phenyl)-acetyl]-phenyl}-methanesulfonamide as an off-white solid. ¹H-NMR (CDCl₃) δ 8.06 (d, J=9.0 Hz, 2H), 7.49 (dd, J=9.3, 2.1 Hz, 1H), 7.42 (d, J=2.1 Hz, 1H), 7.17-7.23 (m, 4H), 7.03 (dd, J=5.1, 3.3 Hz, 1H), 6.90 (d, J=8.4 Hz, 1H), 6.61 (bs, 1H), 4.26 (s, 2H), 3.82 (s, 3H), 3.08 (s, 3H).

Ex-19C: The title compound was prepared from N-{4-[2-(2-methoxy-5-thien-2-yl-phenyl)-acetyl]-phenyl}-methanesulfonamide obtained from Ex-19B in a similar manner as described in Ex-3D. The crude material was purified by slurrying (EtOH/hexanes) and filtration to provide 79 mg (57%) of pure product as a faint orange solid, mp 197-198° C. ¹H-NMR (CDCl₃) δ 7.92 (d, J=8.9 Hz, 2H), 7.63 (d, J=2.1 Hz, 1H), 7.56 (dd, J=8.8, 2.1 Hz, 1H), 7.24-7.26 (m, 2H), 7.20 (d, J=8.9 Hz, 2H), 7.06-7.09 (m, 1H), 6.82 (d, J=8.8 Hz, 1H), 6.64 (bs, 1H), 6.03 (s, 1H), 5.77 (s, 1H), 3.59 (s, 3H), 3.10 (s, 3H). HRMS (EI⁺) m/z: calc. 413.0756, found 413.0766.

### Example 20

### N-{4-[2-(2-Methoxy-5-thien-2-yl-phenyl)-acryloyl]-phenyl}-N-methyl-methanesulfonamide

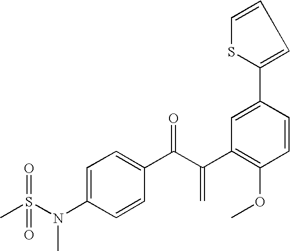

Ex-20: In a 10 mL round-bottom flask N-{4-[2-(2-methoxy-5-thien-2-yl-phenyl)-acryloyl]-phenyl}-methanesulfonamide obtained from Ex-19C (19 mg, 0.046 mmol) was combined with DMF (2.5 mL), Cs₂CO₃ (32 mg, 0.098 mmol), and methyl iodide (30 μL, 0.48 mmol). The resulting solution was heated at 150° C. After 1 h, HPLC indicated the reaction was complete. The reaction was cooled and diluted with H₂O, then extracted with EtOAc. The organic phase was washed with brine, then dried over Na₂SO₄, filtered, concentrated, and dried. The crude material was purified by silica gel chromatography (5-50% EtOAc in hexanes) to provide 10 mg (50%) of pure product as a white solid. 1H-NMR (CDCl₃) δ 7.94 (d, J=8.9 Hz, 2H), 7.63 (d, J=2.1 Hz, 1H), 7.56 (dd, J=8.9, 2.1 Hz, 11H), 7.43 (d, J=8.9 Hz, 2H), 7.24-7.26 (m, 2H), 7.06-7.09 (m, 1H), 6.83 (d, J=8.9 Hz, 1H), 6.06 (s, 1H), 5.78 (s, 1H), 3.61 (s, 3H), 3.37 (s, 3H), 2.87 (s, 3H).

### Example 21

### N-{4-[2-(2-Methoxy-5-thien-2-yl-phenyl)-acryloyl]-phenyl}-isobutyramide

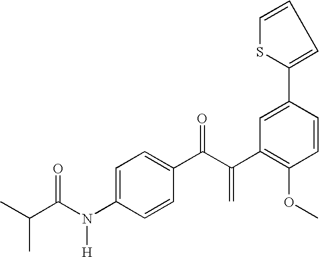

Ex-21A: In a 25 mL round-bottom flask 1-(4-amino-phenyl)-2-(2-methoxy-5-thien-2-yl-phenyl)-ethanone obtained from Ex-19A (0.23 g, 0.71 mmol) was combined with CH₂Cl₂ (8 mL). The solution was cooled in a 0° C. bath and triethylamine (0.11 mL, 0.79 mmol) was added, followed by dropwise addition of isobutyryl chloride (80 μL, 0.76 mmol). The reaction was allowed to warm to room temperature. After 4 h 15 min, HPLC indicated some starting material remained. Additional isobutyryl chloride (15 μL, 0.14 mmol) was added and stirring continued for another 1 h 30 min. HPLC indicated the reaction was complete. The solution was diluted with H₂O and acidified with 1 N HCl, then extracted with CH₂Cl₂. The organic phase was washed with brine, then dried over Na₂SO₄, filtered, concentrated, and dried. The crude material was purified by silica gel chromatography (50% EtOAc in hexanes) to provide 262 mg (94%) of N-{4-[2-(2-methoxy-5-thien-2-yl-phenyl)-acetyl]-phenyl}-isobutyramide as a solid. ¹H-NMR (CDCl₃) δ 8.03 (d, J=8.8 Hz, 2H), 7.63 (d, J=8.8 Hz, 2H), 7.48 (dd, J=8.1, 2.5 Hz, 1H), 7.42 (d, J=2.5 Hz, 1H), 7.18 (t, J=5.2 Hz, 2H), 7.02 (dd, J=5.2, 3.3 Hz, 1H), 6.88 (d, J=8.1 Hz, 1H), 4.27 (s, 2H), 3.81 (s, 3H), 2.53 (septet, J=7.3 Hz, 1H), 1.27 (d, J=7.3 Hz, 6H).

Ex-21B: The title compound was prepared from N-{4-[2-(2-methoxy-5-thien-2-yl-phenyl)-acetyl]-phenyl}-isobutyramide obtained from Ex-21A in a similar manner as described in Ex-3D. The crude material was purified by slurrying (EtOH/hexanes) and filtration to provide 125 mg (46%) of pure product as a faint orange solid, mp 155-158° C. ¹H-NMR (CDCl₃) δ 7.90 (d, J=8.8 Hz, 2H), 7.64 (d, J=2.7 Hz, 1H), 7.58 (d, J=8.8 Hz, 2H), 7.55 (dd, J=8.7, 2.7 Hz, 1H), 7.30 (bs, 1H), 7.23-7.26 (m, 2H), 7.07 (dd, J=4.5, 4.2 Hz, 1H), 6.80 (d, J=8.7 Hz, 1H), 6.01 (s, 1H), 5.76 (s, 1H), 3.57 (s, 3H), 2.53 (septet, J=7.0 Hz, 1H), 1.26 (d, J=7.0 Hz, 6H). HRMS (EI⁺) m/z: calc. 405.1399, found 405.1404.

### Example 22

### 2-(2-Methoxy-5-thien-2-yl-phenyl)-1-[4-(pyrimidin-2-ylamino)-phenyl]-propenone

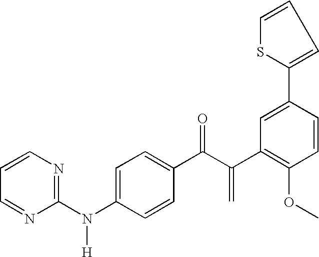

Ex-22A: In a 25 mL round-bottom flask 1-(4-amino-phenyl)-2-(2-methoxy-5-thien-2-yl-phenyl)-ethanone obtained from Ex-19A (298 mg, 0.921 mmol) was combined with 2-bromo pyrimidine (137 mg, 0.862 mmol), K₃PO₄ (254 mg, 1.20 mmol), and dioxane (3.5 mL). The reaction mixture was purged subsurface with nitrogen while adding tris(dibenzylideneacetone) dipalladium (0) (9 mg, 0.01 mmol) and xantphos (18 mg, 0.031 mmol). The reaction was purged several min more, then heated to 100° C. overnight. HPLC indicated small amounts of the starting materials remained. The mixture was diluted with H₂O and extracted with EtOAc. The organic phase was washed with brine, then dried over Na₂SO₄, filtered, concentrated, and dried. The crude material was purified by silica gel chromatography (50% EtOAc in hexanes) followed by slurrying (EtOAc/hexanes) and filtration to provide 172 mg (50%) of semi-pure 2-(2-methoxy-5-thien-2-yl-phenyl)-1-[4-(pyrimidin-2-ylamino)-phenyl]-ethanone (containing ˜8 mol % aniline starting material) as a faint, orange solid. ¹H-NMR (CDCl₃) δ 8.49 (d, J=4.8 Hz, 2H), 8.60 (d, J=9.0 Hz, 2H), 7.74 (d, J=9.0 Hz, 2H), 7.48 (dd, J=8.4, 2.5 Hz, 1H), 7.44 (d, J=2.5 Hz, 1H), 7.37 (bs, 1H), 7.19 (dd, J=5.4, 3.3 Hz, 2H), 7.03 (dd, J=5.4, 3.3 Hz, 1H), 6.84 (d, J=8.4 Hz, 1H), 6.83 (t, J=4.8 Hz, 1H), 4.28 (s, 2H), 3.82 (s, 3H).

Ex-22B: The title compound was prepared from 2-(2-methoxy-5-thien-2-yl-phenyl)-1-[4-(pyrimidin-2-ylamino)-phenyl]-ethanone obtained from Ex-22A in a similar manner as described in Ex-3D with extra acetic acid added (1.04 equiv.). The crude material was purified by slurrying (EtOH/hexanes) and filtration, followed by silica gel chromatography (50% EtOAc in hexanes), followed by slurrying again (EtOAc/EtOH/hexanes) and filtration to provide 56 mg (28%) of pure product as an off-white solid, mp 154-155° C. ¹H-NMR (CDCl₃) δ 8.48 (d, J=4.8 Hz, 2H), 7.93 (d, J=8.7 Hz, 2H), 7.71 (d, J=8.7 Hz, 2H), 7.66 (d, J=2.1 Hz, 1H), 7.55 (dd, J=7.8, 1.8 Hz, 1H), 7.33 (bs, 1H), 7.24-7.26 (m, 2H), 7.07 (dd, J=5.1, 3.6 Hz, 1H), 6.80-6.84 (m, 2H), 6.01 (s, 1H), 5.77 (s, 1H), 3.60 (s, 3H). HRMS (EI⁺) m/z: calc. 413.1198, found 413.1184.

### Example 23

### 2-{4-[2-(2-Methoxy-5-thien-2-yl-phenyl)-acryloyl]-phenylamino}-nicotinic acid ethyl ester

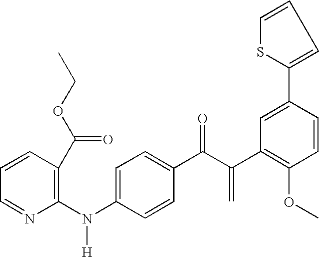

Ex-23A: 2-{4-[2-(2-Methoxy-5-thien-2-yl-phenyl)-acetyl]-phenylamino}-nicotinic acid ethyl ester was prepared from 1-(4-amino-phenyl)-2-(2-methoxy-5-thien-2-yl-phenyl)-ethanone obtained from Ex-19A and ethyl-2-chloronicotinate in a similar manner as described in Ex-22A. The crude material was purified by silica gel chromatography (25% EtOAc in hexanes) followed by slurrying (EtOAc/hexanes/CHCl₃) and filtration to provide 88 mg (24%) of pure product as a yellow solid. ¹H-NMR (CDCl₃) δ 10.58 (s, 1H), 8.44 (dd, J=4.9, 2.1 Hz, 1H), 8.30 (dd, J=7.2, 2.1 Hz, 1H), 8.06 (d, J=8.8 Hz, 2H), 7.86 (d, J=8.8 Hz, 2H), 7.48 (dd, J=8.4, 2.2 Hz, 1H), 7.44 (d, J=2.2 Hz, 1H), 7.18-7.20 (m, 2H), 7.03 (dd, J=5.4, 3.9 Hz, 2H), 6.89 (d, J=8.4 Hz, 1H), 6.83 (dd, J=7.2, 4.9 Hz, 1H), 4.41 (q, J=7.2 Hz, 2H), 4.29 (s, 2H), 3.82 (s, 3H), 1.43 (t, J=7.3 Hz, 3H).

Ex-23B: The title compound was prepared from 2-{4-[2-(2-methoxy-5-thien-2-yl-phenyl)-acetyl]-phenylamino}-nicotinic acid ethyl ester obtained from Ex-23A in a similar manner as described in Ex-3D. The crude material was purified by slurrying (EtOH/hexanes) and filtration to provide 49 mg (54%) of pure product as an off-white solid, mp 149-151° C. ¹H-NMR (CDCl₃) δ 10.56 (s, 1H), 8.43 (dd, J=5.1, 2.1 Hz, 1H), 8.30 (dd, J=7.2, 2.1 Hz, 1H), 7.94 (d, J=8.8 Hz, 2H), 7.82 (d, J=8.8 Hz, 2H), 7.66 (d, J=2.2 Hz, 1H), 7.54 (dd, J=9.3, 2.2 Hz, 1H), 7.24-7.26 (m, 2H), 7.07 (dd, J=5.4, 3.9 Hz, 1H), 6.80-6.84 (m, 2H), 6.01 (s, 1H), 5.77 (s, 1H), 4.40 (q, J=7.2 Hz, 2H), 3.60 (s, 3H), 1.43 (t, J=7.2 Hz, 3H). HRMS (EI⁺) m/z: calc. 484.1457, found 484.1466.

### Example 24

### 2-{4-[2-(2-Methoxy-5-thien-2-yl-phenyl)-acryloyl]-phenylamino}-nicotinic acid

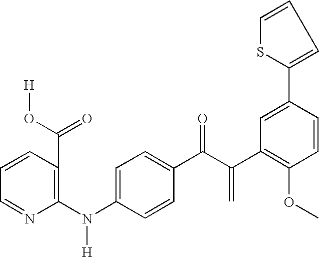

Ex-24A: In a 200 mL round-bottom flask 2-{4-[2-(2-methoxy-5-thien-2-yl-phenyl)-acetyl]-phenylamino}-nicotinic acid ethyl ester obtained from Ex-23A (impure, 723 mg, ˜1.5 mmol) was combined with THF (15 mL) and EtOH (2 mL). 1 N NaOH (10 mL) was added dropwise. The solution was purged subsurface with nitrogen for several min and then stirred at room temperature; HPLC indicated the reaction was incomplete after 1 h 10 min. Additional 1 N NaOH (2 mL) was added and stirring continued. HPLC indicated the reaction was complete after another 2 h 10 min. The solution was acidified to pH 3 with 1 N HCl, diluted with H₂O, and extracted with EtOAc. The organic phase was washed with brine, dried over Na₂SO₄, filtered, and concentrated. The crude material was purified by silica gel chromatography (5% MeOH in CH₂Cl₂) to provide 387 mg (57%) of 2-{4-[2-(2-methoxy-5-thien-2-yl-phenyl)-acetyl]-phenylamino}-nicotinic acid as an orange-yellow solid. ¹H-NMR (DMSO-d₆) δ 8.39-8.41 (m, 1H), 8.29 (d, J=6.3 Hz, 1H), 8.02 (d, J=8.7 Hz, 2H), 7.90 (d, J=8.7 Hz, 2H), 7.54 (dd, J=8.4, 2.8 Hz, 1H), 7.49 (d, J=2.8 Hz, 1H), 7.44 (d, J=4.8 Hz, 1H), 7.35 (dd, J=3.7, 1.5 Hz, 1H), 7.09 (dd, J=4.8, 3.7 Hz, 1H), 7.02 (d, J=8.4 Hz, 1H), 6.92-6.96 (m, 1H), 4.31 (s, 2H), 3.75 (s, 3H).

Ex-24B: The title compound was prepared from 2-{4-[2-(2-methoxy-5-thien-2-yl-phenyl)-acetyl]-phenylamino}-nicotinic acid obtained from Ex-24A in a similar manner as described in Ex-3D. The reaction was driven nearly to completion after 7 days by addition of excess formaldehyde, piperidine, and acetic acid periodically. The crude material was purified by silica gel chromatography (5% MeOH in CH₂Cl₂) to provide 20 mg (19%) of product (containing a small impurity) as a faint yellow solid, mp 234° C. (dec.). ¹H-NMR (DMSO-d₆) δ 10.52 (s, 1H), 8.43 (bs, 1H), 8.30 (d, J=7.5 Hz, 1H), 7.90 (d, J=8.1 HZ, 2H), 7.77 (d, J=8.1 Hz, 2H), 7.62 (d, J=1.5 Hz, 1H), 7.50 (dd, J=8.2, 1.5 Hz, 1H), 7.18-7.20 (m, 2H), 7.01 (dd, J=5.1, 3.9 Hz, 1H), 6.82 (bs, 1H), 6.76 (d, J=8.2 Hz, 1H), 5.99 (s, 1H), 5.75 (s, 1H), 3.55 (s, 3H). HRMS (EI⁺) m/z: calc. 456.1144, found 456.1141.

### Example 25

### 2-(2-Methoxy-5-thien-2-yl-phenyl)-1-[4-(pyrazin-2-ylamino)-phenyl]-propenone

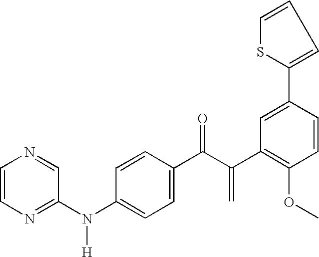

Ex-25A: 2-(2-Methoxy-5-thien-2-yl-phenyl)-1-[4-(pyrazin-2-ylamino)-phenyl]-ethanone was prepared from 1-(4-amino-phenyl)-2-(2-methoxy-5-thien-2-yl-phenyl)-ethanone obtained from Ex-19A and chloropyrazine in a similar manner as described in Ex-22A. The crude material was purified by silica gel chromatography (50% EtOAc in hexanes) to provide 62 mg (13%, 29% BRSM) of semi-pure product as a yellow residue. ¹H-NMR (CDCl₃) δ 8.21 (s, 1H), 8.16-8.17 (m, 1H), 8.02-8.04 (m, 3H), 7.59 (d, J=9.0 Hz, 2H), 7.41-7.46 (m, 2H), 7.38 (bs, 1H), 7.17 (d, J=6.0 Hz, 1H), 7.13 (d, J=2.4 Hz, 1H), 7.00 (dd, J=4.2, 3.0 Hz, 1H), 6.82 (d, J=9.0 Hz, 1H), 4.27 (s, 2H), 3.77 (s, 3H).

Ex-25B: The title compound was prepared from 2-(2-Methoxy-5-thien-2-yl-phenyl)-1-[4-(pyrazin-2-ylamino)-phenyl]-ethanone obtained from Ex-25A in a similar manner as described in Ex-3D. The crude material was purified by slurrying (EtOH/hexanes) and filtration to provide 11 mg (17%) of pure product as a beige solid, mp 178-182° C. ¹H-NMR (CDCl₃) δ 8.29 (s, 1H), 8.19-8.20 (m, 1H), 8.07 (d, J=2.4 Hz, 1H), 7.93 (d, J=9.0 Hz, 2H), 7.65 (d, J=2.1 Hz, 1H), 7.53-7.57 (m, 3H), 7.24-7.26 (m, 2H), 7.08 (t, J=4.2 Hz, 1H), 6.81 (d, J=7.8 Hz, 1H), 6.78 (bs, 1H), 6.02 (s, 1H), 5.78 (s, 1H), 3.60 (s, 3H). HRMS (EI⁺) m/z: calc. 413.1198, found 413.1202.

### Example 26

### 3,5-Dimethyl-isoxazole-4-sulfonic acid {4-[2-(2-methoxy-5-thien-2-yl-phenyl)-acryloyl]-phenyl}-amide

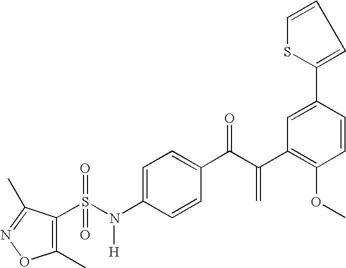

Ex-26A: 3,5-Dimethyl-isoxazole-4-sulfonic acid {4-[2-(2-methoxy-5-thien-2-yl-phenyl)-acetyl]-phenyl}-amide was prepared from 1-(4-amino-phenyl)-2-(2-methoxy-5-thien-2-yl-phenyl)-ethanone obtained from Ex-19A and 3,5-dimethylisoxazole-4-sulfonyl chloride in a similar manner as described in Ex-19B. The crude material was purified by silica gel chromatography (35% EtOAc in hexanes) to provide 347 mg (74%) of pure product as a light yellow solid. ¹H-NMR (CDCl₃) δ 8.00 (d, J=8.4 Hz, 2H), 7.49 (dd, J=8.1, 2.2 Hz, 1H), 7.39 (d, J=2.2 Hz, 1H), 7.20 (d, J=5.4 Hz, 1H), 7.17 (d, J=3.7 Hz, 1H), 7.13 (d, J=8.4 Hz, 2H), 7.03 (dd, J=5.4, 3.7 Hz, 1H), 6.89 (d, J=8.1 Hz, 1H), 6.79 (bs, 1H), 4.24 (s, 2H), 3.80 (s, 3H), 2.56 (s, 3H), 2.34 (s, 3H).

Ex-26B: The title compound was prepared from 3,5-dimethyl-isoxazole-4-sulfonic acid {4-[2-(2-methoxy-5-thien-2-yl-phenyl)-acetyl]-phenyl}-amide obtained from Ex-26A in a similar manner as described in Ex-3D. The crude material was purified by slurrying (EtOH/hexanes/EtOAc) and filtration to provide 114 mg (60%) of pure product as an off-white solid, mp 176-179° C. ¹H-NMR (CDCl₃) δ 7.84 (d, J=9.0 Hz, 2H), 7.63 (d, J=2.2 Hz, 1H), 7.56 (dd, J=8.2, 2.2 Hz, 1H), 7.24-7.26 (m, 2H), 7.08 (d, J=9.0 Hz, 2H), 7.06-7.09 (m, 2H), 6.80 (d, J=8.2 Hz, 1H), 6.79 (bs, 1H), 6.02 (s, 1H), 5.76 (s, 1H), 3.54 (s, 3H), 2.55 (s, 3H), 2.33 (s, 3H). HRMS (EI⁺) m/z: calc. 494.0970, found 494.0969.

### Example 27

### Isoxazole-5-carboxylic acid {4-[2-(2-methoxy-5-thien-2-yl-phenyl)-acryloyl]-phenyl}-amide

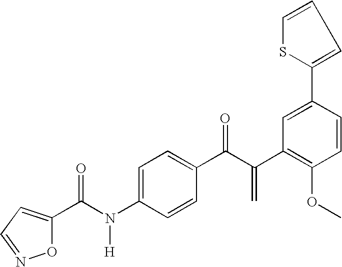

Ex-27A: Isoxazole-5-carboxylic acid {4-[2-(2-methoxy-5-thien-2-yl-phenyl)-acetyl]-phenyl}-amide was prepared from 1-(4-amino-phenyl)-2-(2-methoxy-5-thien-2-yl-phenyl)-ethanone obtained from Ex-19A and isoxazole-5-carbonylchloride in a similar manner as described in Ex-21A. Following workup, the organic phase contained insoluble solids. Filtration of the organics provided 130 mg (45%) of pure product as an off-white solid. ¹H-NMR (DMSO-d₆) δ 11.05 (s, 1H), 8.85 (d, J=1.5 Hz, 1H), 8.09 (d, J=8.5 Hz, 2H), 7.95 (d, J=8.5 Hz, 2H), 7.54 (dd, J=7.9, 2.1 Hz, 1H), 7.49 (d, J=2.1 Hz, 1H), 7.45 (d, J=5.4 Hz, 1H), 7.35 (d, J=3.7 Hz, 1H), 7.33 (d, J=1.5 Hz, 1H), 7.10 (dd, J=5.4, 3.7 Hz, 1H), 7.02 (d, J=7.9 Hz, 1H), 4.35 (s, 2H), 3.75 (s, 3H).

Ex-27B: The title compound was prepared from isoxazole-5-carboxylic acid {4-[2-(2-methoxy-5-thien-2-yl-phenyl)-acetyl]-phenyl}-amide obtained from Ex-27A in a similar manner as described in Ex-3D. MeOH and DMF were added to solubilize the substrate. The crude material was purified by silica gel chromatography (5% EtOAc in hexanes) followed by slurrying (EtOH/hexanes) and filtration to provide 71 mg (53%) of pure product as an off-white solid, mp 132.5-133.5° C. ¹H-NMR (DMSO-d₆) δ 11.02 (s, 1H), 8.84 (d, J=1.5 Hz, 1H), 7.90 (d, J=8.7 Hz, 2H), 7.84 (d, J=8.7 Hz, 2H), 7.68 (d, J=2.2 Hz, 1H), 7.61 (dd, J=8.4, 2.2 Hz, 1H), 7.49-7.52 (m, 2H), 7.31 (d, J=1.5 Hz, 1H), 7.13 (dd, J=5.4, 3.9 Hz, 1H), 7.01 (d, J=8.4 Hz, 1H), 6.14 (s, 1H), 5.72 (s, 1H), 3.53 (s, 3H). HRMS (EI⁺) m/z: calc. 430.0987, found 430.0985.

### Example 28

### 1-Methyl-1H-pyrrole-2-carboxylic acid {4-[2-(2-methoxy-5-thien-2-yl-phenyl)-acryloyl]-phenyl}-amide

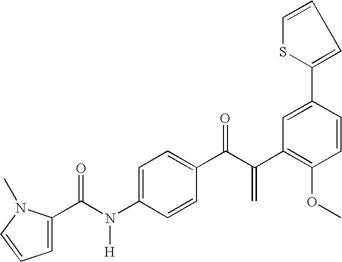

Ex-28A: 1-Methyl-1H-pyrrole-2-carboxylic acid {4-[2-(2-methoxy-5-thien-2-yl-phenyl)-acetyl]-phenyl}-amide was prepared from 1-(4-amino-phenyl)-2-(2-methoxy-5-thien-2-yl-phenyl)-ethanone obtained from Ex-19A and 1-methyl-1H-pyrrole-2-carbonyl chloride in a similar manner as described in Ex-21A. The crude material was purified by silica gel chromatography (35% EtOAc in hexanes) to provide 184 mg (68%) of pure product as a yellow foam. ¹H-NMR (CDCl₃) δ 8.06 (d, J=9.0 Hz, 2H), 7.71 (bs, 1H), 7.67 (d, J=9.0 Hz, 2H), 7.49 (dd, J=8.7, 2.5 Hz, 1H), 7.43 (d, J=2.5 Hz, 1H), 7.19 (d, J=5.2 Hz, 1H), 7.18 (d, J=3.9 Hz, 1H), 7.03 (dd, J=5.2, 3.9 Hz, 1H), 6.89 (d, J=8.7 Hz, 1H), 6.82-6.83 (m, 1H), 6.73 (dd, J=3.9, 1.5 Hz, 1H), 6.16 (dd, J=3.9, 2.7 Hz, 1H), 4.26 (s, 2H), 3.99 (s, 3H), 3.82 (s, 3H).

Ex-28B: The title compound was prepared from 1-methyl-1H-pyrrole-2-carboxylic acid {4-[2-(2-methoxy-5-thien-2-yl-phenyl)-acetyl]-phenyl}-amide obtained from Ex-28A in a similar manner as described in Ex-3D. The crude material was purified by slurrying (EtOAc/hexanes) and filtration to provide 89 mg (47%) of pure product as a light yellow solid, mp 165-168° C. ¹H-NMR (CDCl₃) δ 7.93 (d, J=8.1 Hz, 2H), 7.70 (bs, 1H), 7.65 (d, J=2.1 Hz, 1H), 7.61 (d, J=8.1 Hz, 2H), 7.55 (dd, J=8.7, 2.1 Hz, 1H), 7.24-7.26 (m, 2H), 7.08 (t, J=4.2 Hz, 1H), 6.80-6.83 (m, 2H), 6.73 (dd, J=3.9, 1.5 Hz, 1H), 6.16 (dd, J=4.8, 2.7 Hz, 1H), 6.02 (s, 1H), 5.78 (s, 1H), 3.99 (s, 3H), 3.59 (s, 3H). HRMS (EI⁺) m/z: calc. 442.1351, found 442.1356.

### Example 29

### 1-(2,6-Dimethyl-phenyl)-2-(2-methoxy-5-thien-2-yl-phenyl)-propenone

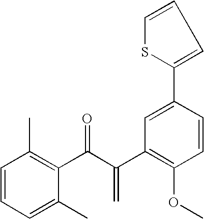

Ex-29A: (tert-Butyl-dimethyl-silanyloxy)-(2,6-dimethyl-phenyl)-acetonitrile was prepared from 2,6-dimethyl benzaldehyde in a similar manner as described in Ex-7A. The crude material was purified by silica gel chromatography (5% EtOAc in hexanes) to provide 5.06 g (98%) of pure product as a faint yellow oil. ¹H-NMR (CDCl₃) δ 7.16 (t, J=7.0 Hz, 1H), 7.04 (d, J=7.0 Hz, 2H), 5.85 (s, 1H), 2.50 (s, 6H), 0.90 (s, 9H), 0.24 (s, 3H), 0.04 (s, 3H).

Ex-29B: 2-(tert-Butyl-dimethyl-silanyloxy)-2-(2,6-dimethyl-phenyl)-3-(2-methoxy-5-thien-2-yl-phenyl)-propionitrile was prepared from (tert-Butyl-dimethyl-silanyloxy)-(2,6-dimethyl-phenyl)-acetonitrile obtained from Ex-29A and 2-(3-bromomethyl-4-methoxy-phenyl)-thiophene obtained from Ex-7D in a similar manner as described in Ex-7E. The crude material was purified by silica gel chromatography (5% EtOAc in hexanes) to provide 0.83 g of impure product as a faint yellow oil. ¹H-NMR (CDCl₃) δ 7.47-7.51 (m, 1H), 7.34 (d, J=2.4 Hz, 1H), 7.18-7.24 (m, 1H), 7.00-7.11 (m, 5H), 6.83 (d, J=9.0 Hz, 1H), 3.69 (s, 3H), 3.62 (d, J=13.2 Hz, 1H), 3.54 (d, J=13.2 Hz, 1H), 0.85 (s, 9H), 0.14 (s, 3H), 0.02 (s, 3H).

Ex-29C: 1-(2,6-Dimethyl-phenyl)-2-(2-methoxy-5-thien-2-yl-phenyl)-ethanone was prepared from 2-(tert-butyl-dimethyl-silanyloxy)-2-(2,6-dimethyl-phenyl)-3-(2-methoxy-5-thien-2-yl-phenyl)-propionitrile obtained from Ex-29B in a similar manner as described in Ex-7F. The crude material was purified by silica gel chromatography (10% EtOAc in hexanes) to provide 261 mg (2 steps, 28%) of pure product as a white semi-solid. ¹H-NMR (CDCl₃) δ 7.51 (dd, J=8.2, 2.2 Hz, 1H), 7.38 (d, J=2.2 Hz, 1H), 7.14-7.23 (m, 3H), 7.01-7.07 (s, 3H), 6.90 (d, J=8.2 Hz, 1H), 4.07 (s, 2H), 3.79 (s, 3H), 2.30 (s, 6H).

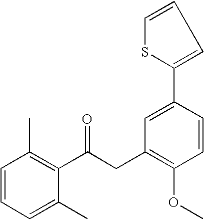

### 1-(2,6-Dimethyl-phenyl)-2-(2-methoxy-5-thien-2-yl-phenyl)-ethanone

Ex-29D: The title compound was prepared from 1-(2,6-dimethyl-phenyl)-2-(2-methoxy-5-thien-2-yl-phenyl)-ethanone obtained from Ex-29C in a similar manner as described in Ex-7G. The crude material was purified by silica gel chromatography (5% EtOAc in hexanes) to provide 175 mg (65%) of pure product as a sticky, white foam. ¹H-NMR (CDCl₃) δ 7.59 (dd, J=8.4, 2.2 Hz, 1H), 7.41 (d, J=2.2 Hz, 1H), 7.17-7.25 (m, 3H), 7.04-7.06 (m, 3H), 6.96 (d, J=8.4 Hz, 1H), 6.20 (s, 1H), 5.95 (s, 1H), 3.83 (s, 3H), 2.31 (s, 6H). HRMS (EI⁺) m/z: calc. 349.1262, found 349.1267. Anal. calculated for C₂₂H₂₀O₂S: C, 75.83; H, 5.78; S, 9.20. Found: C, 75.67; H, 5.73; S, 9.15%.

### Example 30

### 4-[1-(2,6-Dimethoxy-3-thien-2-yl-benzoyl)-vinyl]-benzoic acid

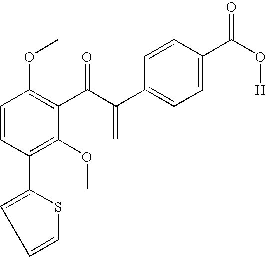

Ex-30A: In a 1 L round-bottom flask 2,6-dimethoxybenzaldehyde (50.00 g, 300.9 mmol) was combined with CH₂Cl₂ (716 mL). The solution was cooled in an ice bath and bromine (17.7 mL, 346.0 mmol) was added dropwise over 30 mins. After stirring for 1 hr, the reaction was quenched with H₂O-diluted 0.5 M sodium thiosulfate, which stirred for an additional 30 min. The organic phase was washed with H₂O-diluted 0.5 N NaOH followed by ½-saturated brine. The organics were dried over Na₂SO₄, filtered, and concentrated. The crude material was purified by crystallization (20% EtOH/H₂O) to provide 44.66 g (60%) of 3-bromo-2,6-dimethoxy-benzaldehyde as white crystals. 1H-NMR (CDCl₃) δ 10.41 (s, 1H), 7.68 (d, J=9.1 Hz, 1H), 6.70 (d, J=9.1 Hz, 1H), 3.91 (s, 3H), 3.90 (s, 3H).

Ex-30AA: (alterative procedure) In a 200 mL round-bottom flask 2,6-dimethoxybenzaldehyde (4.16 g, 25.0 mmol) was combined with CH₂Cl₂ (60 mL). The solution was cooled in an ice bath and bromine (1.36 mL, 26.5 mmol) was added dropwise over 25 min. After stirring for 1 h, the reaction was diluted with ice and extracted with CH₂Cl₂. The organic phase was washed with H₂₀, 1 N NaOH followed by ½-saturated brine. The organics were dried over Na₂SO₄, filtered, concentrated, and dried. The crude material was purified by crystallization (EtOH/H₂O) to provide 2.38 g (39%) of 3-bromo-2,6-dimethoxy-benzaldehyde as off-white crystals.

Ex-30B: (3-Bromo-2,6-dimethoxy-phenyl)-(tert-butyl-dimethyl-silanyloxy)-acetonitrile was prepared from 3-bromo-2,6-dimethoxy-benzaldehyde obtained from Ex-30A or Ex-30AA in a similar manner as described in Ex-7A. The crude material was purified by silica gel chromatography (2 columns at 5% EtOAc in hexanes) to provide 64.82 g (81%) of pure product as a white solid. ¹H-NMR (CDCl₃) δ 7.52 (d, J=9.1 Hz, 1H), 6.61 (d, J=9.1 Hz, 1H), 5.99 (s, 1H), 3.98 (s, 3H), 3.86 (s, 3H), 0.88 (s, 9H), 0.23 (s, 3H), 0.08 (s, 3H).

Ex-30C: In a 250 mL round-bottom flask (3-bromo-2,6-dimethoxy-phenyl)-(tert-butyl-dimethyl-silanyloxy)-acetonitrile obtained from Ex-30B (9.99 g, 25.9 mmol) was combined with THF (86 mL). The solution was cooled in a −78° C. bath and LHMDS (1 M in THF, 28.5 mL) was added dropwise. After 5 min, the cold bath was removed. After stirring an additional 3 min, the solution was placed in a 0° C. bath and stirred for 2.5 h. In a 200 mL round-bottom flask methyl (4-bromomethyl) benzoate (5.45 g, 23.8 mmol) was combined with THF (69 mL) and the solution was cooled in a 0° C. bath. Via cannula, the anion solution was transferred to the 250 mL flask in a slow stream. The resulting solution was allowed to warm to room temperature immediately. After 10 min, HPLC indicated very little cyanohydrin remained. After 1.5 h the solution was diluted with aqueous NH₄Cl and extracted with EtOAc. The organic phase was washed with brine, dried over Na₂SO₄, filtered, concentrated, and dried. The crude material was purified by silica gel chromatography (15% EtOAc in hexanes) to provide 11.65 g (92%) of 4-[2-(3-bromo-2,6-dimethoxy-phenyl)-2-(tert-butyl-dimethyl-silanyloxy)-2-cyano-ethyl]-benzoic acid methyl ester as a colorless oil/foam. ¹H-NMR (CDCl₃) δ 7.87 (d, J=8.7 Hz, 2H), 7.50 (d, J=8.8 Hz, 1H), 7.22 (d, J=8.7 Hz, 2H), 6.64 (d, J=8.8 Hz, 1H), 3.96 (d, J=12.3 Hz, 1H), 3.89 (s, 3H), 3.88 (s, 3H), 3.58 (d, J=12.3 Hz, 1H), 3.37 (s, 3H), 0.86 (s, 9H), 0.13 (s, 3H), −0.20 (s, 3H).

Ex-30D: 4-[2-(3-Bromo-2,6-dimethoxy-phenyl)-2-oxo-ethyl]-benzoic acid methyl ester was prepared from 4-[2-(3-bromo-2,6-dimethoxy-phenyl)-2-(tert-butyl-dimethyl-silanyloxy)-2-cyano-ethyl]-benzoic acid methyl ester obtained from Ex-30C in a similar manner as described in Ex-7F. The crude material was purified by silica gel chromatography (25% EtOAc in hexanes) to provide 8.26 g (96%) of semi-pure product as a yellow solid. ¹H-NMR (CDCl₃) δ 7.98 (d, J=8.1 Hz, 2H), 7.48 (d, J=8.7 Hz, 1H), 7.29 (d, J=8.1 Hz, 2H), 6.57 (d, J=8.7 Hz, 1H), 4.11 (s, 2H), 3.91 (s, 3H), 3.80 (s, 3H), 3.76 (s, 3H).

Ex-30E: In a 200 mL round-bottom flask 4-[2-(3-bromo-2,6-dimethoxy-phenyl)-2-oxo-ethyl]-benzoic acid methyl ester obtained from Ex-30D (8.26 g, 21.00 mmol) was combined with thiophene 2-boronic acid (3.39 g, 26.5 mmol) and THF (85 mL). The resulting solution was purged subsurface with nitrogen while adding KF (3.66 g, 63.0 mmol) and bis-(tri-t-butylphosphine) palladium (0) (215 mg, 0.42 mmol). The solution was purged another 5 min, then heated to reflux for 2.5 h, at which point HPLC indicated the reaction was complete. The cooled reaction mixture was diluted with H₂O and extracted with EtOAc. The organic phase was washed with brine, dried over Na₂SO₄, filtered, concentrated, and dried. The crude material was purified by silica gel chromatography (2 columns at 15-20% EtOAc in hexanes) to provide 5.40 g (65%) of 4-[2-(2,6-dimethoxy-3-thien-2-yl-phenyl)-2-oxo-ethyl]-benzoic acid methyl ester as a yellow oil. ¹H-NMR (CDCl₃) δ 7.99 (d, J=7.8 Hz, 2H), 7.54 (d, J=8.5 Hz, 1H), 7.33-7.36 (m, 4H), 7.08 (dd, J=5.1, 3.6 Hz, 1H), 6.70 (d, J=8.5 Hz, 1H), 4.18 (s, 2H), 3.91 (s, 3H), 3.79 (s, 3H), 3.58 (s, 3H).

Ex-30F: In a 1L mL round-bottom flask potassium tert-butoxide (5.70 g, 50.4 mmol) was combined with THF (75 mL). The solution was heated to 60° C. and treated dropwise over a 20 min period with a solution of 4-[2-(2,6-dimethoxy-3-thien-2-yl-phenyl)-2-oxo-ethyl]-benzoic acid methyl ester obtained from Ex-30E (5.00 g, 12.6 mmol) in THF (25 mL). After stirring 30 minutes, HPLC indicated that the reaction was 99% complete. The reaction was diluted with water followed by an extraction with EtOAc. The aqueous portion was acidified with concentrated HCl acid to a pH of 1, then extracted with EtOAc. The organic phase was washed with ½-saturated brine, dried over Na₂SO₄, filtered, and concentrated to provide 4.53 g (94%) of 4-[2-(2,6-dimethoxy-3-thien-2-yl-phenyl)-2-oxo-ethyl]-benzoic acid as an off-white solid. ¹H-NMR (DMSO-d₆) δ 12.90 (bs, 1H), 7.90 (d, J=8.4 Hz, 2H), 7.71 (d, J=9.0 Hz, 1H), 7.57 (d, J=5.0 Hz, 1H), 7.46 (d, J=3.6 Hz, 1H), 7.35 (d, J=8.1 Hz, 2H), 7.13 (dd, J=5.0, 3.6 Hz, 1H), 6.98 (d, J=8.4 Hz, 1H), 4.21 (s, 2H), 3.83 (s, 3H), 3.54 (s, 3H).

Ex-30FF: In a 200 mL round-bottom flask 4-[2-(2,6-dimethoxy-3-thien-2-yl-phenyl)-2-oxo-ethyl]-benzoic acid methyl ester obtained from Ex-30E (5.39 g, 13.59 mmol) was combined with THF (100 mL) and MeOH (20 mL). The solution was purged subsurface with nitrogen. 1 N NaOH (28.3 mL) was added dropwise and purging continued for several more min. The solution was stirred at room temperature for 1.5 h, at which point HPLC indicated incomplete conversion. Additional 1 N NaOH (5 mL) was added and the reaction was heated at 35-45° C. for 3 h 10 min, at which point HPLC indicated the reaction was complete. The solution was cooled in an ice bath and acidified to pH˜2 with 1 N HCl, then extracted with CH₂Cl₂ (with a small amount of MeOH added). The organic phase was washed with ½-saturated brine, dried over Na₂SO₄, filtered, concentrated, and dried. The crude material was purified by silica gel chromatography (5% MeOH in CH₂Cl₂) to provide 4.86 g (94%) of 4-[2-(2,6-dimethoxy-3-thien-2-yl-phenyl)-2-oxo-ethyl]-benzoic acid as an off-white solid.

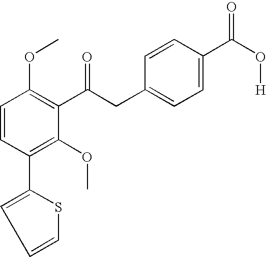

### 4-[2-(2,6-dimethoxy-3-thien-2-yl-phenyl)-2-oxo-ethyl]-benzoic acid

Ex-30G: In a 500 mL round-bottom flask 4-[2-(2,6-dimethoxy-3-thien-2-yl-phenyl)-2-oxo-ethyl]-benzoic acid obtained from Ex-30F or Ex-30FF (20.00 g, 52.3 mmol) was combined with THF (150 mL) and treated with 37 wt % formaldehyde in water (58 mL, 782.4 mmol), piperidine (724.0 μL, 7.32 mmol), and acetic acid (724.0 μL, 12.6 mmol). The reaction mixture was heated at 65° C. for 1 hr and 20 min and reaction completeness and product were confirmed by ¹H-NMR. The reaction mixture was diluted with water and was acidified with 1N HCl to a pH of 2 followed by an extraction with EtOAc. The organic phase was washed with ½-saturated brine, dried over Na₂SO₄, filtered, and concentrated. The crude material was purified by swishing in 50% acetone/water to provide 17.42 g (84%) of 4-[1-(2,6-dimethoxy-3-thien-2-yl-benzoyl)-vinyl]-benzoic acid as an off-white solid, mp 193-195° C. ¹H-NMR (CDCl₃) δ 8.14 (d, J=7.8 Hz, 2H), 7.60 (d, J=7.8 Hz, 2H), 7.60 (d, J=8.4 Hz, 1H), 7.37 (d, J=3.6 Hz, 1H), 7.33 (dd, J=5.1, 1.8 Hz, 1H), 7.09 (dd, J=5.1, 3.6 Hz, 1H), 6.77 (d, J=8.4 Hz, 1H), 6.28 (s, 1H), 6.08 (s, 1H), 3.83 (s, 3H), 3.63 (s, 3H). HRMS (EI⁺) m/z: calc. 394.0875, found 394.0869. Anal. calculated for C₂₂H₁₈O₅S: C, 66.99; H, 4.60; S, 8.13. Found: C, 67.00; H, 4.69; S, 8.07%.

Ex-30GG: The title compound was prepared from 4-[2-(2,6-dimethoxy-3-thien-2-yl-phenyl)-2-oxo-ethyl]-benzoic acid obtained from Ex-30F or Ex-30FF in a similar manner as described in Ex-7E. The crude material was purified by slurrying (EtOH/hexanes) and filtration to provide 157 mg (75%) of pure product as an off-white solid.

### Example 31

### 1-(2,6-Dimethoxy-3-thien-2-yl-phenyl)-2-(2-methoxy-phenyl)-propenone

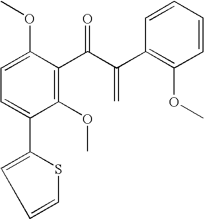

Ex-31A: (2-Methoxy-phenyl)-methanol was prepared from 2-methoxy benzaldehyde in a similar manner as described in Ex-7C. The resulting colorless oil was used without purification (1.94 g, 96%). ¹H-NMR (CDCl₃) δ 7.27 (d, J=7.5 Hz, 2H), 6.88-6.97 (m, 2H), 4.68 (d, J=5.7 Hz, 2H), 3.87 (s, 3H).

Ex-31B: 1-Bromomethyl-2-methoxy-benzene was prepared from (2-methoxy-phenyl)-methanol obtained from Ex-31A in a similar manner as described in Ex-7D. The crude material was purified by silica gel chromatography (12% EtOAc in hexanes) to provide 1.29 g of pure product as a colorless oil. ¹H-NMR (CDCl₃) δ 7.27-7.34 (m, 2H), 6.87-6.95 (m, 2H), 4.58 (s, 2H), 3.90 (s, 3H).

Ex-31C: 2-(3-Bromo-2,6-dimethoxy-phenyl)-2-(tert-butyl-dimethyl-silanyloxy)-3-(2-methoxy-phenyl)-propionitrile was prepared from 1-bromomethyl-2-methoxy-benzene obtained from Ex-31B and (3-bromo-2,6-dimethoxy-phenyl)-(tert-butyl-dimethyl-silanyloxy)-acetonitrile obtained from Ex-30B in a similar manner as described in Ex-7E. The crude material was purified by silica gel chromatography (15% EtOAc in hexanes) to provide 871 mg of semi-pure product as a faint yellow, viscous oil. ¹H-NMR (CDCl₃) δ 7.46 (d, J=9.3 Hz, 1H), 7.23 (dd, J=7.5, 1.5 Hz, 11H), 7.17 (dt, J=7.5, 1.5 Hz, 11H), 6.84 (dt, J=7.5, 1.5 Hz, 1H), 6.74 (d, J=7.5 Hz, 1H), 6.60 (d, J=9.3 Hz, 11H), 4.09 (d, J=12.0 Hz, 1H), 3.86 (s, 3H), 3.56 (s, 3H), 3.48 (d, J=12.0 Hz, 1H), 3.33 (s, 3H), 0.89 (s, 9H), 0.16 (s, 3H), −0.14 (s, 3H).

Ex-31D: 1-(3-Bromo-2,6-dimethoxy-phenyl)-2-(2-methoxy-phenyl)-ethanone was prepared from 2-(3-bromo-2,6-dimethoxy-phenyl)-2-(tert-butyl-dimethyl-silanyloxy)-3-(2-methoxy-phenyl)-propionitrile obtained from Ex-31C in a similar manner as described in Ex-7F. The crude material was purified by silica gel chromatography (12% EtOAc in hexanes) to provide 463 mg (2 steps, 39%) of pure product as a colorless oil. ¹H-NMR (CDCl₃) δ 7.45 (d, J=9.0 Hz, 1H), 7.22 (dd, J=7.5, 1.9 Hz, 1H), 7.16 (dd, J=7.5, 1.9 Hz, 1H), 6.83-6.93 (m, 2H), 6.55 (d, J=9.0 Hz, 1H), 4.10 (s, 2H), 3.82 (s, 3H), 3.76 (s, 3H), 3.75 (s, 3H).

Ex-31E: 1-(2,6-Dimethoxy-3-thien-2-yl-phenyl)-2-(2-methoxy-phenyl)-ethanone was prepared from 1-(3-bromo-2,6-dimethoxy-phenyl)-2-(2-methoxy-phenyl)-ethanone obtained from Ex-31D and thiophene 2-boronic acid in a similar manner as described in Ex-30E. The crude material was purified by silica gel chromatography (12% EtOAc in hexanes) to provide 436 mg (93%) of pure product as a colorless oil. ¹H-NMR (CDCl₃) δ 7.52 (d, J=8.8 Hz, 1H), 7.31-7.35 (m, 2H), 7.19-7.23 (m, 2H), 7.07 (dd, J=5.1, 3.6 Hz, 1H), 6.91 (t, J=7.2 Hz, 1H), 6.84 (d, J=8.7 Hz, 1H), 6.67 (d, J=8.8 Hz, 1H), 4.16 (s, 2H), 3.78 (s, 3H), 3.77 (s, 3H), 3.62 (s, 3H).

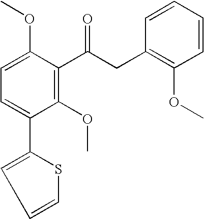

### 1-(2,6-Dimethoxy-3-thien-2-yl-phenyl)-2-(2-methoxy-phenyl)-ethanone

Ex-31F: The title compound was prepared from 1-(2,6-dimethoxy-3-thien-2-yl-phenyl)-2-(2-methoxy-phenyl)-ethanone obtained from Ex-31E in a similar manner as described in Ex-7G. The crude material was purified by silica gel chromatography (15% EtOAc in hexanes) to provide 289 mg (65%) of pure product as a sticky, white foam. 1H-NMR (CDCl₃) δ 7.55 (d, J=8.4 Hz, 1H), 7.37 (dd, J=3.6, 1.5 Hz, 1H), 7.23-7.33 (m, 3H), 7.07 (dd, J=5.4, 3.6 Hz, 1H), 6.96 (t, J=7.8 Hz, 1H), 6.87 (d, J=7.8 Hz, 1H), 6.69 (d, J=8.4 Hz, 1H), 6.16 (s, 1H), 6.10 (s, 1H), 3.79 (s, 3H), 3.78 (s, 3H), 3.67 (s, 3H). HRMS (EI⁺) m/z: calc. 380.1084, found 380.1084. Anal. calculated for C₂₂H₂₀O₄S: C, 69.45; H, 5.30; S, 8.43. Found: C, 69.15; H, 5.34; S, 8.35%.

### Example 32

### 4-[1-(2-Chloro-6-methyl-benzoyl)-vinyl]-3-thien-2-yl-benzoic acid

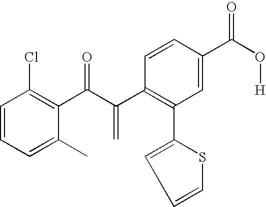

Ex-32A: In a 100 mL round-bottom flask 2-chloro-6-methyl-benzonitrile (2.01 g, 13.3 mmol) was combined with THF (28 mL). Di-isobutyl aluminum hydride (1 M in THF, 14.6 mL) was added quickly dropwise. After addition was complete, the reaction was heated to 45° C. overnight, at which point HPLC indicated only 25% conversion. Additional DIBAL-H (6.7 mL) was added and the reaction was heated at 45° C. for another 2 h, at which point HPLC indicated the reaction was still incomplete while impurities were forming. The reaction was cooled in an ice bath and MeOH was added, followed by H₂O, then 1 N HCl until the pH was 3. The solution was extracted with EtOAc. The organic phase was washed with 2 N Na₂CO₃, then brine, dried over Na₂SO₄, filtered, concentrated, and dried. The crude material was purified by silica gel chromatography (2.5% EtOAc in hexanes) to provide 0.48 g (23%) of 2-chloro-6-methyl-benzaldehyde as a faint yellow oil. ¹H-NMR (CDCl₃) δ 10.66 (s, 1H), 7.26-7.39 (m, 2H), 7.16 (d, J=7.2 Hz, 1H), 2.59 (s, 3H).

Ex-32B: (tert-Butyl-dimethyl-silanyloxy)-(2-chloro-6-methyl-phenyl)-acetonitrile was prepared from 2-chloro-6-methyl-benzaldehyde obtained from Ex-32A in a similar manner as described in Ex-7A. The crude material was purified by silica gel chromatography (5% EtOAc in hexanes, then a second column with 2.5% EtOAc in hexanes) to provide 1.00 g (61%) of pure product as a colorless oil. ¹H-NMR (CDCl₃) δ 7.20-7.25 (m, 2H), 7.14-7.18 (m, 1H), 6.29 (s, 1H), 2.66 (s, 3H), 0.89 (s, 9H), 0.25 (s, 3H), 0.04 (s, 3H).

Ex-32C: In a 500 mL round-bottom flask 3-bromo-4-methyl-benzoic acid (25.0 g, 116.3 mmol), was combined with MeOH (250 mL) and H₂SO₄ (0.5 mL) was added. The resulting solution was heated to 60° C. and aged overnight. The reaction was cooled to room temperature, concentrated to ⅕ the volume, diluted with EtOAc and saturated NaHCO₃ and the layers were cut. The organic layer was washed with H₂O, dried with MgSO₄ and concentrated to dryness. The crude was purified by crystallization from EtOAc and hexanes to provide 24.8 g (93%) of 3-bromo-4-methyl-benzoic acid methyl ester. ¹H-NMR (CDCl₃) δ 8.18 (d, J=2.1 Hz, 11H), 7.85 (dd, J=8.1, 1.9 Hz, 1H), 7.28 (d, J=8.1 Hz, 11H), 3.90 (s, 3H), 2.44 (s, 3H).

Ex-32D: In a 500 mL round-bottom flask 3-bromo-4-methyl-benzoic acid methyl ester (24.8 g, 108.3 mmol) obtained from Ex-32C, N-bromosuccinimide (21 g, 118.0 mmol), and AIBN (300 mg, 1.83 mmol) was combined with CCl₄ (250 mL). The resulting solution was heated to reflux and aged for 4 h. The reaction was cooled to room temperature, the precipitate was filtered and rinsed with fresh CCl₄. The combined filtrate was concentrated to dryness and then purified by silica gel chromatography (5% EtOAc in hexanes) to provide 16.8 g (50%) of 3-bromo-4-bromomethyl-benzoic acid methyl ester. ¹H-NMR (CDCl₃) δ 8.24 (d, J=1.3 Hz, 1H), 7.95 (dd, J=8.1, 1.3 Hz, 1H), 7.52 (d, J=8.1 Hz, 1H), 4.61 (s, 2H), 3.93 (s, 3H).

Ex-32E: 3-Bromo-4-[2-(tert-butyl-dimethyl-silanyloxy)-2-(2-chloro-6-methyl-phenyl)-2-cyano-ethyl]-benzoic acid methyl ester was prepared from 3-bromo-4-bromomethyl-benzoic acid methyl ester obtained from Ex-32D and (tert-butyl-dimethyl-silanyloxy)-(2-chloro-6-methyl-phenyl)-acetonitrile obtained from Ex-32B in a similar manner as described in Ex-7E. The crude material was purified by silica gel chromatography (10% EtOAc in hexanes) to provide 1.03 g (63%) of pure product as a viscous oil. ¹H-NMR (CDCl₃) δ 8.25 (d, J=1.3 Hz, 1H), 7.90 (dd, J=7.7, 1.3 Hz, 1H), 7.50 (d, J=7.7 Hz, 1H), 7.28 (d, J=7.7 Hz, 1H), 7.16 (t, J=7.7 Hz, 1H), 7.07 (d, J=7.7 Hz, 1H), 3.96 (d, J=13.7 Hz, 1H), 3.93 (s, 3H), 3.81 (d, J=13.7 Hz, 1H), 2.66 (s, 3H), 0.87 (s, 9H), 0.15 (s, 3H), 0.01 (s, 3H).

Ex-32F: 3-Bromo-4-[2-(2-chloro-6-methyl-phenyl)-2-oxo-ethyl]-benzoic acid methyl ester was prepared from 3-bromo-4-[2-(tert-butyl-dimethyl-silanyloxy)-2-(2-chloro-6-methyl-phenyl)-2-cyano-ethyl]-benzoic acid methyl ester obtained from Ex-32E in a similar manner as described in Ex-7F. The crude material was purified by silica gel chromatography (10% EtOAc in hexanes) to provide 669 mg (89%) of pure product as a colorless oil. ¹H-NMR (CDCl₃) δ 8.28 (d, J=1.3 Hz, 1H), 7.96 (dd, J=7.9, 1.3 Hz, 1H), 7.40 (d, J=7.9 Hz, 1H), 7.25 (dd, J=5.1, 1.2 Hz, 2H), 7.11-7.14 (m, 1H), 4.42 (s, 2H), 3.93 (s, 3H), 2.26 (s, 3H).

Ex-32G: 4-[2-(2-Chloro-6-methyl-phenyl)-2-oxo-ethyl]-3-thien-2-yl-benzoic acid methyl ester was prepared from 3-bromo-4-[2-(2-chloro-6-methyl-phenyl)-2-oxo-ethyl]-benzoic acid methyl ester obtained from Ex-32F and thiophene 2-boronic acid in a similar manner as described in Ex-30E. The crude material was purified by silica gel chromatography (10% EtOAc in hexanes) to provide 608 mg (90%) of semi-pure product as a sticky syrup. ¹H-NMR (CDCl₃) δ 8.10 (d, J=1.6 Hz, 1H), 8.02 (dd, J=8.1, 1.6 Hz, 1H), 7.44 (d, J=8.1 Hz, 1H), 7.35 (dd, J=5.1, 1.2 Hz, 1H), 7.18-7.20 (m, 2H), 7.03-7.06 (m, 2H), 6.99-7.00 (m, 1H), 4.32 (s, 2H), 3.93 (s, 3H), 1.98 (s, 3H).

Ex-32H: 4-[2-(2-Chloro-6-methyl-phenyl)-2-oxo-ethyl]-3-thien-2-yl-benzoic acid was prepared from 4-[2-(2-chloro-6-methyl-phenyl)-2-oxo-ethyl]-3-thien-2-yl-benzoic acid methyl ester obtained from Ex-32G in a similar manner as described in Ex-30FF. The crude material was purified by silica gel chromatography (5% MeOH in CH₂Cl₂) to provide semi-pure beige solids which were further purified by slurrying (EtOH/hexanes) and filtration to provide 130 mg (33%) of pure product. ¹H-NMR (DMSO-d₆) δ 7.95 (dd, J=8.0, 1.8 Hz, 1H), 7.90 (d, J=1.8 Hz, 1H), 7.65 (dd, J=4.5, 0.9 Hz, 1H), 7.48 (d, J=8.0 Hz, 1H), 7.32-7.34 (m, 2H), 7.23-7.24 (m, 1H), 7.10-7.15 (m, 2H), 4.41 (s, 2H), 2.01 (s, 3H).

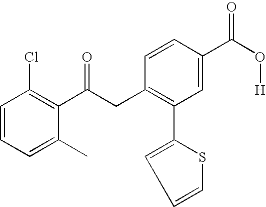

### 4-[2-(2-Chloro-6-methyl-phenyl)-2-oxo-ethyl]-3-thien-2-yl-benzoic acid

Ex-321: In a 10 mL round-bottom flask 4-[2-(2-chloro-6-methyl-phenyl)-2-oxo-ethyl]-3-thien-2-yl-benzoic acid obtained from Ex-32H (108 mg, 0.291 mmol) was combined with N,N,N′,N′-tetramethyl diaminomethane (3.3 mL, 24.4 mmol). Acetic anhydride (0.55 mL, 5.82 mmol) was added and the resulting solution heated at 50° C. overnight, at which point NMR indicated a small amount of starting material remained. Additional acetic anhydride (50 μL) was added and the solution heated another few hours, at which point NMR indicated the reaction was complete. The solution was cooled in an ice bath and 1 N HCl was added until the pH was ˜1. The whitish mix was extracted with CH₂Cl₂ (with a small amount of MeOH added). The organic phase was washed with 12-saturated brine, dried over Na₂SO₄, filtered, concentrated, and dried. The crude material was purified by silica gel chromatography (6.5% MeOH in CH₂Cl₂) to provide 72 mg (65%) of the title compound as a white foam, mp 88° C. (dec.). ¹H-NMR (DMSO-d₆) δ 7.98-8.01 (m, 2H), 7.59 (dd, J=5.1, 1.8 Hz, 1H), 7.42 (d, J=7.8 Hz, 1H), 7.36-7.38 (m, 2H), 7.25-7.28 (m, 1H), 7.05-7.10 (m, 2H), 6.40 (s, 1H), 5.95 (s, 1H), 1.99 (s, 3H). HRMS (EI⁺) m/z: calc. 383.0509, found 383.0514. Anal. calculated for C₂₁H₁₅ClO₃S: C, 65.88; H, 3.95; S, 8.38. Found: C, 65.00; H, 4.02; S, 8.11%.

### Example 33

### 4-[1-(2,6-Dimethyl-benzoyl)-vinyl]-3-thien-2-yl-benzoic acid

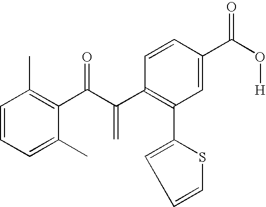

Ex-33A: 3-Bromo-4-[2-(tert-butyl-dimethyl-silanyloxy)-2-cyano-2-(2,6-dimethyl-phenyl)-ethyl]-benzoic acid methyl ester was prepared from 3-bromo-4-bromomethyl-benzoic acid methyl ester obtained from Ex-32D and (tert-butyl-dimethyl-silanyloxy)-(2,6-dimethyl-phenyl)-acetonitrile obtained from Ex-29A in a similar manner as described in Ex-7E. The crude material was purified by silica gel chromatography (10% EtOAc in hexanes) to provide 1.98 g (60%) of semi-pure product as a yellow oil. 1H-NMR (CDCl₃) δ 8.27 (d, J=1.6 Hz, 11H), 7.90 (dd, J=7.6, 1.6 Hz, 11H), 7.44 (d, J=7.6 Hz, 1H), 7.12 (t, J=7.7 Hz, 1H), 7.01 (d, J=7.7 Hz, 2H), 3.93 (s, 3H), 3.81 (d, J=13.5 Hz, 1H), 3.65 (d, J=13.5 Hz, 1H), 2.60 (s, 6H), 0.85 (s, 9H), 0.13 (s, 3H), −0.03 (s, 3H).

Ex-33B: 3-Bromo-4-[2-(2,6-dimethyl-phenyl)-2-oxo-ethyl]-benzoic acid methyl ester was prepared from 3-bromo-4-[2-(tert-butyl-dimethyl-silanyloxy)-2-cyano-2-(2,6-dimethyl-phenyl)-ethyl]-benzoic acid methyl ester obtained from Ex-33A in a similar manner as described in Ex-7F. The crude material was purified by silica gel chromatography (10% EtOAc in hexanes) to provide 0.92 g (65%) of pure product as a white solid. ¹H-NMR (CDCl₃) δ 8.28 (d, J=1.3 Hz, 1H), 7.96 (dd, J=7.9, 1.3 Hz, 1H), 7.34 (d, J=7.9 Hz, 1H), 7.20 (t, J=8.1 Hz, 1H), 7.04 (d, J=8.1 Hz, 2H), 4.29 (s, 2H), 3.93 (s, 3H), 2.31 (s, 6H).

Ex-33C: 4-[2-(2,6-Dimethyl-phenyl)-2-oxo-ethyl]-3-thien-2-yl-benzoic acid methyl ester was prepared from 3-bromo-4-[2-(2,6-dimethyl-phenyl)-2-oxo-ethyl]-benzoic acid methyl ester obtained from Ex-33B and thiophene 2-boronic acid in a similar manner as described in Ex-30E. The crude material was purified by silica gel chromatography (10% EtOAc in hexanes) to provide 757 mg (93%) of pure product as a faint orange solid. ¹H-NMR (CDCl₃) δ 8.09 (d, J=1.5 Hz, 1H), 8.02 (dd, J=8.1, 1.5 Hz, 1H), 7.38 (d, J=8.1 Hz, 1H), 7.35 (d, J=5.1 Hz, 1H), 7.14 (t, J=7.8 Hz, 1H), 7.04 (dd, J=5.1, 3.9 Hz, 1H), 6.96 (d, J=8.1 Hz, 2H), 6.93-6.95 (m, 1H), 4.21 (s, 2H), 3.92 (s, 3H), 2.05 (s, 6H).

Ex-33D: 4-[2-(2,6-Dimethyl-phenyl)-2-oxo-ethyl]-3-thien-2-yl-benzoic acid was prepared from 4-[2-(2,6-dimethyl-phenyl)-2-oxo-ethyl]-3-thien-2-yl-benzoic acid methyl ester obtained from Ex-33C in a similar manner as described in Ex-30FF. The crude material was purified by silica gel chromatography (6.5% MeOH in CH₂Cl₂) to provide 205 mg (72%) of pure product as a beige solid. ¹H-NMR (DMSO-d₆) δ 7.95 (dd, J=8.1, 1.5 Hz, 1H), 7.89 (d, J=1.5 Hz, 1H), 7.65 (dd, J=5.1, 1.2 Hz, 1H), 7.47 (d, J=8.1 Hz, 1H), 7.11-7.21 (m, 3H), 7.03 (d, J=7.8 Hz, 2H), 4.33 (s, 2H), 1.99 (s, 6H).

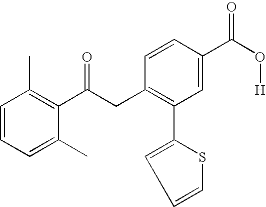

### 4-[2-(2,6-Dimethyl-phenyl)-2-oxo-ethyl]-3-thien-2-yl-benzoic acid

Ex-33E: The title compound was prepared from 4-[2-(2,6-dimethyl-phenyl)-2-oxo-ethyl]-3-thien-2-yl-benzoic acid obtained from Ex-33D in a similar manner as described in Ex-32I. The crude material was purified by silica gel chromatography (6.5% MeOH in CH₂Cl₂, then a second column with 4% MeOH in CH₂Cl₂) to provide 176 mg of semi-pure material which was further purified by slurrying (EtOH/hexanes) and filtration to provide 79 mg (42%) of pure product as a white solid, mp 186.5-188° C. ¹H-NMR (DMSO-d₆) δ 7.98-8.00 (m, 2H), 7.59 (dd, J=5.1, 1.8 Hz, 1H), 7.41 (d, J=7.8 Hz, 1H), 7.22 (t, J=6.9 Hz, 1H), 7.05-7.08 (m, 4H), 6.39 (s, 1H), 5.91 (s, 1H), 1.97 (s, 6H). HRMS (EI⁺) m/z: calc. 363.1055, found 363.1060. Anal. calculated for C₂₂H₁₈O₃S: C, 72.90; H, 5.01; S, 8.85. Found: C, 72.37; H, 4.95; S, 8.48%.

### Example 34

### 2-(3,5-Di-tert-butyl-4-hydroxy-phenyl)-1-(2,6-dimethoxy-3-thien-2-yl-phenyl)-propenone

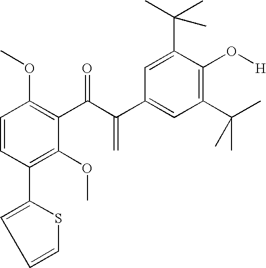

Ex-34A: In a 200 mL round-bottom flask 3,5-di-tert-butyl-4-hydroxy-benzaldehyde (4.5 g, 19.2 mmol) was combined with 1,2-dichloroethane (45 mL). Diisopropyl ethylamine (4.0 mL, 23 mmol) was added, followed by 2-(trimethylsilyl)ethoxymethyl chloride (3.75 mL, 21.2 mmol) and the reaction was heated at reflux for 4 h. Additional diisopropyl ethylamine (0.7 mL, 4 mmol) and 2-(trimethylsilyl)ethoxymethyl chloride (0.5 mL, 2.8 mmol) were added and the reaction was heated at reflux overnight. The reaction was cooled to room temperature, diluted with CH₂Cl₂, and washed with H₂O. The organic phase was dried over Na₂SO₄, filtered, concentrated, and dried. The crude material was purified by silica gel chromatography (10% EtOAc in hexanes) to provide 5.76 g (81%) of semi-pure 3,5-di-tert-butyl-4-(2-trimethylsilanyl-ethoxymethoxy)-benzaldehyde as an orange oil. ¹H-NMR (CDCl₃) δ 9.91 (s, 1H), 7.80 (s, 2H), 4.96 (s, 2H), 3.93 (t, J=8.2 Hz, 2H), 1.47 (s, 18H), 1.04 (t, J=8.2 Hz, 2H), 0.06 (s, 9H).

Ex-34B: [3,5-Di-tert-butyl-4-(2-trimethylsilanyl-ethoxymethoxy)-phenyl]-methanol was prepared from 3,5-di-tert-butyl-4-(2-trimethylsilanyl-ethoxymethoxy)-benzaldehyde obtained from Ex-34A in a similar manner as described in Ex-7C. The crude material was purified by silica gel chromatography (10% EtOAc in hexanes) to provide 3.75 g (65%) of pure product as a yellow oil. ¹H-NMR (CDCl₃) δ 7.26 (s, 2H), 4.93 (s, 2H), 4.61 (d, J=5.7 Hz, 2H), 3.92 (d, J=8.2 Hz, 2H), 1.45 (s, 18H), 1.04 (t, J=8.2 Hz, 2H), 0.06 (s, 9H).

Ex-34C: [2-(4-Bromomethyl-2,6-di-tert-butyl-phenoxymethoxy)-ethyl]-trimethyl-silane was prepared from [3,5-di-tert-butyl-4-(2-trimethylsilanyl-ethoxymethoxy)-phenyl]-methanol obtained from Ex-34B in a similar manner as described in Ex-7D. The crude material was purified by silica gel chromatography (2-4% EtOAc in hexanes) to provide 1.53 g (67%) of semi-pure product as a dark orange oil which was used in the following reaction immediately. ¹H-NMR (CDCl₃) δ 7.27 (s, 2H), 4.92 (s, 2H), 4.48 (s, 2H), 3.91 (t, J=8.5 Hz, 2H), 1.44 (s, 18H), 1.03 (t, J=8.5 Hz, 2H), 0.06 (s, 9H).

Ex-34D: 2-(3-Bromo-2,6-dimethoxy-phenyl)-2-(tert-butyl-dimethyl-silanyloxy)-3-[3,5-di-tert-butyl-4-(2-trimethylsilanyl-ethoxymethoxy)-phenyl]-propionitrile was prepared from [2-(4-bromomethyl-2,6-di-tert-butyl-phenoxymethoxy)-ethyl]-trimethyl-silane obtained from Ex-34C and (3-bromo-2,6-dimethoxy-phenyl)-(tert-butyl-dimethyl-silanyloxy)-acetonitrile obtained from Ex-30B in a similar manner as described in Ex-7E. The crude material was purified by silica gel chromatography (5% EtOAc in hexanes) to provide 1.32 g (50%) of semi-pure product as a yellow-orange foam. ¹H-NMR (CDCl₃) δ 7.47 (d, J=8.7 Hz, 1H), 6.91 (s, 2H), 6.64 (d, J=8.7 Hz, 1H), 4.77 (s, 2H), 4.10 (d, J=12.7 Hz, 1H), 3.90 (s, 3H), 3.88 (t, J=8.7 Hz, 2H), 3.26 (d, J=12.7 Hz, 1H), 2.98 (s, 3H), 1.28 (s, 18H), 1.01 (t, J=8.7 Hz, 2H), 0.89 (s, 9H), 0.19 (s, 3H), 0.04 (s, 9H), −0.19 (s, 3H).

Ex-34E: 1-(3-Bromo-2,6-dimethoxy-phenyl)-2-[3,5-di-tert-butyl-4-(2-trimethylsilanyl-ethoxymethoxy)-phenyl]-ethanone was prepared from 2-(3-bromo-2,6-dimethoxy-phenyl)-2-(tert-butyl-dimethyl-silanyloxy)-3-[3,5-di-tert-butyl-4-(2-trimethylsilanyl-ethoxymethoxy)-phenyl]-propionitrile obtained from Ex-34D in a similar manner as described in Ex-7F. The crude material was purified by silica gel chromatography (10% EtOAc in hexanes) to provide 0.51 g (48%) of pure product as an orange oil. ¹H-NMR (CDCl₃) δ 7.42 (d, J=9.1 Hz, 1H), 7.02 (s, 2H), 6.50 (d, J=8.1 Hz, 1H), 4.85 (s, 2H), 3.95 (s, 2H), 3.90 (t, J=8.6 Hz, 2H), 3.75 (s, 3H), 3.69 (s, 3H), 1.38 (s, 18H), 1.03 (t, J=8.6 Hz, 2H), 0.05 (s, 9H).

Ex-34F: In a 25 mL round-bottom flask 1-(3-bromo-2,6-dimethoxy-phenyl)-2-[3,5-di-tert-butyl-4-(2-trimethylsilanyl-ethoxymethoxy)-phenyl]-ethanone obtained from Ex-34E (395 mg, 0.665 mmol) was combined with CH₂Cl₂ (6.5 mL). The solution was cooled in an ice bath. Boron trifluoride diethyl etherate (0.34 mL, 2.68 mmol) was added dropwise. After stirring for 1 h at 0° C., TLC indicated the reaction was complete. The reaction was diluted with H₂O and 1 N HCl, then extracted with CH₂Cl₂. The organic phase was washed with ±2-saturated brine, dried over Na₂SO₄, filtered, concentrated, and dried. The crude material was purified by silica gel chromatography (15% EtOAc in hexanes) to provide 228 mg (74%) of 1-(3-bromo-2,6-dimethoxy-phenyl)-2-(3,5-di-tert-butyl-4-hydroxy-phenyl)-ethanone as an orange foam. ¹H-NMR (CDCl₃) δ 7.43 (d, J=8.7 Hz, 1H), 6.93 (s, 2H), 6.50 (d, J=8.7 Hz, 1H), 5.08 (s, 1H), 3.94 (s, 2H), 3.76 (s, 3H), 3.67 (s, 3H), 1.38 (s, 18H).

Ex-34G: 2-(3,5-Di-tert-butyl-4-hydroxy-phenyl)-1-(2,6-dimethoxy-3-thien-2-yl-phenyl)-ethanone was prepared from 1-(3-bromo-2,6-dimethoxy-phenyl)-2-(3,5-di-tert-butyl-4-hydroxy-phenyl)-ethanone obtained from Ex-34F and thiophene 2-boronic acid in a similar manner as described in Ex-30E. The crude material was purified by silica gel chromatography (15% EtOAc in hexanes) to provide 218 mg (95%) of pure product as an orange solid. ¹H-NMR (CDCl₃) δ 7.50 (d, J=8.4 Hz, 1H), 7.31-7.33 (m, 2H), 7.07 (t, J=4.2 Hz, 1H), 6.97 (s, 2H), 6.64 (d, J=8.4 Hz, 1H), 5.06 (s, 1H), 4.01 (s, 2H), 3.72 (s, 3H), 3.53 (s, 3H), 1.38 (s, 18H).

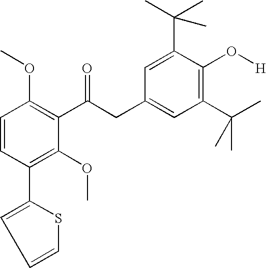

### 2-(3,5-Di-tert-butyl-4-hydroxy-phenyl)-1-(2,6-dimethoxy-3-thien-2-yl-phenyl)-ethanone

Ex-34H: The title compound was prepared from 2-(3,5-di-tert-butyl-4-hydroxy-phenyl)-1-(2,6-dimethoxy-3-thien-2-yl-phenyl)-ethanone obtained from Ex-34G in a similar manner as described in Ex-7G. The reaction could not be driven to 100% conversion by addition of formaldehyde, piperidine, and AcOH. The crude material was purified by silica gel chromatography (15% EtOAc in hexanes) to provide 104 mg (52%) of product (which contained ˜6 mol % of inseparable starting material) as an off-white solid, mp 130-132.5° C. ¹H-NMR (CDCl₃) δ 7.56 (d, J=8.4 Hz, 1H), 7.37 (d, J=3.7 Hz, 1H), 7.32 (d, J=5.1 Hz, 1H), 7.28 (s, 2H), 7.08 (dd, J=5.1, 3.7 Hz, 1H), 6.73 (d, J=8.4 Hz, 1H), 6.09 (s, 1H), 5.90 (s, 1H), 5.27 (s, 1H), 3.78 (s, 3H), 3.64 (s, 3H), 1.45 (s, 18H). HRMS (EI⁺) m/z: calc. 479.2256, found 479.2268. Anal. calculated for C₂₉H₃₄O₄S: C, 72.77; H, 7.16; S, 6.70. Found: C, 71.39; H, 7.08; S, 6.46%.

### Example 35

### 2-{4-[1-(2,6-Dimethoxy-3-thien-2-yl-benzoyl)-vinyl]-phenoxy}-2-methyl-propionic acid

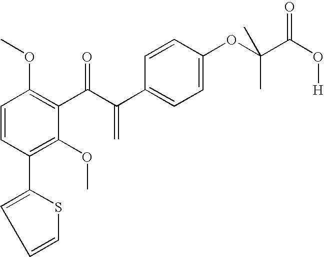

Ex-35A: In a 250 mL round-bottom flask 4-hydroxy-benzaldehyde (5.03 g, 41.2 mmol) was combined with dimethylformamide (65 mL). Potassium carbonate (6.29 g, 45.5 mmol) and ethyl-2-bromoisobutyrate (6.0 mL, 41 mmol) were added and the reaction was heated at 80° C. for 30 min, at which point additional ethyl-2-bromoisobutyrate (6.0 mL, 41 mmol) was added. The reaction was heated for 1 h, at which point additional ethyl-2-bromoisobutyrate (6.0 mL, 41 mmol) was added, and the reaction was left heating overnight. HPLC indicated the reaction was incomplete; additional potassium carbonate (6.29 g, 45.5 mmol) and ethyl-2-bromoisobutyrate (6.0 mL, 41 mmol) were added in portions to the reaction over 8 h. The reaction was again left heating overnight, at which point HPLC indicated starting material remained. The reaction was diluted with H₂O and EtOAc. The phases were separated, and the aqueous phase was extracted with EtOAc. The organic phase was washed with 2 N Na₂CO₃ mixed with brine, dried over Na₂SO₄, filtered, concentrated, and dried. The crude material was purified by silica gel chromatography (25% EtOAc in hexanes) to provide 8.99 g (92%) of semi-pure 2-(4-formyl-phenoxy)-2-methyl-propionic acid ethyl ester as a yellow oil. ¹H-NMR (CDCl₃) δ 9.88 (s, 1H), 7.78 (d, J=8.7 Hz, 2H), 6.89 (d, J=8.7 Hz, 2H), 4.23 (q, J=7.2 Hz, 2H), 1.67 (s, 6H), 1.21 (t, J=7.2 Hz, 3H).

Ex-35B: 2-(4-Hydroxymethyl-phenoxy)-2-methyl-propionic acid ethyl ester was prepared from 2-(4-formyl-phenoxy)-2-methyl-propionic acid ethyl ester obtained from Ex-35A in a similar manner as described in Ex-7C. The crude material was purified by silica gel chromatography (25-30% EtOAc in hexanes) to provide 7.79 g (86%) of pure product as a colorless oil. ¹H-NMR (CDCl₃) δ 7.24 (d, J=7.5 Hz, 2H), 6.83 (d, J=7.5 Hz, 2H), 4.61 (d, J=6.0 Hz, 2H), 4.23 (q, J=7.2 Hz, 2H), 1.58 (s, 6H), 1.25 (t, J=7.2 Hz, 3H).

Ex-35C: 2-(4-Bromomethyl-phenoxy)-2-methyl-propionic acid ethyl ester was prepared from 2-(4-hydroxymethyl-phenoxy)-2-methyl-propionic acid ethyl ester obtained from Ex-35B in a similar manner as described in Ex-7D. The crude material was purified by silica gel chromatography (15% EtOAc in hexanes) to provide 318 mg (82%) of pure product as a colorless oil which was stored in the freezer. ¹H-NMR (CDCl₃) δ 7.26 (d, J=9.0 Hz, 2H), 6.78 (d, J=9.0 Hz, 2H), 4.47 (s, 2H), 4.23 (q, J=7.2 Hz, 2H), 1.60 (s, 6H), 1.24 (t, J=7.2 Hz, 3H).

Ex-35D: In a 25 mL round-bottom flask (3-bromo-2,6-dimethoxy-phenyl)-(tert-butyl-dimethyl-silanyloxy)-acetonitrile obtained from Ex-30B (933 mg, 2.41 mmol) was combined with THF (7.5 mL). The solution was cooled in a −78° C. bath and LHMDS (1 M in THF, 2.75 mL) was added dropwise. After 10 min, the cold bath was removed. After stirring an additional 3 min, the solution was placed in a 0° C. bath and stirred for 25 min, then placed back in a −78° C. bath. In a 50 mL round-bottom flask 2-(4-bromomethyl-phenoxy)-2-methyl-propionic acid ethyl ester obtained from Ex-35C (674 mg, 2.24 mmol) was combined with THF (5 mL) and the solution was cooled to −78° C. Via cannula, the anion solution was transferred to the 50 mL flask in a slow stream. The resulting solution was stirred at −78° C. for 12 min, then allowed to warm to room temperature. After 1 h 15 min, HPLC indicated large quantities of both starting materials remained. After 3 h, the reaction was cooled in a −78° C. bath and additional LHMDS (1 M in THF, 0.60 mL) was added dropwise and the reaction was left stirring overnight. The solution was diluted with aqueous NH₄Cl and extracted with EtOAc. The organic phase was washed with brine, dried over Na₂SO₄, filtered, concentrated, and dried. The crude material was purified by silica gel chromatography (2% EtOAc in hexanes) to provide 400 mg (29%) of semi-pure 2-{4-[2-(3-bromo-2,6-dimethoxy-phenyl)-2-(tert-butyl-dimethyl-silanyloxy)-2-cyano-ethyl]-phenoxy}-2-methyl-propionic acid ethyl ester as a colorless oil. ¹H-NMR (CDCl₃) δ 7.48 (d, J=9.1 Hz, 1H), 6.97 (d, J=8.2 Hz, 2H), 6.57 (d, J=8.2 Hz, 2H), 6.62 (d, J=9.1 Hz, 1H), 4.19 (q, J=6.9 Hz, 2H), 3.94 (d, J=13.2 Hz, 1H), 3.87 (s, 3H), 3.38 (d, J=13.2 Hz, 1H), 3.25 (s, 3H), 1.52 (s, 6H), 1.23 (t, J=6.9 Hz, 3H), 0.86 (s, 9H), 0.23 (s, 3H), −0.20 (s, 3H).

Ex-35E: 2-{4-[2-(3-Bromo-2,6-dimethoxy-phenyl)-2-oxo-ethyl]-phenoxy}-2-methyl-propionic acid ethyl ester was prepared from 2-{4-[2-(3-bromo-2,6-dimethoxy-phenyl)-2-(tert-butyl-dimethyl-silanyloxy)-2-cyano-ethyl]-phenoxy}-2-methyl-propionic acid ethyl ester obtained from Ex-35D in a similar manner as described in Ex-7F. The crude material was purified by silica gel chromatography (20% EtOAc in hexanes) to provide 277 mg (72%) of pure product as a colorless oil. ¹H-NMR (CDCl₃) δ 7.45 (d, J=8.7 Hz, 1H), 7.06 (d, J=8.2 Hz, 2H), 6.77 (d, J=8.2 Hz, 2H), 6.54 (d, J=8.7 Hz, 1H), 4.22 (q, J=6.3 Hz, 2H), 3.98 (s, 2H), 3.77 (s, 3H), 3.73 (s, 3H), 1.57 (s, 6H), 1.25 (q, J=6.3 Hz, 3H).

Ex-35F: 2-{4-[2-(2,6-Dimethoxy-3-thien-2-yl-phenyl)-2-oxo-ethyl]-phenoxy}-2-methyl-propionic acid ethyl ester was prepared from 2-{4-[2-(3-bromo-2,6-dimethoxy-phenyl)-2-oxo-ethyl]-phenoxy}-2-methyl-propionic acid ethyl ester obtained from Ex-35E and thiophene 2-boronic acid in a similar manner as described in Ex-30E. The reaction could not be forced to completion. The crude material was purified by silica gel chromatography (20% EtOAc in hexanes) to provide 239 mg of product containing ˜18 mol % unreacted starting material. The isolated material was re-subjected to the reaction conditions. The crude material was purified by silica gel chromatography (20% EtOAc in hexanes) to provide 214 mg (77%) of pure product as a colorless oil. ¹H-NMR (CDCl₃) δ 7.52 (d, J=8.1 Hz, 1H), 7.31-7.34 (m, 2H), 7.11 (d, J=7.5 Hz, 2H), 7.07 (dd, J=5.1, 3.6 Hz, 1H), 6.78 (d, J=7.5 Hz, 2H), 6.68 (d, J=8.1 Hz, 1H), 4.21 (q, J=7.6 Hz, 2H), 4.05 (s, 2H), 3.77 (s, 3H), 3.56 (s, 3H), 1.57 (s, 6H), 1.24 (t, J=7.6 Hz, 2H).

Ex-35G: In a 200 mL round-bottom flask 2-{4-[2-(2,6-dimethoxy-3-thien-2-yl-phenyl)-2-oxo-ethyl]-phenoxy}-2-methyl-propionic acid ethyl ester obtained from Ex-35F (214 mg, 0.457 mmol) was combined with THF (5 mL) and EtOH (1 mL). The solution was purged subsurface with nitrogen as 1 N LiOH (1 mL) was added. The reaction was stirred at room temperature for 5 h 20 min, at which point HPLC indicated starting material had been consumed and multiple impurities had formed. The solution was acidified to pH˜2 with 1 N HCl, diluted with H₂O, and extracted with CH₂Cl₂. The organic phase was washed with 12-saturated brine, dried over Na₂SO₄, filtered, concentrated, and dried. The crude material was purified by silica gel chromatography (5-12% MeOH in CH₂Cl₂) to provide 69 mg (34%) of 2-{4-[2-(2,6-dimethoxy-3-thien-2-yl-phenyl)-2-oxo-ethyl]-phenoxy}-2-methyl-propionic acid as a yellow-white residue. ¹H-NMR (DMSO-d₆) δ 7.70 (d, J=8.7 Hz, 1H), 7.56 (d, J=5.1 Hz, 1H), 7.45 (d, J=4.2 Hz, 1H) 7.12 (dd, J=5.1, 4.2 Hz, 1H), 7.05 (d, J=9.1 Hz, 2H), 6.96 (d, J=8.7 Hz, 1H), 6.78 (d, J=9.1 Hz, 2H), 4.00 (s, 2H), 3.81 (s, 3H), 3.50 (s, 3H), 1.46 (s, 6H).

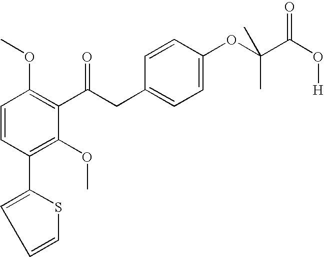

### 2-{4-[2-(2,6-Dimethoxy-3-thien-2-yl-phenyl)-2-oxo-ethyl]-phenoxy}-2-methyl-propionic acid

Ex-35H: The title compound was prepared from 2-{4-[2-(2,6-dimethoxy-3-thien-2-yl-phenyl)-2-oxo-ethyl]-phenoxy}-2-methyl-propionic acid obtained from Ex-35G in a similar manner as described in Ex-7G. The reaction could not be driven to 100% conversion by addition of formaldehyde, piperidine, and AcOH. The crude material was purified by silica gel chromatography (5-50% MeOH in CH₂Cl₂) to provide 12 mg (17%) of semi-pure product (˜90% pure) as a beige solid, mp 138° C. (dec.). ¹H-NMR (DMSO-d₆) δ 7.72 (d, J=8.1 Hz, 1H), 7.56 (d, J=5.1 Hz, 1H), 7.47 (d, J=4.0 Hz, 1H), 7.24 (d, J=8.0 Hz, 2H), 7.13 (dd, J=5.1, 4.0 Hz, 1H), 6.99 (d, J=8.1 Hz, 1H), 6.83 (d, J=8.0 Hz, 2H), 6.20 (s, 1H), 5.71 (s, 1H), 3.78 (s, 3H), 3.52 (s, 3H), 1.43 (s, 6H). HRMS (EI⁺) m/z: calc. 453.1372, found 453.1371.

### Example 36

### 4-[1-(2,6-Dimethoxy-benzoyl)-vinyl]-3-thien-2-yl-benzoic acid

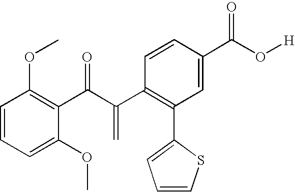

Ex-36A: In a 50 mL round-bottom flask 2,6-dimethoxy-benzaldehyde (5.0 g, 30.1 mmol) was combined with KCN (8 g, 122.85 mmol), MeCN (25 mL), ZnI₂ (200 mg, 0.63 mmol), and TBSCl (5.5 g, 36.5 mmol) and the resulting mixture was stirred rapidly and heated to 67° C. After aging overnight the reaction was concentrated to ⅓ volume, diluted with ether and filtered. The filtrate was diluted with EtOAc, washed with NaHCO₃, dried over Na₂SO₄, filtered, and concentrated to dryness. The crude material was purified by silica gel chromatography (5% EtOAc in hexanes) to provide 8.72 g (95%) of (tert-butyl-dimethyl-silanyloxy)-(2,6-dimethoxy-phenyl)-acetonitrile. ¹H-NMR (CDCl₃) δ 7.2 (d, J=8.3 Hz, 1H), 6.56 (d, J=8.3 Hz, 2H), 6.09 (s, 1H), 3.88 (s, 6H), 0.86 (s, 9H), 0.16 (s, 3H), −0.01 (s, 3H).

Ex-36B: 3-Bromo-4-[2-(tert-butyl-dimethyl-silanyloxy)-2-cyano-2-(2,6-dimethoxy-phenyl)-ethyl]-benzoic acid methyl ester was prepared from (tert-butyl-dimethyl-silanyloxy)-(2,6-dimethoxy-phenyl)-acetonitrile obtained from Ex-36A and 3-bromo-4-bromomethyl-benzoic acid methyl ester obtained from Ex-32D in a similar manner as described in Ex-7E. The crude material was purified by silica gel chromatography (8-20% EtOAc in hexanes) to provide 1.12 g (21%) of semi-pure product. ¹H-NMR (CDCl₃) δ 8.14-8.29 (m, 1H), 7.98-8.04 (m, 1H), 7.56 (d, J=8.1 Hz, 1H), 7.24-7.29 (m, 1H), 6.54 (d, J=8.4 Hz, 2H), 4.05 (d, J=14.7 Hz, 1H), 3.90 (s, 3H), 3.88-3.90 (m, 1H), 3.78 (s, 6H), 0.85 (s, 9H), 0.05 (s, 3H), −0.15 (s, 3H).

Ex-36C: 3-Bromo-4-[2-(2,6-dimethoxy-phenyl)-2-oxo-ethyl]-benzoic acid methyl ester was prepared from 3-bromo-4-[2-(tert-butyl-dimethyl-silanyloxy)-2-cyano-2-(2,6-dimethoxy-phenyl)-ethyl]-benzoic acid methyl ester obtained from Ex-36B in a similar manner as described in Ex-7F. The crude material was purified by silica gel chromatography (20-30% EtOAc in hexanes) to provide 278 mg (35%) of pure product as a colorless oil. ¹H-NMR (CDCl₃) δ 8.22 (d, J=2.0 Hz, 1H), 7.90 (dd, J=8.2, 2.0 Hz, 1H), 7.35 (d, J=8.2 Hz, 1H), 7.26 (t, J=7.6 Hz, 1H), 6.52 (d, J=7.6 Hz, 2H), 4.31 (s, 2H), 3.91 (s, 3H), 3.77 (s, 6H).

Ex-36D: 4-[2-(2,6-Dimethoxy-phenyl)-2-oxo-ethyl]-3-thien-2-yl-benzoic acid methyl ester was prepared from 3-Bromo-4-[2-(2,6-dimethoxy-phenyl)-2-oxo-ethyl]-benzoic acid methyl ester obtained from Ex-36C and thiophene 2-boronic acid in a similar manner as described in Ex-30E. The crude material was purified by silica gel chromatography (20-30% EtOAc in hexanes) to provide 216 mg (83%) of pure product. ¹H-NMR (CDCl₃) δ 8.08 (d, J=1.3 Hz, 1H), 7.95 (dd, J=7.9, 1.3 Hz, 1H), 7.39 (d, J=7.9 Hz, 1H), 7.35 (dd, J=3.6, 2.4 Hz, 1H), 7.24 (t, J=8.4 Hz, 1H), 7.05-7.07 (m, 2H), 6.50 (d, J=8.4 Hz, 2H), 4.25 (s, 2H), 3.91 (s, 3H), 3.70 (s, 6H).

Ex-36E: 4-[2-(2,6-Dimethoxy-phenyl)-2-oxo-ethyl]-3-thien-2-yl-benzoic acid was prepared from 4-[2-(2,6-dimethoxy-phenyl)-2-oxo-ethyl]-3-thien-2-yl-benzoic acid methyl ester obtained from Ex-36D in a similar manner as described in Ex-30FF. The crude material was purified by silica gel chromatography (5% MeOH in CH₂Cl₂) to provide 44 mg (58%) of pure product as a white solid. ¹H-NMR (DMSO-d₆) δ 7.89-7.91 (m, 2H), 7.66 (d, J=5.4 Hz, 1H), 7.33-7.37 (m, 2H), 7.19 (dd, J=4.5, 3.3 Hz, 1H), 7.09 (d, J=2.4 Hz, 1H), 6.68 (d, J=8.7 Hz, 2H), 4.20 (s, 2H), 3.71 (s, 6H). 4-[2-(2,6-Dimethoxy-phenyl)-2-oxo-ethyl]-3-thien-2-yl-benzoic acid

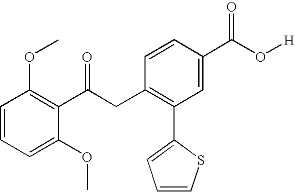

Ex-36F: The title compound was prepared from 4-[2-(2,6-dimethoxy-phenyl)-2-oxo-ethyl]-3-thien-2-yl-benzoic acid obtained from Ex-36E in a similar manner as described in Ex-321. The crude material was purified by silica gel chromatography (5% MeOH in CH₂Cl₂) followed by slurrying (EtOH/hexanes) and filtration to provide 19 mg (29%) of product as a solid, mp 192-195° C. ¹H-NMR (DMSO-d₆) δ 7.99 (d, J=1.3 Hz, 1H), 7.93 (dd, J=6.7, 1.3 Hz, 1H), 7.58 (d, J=6.7 Hz, 11H), 7.24-7.38 (m, 2H), 7.10-7.13 (m, 2H), 6.68 (d, J=8.1 Hz, 2H), 6.05 (s, 1H), 5.96 (s, 1H), 3.70 (s, 6H). EI-MS: 394 (M+) Anal. calculated for C₂₂H₁₈O₅S: C, 66.99; H, 4.60; S, 8.13. Found: C, 65.41; H, 4.50; S, 7.90%.

### Example 37

### 2-{4-[1-(2,6-Dimethoxy-benzoyl)-vinyl]-2-thien-2-yl-phenoxy}-2-methyl-propionic acid

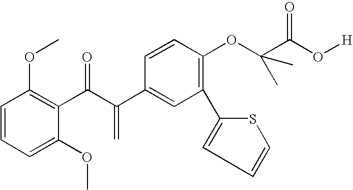

Ex-37A: A solution of 3-bromo-4-hydroxybenzaldehyde (1.0 g, 4.97 mmol), 2-bromo-2-methyl-propionic acid ethyl ester (1.84 mL, 12.4 mmol) and K₂CO₃ (2.1 g, 14.9 mmol) in DMF (30 mL) was heated at 120° C. for 4.5 h. The solvent was removed under reduced pressure. Water was added to the residue. The aqueous solution was extracted with EtOAc. The combined EtOAc was washed with NaHCO₃ (saturated), brine (10%), dried over Na₂SO₄ and concentrated. The crude product was purified by silica gel chromatography (30% EtOAc in hexanes) to afford 1.15 g (73%) of 2-(2-bromo-4-formyl-phenoxy)-2-methyl-propionic acid ethyl ester. ¹H NMR (CDCl₃) δ 9.84 (s, 1H), 8.09 (d, J=1.8 Hz, 1H), 7.70 (dd, J=8.7, 1.8 Hz, 1H), 6.83 (d, J=8.7 Hz, 1H), 4.24 (q, J=6.9 Hz, 2H), 1.71 (s, 6H), 1.23 (t, J=6.9 Hz, 3H).

Ex-37B: A solution of 2-(2-bromo-4-formyl-phenoxy)-2-methyl-propionic acid ethyl ester obtained from Ex-37A (4.86 g, 15.4 mmol) in THF (100 mL) was degassed with nitrogen for 20 min followed by sequential addition of 2-thiophene boronic acid (2.96 g, 23.13 mmol), bis(tri-tert-butylphosphine)palladium (0) (0.24 g, 0.46 mmol) and KF (2.2 g, 37.0 mmol). The reaction mixture was heated at 65° C. for 1 h, diluted with water and partitioned. The aqueous solution was extracted with EtOAc. The combined organic solution was filtered through a pad of Celite, washed with Na₂CO₃, brine, dried over Na₂SO₄, filtered and concentrated. The crude product (5.42 g) was combined with a crude product from another batch (1.33 g) with similar purity and purified by silica gel chromatography (30% EtOAc in hexanes) to afford 6.14 g 2-(4-formyl-2-thien-2-yl-phenoxy)-2-methyl-propionic acid ethyl ester. ¹H NMR (CDCl₃) δ 9.93 (s, 1H), 8.19 (d, J=1.1 Hz, 1H), 7.67 (dd, J=8.1, 2.5 Hz, 1H), 7.59 (d, J=3.6 Hz, 1H), 7.39 (d, J=4.9 Hz, 1H), 7.12 (m, 1H), 6.82 (d, J=8.1 Hz, 1H), 4.24 (q, J=7.5 Hz, 2H), 1.76 (s, 6H), 1.21 (t, J=7.5 Hz, 3H).

Ex-37C: To a solution of 2-(4-formyl-2-thien-2-yl-phenoxy)-2-methyl-propionic acid ethyl ester obtained from Ex-37B (3.81 g, 12.0 mmol) in a mixture of THF (40 mL) and EtOH (34 mL) was added NaBH₄ (0.50 g, 13.16 mmol) at 0° C. The reaction mixture was stirred at same temperature for 2 h and diluted with water (100 mL) and EtOAc. The resulting aqueous solution was extracted with EtOAc. The combined EtOAc was washed with brine, dried over Na₂SO₄, filtered and concentrated. The crude 2-(4-hydroxymethyl-2-thien-2-yl-phenoxy)-2-methyl-propionic acid ethyl ester (3.78 g) was used without further purification. ¹H NMR (CDCl₃) δ 7.64 (d, J=1.7 Hz, 1H), 7.50 (dd, J=3.7, 1.7 Hz, 1H), 7.33 (d, J=5.3 Hz, 1H), 7.14 (dd, J=8.0, 1.9 Hz, 1H), 7.08 (m, 1H), 6.75 (d, J=7.4 Hz, 1H), 4.64 (s, 2H), 4.25 (q, J=7.2 Hz, 2H), 3.71 (m, 1H), 1.64 (s, 6H), 1.25 (t, J=7.2 Hz, 3H).

Ex-37D: 2-(4-Bromomethyl-2-thien-2-yl-phenoxy)-2-methyl-propionic acid ethyl ester was prepared from 2-(4-hydroxymethyl-2-thien-2-yl-phenoxy)-2-methyl-propionic acid ethyl ester obtained from Ex-37C in a similar manner as described in Ex-7D. The crude material was purified by silica gel chromatography (10-12% EtOAc in hexanes) to provide 8.32 g (83%) of pure product as a faint yellow, semi-crystalline solid. ¹H-NMR (CDCl₃) δ 7.66 (d, J=2.5 Hz, 1H), 7.50 (dd, J=3.6, 1.2 Hz, 1H), 7.34 (d, J=4.7 Hz, 1H), 7.16 (dd, J=7.9, 2.5 Hz, 1H), 7.08 (dd, J=4.7, 3.6 Hz, 1H), 6.71 (d, J=7.9 Hz, 1H), 4.50 (s, 2H), 4.12 (q, J=7.2 Hz, 2H), 1.67 (s, 6H), 1.26 (t, J=7.3 Hz, 3H).

Ex-37E: 2-{4-[2-(tert-Butyl-dimethyl-silanyloxy)-2-cyano-2-(2,6-dimethoxy-phenyl)-ethyl]-2-thien-2-yl-phenoxy}-2-methyl-propionic acid ethyl ester was prepared from 2-(4-bromomethyl-2-thien-2-yl-phenoxy)-2-methyl-propionic acid ethyl ester obtained from Ex-37D and (tert-butyl-dimethyl-silanyloxy)-(2,6-dimethoxy-phenyl)-acetonitrile obtained from Ex-36A in a similar manner as described in Ex-30C. The crude material was purified by silica gel chromatography (10% EtOAc in hexanes) to provide 6.82 g (68%) of pure product as a faint yellow, viscous oil. ¹H-NMR (CDCl₃) δ 7.33 (d, J=2.1 Hz, 1H), 7.26-7.28 (m, 2H), 7.22 (t, J=8.1 Hz, 1H), 7.02 (d, J=3.6 Hz, 1H), 7.01 (d, J=2.7 Hz, 1H), 6.63 (d, J=8.1 Hz, 1H), 6.53 (d, J=8.1 Hz, 2H), 4.12 (q, J=7.2 Hz, 2H), 3.72 (s, 6H), 3.67 (d, J=13.0 Hz, 1H), 3.53 (d, J=13.0 Hz, 1H), 1.57 (s, 6H), 1.25 (t, J=7.2 Hz, 3H), 0.85 (s, 9H), 0.09 (s, 3H), −0.18 (s, 3H).

Ex-37F: 2-{4-[2-(2,6-Dimethoxy-phenyl)-2-oxo-ethyl]-2-thien-2-yl-phenoxy}-2-methyl-propionic acid ethyl ester was prepared from 2-{4-[2-(tert-butyl-dimethyl-silanyloxy)-2-cyano-2-(2,6-dimethoxy-phenyl)-ethyl]-2-thien-2-yl-phenoxy}-2-methyl-propionic acid ethyl ester obtained from Ex-37E in a similar manner as described in Ex-7F. The crude material was purified by silica gel chromatography (20-35% EtOAc in hexanes) to provide 4.57 g (87%) of pure product as a white solid. ¹H-NMR (CDCl₃) δ 7.44 (d, J=2.1 Hz, 1H), 7.40 (dd, J=3.9, 1.6 Hz, 1H), 7.30 (dd, J=5.1, 1.6 Hz, 1H), 7.21 (t, J=8.2 Hz, 1H), 7.05 (dd, J=5.1, 3.9 Hz, 1H), 6.97 (dd, J=8.5, 2.1 Hz, 1H), 6.68 (d, J=8.5 Hz, 1H), 6.50 (d, J=8.2 Hz, 2H), 4.23 (q, J=6.7 Hz, 2H), 3.99 (s, 2H), 3.78 (s, 6H), 1.60 (s, 3H), 1.24 (t, J=6.7 Hz, 3H).

Ex-37G: 2-{4-[2-(2,6-Dimethoxy-phenyl)-2-oxo-ethyl]-2-thien-2-yl-phenoxy}-2-methyl-propionic acid was prepared from 2-{4-[2-(2,6-dimethoxy-phenyl)-2-oxo-ethyl]-2-thien-2-yl-phenoxy}-2-methyl-propionic acid ethyl ester obtained from Ex-37F in a similar manner as described in Ex-35G. The reaction was run at 60° C. The crude material was purified by silica gel chromatography (8% MeOH in CH₂Cl₂) to provide 2.93 g (68%) of semi-pure product as a faint yellow solid. ¹H-NMR (DMSO-d₆) δ 7.54 (d, J=5.2 Hz, 1H), 7.48 (dd, J=3.7, 1.8 Hz, 1H), 7.45 (d, J=2.0 Hz, 1H), 7.32 (t, J=8.5 Hz, 1H), 7.11 (dd, J=5.2, 3.7 Hz, 1H), 6.98 (dd, J=8.2, 2.0 Hz, 1H), 6.74 (d, J=8.2 Hz, 1H), 6.68 (d, J=8.5 Hz, 2H), 3.92 (s, 2H), 3.72 (s, 6H), 1.57 (s, 6H).

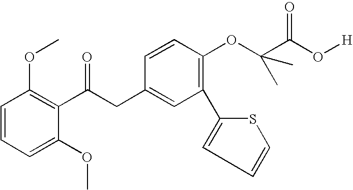

### 2-[4-[2-(2,6-Dimethoxy-phenyl)-2-oxo-ethyl]-2-thien-2-yl-phenoxy]-2-methyl-propionic acid

Ex-37H: The title compound was prepared from 2-{4-[2-(2,6-dimethoxy-phenyl)-2-oxo-ethyl]-2-thien-2-yl-phenoxy}-2-methyl-propionic acid obtained from Ex-37G in a similar manner as described in Ex-7G. The crude material was purified by silica gel chromatography (3-4% MeOH in CH₂Cl₂, followed by a second column with 2.5% MeOH in CH₂Cl₂, followed by a third column with 4% MeOH in CH₂Cl₂) to provide 0.50 g (17%) of product as a faint yellow solid (which contained ˜2.4 wt % EtOAc), mp 56.5° C. (slow dec). ¹H-NMR (DMSO-d₆) δ 7.68 (d, J=2.1 Hz, 1H), 7.57 (d, J=5.1 Hz, 1H), 7.53 (d, J=3.6 Hz, 1H), 7.38 (t, J=8.1 Hz, 1H), 7.25 (dd, J=8.2, 2.1 Hz, 1H), 7.11-7.14 (m, 1H), 6.81 (d, J=8.2 Hz, 1H), 6.74 (d, J=8.1 Hz, 2H), 6.21 (s, 1H), 5.74 (s, 1H), 3.74 (s, 6H), 1.63 (s, 6H). HRMS (EI⁺) m/z: calc. 452.1294, found 452.1305. Anal. calculated for C₂₅H₂₄O₆S: C, 66.35; H, 5.35; S, 7.09. Found: C, 65.80; H, 5.61; S, 6.65%.

### Example 38

### 1-(2,6-Dimethyl-phenyl)-2-(2-thien-2-yl-phenyl)-propenone

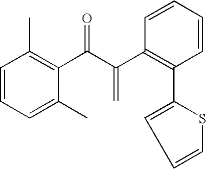

Ex-38A: 3-(2-Bromo-phenyl)-2-(tert-butyl-dimethyl-silanyloxy)-2-(2,6-dimethyl-phenyl)-propionitrile was prepared from (tert-butyl-dimethyl-silanyloxy)-(2,6-dimethyl-phenyl)-acetonitrile obtained from Ex-29A and 1-bromo-2-bromomethyl-benzene in a similar manner as described in Ex-30C. The crude material was purified by silica gel chromatography (3% EtOAc in hexanes) to provide 6.11 g (82%) of semi-pure product as a yellow oil. ¹H-NMR (CDCl₃) δ 7.58 (dd, J=6.9, 1.2 Hz, 1H), 7.36 (dd, J=7.5, 1.2 Hz, 1H), 7.25-7.28 (m, 1H), 7.13-7.18 (m, 1H), 7.10 (d, J=7.2 Hz, 1H), 7.01 (d, J=7.8 Hz, 2H), 3.73 (d, J=13.9 Hz, 1H), 3.62 (d, J=13.9 Hz, 1H), 2.59 (s, 6H), 0.85 (s, 9H), 0.14 (s, 3H), −0.01 (s, 3H).

Ex-38B: 2-(2-Bromo-phenyl)-1-(2,6-dimethyl-phenyl)-ethanone was prepared from 3-(2-bromo-phenyl)-2-(tert-butyl-dimethyl-silanyloxy)-2-(2,6-dimethyl-phenyl)-propionitrile obtained from Ex-38A in a similar manner as described in Ex-7F. The crude material was purified by silica gel chromatography (5% EtOAc in hexanes) to provide 2.89 g (70%) of pure product as a white solid. ¹H-NMR (CDCl₃) δ 7.60 (d, J=7.8 Hz, 1H), 7.25-7.34 (m, 2H), 7.14-7.22 (m, 2H), 7.03 (d, J=8.1 Hz, 2H), 4.25 (s, 2H), 2.31 (s, 6H).

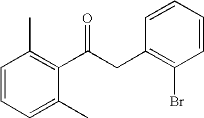

### 2-(2-Bromo-phenyl)-1-(2,6-dimethyl-phenyl)-ethanone

Ex-38C: 2-(2-Bromo-phenyl)-1-(2,6-dimethyl-phenyl)-propenone was prepared from 2-(2-bromo-phenyl)-1-(2,6-dimethyl-phenyl)-ethanone obtained from Ex-38B in a similar manner as described in Ex-7G. The crude material was purified by silica gel chromatography (5% EtOAc in hexanes) to provide 162 mg (78%) of pure product as a semi-crytalline white solid. ¹H-NMR (CDCl₃) δ 7.65-7.68 (m, 1H), 7.34-7.39 (m, 1H), 7.19-7.27 (m, 3H), 7.06 (d, J=7.8 Hz, 2H), 6.23 (s, 1H), 6.10 (s, 1H), 2.32 (s, 6H).

Ex-38D: The title compound was prepared from 2-(2-bromo-phenyl)-1-(2,6-dimethyl-phenyl)-propenone obtained from Ex-38C and thiophene 2-boronic acid in a similar manner as described in Ex-30E. The crude material was purified by silica gel chromatography (10% EtOAc in hexanes) to provide 93 mg (78%) of pure product as a yellow oil. ¹H-NMR (CDCl₃) δ 7.48-7.51 (m, 1H), 7.39-7.42 (m, 2H), 7.26-7.29 (m, 2H), 7.12-7.17 (m, 1H), 6.97-6.99 (m, 4H), 6.14 (s, 1H), 5.92 (s, 1H), 2.06 (s, 6H). HRMS (EI⁺) m/z: calc. 318.1078, found 318.1074. Anal. calculated for C₂₁H₁₈OS: C, 79.21; H, 5.70; S, 10.07. Found: C, 78.32; H, 5.79; S, 9.55%.

### Example 39

### 1-(2,6-Dimethyl-phenyl)-2-[2-(2-methoxy-pyridin-3-yl)-phenyl]-propenone

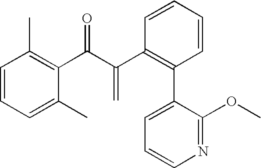

Ex-39: The title compound was prepared from 2-(2-bromo-phenyl)-1-(2,6-dimethyl-phenyl)-propenone obtained from Ex-38C and 2-methoxypyridine-3-boronic acid in a similar manner as described in Ex-30E. The crude material was purified by silica gel chromatography (12% EtOAc in hexanes) to provide 84 mg (31%) of pure product as a faint yellow syrup. ¹H-NMR (CDCl₃) δ 8.12 (dd, J=5.4, 2.1 Hz, 1H), 7.52 (dd, J=6.9, 1.5 Hz, 1H), 7.43-7.46 (m, 2H), 7.26-7.32 (m, 2H), 7.12 (t, J=7.9 Hz, 1H), 6.93 (d, J=7.9 Hz, 2H), 6.89 (dd, J=7.5, 5.1 Hz, 1H), 6.13 (d, J=1.6 Hz, 1H), 5.80 (d, J=1.6 Hz, 1H), 3.85 (s, 3H), 1.89 (s, 6H). HRMS (EI⁺) m/z: calc. 343.1572, found 343.1572.

### Example 40

### 1-(2,6-Dimethoxy-3-thien-2-yl-phenyl)-2-(4-trifluoromethyl-phenyl)-propenone

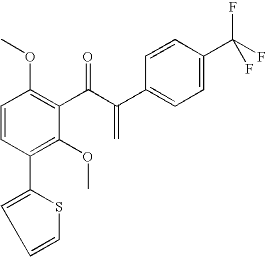

Ex-40A: 2-(3-Bromo-2,6-dimethoxy-phenyl)-2-(tert-butyl-dimethyl-silanyloxy)-3-(4-trifluoromethyl-phenyl)-propionitrile was prepared from (3-bromo-2,6-dimethoxy-phenyl)-(tert-butyl-dimethyl-silanyloxy)-acetonitrile obtained from Ex-30B and 1-bromomethyl-4-trifluoromethyl-benzene in a similar manner as described in Ex-7E. The crude material was purified by silica gel chromatography (20% EtOAc in hexanes) to provide 1.84 g (96%) of semi-pure product as a yellow oil. ¹H-NMR (CDCl₃) δ 7.52 (d, J=9.0 Hz, 1H), 7.48 (d, J=8.9 Hz, 2H), 7.29 (d, J=8.9 Hz, 2H), 6.64 (d, J=9.0 Hz, 1H), 3.89-3.94 (m, 7H), 3.61 (d, J=12.9 Hz, 1H), 0.85 (s, 9H), 0.12 (s, 3H), −0.20 (s, 3H).

Ex-40B: 1-(3-Bromo-2,6-dimethoxy-phenyl)-2-(4-trifluoromethyl-phenyl)-ethanone was prepared from 2-(3-bromo-2,6-dimethoxy-phenyl)-2-(tert-butyl-dimethyl-silanyloxy)-3-(4-trifluoromethyl-phenyl)-propionitrile obtained from Ex-40A in a similar manner as described in Ex-7F. The crude material was purified by silica gel chromatography (20% EtOAc in hexanes) to provide 1.04 g (76%) of semi-pure product as a pink oil. ¹H-NMR (CDCl₃) δ 7.57 (d, J=8.1 Hz, 2H), 7.50 (d, J=8.5 Hz, 1H), 7.34 (d, J=8.1 Hz, 2H), 6.59 (d, J=8.5 Hz, 1H), 4.12 (s, 2H), 3.81 (s, 3H), 3.76 (s, 3H).

Ex-40C: 1-(2,6-Dimethoxy-3-thien-2-yl-phenyl)-2-(4-trifluoromethyl-phenyl)-ethanone was prepared from 1-(3-bromo-2,6-dimethoxy-phenyl)-2-(4-trifluoromethyl-phenyl)-ethanone obtained from Ex-40B and thiophene 2-boronic acid in a similar manner as described in Ex-30E. The crude material was purified by silica gel chromatography (15% EtOAc in hexanes) to provide 667 mg (64%) of pure product as an orange oil. ¹H-NMR (CDCl₃) δ 7.55-7.60 (m, 3H), 7.33-7.40 (m, 4H), 7.08 (dd, J=5.4, 3.9 Hz, 1H), 6.72 (d, J=9.0 Hz, 1H), 4.18 (s, 2H), 3.80 (s, 3H), 3.59 (s, 3H).

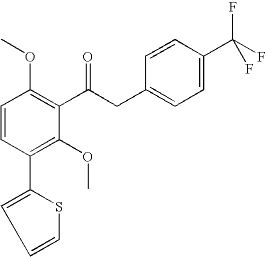

### 1-(2,6-Dimethoxy-3-thien-2-yl-phenyl)-2-(4-trifluoromethyl-phenyl)-ethanone

Ex-40D: The title compound was prepared from 1-(2,6-dimethoxy-3-thien-2-yl-phenyl)-2-(4-trifluoromethyl-phenyl)-ethanone obtained from Ex-40C in a similar manner as described in Ex-7G. The crude material was purified by silica gel chromatography (15% EtOAc in hexanes) to provide 338 mg (49%) of pure product as a white solid, mp 102-105° C. ¹H-NMR (DMSO-d₆) δ 7.80 (d, J=8.4 Hz, 2H), 7.76 (d, J=8.7 Hz, 1H), 7.65 (d, J=8.4 Hz, 2H), 7.58 (dd, J=5.5, 1.3 Hz, 1H), 7.48 (dd, J=5.2, 1.3 Hz, 1H), 7.13 (dd, J=5.5, 5.2 Hz, 1H), 7.02 (d, J=8.7 Hz, 1H), 6.47 (s, 1H), 6.00 (s, 1H), 3.80 (s, 3H), 3.54 (s, 3H). HRMS (EI⁺) m/z: calc. 418.0851, found 418.0846. Anal. calculated for C₂₂H₁₇F₃O₃S: C, 62.07; H, 4.22; F, 14.02; S, 7.89. Found: C, 62.39; H, 4.08; F, 14.04; S, 7.58%.

### Example 41

### 1-(2,6-Dimethoxy-3-thien-2-yl-phenyl)-2-(4-nitrophenyl)-propenone

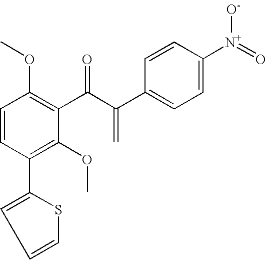

Ex-41A: 2-(3-Bromo-2,6-dimethoxy-phenyl)-2-(tert-butyl-dimethyl-silanyloxy)-3-(4-nitro-phenyl)-propionitrile was prepared from (3-bromo-2,6-dimethoxy-phenyl)-(tert-butyl-dimethyl-silanyloxy)-acetonitrile obtained from Ex-30B and 1-bromomethyl-4-nitro-benzene in a similar manner as described in Ex-30C. The crude material was purified by silica gel chromatography (EtOAc in hexanes) to provide 0.73 g (42%) of semi-pure product. ¹H-NMR (CDCl₃) δ 8.10 (d, J=8.6 Hz, 2H), 7.53 (d, J=8.8 Hz, 1H), 7.38 (d, J=8.6 Hz, 2H), 6.65 (d, J=8.8 Hz, 1H), 3.90 (s, 3H), 3.77-3.82 (m, 2H), 3.59 (s, 3H), 0.84 (s, 9H), 0.10 (s, 3H), −0.20 (s, 3H).

Ex-41B: 1-(3-Bromo-2,6-dimethoxy-phenyl)-2-(4-nitro-phenyl)-ethanone was prepared from 2-(3-bromo-2,6-dimethoxy-phenyl)-2-(tert-butyl-dimethyl-silanyloxy)-3-(4-nitro-phenyl)-propionitrile obtained from Ex-41A in a similar manner as described in Ex-7F. The crude material was purified by silica gel chromatography (25% EtOAc in hexanes) to provide 439 mg (83%) of semi-pure product as a yellow solid. ¹H-NMR (CDCl₃) δ 8.18 (d, J=9.1 Hz, 2H), 7.51 (d, J=8.7 Hz, 1H), 7.40 (d, J=9.1 Hz, 2H), 6.61 (d, J=8.7 Hz, 1H), 4.16 (s, 2H), 3.82 (s, 3H), 3.81 (s, 3H).

Ex-41C: 1-(2,6-Dimethoxy-3-thien-2-yl-phenyl)-2-(4-nitro-phenyl)-ethanone was prepared from 1-(3-bromo-2,6-dimethoxy-phenyl)-2-(4-nitro-phenyl)-ethanone obtained from Ex-41B and thiophene 2-boronic acid in a similar manner as described in Ex-30E. The crude material was purified by silica gel chromatography (20% EtOAc in hexanes) to provide 260 mg (59%) of pure product. ¹H-NMR (CDCl₃) δ 8.20 (d, J=8.8 Hz, 2H), 7.56 (d, J=8.8 Hz, 1H), 7.44 (d, J=8.8 Hz, 2H), 7.34 (d, J=4.5 Hz, 2H), 7.09 (dd, J=4.5, 4.5 Hz, 1H), 6.74 (d, J=8.8 Hz, 1H), 4.24 (s, 2H), 3.83 (s, 3H), 3.59 (s, 3H).

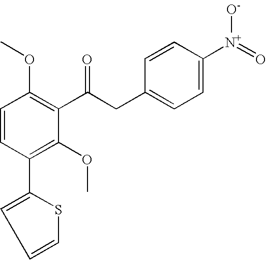

### 1-(2,6-Dimethoxy-3-thien-2-yl-phenyl)-2-(4-nitro-phenyl)-ethanone

Ex-41D: The title compound was prepared from 1-(2,6-dimethoxy-3-thien-2-yl-phenyl)-2-(4-nitro-phenyl)-ethanone obtained from Ex-41C in a similar manner as described in Ex-7G. The crude material was purified by silica gel chromatography (EtOAc in hexanes) to provide 180 mg (67%) of pure product as a solid, mp 114-118° C. ¹H-NMR (CDCl₃) δ 8.25 (d, J=9.0 Hz, 2H), 7.66 (d, J=9.0 Hz, 2H), 7.60 (d, J=8.8 Hz, 1H), 7.37 (dd, J=3.6, 1.2 Hz, 1H), 7.34 (d, J=5.1 Hz, 1H), 7.04 (dd, J=5.1, 3.6 Hz, 1H), 6.77 (d, J=8.8 Hz, 1H), 6.30 (s, 1H), 6.12 (s, 1H), 3.83 (s, 3H), 3.62 (s, 3H). HRMS (EI⁺) m/z: calc. 395.0827, found 398.0833. Anal. calculated for C₂₁H₁₇NO₅S: C, 63.79; H, 4.33; N, 3.54; S, 8.11. Found: C, 63.94; H, 4.51; N, 3.47; S, 7.94%.

### Example 42

### 2-(2,6-Dichloro-phenyl)-1-(2,6-dimethoxy-3-thien-2-yl-phenyl)-propenone

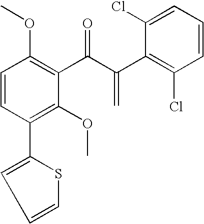

Ex-42A: 2-(3-Bromo-2,6-dimethoxy-phenyl)-2-(tert-butyl-dimethyl-silanyloxy)-3-(2,6-dichloro-phenyl)-propionitrile was prepared from (3-bromo-2,6-dimethoxy-phenyl)-(tert-butyl-dimethyl-silanyloxy)-acetonitrile obtained from Ex-30B and 2-bromomethyl-1,3-dichloro-benzenein a similar manner as described in Ex-30C. The crude material was purified by silica gel chromatography (10% EtOAc in hexanes) to provide 738 mg (38%) of semi-pure product as a white solid. ¹H-NMR (CDCl₃) δ 7.52 (d, J=8.8 Hz, 1H), 7.33 (d, J=8.0 Hz, 2H), 7.14 (t, J=8.0 Hz, 1H), 6.61 (d, J=8.8 Hz, 1H), 3.99 (d, J=9.0 Hz, 1H), 3.89 (s, 3H), 3.84 (d, J=9.0 Hz, 1H), 3.82 (s, 3H), 0.82 (s, 9H), 0.06 (s, 3H), −0.13 (s, 3H).

Ex-42B: 1-(3-Bromo-2,6-dimethoxy-phenyl)-2-(2,6-dichloro-phenyl)-ethanone was prepared from 2-(3-bromo-2,6-dimethoxy-phenyl)-2-(tert-butyl-dimethyl-silanyloxy)-3-(2,6-dichloro-phenyl)-propionitrile obtained from Ex-42A in a similar manner as described in Ex-7F. The crude material was purified by silica gel chromatography (10% EtOAc in hexanes) to provide 302 mg (55%) of pure product as a white solid. ¹H-NMR (CDCl₃) δ 7.51 (d, J=9.0 Hz, 1H), 7.35 (d, J=7.8 Hz, 2H), 7.18 (t, J=7.8 Hz, 1H), 6.64 (d, J=9.0 Hz, 1H), 4.53 (s, 2H), 3.86 (s, 3H), 3.83 (s, 3H).

Ex-42C: 2-(2,6-Dichloro-phenyl)-1-(2,6-dimethoxy-3-thien-2-yl-phenyl)-ethanone was prepared from 1-(3-bromo-2,6-dimethoxy-phenyl)-2-(2,6-dichloro-phenyl)-ethanone obtained from Ex-42B and thiophene 2-boronic acid in a similar manner as described in Ex-30E. The reaction could not be forced to completion. The crude material was purified by silica gel chromatography (25% EtOAc in hexanes) to provide product containing unreacted starting material. The isolated material was re-subjected to the reaction conditions. The crude material was purified by silica gel chromatography (10% EtOAc in hexanes) to provide 225 mg (74%) of pure product as a white solid. ¹H-NMR (CDCl₃) δ 7.57 (d, J=8.8 Hz, 1H), 7.32-7.38 (m, 4H), 7.18 (t, J=7.8 Hz, 1H), 7.08 (dd, J=5.4, 3.6 Hz, 1H), 6.75 (d, J=8.8 Hz, 1H), 4.59 (s, 2H), 3.85 (s, 3H), 3.65 (s, 3H).

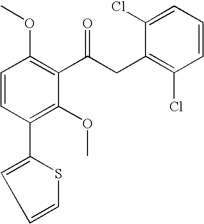

### 2-(2,6-Dichloro-phenyl)-1-(2,6-dimethoxy-3-thien-2-yl-phenyl)-ethanone

Ex-42D: The title compound was prepared from 2-(2,6-dichloro-phenyl)-1-(2,6-dimethoxy-3-thien-2-yl-phenyl)-ethanone obtained from Ex-42C in a similar manner as described in Ex-321. The crude material was purified by silica gel chromatography (10% EtOAc in hexanes, then a second column with 5% EtOAc in hexanes) followed by slurrying (EtOH/hexanes) and filtration to provide 47 mg (22%) of pure product as a solid, mp 152-154° C. ¹H-NMR (CDCl₃) δ 7.60 (d, J=9.0 Hz, 1H), 7.37-7.40 (m, 3H), 7.32 (d, J=5.2 Hz, 1H), 7.23 (dd, J=8.7, 7.2 Hz, 1H), 7.08 (dd, J=5.2, 3.6 Hz, 1H), 6.77 (d, J=9.0 Hz, 1H), 6.39 (s, 1H), 6.24 (s, 1H), 3.82 (s, 3H), 3.64 (s, 3H). HRMS (EI⁺) m/z: calc. 418.0197, found 418.0190. Anal. calculated for C₂₁H₁₆Cl₂O₃S: C, 60.15; H, 3.85; Cl, 16.91; S, 7.65. Found: C, 61.15; H, 3.89; Cl, 16.68; S, 7.50%.

### Example 43

### 1-(2,6-Dimethoxy-phenyl)-2-(2-thien-2-yl-phenyl)-propenone

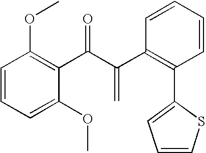

Ex-43A: 3-(2-Bromo-phenyl)-2-(tert-butyl-dimethyl-silanyloxy)-2-(2,6-dimethoxy-phenyl)-propionitrile was prepared from (tert-butyl-dimethyl-silanyloxy)-(2,6-dimethoxy-phenyl)-acetonitrile obtained from Ex-36A and 1-bromo-2-bromomethyl-benzene in a similar manner as described in Ex-30C. The crude material was purified by silica gel chromatography (10% EtOAc in hexanes) to provide 946 mg of impure product as a colorless oil. ¹H-NMR (CDCl₃) δ 7.07-7.65 (m, 5H), 6.54-6.59 (m, 2H), 3.71-4.01 (m, 8H), 0.86-0.89 (m, 9H), −0.15 (m, 6H).

Ex-43B: 2-(2-Bromo-phenyl)-1-(2,6-dimethoxy-phenyl)-ethanone was prepared from 3-(2-bromo-phenyl)-2-(tert-butyl-dimethyl-silanyloxy)-2-(2,6-dimethoxy-phenyl)-propionitrile obtained from Ex-43A in a similar manner as described in Ex-7F. The crude material was purified by silica gel chromatography (20-25% EtOAc in hexanes) to provide 82 mg (2 steps, 4%) of pure product as a colorless oil. ¹H-NMR (CDCl₃) δ 7.54 (d, J=9.0 Hz, 1H), 7.22-7.31 (m, 3H), 7.06-7.12 (m, 1H), 6.52 (d, J=7.5 Hz, 2H), 4.27 (s, 2H), 3.77 (s, 6H).

Ex-43C: 1-(2,6-Dimethoxy-phenyl)-2-(2-thien-2-yl-phenyl)-ethanone was prepared from 2-(2-bromo-phenyl)-1-(2,6-dimethoxy-phenyl)-ethanone obtained from Ex-43B and thiophene 2-boronic acid in a similar manner as described in Ex-30E. The reaction could not be forced to completion. The crude material was purified by silica gel chromatography (25% EtOAc in hexanes) to provide product containing unreacted starting material. The isolated material was re-subjected to the reaction conditions. The crude material was purified by silica gel chromatography (10% EtOAc in hexanes) to provide 44 mg (54%) of pure product as a white solid, mp 70-73° C. ¹H-NMR (CDCl₃) δ 7.61 (s, 1H), 7.12-7.33 (m, 6H), 7.04 (m, 1H), 6.50 (d, J=8.4 Hz, 2H), 4.21 (s, 2H), 3.71 (s, 6H).

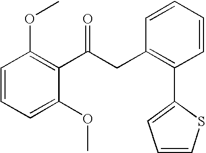

### 1-(2,6-Dimethoxy-phenyl)-2-(2-thien-2-yl-phenyl)-ethanone

Ex-43D: The title compound was prepared from 1-(2,6-dimethoxy-phenyl)-2-(2-thien-2-yl-phenyl)-ethanone obtained from Ex-43C in a similar manner as described in Ex-32I. The crude material was purified by silica gel chromatography (15-20% EtOAc in hexanes) to provide 22 mg (48%) of pure product as a white solid, mp 70-73° C. 1H-NMR (CDCl₃) δ 7.46-7.49 (m, 1H), 7.24-7.35 (m, 5H), 7.09 (d, J=4.2 Hz, 1H), 6.99 (dd, J=5.4, 3.3 Hz, 1H), 6.50 (d, J=8.1 Hz, 2H), 6.05 (s, 1H), 5.91 (s, 1H), 3.71 (s, 6H). HRMS (EI⁺) m/z: calc. 350.0977, found 350.0981. Anal. calculated for C₂₁H₁₈O₃S: C, 71.98; H, 5.18; S, 9.15. Found: C, 71.63; H, 5.61; S, 8.71%.

### Example 44

### 2-{3-[1-(2,6-Dimethoxy-3-thien-2-yl-benzoyl)-vinyl]-phenoxy}-2-methyl-propionic acid

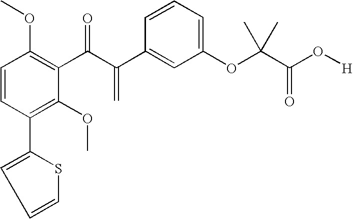

Ex-44A: 2-(3-Formyl-phenoxy)-2-methyl-propionic acid ethyl ester was prepared from 3-hydroxy benzaldehyde and ethyl-2-bromoisobutyrate in a similar manner as described in Ex-35A. The crude material was purified by silica gel chromatography (5-10% EtOAc in hexanes) to provide 12.83 g (66%) of pure product as a clear, colorless oil. ¹H-NMR (CDCl₃) δ 9.95 (s, 1H), 7.51 (dd, J=7.8, 2.5 Hz, 1H), 7.42 (t, J=7.8 Hz, 1H), 7.32 (dd, J=2.5, 1.2 Hz, 1H), 7.13 (dt, J=7.8, 1.2 Hz, 1H), 4.25 (q, J=6.6 Hz, 2H), 1.64 (s, 6H), 1.26 (t, J=6.6 Hz, 3H).

Ex-44B: 2-(3-Hydroxymethyl-phenoxy)-2-methyl-propionic acid ethyl ester was prepared from 2-(3-formyl-phenoxy)-2-methyl-propionic acid ethyl ester obtained from Ex-44A in a similar manner as described in Ex-7C. The resulting clear, colorless oil was used without purification (12.74 g, 99%). ¹H-NMR (CDCl₃) δ 7.26 (t, J=7.4 Hz, 1H), 7.01 (d, J=7.4 Hz, 1H), 6.92 (d, J=2.1 Hz, 1H), 6.78 (dd, J=7.4, 2.1 Hz, 1H), 4.67 (s, 2H), 4.27 (q, J=6.6 Hz, 2H), 1.63 (s, 6H), 1.29 (t, J=6.6 Hz, 3H).

Ex-44C: 2-(3-Bromomethyl-phenoxy)-2-methyl-propionic acid ethyl ester was prepared from 2-(3-hydroxymethyl-phenoxy)-2-methyl-propionic acid ethyl ester obtained from Ex-44B in a similar manner as described in Ex-7D. The crude material was purified by silica gel chromatography (8% EtOAc in hexanes) to provide 14.99 g (93%) of pure product as a clear, colorless oil. ¹H-NMR (CDCl₃) δ 7.20 (t, J=8.0 Hz, 1H), 7.00 (d, J=8.0 Hz, 1H), 6.88 (dd, J=2.4, 1.5 Hz, 1H), 6.75 (dd, J=8.0, 2.4 Hz, 1H), 4.42 (s, 2H), 4.24 (q, J=6.7 Hz, 2H), 1.61 (s, 6H), 1.25 (t, J=6.7 Hz, 3H).

Ex-44D: 2-{3-[2-(3-Bromo-2,6-dimethoxy-phenyl)-2-(tert-butyl-dimethyl-silanyloxy)-2-cyano-ethyl]-phenoxy}-2-methyl-propionic acid ethyl ester was prepared from 2-(3-bromomethyl-phenoxy)-2-methyl-propionic acid ethyl ester obtained from Ex-44C and (3-bromo-2,6-dimethoxy-phenyl)-(tert-butyl-dimethyl-silanyloxy)-acetonitrile obtained from Ex-30B in a similar manner as described in Ex-7E. The anion solution was stirred at 0° C. for 3¼ h prior to addition of the benzyl bromide. The crude material was purified by silica gel chromatography (10% EtOAc in hexanes) to provide 10.42 g (80%) of pure product as a clear, yellow oil. ¹H-NMR (CDCl₃) δ 7.47 (d, J=9.6 Hz, 1H), 7.08 (t, J=7.4 Hz, 1H), 6.84 (d, J=7.4 Hz, 1H), 6.70 (dd, J=7.4, 1.8 Hz, 1H), 6.65 (d, J=9.6 Hz, 1H), 6.54 (d, J=1.8 Hz, 1H), 4.16-4.22 (m, 2H), 4.02 (d, J=12.6 Hz, 1H), 3.90 (s, 3H), 3.36 (d, J=12.6 Hz, 1H), 3.23 (s, 3H), 1.58 (s, 3H), 1.34 (s, 3H), 1.26 (t, J=7.5 Hz, 3H), 0.87 (s, 9H), 0.17 (s, 3H), −0.21 (s, 3H).

Ex-44F: 2-{3-[2-(3-Bromo-2,6-dimethoxy-phenyl)-2-oxo-ethyl]-phenoxy}-2-methyl-propionic acid ethyl ester was prepared from 2-{3-[2-(3-bromo-2,6-dimethoxy-phenyl)-2-(tert-butyl-dimethyl-silanyloxy)-2-cyano-ethyl]-phenoxy}-2-methyl-propionic acid ethyl ester obtained from Ex-44E in a similar manner as described in Ex-7F. The crude material was purified by silica gel chromatography (25% EtOAc in hexanes) to provide 7.20 g (90%) of pure product as a clear, light yellow oil. ¹H-NMR (CDCl₃) δ 7.46 (d, J=9.6 Hz, 1H), 7.16 (dd, J=9.0, 7.6 Hz, 1H), 6.85 (d, J=7.6 Hz, 1H), 6.71-6.74 (m, 2H), 6.57 (d, J=9.0 Hz, 1H), 4.23 (q, J=6.9 Hz, 2H), 3.99 (s, 2H), 3.77 (s, 3H), 3.76 (s, 3H), 1.57 (s, 6H), 1.25 (t, J=6.9 Hz, 3H).

Ex-44G: 2-{3-[2-(2,6-Dimethoxy-3-thien-2-yl-phenyl)-2-oxo-ethyl]-phenoxy}-2-methyl-propionic acid ethyl ester was prepared from 2-{3-[2-(3-bromo-2,6-dimethoxy-phenyl)-2-oxo-ethyl]-phenoxy}-2-methyl-propionic acid ethyl ester obtained from Ex-44F and thiophene 2-boronic acid in a similar manner as described in Ex-30E. The crude material was purified by silica gel chromatography (20% EtOAc in hexanes) to provide 3.41 g (93%) of pure product as a light orange oil. ¹H-NMR (CDCl₃) δ 7.53 (d, J=9.0 Hz, 1H), 7.31-7.35 (m, 2H), 7.17 (t, J=8.2 Hz, 1H), 7.07 (dd, J=5.1, 3.9 Hz, 1H), 6.89 (d, J=8.2 Hz, 1H), 6.71-6.76 (m, 2H), 6.71 (d, J=9.0 Hz, 1H), 4.22 (q, J=6.9 Hz, 2H), 4.06 (s, 2H), 3.80 (s, 3H), 3.55 (s, 3H), 1.57 (s, 6H), 1.25 (t, J=6.9 Hz, 3H).

Ex-44H: 2-{3-[2-(2,6-Dimethoxy-3-thien-2-yl-phenyl)-2-oxo-ethyl]-phenoxy}-2-methyl-propionic acid was prepared from 2-{3-[2-(2,6-dimethoxy-3-thien-2-yl-phenyl)-2-oxo-ethyl]-phenoxy}-2-methyl-propionic acid ethyl ester obtained from Ex-44G in a similar manner as described in Ex-35G. The reaction was performed at 60° C. The crude material was purified by silica gel chromatography (5-10% MeOH in CH₂Cl₂) to provide 2.30 g (77%) of pure product as a yellow foam. ¹H-NMR (CDCl₃) δ 9.15 (bs, 1H), 7.52 (d, J=8.8 Hz, 1H), 7.30-7.33 (m, 2H), 7.20 (t, J=8.1 Hz, 1H), 7.06 (dd, J=5.1, 3.6 Hz, 1H), 6.95 (d, J=8.1 Hz, 1H), 6.80-6.85 (m, 2H), 6.69 (d, J=8.8 Hz, 1H), 4.08 (s, 2H), 3.75 (s, 3H), 3.51 (s, 3H), 1.53 (s, 6H).

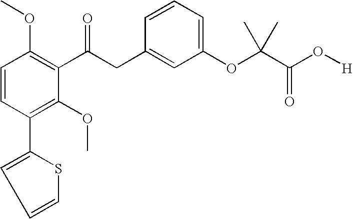

### 2-{3-[2-(2,6-Dimethoxy-3-thien-2-yl-phenyl)-2-oxo-ethyl]-phenoxy}-2-methyl-propionic acid

Ex-44I: The title compound was prepared from 2-{3-[2-(2,6-dimethoxy-3-thien-2-yl-phenyl)-2-oxo-ethyl]-phenoxy}-2-methyl-propionic acid obtained from Ex-44H in a similar manner as described in Ex-7G. The crude material was purified by silica gel chromatography (2 columns with 5% MeOH in CH₂Cl₂) to provide 1.06 g (45%) of product as a light yellow solid which contained hexanes; heating to 49-52° C. caused formation of a glass-like solid with mp 79-94° C. ¹H-NMR (CDCl₃) δ 7.58 (d, J=9.0 Hz, 1H), 7.36 (dd, J=3.6, 1.5 Hz, 1H), 7.29-7.33 (m, 2H), 7.18-7.21 (m, 1H), 7.06-7.09 (m, 2H), 6.93 (dd, J=8.1, 0.9 Hz, 1H), 6.75 (d, J=9.0 Hz, 1H), 6.18 (s, 1H), 5.98 (s, 1H), 3.80 (s, 3H), 3.61 (s, 3H), 1.61 (s, 6H). HRMS (EI⁺) m/z: calc. 452.1294, found 452.1282. Anal. calculated for C₂₅H₂₄O₆S: C, 68.55; H, 5.34; S, 6.54. Found: C, 66.25; H, 5.58; S, 6.80%.

### Example 45

### 2-{3-[1-(3-Benzo[b]thien-2-yl-2,6-dimethoxy-benzoyl)-vinyl]-phenoxy}-2-methyl-propionic acid

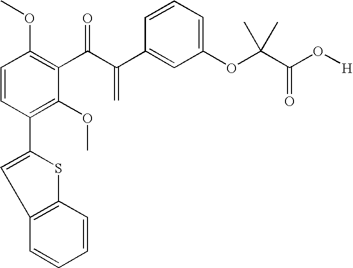

Ex-45A: 2-{3-[2-(3-Benzo[b]thien-2-yl-2,6-dimethoxy-phenyl)-2-oxo-ethyl]-phenoxy}-2-methyl-propionic acid ethyl ester was prepared from 2-{3-[2-(3-bromo-2,6-dimethoxy-phenyl)-2-oxo-ethyl]-phenoxy}-2-methyl-propionic acid ethyl ester obtained from Ex-44F and benzothiophene-2-boronic acid in a similar manner as described in Ex-30E. The crude material was purified by silica gel chromatography (2 columns with 20% EtOAc in hexanes) to provide 3.40 g (86%) of pure product as a viscous, light orange oil. ¹H-NMR (CDCl₃) δ 782-7.85 (m, 2H), 7.76-7.79 (m, 1H), 7.58-7.63 (m, 2H), 7.29-7.38 (m, 2H), 7.18 (t, J=7.5 Hz, 1H), 6.90 (d, J=7.5 Hz, 1H), 6.72-6.78 (m, 3H), 4.22 (q, J=7.4 Hz, 2H), 4.08 (s, 2H), 3.82 (s, 3H), 3.60 (s, 3H), 1.57 (s, 6H), 1.24 (t, J=7.4 Hz, 3H).

Ex-45B: 2-{3-[2-(3-Benzo[b]thien-2-yl-2,6-dimethoxy-phenyl)-2-oxo-ethyl]-phenoxy}-2-methyl-propionic acid was prepared from 2-{3-[2-(3-benzo[b]thien-2-yl-2,6-dimethoxy-phenyl)-2-oxo-ethyl]-phenoxy}-2-methyl-propionic acid ethyl ester obtained from Ex-45A in a similar manner as described in Ex-35G. The reaction was performed at 60° C. The crude material was purified by silica gel chromatography (5-10% MeOH in CH₂Cl₂) to provide 2.64 g (82%) of pure product as a faint yellow foam. ¹H-NMR (CDCl₃) δ 7.82-7.85 (m, 1H), 7.76-7.79 (m, 1H), 7.58-7.63 (m, 2H), 7.31-7.36 (m, 2H), 7.21-7.26 (m, 1H), 7.00 (d, J=7.8 Hz, 1H), 6.83-6.88 (m, 2H), 6.75 (d, J=8.7 Hz, 1H), 4.10 (s, 2H), 3.82 (s, 3H), 3.60 (s, 3H), 1.56 (s, 6H).

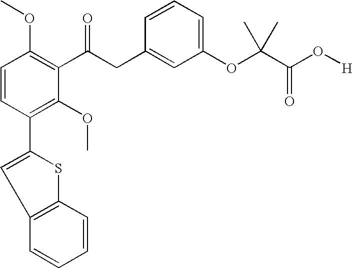

### 2-{3-[2-(3-Benzo[b]thien-2-yl-2,6-dimethoxy-phenyl)-2-oxo-ethyl]-phenoxy}-2-methyl-propionic acid

Ex-45C: The title compound was prepared from 2-{3-[2-(3-benzo[b]thien-2-yl-2,6-dimethoxy-phenyl)-2-oxo-ethyl]-phenoxy}-2-methyl-propionic acid obtained from Ex-45B in a similar manner as described in Ex-7G. The crude material was purified by silica gel chromatography (2 columns with 5% MeOH in CH₂Cl₂), followed by slurrying (hexanes with a small amount of CH₂Cl₂ added) and filtration to provide 1.72 g (64%) of pure product as an off-white solid, mp 135-137° C. ¹H-NMR (CDCl₃) δ 7.83 (dd, J=7.5, 1.5 Hz, 1H), 7.77 (dd, J=6.9, 2.7 Hz, 1H), 7.66 (d, J=8.4 Hz, 1H), 7.61 (s, 1H), 7.30-7.36 (m, 3H), 7.20 (d, J=8.7 Hz, 1H), 7.09-7.11 (m, 1H), 6.94 (dd, J=8.1, 1.2 Hz, 1H), 6.78 (d, J=9.0 Hz, 1H), 6.20 (s, 1H), 6.00 (s, 1H), 3.82 (s, 3H), 3.66 (s, 3H), 1.62 (s, 6H). HRMS (EI⁺) m/z: calc. 502.1450, found 502.1442. Anal. calculated for C₂₉H₂₆O₆S: C, 69.30; H, 5.21; S, 6.38. Found: C, 69.33; H, 5.28; S, 6.34%.

### Example 46

### 1-(5-Benzo[b]thien-2-yl-2,4-dimethoxy-phenyl)-2-(4-methoxy-phenyl)-propenone

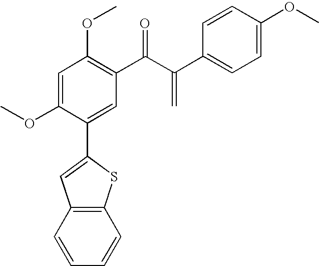

Ex-46A: 1-Bromo-2,4-dimethoxybenzene (1.58 g, 7.27 mmol), 4-methoxyphenyl acetyl chloride (1.11 mL, 7.26 mmol) and CH₂Cl₂ (20 mL) were sequentially charged into a clean reaction vessel and the resulting solution was cooled to −20° C. AlCl₃ (1.1 g, 8.25 mmol) was then added over 20 min and aged at −15° C. After an additional 10 min, the reaction was poured into 75 mL cold H₂O and extracted with CH₂Cl₂. The organic cut was dried with MgSO₄, filtered and concentrated to dryness. The crude product was dissolved in hot EtOAc (5 mL) and crystallized upon heptane (15 mL) addition. The product was filtered and then dried under vacuum affording 1.9 g (71% yield) of desired 1-(5-bromo-2,4-dimethoxy-phenyl)-2-(4-methoxy-phenyl)-ethanone. ¹H-NMR (300 MHz, CDCl₃): δ 7.99 (s, 1H), 7.12 (d, J=9.0 Hz, 2H), 6.84 (d, J=9.0 Hz, 2H), 6.43 (s, 1H), 4.19 (s, 2H), 3.95 (s, 3H), 3.94 (s, 3H), 3.78 (s, 3H).

Ex-46B: 1-(5-Bromo-2,4-dimethoxy-phenyl)-2-(4-methoxy-phenyl)-ethanone obtained from Ex-46A (1.87 g, 5.12 mmol), benzothiophene-2-boronic acid (0.95 g, 5.34 mmol) and THF (15 mL) were sequentially charged into a clean reaction vessel fitted with a reflux condenser and nitrogen inlet adapter. KF (625 mg, 10.76 mmol) and Pd(^{t}Bu₃P)₂ (28 mg, 0.055 mmol) were added and the solution was immediately heated to 60° C. and aged for 1.5 h. The reaction was diluted with H₂O and extracted with EtOAc. The layers were cut and the organic layer was dried with MgSO₄ and concentrated to dryness. The crude solid was dissolved in EtOAc (5 mL) and crystallized upon heptane (10 mL) addition. The product was filtered and then dried under vacuum affording 1.92 g (90% yield) of desired 1-(5-benzo[b]thien-2-yl-2,4-dimethoxy-phenyl)-2-(4-methoxy-phenyl)-ethanone. ¹H-NMR (DMSO-d₆) δ 7.98 (s, 1H), 7.88 (d, J=7.5 Hz, 1H), 7.77-7.80 (m, 2H), 7.25-7.34 (m, 2H), 7.09 (d, J=9.3 Hz, 2H), 6.80-6.83 (m, 3H), 4.17 (s, 2H), 4.00 (s, 3H), 3.99 (s, 3H), 3.67 (s, 3H).

Ex-46C: 1-(5-benzo[b]thien-2-yl-2,4-dimethoxy-phenyl)-2-(4-methoxy-phenyl)-ethanone obtained from Ex-46B (1.68 g, 4.01 mmol) and N,N,N′,N′-tetramethyl diaminomethane (10 mL) were cooled to 15° C. under nitrogen. Acetic anhydride (10 mL) was added over 20 min and the resulting solution was heated to 90° C. and aged for 1.5 h. The reaction was poured into 100 mL H₂O and extracted with EtOAc (100 mL). The resulting organic cut was dried over MgSO₄, filtered, and concentrated to dryness. The crude solid was purified by silica gel chromatography (10% EtOAc in hexanes) to provide 730 mg (42%) of the title compound, mp 166-168° C. ¹H-NMR (CDCl₃) δ 8.01 (s, 1H), 7.81 (d, J=8.5 Hz, 1H), 7.77 (d, J=8.5, 1H), 7.65 (s, 1H), 7.26-7.37 (m, 4H), 6.87 (d, J=9.0 Hz, 2H), 6.47 (s, 1H), 5.84 (s, 1H), 5.66 (s, 1H), 4.00 (s, 3H), 3.81 (s, 3H), 3.76 (s, 3H). HRMS (EI⁺) m/z: calc. 430.1239, found 430.1243.

### Example 47

### 1-(5-Benzo[b]thien-2-yl-2,4-dimethoxy-phenyl)-2-(4-fluoro-phenyl)-propenone

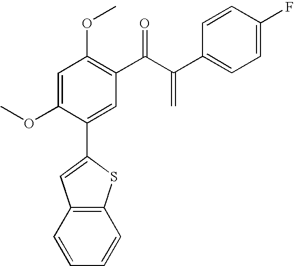

Ex-47A: 1-Bromo-2,4-dimethoxybenzene (1.58 g, 7.27 mmol), 4-fluorophenyl acetyl chloride (1.00 mL, 7.42 mmol) and CH₂Cl₂ (20 mL) were sequentially charged into a clean reaction vessel and the resulting solution was cooled to 3° C. AlCl₃ (2.0 g, 15.0 mmol) was then added over 20 min and aged at 8° C. After an additional 5 min, the reaction was poured into 75 mL cold H₂O and extracted with EtOAc. The organic cut was washed with saturated NaHCO₃, dried with MgSO₄, filtered and concentracted to dryness. The crude product was dissolved in EtOAc and crystallized upon hexane addition. The product was filtered and then further purified by silica gel chromatography (20% EtOAc in hexanes) to afford 0.78 g (30% yield) of desired 1-(5-bromo-2,4-dimethoxy-phenyl)-2-(4-fluoro-phenyl)-ethanone. ¹H-NMR (CDCl₃) δ 8.00 (s, 1H), 7.13-7.18 (m, 2H), 6.95-7.01 (m, 2H), 6.44 (s, 1H), 4.22 (s, 2H), 3.95 (s, 3H), 3.94 (s, 3H).

Ex-47B: 1-(5-Bromo-2,4-dimethoxy-phenyl)-2-(4-fluoro-phenyl)-ethanone obtained from Ex-47A (0.75 g, 2.12 mmol), benzothiophene-2-boronic acid (0.40 g, 2.25 mmol) and THF (8 mL) were sequentially charged into a clean reaction vessel fitted with a reflux condenser and nitrogen inlet adapter. KF (260 mg, 4.48 mmol) and Pd(^{t}Bu₃P)₂ (11 mg, 0.022 mmol) were added and the solution was immediately heated to 60° C. and aged for 1 h. The reaction was diluted with H₂O and extracted with EtOAc. The layers were cut and the organic layer was dried with MgSO₄ and concentrated to dryness. The crude solid was dissolved in EtOAc and crystallized upon hexane addition. The product was filtered and then dried under vacuum affording 0.75 g (87% yield) of desired 1-(5-benzo[b]thien-2-yl-2,4-dimethoxy-phenyl)-2-(4-fluoro-phenyl)-ethanone. ¹H-NMR (CDCl₃) δ 8.21 (s, 1H), 7.81 (d, J=8.2 Hz, 1H), 7.75 (d, J=8.2, 1H), 7.66 (s, 1H), 7.28-7.33 (m, 2H), 7.18-7.23 (m, 2H), 6.97-7.03 (m, 2H), 6.53 (s, 1H), 4.28 (s, 2H), 4.03 (s, 3H), 3.99 (s, 3H).

Ex-47C: To a solution of 1-(5-benzo[b]thien-2-yl-2,4-dimethoxy-phenyl)-2-(4-fluoro-phenyl)-ethanone obtained from Ex-47B (0.75 g, 1.85 mmol) and N,N,N′,N′-tetramethyl diaminomethane (10 mL) at room temperature was added acetic anhydride (10 mL). The resulting solution was heated to 90° C. and aged for 3 h. The reaction was poured into 100 mL H₂O and extracted with EtOAc (100 mL). The resulting organic cut was washed with 1N HCl, dried over MgSO₄, filtered, and concentrated to dryness. The crude solid was purified by silica gel chromatography (75% EtOAc in hexanes) to provide 450 mg (60%) of the title compound, mp 150-152° C. ¹H-NMR (CDCl₃) δ 8.00 (s, 1H), 7.81 (d, J=7.3 Hz, 1H), 7.76 (d, J=7.3, 1H), 7.66 (s, 1H), 7.26-7.41 (m, 4H), 7.00-7.06 (m, 2H), 6.47 (s, 1H), 5.88 (s, 1H), 5.75 (s, 1H), 4.01 (s, 3H), 3.76 (s, 3H). HRMS (EI⁺) m/z: calc. 418.1039, found 418.1032.

### Example 48

### 4-[2-(3,4-Dimethoxy-5-thien-2-yl-phenyl)-acryloyl]-benzoic acid methyl ester

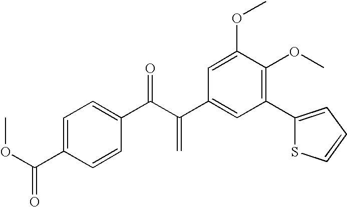

Ex-48A: 3-Bromo-4-hydroxy-5-methoxy-benzaldehyde (20 g, 86.6 mmol) was dissolved in DMF (200 mL) and potassium carbonate (15 g, 109 mmol) was added. Methyl iodide (13.5 g, 95.1 mmol) was added and the solution was aged at room temperature overnight. The crude reaction was diluted with EtOAc and H₂O and the layers were cut. The organic layer was washed sequentially with 1 N HCl, H₂O and then brine. The resulting organic was dried with MgSO₄ and concentrated on the rotavap. The crude material was purified by silica gel chromatography (10% EtOAc in hexanes) to provide 17.6 g (83%) of 3-bromo-4,5-dimethoxy-benzaldehyde. ¹H-NMR (CDCl₃) δ 9.84 (s, 1H), 7.65 (d, J=1.5 Hz, 1H), 7.39 (d, J=1.5 Hz, 1H), 3.95 (s, 3H), 3.93 (s, 3H).

Ex-48B: In a 500 mL round-bottom flask 3-bromo-4,5-dimethoxy-benzaldehyde obtained from Ex-48A (17 g, 69.4 mmol) was combined with thiophene 2-boronic acid (9.4 g, 73.5 mmol) and THF (200 mL). The resulting solution was purged subsurface with nitrogen for 10 min followed by the addition of KF (8.46 g, 145.6 mmol) and Pd(^{t}Bu₃P)₂ (355 mg, 0.695 mmol). The solution was then heated to reflux and aged for 3.5 h. To the hot reaction was added 600 μL H₂O and then the reaction was aged for an additional 1.5 h. HPLC indicated the reaction was not complete therefore another 600 μL H₂O was added. The reaction mixture was diluted with 1 N HCl and extracted with EtOAc. The organic phase was dried over MgSO₄, filtered, and concentrated onto silica gel. The crude material was purified by silica gel chromatography (20% EtOAc in hexanes) to provide 16.5 g (96%) of 3,4-dimethoxy-5-thien-2-yl-benzaldehyde. ¹H-NMR (CDCl₃) δ 9.94 (s, 1H), 7.78 (d, J=1.5 Hz, 1H), 7.57 (dd, J=3.6, 1.5 Hz, 1H), 7.40 (d, J=5.1, 1H), 7.35 (d, J=1.2 Hz, 1H), 6.85 (dd, J=5.1, 3.6 Hz, 1H), 3.96 (s, 3H), 3.93 (s, 3H).

Ex-48C: In a 500 mL round-bottom flask 3,4-dimethoxy-5-thien-2-yl-benzaldehyde obtained from Ex-48B (16.4 g, 66.05 mmol) was combined with THF (240 mL) and EtOH (200 mL). NaBH₄ (2.6 g, 68.73 mmol) was added in 3 portions over 15 min. The mixture was poured into H₂O and extracted with CH₂Cl₂. The organic phase was washed with H₂O (3×), dried over MgSO₄, filtered, concentrated onto silica gel. The crude material was purified by silica gel chromatography (30% EtOAc in hexanes) to provide 15.68 g (95%) of (3,4-dimethoxy-5-thien-2-yl-phenyl)-methanol. ¹H-NMR (CDCl₃) δ 7.49 (dd, J=3.7, 1.6 Hz, 1H), 7.34 (dd, J=5.1, 1.6 Hz, 1H), 7.21 (d, J=2.1, 1H), 7.06-7.09 (m, 1H), 6.85 (d, J=2.2 Hz, 1H), 4.63 (d, J=5.3 Hz, 2H), 3.87 (s, 3H), 3.81 (s, 3H), 2.15-2.19 (m, 1H).

Ex-48D: In a 500 mL round-bottom flask (3,4-dimethoxy-5-thien-2-yl-phenyl)-methanol obtained from Ex-48C (14.35 g, 57.33 mmol) was combined with CH₂Cl₂ (280 mL) and carbon tetrabromide (19.7 g, 59.4 mmol). The solution was cooled in a −5° C. bath and triphenylphosphine (15.8 g, 60.24 mmol) was added in 3 portions over 5 min. The reaction was stirred for 1 h and then concentrated onto silica gel. The crude material was purified by silica gel chromatography (30% EtOAc in hexanes) to provide 16.5 g (92%) of 2-(5-bromomethyl-2,3-dimethoxy-phenyl)-thiophene. ¹H-NMR (CDCl₃) δ 7.51 (dd, J=3.2, 1.8 Hz, 1H), 7.36 (dd, J=5.2, 1.8 Hz, 1H), 7.29 (d, J=3.2, 1H), 7.08-7.11 (m, 1H), 6.87 (d, J=0.9 Hz, 1H), 4.50 (s, 3H), 3.92 (s, 3H), 3.83 (s, 3H).

Ex-48E: In a 100 mL round-bottom flask 4-[(tert-butyl-dimethyl-silanyloxy)-cyano-methyl]-benzoic acid methyl ester obtained from Ex-1A (6.0 g, 19.65 mmol) was combined with THF (40 mL). The solution was cooled in a −78° C. bath and LHMDS (1 M in THF, 21 mL, 21 mmol) was added dropwise over 10 min. The resulting solution was stirred at −78° C. for 20 min. In a 200 mL round-bottom flask freshly-prepared 2-(5-bromomethyl-2,3-dimethoxy-phenyl)-thiophene obtained from Ex-48D (6.15 g, 19.64 mmol) was combined with THF (40 mL) and the solution was cooled in a −78° C. bath. Via cannula, the anion solution was transferred to the 200 mL flask in a slow stream over 20 min. The cold bath was removed allowing the reaction to slowly warm to 0° C. After 1 h, the solution was diluted with aqueous NH₄Cl and extracted with EtOAc. The organic phase was dried over Na₂SO₄, filtered, concentrated to dryness. The crude material was purified by silica gel chromatography (10% EtOAc in hexanes) to provide 6.98 g (70%) of 4-[1-(tert-butyl-dimethyl-silanyloxy)-1-cyano-2-(3,4-dimethoxy-5-thien-2-yl-phenyl)-ethyl]-benzoic acid methyl ester. ¹H-NMR (CDCl₃) δ 8.07 (d, J=8.9 Hz, 2H), 7.59 (d, J=8.9 Hz, 2H), 7.37 (d, J=3.0 Hz, 1H), 7.32 (d, J=5.2 Hz, 1H), 7.05 (dd, J=5.2, 3.0 Hz, 1H), 6.99 (d, J=1.8 Hz, 1H), 6.66 (d, J=1.8 Hz, 1H), 3.94 (s, 3H), 3.80 (s, 3H), 3.79 (s, 3H), 3.11-3.26 (m, 2H), 0.89 (s, 9H), 0.04 (s, 3H), −0.08 (s, 3H).

Ex-48F: In a 100 mL round-bottom flask 4-[1-(tert-butyl-dimethyl-silanyloxy)-1-cyano-2-(3,4-dimethoxy-5-thien-2-yl-phenyl)-ethyl]-benzoic acid methyl ester obtained from Ex-48E (6.61 g, 12.29 mmol) was combined with THF (50 mL). The solution was cooled in a 0° C. bath and TBAF (1 M in THF, 12.5 mL, 12.5 mmol) was added dropwise. After 30 min, the reaction mixture was diluted with aqueous NaHCO₃ and extracted with EtOAc. The organic phase was washed with H₂O and then brine, dried over MgSO₄, filtered and concentrated to dryness. The crude material was dissolved in EtOAc and the crystallized upon hexane addition. The product was filtered and dried under vacuum to afford 4.65 g (96%) of 4-[2-(3,4-dimethoxy-5-thien-2-yl-phenyl)-acetyl]-benzoic acid methyl ester. ¹H-NMR (CDCl₃) δ 8.14 (d, J=7.6 Hz, 2H), 8.07 (d, J=7.6 Hz, 2H), 7.46 (dd, J=3.6, 1.6 Hz, 1H), 7.34 (dd, J=5.1, 1.6 Hz, 1H), 7.14 (d, J=2.2, 1H), 7.07 (dd, J=5.1, 3.6 Hz, 1H), 6.73 (d, J=2.2 Hz, 1H), 4.29 (s, 2H), 3.95 (s, 3H), 3.87 (s, 3H), 3.80 (s, 3H).

Ex-48G: In a 25 mL round-bottom flask 4-[2-(3,4-dimethoxy-5-thien-2-yl-phenyl)-acetyl]-benzoic acid methyl ester obtained from Ex-48F (4.5 g, 11.4 mmol) was combined with MeOH (12 mL), formaldehyde (37 wt % in H₂O, 3 mL, 40.3 mmol), piperidine (150 μL), and AcOH (150 μL). The solution was heated at 60° C. and aged for 30 min. The solution was diluted with H₂O, and extracted with EtOAc. The organic phase was washed with 1N HCl, NaHCO₃, and then H₂O. The resulting organic was then dried over MgSO₄, filtered, concentrated to dryness. The crude material was purified by crystallization with EtOAc and hexane followed by filtration to provide 3.3 g (72%) of the title compound, mp 102-103° C. ¹H-NMR (CDCl₃) δ 8.10 (d, J=8.9 Hz, 2H), 7.95 (d, J=8.9 Hz, 2H), 7.45 (dd, J=3.5, 1.6 Hz, 1H), 7.35 (dd, J=5.2, 1.6 Hz, 1H), 7.30 (d, J=2.1, 1H), 7.07 (dd, J=5.2, 3.5 Hz, 1H), 6.89 (d, J=2.1 Hz, 1H), 6.14 (s, 1H), 5.71 (s, 1H), 3.95 (s, 3H), 3.87 (s, 3H), 3.83 (s, 3H). HRMS (EI⁺) m/z: calc. 408.1031, found 408.1036.

### Example 49

### 4-[2-(3,4-Dimethoxy-5-thien-2-yl-phenyl)-acryloyl]-benzoic acid

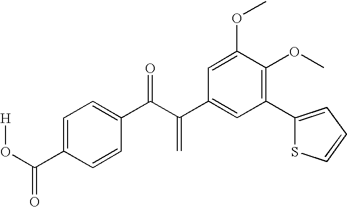

Ex-49A: In a 200 mL round-bottom flask 4-[2-(3,4-dimethoxy-5-thien-2-yl-phenyl)-acryloyl]-benzoic acid methyl ester obtained from Ex-48F (6.34 g, 15.99 mmol) was combined with THF (50 mL) and MeOH (50 mL). 1 N NaOH (32 mL, 32 mmol) was added in 1 portion and the solution was stirred at room temperature for 1.5 h. The reaction was diluted with EtOAc and H₂O and the layers were cut. The organic layer was extracted with additional H₂O. The combined aqueous cuts were then acidified to pH 2 with 1 N HCl and extracted with EtOAc. The organic phase was dried over MgSO₄, filtered, and concentrated to dryness. The crude material was purified by slurrying (EtOAc) and filtration to afford 5.13 g (84%) of 4-[2-(3,4-dimethoxy-5-thien-2-yl-phenyl)-acetyl]-benzoic acid. ¹H-NMR (DMSO-d₆) δ 8.12 (d, J=8.1 Hz, 2H), 8.05 (d, J=8.1 Hz, 2H), 7.49-7.54 (m, 2H), 7.22 (d, J=1.5, 1H), 7.07 (dd, J=5.1, 3.0 Hz, 1H), 6.89 (d, J=2.2 Hz, 11H), 4.41 (s, 2H), 3.78 (s, 3H), 3.69 (s, 3H).

Ex-49B: In a 10 mL round-bottom flask 4-[2-(3,4-dimethoxy-5-thien-2-yl-phenyl)-acetyl]-benzoic acid obtained from Ex-49A (4.0 g, 10.46 mmol) was combined with THF (24 mL), formaldehyde (37 wt % in H₂O, 2.8 mL, 37.6 mmol) and piperidine (160 μL, 1.61 mmol). The solution was heated at 40° C. and aged for 4 h. The solution was diluted with 1 N HCl and extracted with EtOAc. The organic phase was dried over MgSO₄, filtered and concentrated to dryness. The crude material was purified by slurrying (EtOAc and then EtOH) and filtration to provide 3.4 g (85%) of the title compound, mp 156-157° C. ¹H-NMR (DMSO-d₆) δ 13.3 (s, 1H), 8.04 (d, J=8.1 Hz, 2H), 7.90 (d, J=8.1 Hz, 2H), 7.51-7.55 (m, 2H), 7.28 (d, J=1.2, 1H), 7.06 (m, 1H), 7.03 (d, J=2.2 Hz, 1H), 6.35 (s, 1H), 5.68 (s, 1H), 3.81 (s, 3H), 3.73 (s, 3H). HRMS (EI⁺) m/z: calc. 394.0875, found 394.0879.

### Example 50

### 4-[2-(3,4-Dimethoxy-5-thien-2-yl-phenyl)-acryloyl]-N-(2-morpholin-4-yl-ethyl)-benzamide

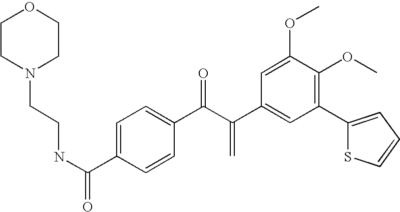

Ex-50A: In a 25 mL round-bottom flask 4-[2-(3,4-dimethoxy-5-thien-2-yl-phenyl)-acetyl]-benzoic acid obtained from Ex-49A (710 mg, 1.86 mmol) was combined with CH₂Cl₂ (10 mL). 2-Morpholin-4-yl-ethylamine (250 μL, 1.91 mmol) was added followed by 1-(3-dimethylaminopropyl)-3-ethylcarbodiimide hydrochloride (375 mg, 1.96 mmol) and the solution was stirred at room temperature for 45 min. Additional 2-morpholin-4-yl-ethylamine (250 μL, 1.91 mmol) was added followed by 1-(3-dimethylaminopropyl)-3-ethylcarbodiimide hydrochloride (375 mg, 1.96 mmol) and the resulting solution was aged for 1 h at room temperature. The reaction was diluted with EtOAc and H₂O and the layers were cut. The organic layer was dried over MgSO₄, filtered, and concentrated onto silica gel. The crude material was purified by silica gel chromatography (30% EtOAc in hexanes) to afford 715 mg (78%) of 4-[2-(3,4-dimethoxy-5-thien-2-yl-phenyl)-acetyl]-N-(2-morpholin-4-yl-ethyl)-benzamide. ¹H-NMR (DMSO-d₆) δ 8.55 (t, J=5.6 Hz, 1H), 8.11 (d, J=8.5 Hz, 2H), 7.92 (d, J=8.5 Hz, 2H), 7.49-7.53 (m, 2H), 7.23 (d, J=2.1, 1H), 7.08 (dd, J=5.3, 3.5 Hz, 1H), 6.89 (d, J=1.6 Hz, 1H), 4.39 (s, 2H), 3.78 (s, 3H), 3.69 (s, 3H), 3.53 (t, J=4.4 Hz, 4H), 3.24-3.30 (m, 2H), 2.45-2.47 (m, 2H), 2.37 (t, J=4.4 Hz, 4H).

Ex-50B: In a 10 mL round-bottom flask 4-[2-(3,4-dimethoxy-5-thien-2-yl-phenyl)-acetyl]-N-(2-morpholin-4-yl-ethyl)-benzamide obtained from Ex-50A (700 mg, 1.42 mmol) was combined with THF (4 mL), formaldehyde (37 wt % in H₂O, 375 μL, 5.04 mmol), piperidine (20 μL, 0.20 mmol) and AcOH (20 μL, 0.35 mmol). The solution was heated at 40° C. and aged for 3.25 h. Additional formaldehyde (37 wt % in H₂O, 150 uL) was added and the resulting solution was aged at 40° C. overnight. The solution was diluted with H₂O and extracted with EtOAc. The organic phase was dried over MgSO₄, filtered and concentrated onto silica gel. The crude material was purified by silica gel chromatography (5% MeOH in CH₂Cl₂) to provide 100 mg (15%) of the title compound, mp 84-87° C. ¹H-NMR (CDCl₃) δ 7.98 (d, J=8.5 Hz, 2H), 7.83 (d, J=8.5 Hz, 2H), 7.46 (d, J=3.5 Hz, 1H), 7.37 (d, J=5.1 Hz, 2H), 7.31 (d, J=2.0, 1H), 7.06-7.09 (m, 1H), 6.89 (d, J=2.0 Hz, 1H), 6.80 (s, 1H), 6.13 (s, 1H), 5.70 (s, 1H), 3.88 (s, 3H), 3.83 (s, 3H), 3.73 (t, J=4.6 Hz, 4H), 3.58-3.60 (m, 2H), 2.61 (t, J=5.9 Hz, 2H), 2.51 (t, J=4.6 Hz, 4H). HRMS (EI⁺) m/z: calc. 506.1875, found 506.1873.

### Example 51

### 2-(5-Benzo[b]thien-2-yl-2,4-dimethoxy-phenyl)-1-(4-fluoro-phenyl)-propenone

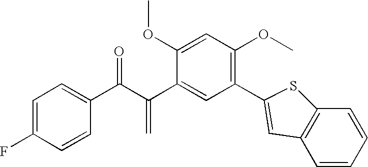

Ex-51A: 5-bromo-2,4-dimethoxybenzaldehyde (4.9 g, 20.0 mmol) was dissolved in ethylene glycol dimethyl ether (50 mL). Tetrakis(triphenylphosphine) palladium (0) (2.32 g, 2 mmol) was added, and the mixture was stirred at room temperature under nitrogen for 5 min. Benzo[b]thiophene-2-boronic acid (4.27 g, 24 mmol) and sodium carbonate solution (2 M, 20 mL) were added. The mixture was stirred at reflux under nitrogen for 24 hours. Upon cooling to room temperature, the mixture was poured into water and extracted with ethyl acetate. The organic phase was dried over Na₂SO₄ and evaporated. Silica gel chromatography (hexane/ethyl acetate 2:1 then 1:1) gave 4.75 g (83%) of the desired 5-(benzo[b]thien-2-yl)-2,4-dimethoxybenzaldehyde. ¹H NMR (CDCl₃) δ 10.36 (s, 1H), 8.20 (s, 1H), 7.78-7.83 (m, 2H), 7.68 (s, 1H), 7.27-7.36 (m, 2H), 6.54 (s, 1H), 4.06 (s, 3H), 4.00 (s, 3H).

Ex-51AA: (alternative procedure) 5-bromo-2,4-dimethoxybenzaldehyde (20 g), benzo[b]thiophene-2-boronic acid (16 g) and THF (200 mL) were sequentially charged into a clean reaction vessel fitted with a reflux condenser, mechanical stirrer and nitrogen inlet adapter. Nitrogen was bubbled into the resulting solution for 20 min followed by the sequential addition of KF (10 g) and Pd(^{t}Bu₃P)₂ (0.417 g). The solution was immediately heated to 60° C. and aged for 1.5 h. Upon completion, as determined by HPLC, the reaction was diluted with H₂O (200 mL) and transferred to a separatory funnel containing EtOAc (200 mL) and H₂O (200 mL). The layers were cut and the aqueous layer was extracted with EtOAc (100 mL). The combined organic cuts were filtered through a pre-washed pad of solka floc (5 g). The pad of solka floc and spent catalyst were washed with fresh EtOAc (200 mL) and this wash combined with the batch. The resultant filtrate was batch concentrated and solvent switched to 33 wt % 5-(benzo[b]thien-2-yl)-2,4-dimethoxybenzaldehyde in THF in preparation for crystallization. (Note: The internal temperature during batch concentration should be kept above 45° C. to prevent premature crystallization.) The resulting THF solution of 5-(benzo[b]thien-2-yl)-2,4-dimethoxybenzaldehyde was then charged with heptane (20 mL) and slowly cooled to ambient temperature. Crystallization was then completed with the slow addition of heptane (175 mL) and cooling to 4° C. After aging for 1 h, the batch was filtered and then dried on the filter funnel under a stream of N₂. The semi-wet cake was then transferred to clean trays and dried to a constant weight in the vacuum oven (40° C., 20 in Hg) affording 23.74 g (97% yield) of desired 5-(benzo[b]thien-2-yl)-2,4-dimethoxybenzaldehyde as a light orange crystalline solid, mp 134-136° C. HPLC assay of this solid indicated >99.9 LCAP. ¹H-NMR identical as above.

Ex-51AAA: (An alternative procedure) 5-bromo-2,4-dimethoxybenzaldehyde (2150 g, 8.77 mol) was charged to a 72-L reactor followed by THF (13.0 L). The mixture was stirred for 25 min under an argon sparge. KF (1290 g, 22.20 mol) was added to the reactor and the batch heated to 65° C. under a nitrogen atmosphere, which resulted in a yellow-brown suspension. A solution of Pd(^{t}Bu₃P)₂ (4.3 g, 8.4 mmol) in THF (110 mL) was sparged with argon for 21 minutes and was then added to the reactor resulting in a dark green suspension. A solution of benzothiophene-2-boronic acid (1634 g, 9.18 mol) in THF (8.6 L) was sparged with argon for 21 minutes, and then added to the hot suspension via an addition funnel. The addition rate was approximately 100 mL/min and the total addition required 85 minutes. During the addition, the suspension became lighter in color and ended as a yellow suspension. After 3.8 L of the boronic acid solution had been added, the suspension began to reflux more vigorously and the addition was suspended until the reflux had returned to normal (approximately 3 minutes). The suspension was maintained at 65° C. for 1 hour after the addition was complete, sampled for HPLC analysis and the heat discontinued.

Water (4.3 L) was added to the cooled batch (≦30° C.) and the mixture stirred for 30 minutes and allowed to settle for 25 minutes. The organic phase was washed with saturated sodium chloride solution (6.5 L) for 31 minutes, settled for 20 minutes and the aqueous phase separated. The organic phase was dried with sodium sulfate (1075 g) for 70 minutes. A filter pad was prepared from celite 545 (1075 g) and THF (3.8 L) and the THF discarded. The contents of the 72-L reactor were transferred to the filter pad and the mixture filtered under vacuum. Once the transfer was complete, the reactor was rinsed with THF (3.2 L) and the rinse used to wash the filter cake. The orange organic phases were concentrated in vacuo at 35° C. The wet solid was dried in a vacuum oven (25° C., 30 in Hg) for 15 hours, 32 minutes and weighed. Drying was continued for a further 4 hours at which point the weight was constant and the crude dry product transferred to two amber glass containers and blanketed with nitrogen affording 2542 g (97% of theory) of crude product. Crystallization from THF/heptane results in analytically pure 5-(benzo[b]thien-2-yl)-2,4-dimethoxybenzaldehyde.

Ex-51B: In a 250 mL round-bottom flask 5-(benzo[b]thien-2-yl)-2,4-dimethoxybenzaldehyde obtained from Ex-51A, Ex-51AA or Ex-51AAA (10.0 g, 33.52 mmol) was combined with CH₂Cl₂ (100 mL) and carbon tetrabromide (11.68 g, 35.22 mmol). The solution was cooled in a 3° C. bath and triphenylphosphine (18.5 g, 70.53 mmol) was added in 3 portions over 10 min. The cooling bath was removed and the reaction was aged for 50 min. The resulting reaction mixture was diluted with hexanes and filtered. The filter cake was washed with a 1:1 solution of ether and hexane. The combined filtrate was concentrated to a crude solid and then purified by silica gel chromatography (10% EtOAc in hexanes) to provide 6.2 g (41%) of 2-[5-(2,2-dibromo-vinyl)-2,4-dimethoxy-phenyl]-benzo[b]thiophene. ¹H-NMR (CDCl₃) δ 8.15 (s, 1H), 7.82 (d, J=7.4 Hz, 1H), 7.77 (d, J=7.4 Hz, 1H), 7.66 (s, 1H), 7.58 (s, 1H), 7.29-7.37 (m, 2H), 6.50 (s, 1H), 3.99 (s, 3H), 3.90 (s, 3H).

Ex-51C: In a 25 mL round-bottom flask 2-[5-(2,2-dibromo-vinyl)-2,4-dimethoxy-phenyl]-benzo[b]thiophene obtained from Ex-51B (1.0 g, 2.20 mmol) was combined with DMF (7 mL), H₂O (2.5 mL) and piperidine (1.1 mL, 11.11 mmol). The solution was heated to 100° C. and aged for 3 h. The reaction was cooled to room temperature, diluted with EtOAc and H₂O and the layers were cut. The organic layer was dried with MgSO₄, filtered and concentrated to dryness. Purification by silica gel chromatography (50% EtOAc in hexanes) provided 0.47 g (54%) of 2-(5-benzo[b]thien-2-yl-2,4-dimethoxy-phenyl)-1-piperidin-1-yl-ethanone. ¹H-NMR (CDCl₃) δ 7.80 (d, J=7.3 Hz, 1H), 7.74 (d, J=7.3 Hz, 1H), 7.63 (s, 1H), 7.54 (s, 1H), 7.23-7.34 (m, 2H), 6.55 (s, 1H), 3.97 (s, 3H), 3.89 (s, 3H), 3.67 (s, 2H), 3.60 (t, J=4.9 Hz, 2H), 3.41 (t, J=4.9 Hz, 2H), 1.56-1.61 (m, 4H), 1.45-1.46 (m, 2H).

Ex-51D: In a 25 mL round-bottom flask 2-(5-benzo[b]thien-2-yl-2,4-dimethoxy-phenyl)-1-piperidin-1-yl-ethanone obtained from Ex-51C (430 mg, 1.09 mmol) was combined with THF (5 mL) and the resulting solution was cooled to −78° C. 4-Fluoro-phenylmagnesium bromide (2 M in Et₂O, 0.60 mL, 1.2 mmol) was added dropwise over 10 min. After 10 min at −78° C., additional 4-fluoro-phenylmagnesium bromide (2 M in Et₂O, 0.55 mL, 1.1 mmol) was added and the resulting solution was aged. After 1.75 h the cooling bath was removed and the reaction was allowed to warm to room temperature overnight. HPLC analysis indicated the reaction was still incomplete therefore additional 4-fluoro-phenylmagnesium bromide (2 M in Et₂O, 0.3 mL, 0.6 mmol) was added and the reaction was aged for 30 min. The reaction was quenched with saturated NH₄Cl and then extracted with EtOAc. The organic phase was dried over MgSO₄, filtered and concentrated onto silica gel. The crude material was purified by silica gel chromatography (50% EtOAc in hexanes) to provide 120 mg (30%) of 2-(5-benzo[b]thien-2-yl-2,4-dimethoxy-phenyl)-1-(4-fluoro-phenyl)-ethanone. ¹H-NMR (DMSO-d₆) δ 8.07-8.12 (m, 2H), 7.87 (d, J=7.4 Hz, 1H), 7.75 (d, J=7.4 Hz, 1H), 7.71 (s, 1H), 7.58 (s, 1H), 7.23-7.36 (m, 4H), 6.77 (s, 1H), 4.28 (s, 2H), 3.95 (s, 3H), 3.77 (s, 3H).

Ex-51E: To a solution of 2-(5-benzo[b]thien-2-yl-2,4-dimethoxy-phenyl)-1-(4-fluoro-phenyl)-ethanone obtained from Ex-51D (90 mg, 0.22 mmol) and N,N,N′,N′-tetramethyl diaminomethane (1 mL) at room temperature was added acetic anhydride (1 mL). The resulting solution was heated to 40° C. and aged overnight. The reaction was poured into 25 mL 1 N HCl and extracted with EtOAc (25 mL). The resulting organic cut was dried over MgSO₄, filtered, and concentrated onto silica gel. The crude was purified by silica gel chromatography (30% EtOAc in hexanes) to provide 80 mg (90%) of the title compound, mp 142-144° C. ¹H-NMR (CDCl₃) δ 7.90-7.96 (m, 2H), 7.76-7.84 (m, 2H), 7.74 (s, 1H), 7.68 (s, 1H), 7.29-7.37 (m, 2H), 7.06-7.12 (m, 2H), 6.47 (s, 1H), 5.99 (s, 1H), 5.72 (s, 1H), 3.97 (s, 3H), 3.63 (s, 3H). HRMS (EI⁺) m/z: calc. 418.1039, found 418.1042.

### Example 52

### 1-(2-Methoxy-5-thien-2-yl-phenyl)-2-(4-nitro-phenyl)-propenone

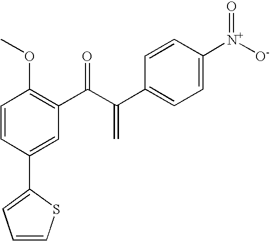

Ex-52A: 1-(5-Bromo-2-hydroxy-phenyl)-2-(4-nitro-phenyl)-ethanone was prepared from 1-bromo-4-methoxy-benzene and (4-nitro-phenyl)-acetyl chloride in a similar manner as described in Ex-46A. The crude material was purified by crystallization from EtOAc and hexanes to provide 14.5 g (52%) of pure product. 1H-NMR (DMSO-d₆) δ 11.86 (s, 1H), 8.24 (d, J=8.5 Hz, 2H), 7.94 (d, J=2.4 Hz, 1H), 7.58 (dd, J=8.6, 2.0 Hz, 1H), 7.43 (d, J=7.9 Hz, 2H), 6.93 (d, J=8.5 Hz, 1H), 4.42 (s, 2H).

Ex-52B: 4-Bromo-2-[1-hydroxy-2-(4-nitro-phenyl)-ethyl]-phenol was prepared from 1-(5-bromo-2-hydroxy-phenyl)-2-(4-nitro-phenyl)-ethanone obtained from Ex-52A in a similar manner as described in Ex-1C. The crude product was concentrated to dryness and used without futher purification

Ex-52C: 4-Bromo-2-[1-hydroxy-2-(4-nitro-phenyl)-ethyl]-phenol obtained from Ex-52B (5.0 g, 14.9 mmol) was dissolved in acetone (75 mL) and potassium carbonate (3.2 g, 23.2 mmol) was added. Methyl sulfate (1.5 mL, 15.9 mmol) was added and the solution was heated to reflux and aged for 50 min. The crude reaction was diluted with EtOAc and H₂O and the layers were cut. The organic layer was washed with 1 N HCl and concentrated to dryness. The crude 1-(5-bromo-2-methoxy-phenyl)-2-(4-nitro-phenyl)-ethanol was used without further purification.

Ex-52D: In a 500 mL round-bottom flask 1-(5-bromo-2-methoxy-phenyl)-2-(4-nitro-phenyl)-ethanol obtained from Ex-52C (5.24 g, 14.8 mmol) was dissolved in CH₂Cl₂ (200 mL). Pyridinium dichromate (11.0 g, 29.24 mmol) was added and the reaction was stirred at room temperature overnight. Additional pyridinium dichromate (5.5 g, 14.6 mmol) was added, the reaction was heated to 40° C. and then aged for 5 h. The reaction was cooled to room temperature and then filtered through a pad of silica gel. The silica gel was rinsed with excess CH₂Cl₂ and the combined organic was concentrated onto silica gel. Purification by silica gel chromatography (50% EtOAc in hexanes) followed by crystallization with EtOAc and hexanes provided 1.9 g (36%) of 1-(5-bromo-2-hydroxy-phenyl)-2-(4-nitro-phenyl)-ethanone as a white solid. ¹H-NMR (DMSO-d₆) δ 8.14 (d, J=9.1 Hz, 2H), 7.65-7.71 (m, 2H), 7.46 (d, J=8.0 Hz, 2H), 7.15 (d, J=9.1 Hz, 1H), 4.42 (s, 2H), 3.87 (s, 3H).

Ex-52E: 1-(2-Methoxy-5-thien-2-yl-phenyl)-2-(4-nitro-phenyl)-ethanone was prepared from 1-(5-bromo-2-hydroxy-phenyl)-2-(4-nitro-phenyl)-ethanone obtained from Ex-52D and thiophene 2-boronic acid in a similar manner as described in Ex-46×. The crude material was purified by silica gel chromatography (50% EtOAc in hexanes) to provide 1.57 g (84%) of pure product. ¹H-NMR (DMSO-d₆) δ 8.14 (d, J=8.5 Hz, 2H), 7.78-7.84 (m, 2H), 7.40-7.51 (m, 4H), 7.22 (d, J=9.2 Hz, 1H), 7.08 (dd, J=4.9, 3.5 Hz, 1H), 4.46 (s, 2H), 3.91 (s, 3H).

Ex-52F: The title compound was prepared from 1-(2-methoxy-5-thien-2-yl-phenyl)-2-(4-nitro-phenyl)-ethanone obtained from Ex-52E in a similar manner as described in Ex-3D. The crude material was purified by silica gel chromatography (10% EtOAc in hexanes) to provide 58 mg (56%) of product. ¹H-NMR (DMSO-d₆) δ 8.20 (d, J=8.7 Hz, 2H), 7.76 (dd, J=9.3, 2.0 Hz, 1H), 7.70 (d, J=2.7 Hz, 1H), 7.64 (d, J=8.7 Hz, 2H), 7.44-7.48 (m, 2H), 7.07-7.13 (m, 2H), 6.31 (s, 1H), 5.92 (s, 1H), 3.66 (s, 3H).

### Example 53

### 4-[1-(2-Methoxy-5-thien-2-yl-benzoyl)-vinyl]-benzonitrile

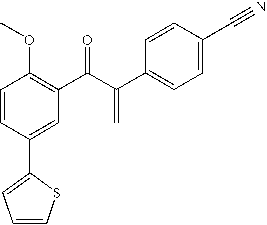

Ex-53A: In a 25 mL round-bottom flask 2-methoxy-5-thien-2-yl-benzaldehyde obtained from Ex-7B (1.5 g, 6.87 mmol) was added to anhydrous THF (10 mL) and the resulting solution was cooled to −30° C. 4-Cyano-benzylzinc bromide (0.5 M in THF, 13.8 mL, 6.9 mmol) was added over 5 min and the reaction was slowly warmed to 0° C. After 2 h the reaction was diluted with EtOAc and 1 N HCl. The layers were cut and the organic layer was diluted with hexanes and filtered through a pad of silica gel. After rinsing the silica gel with additional EtOAc and hexanes the combined organic was concentrated to dryness. The crude 4-[2-hydroxy-2-(2-methoxy-5-thien-2-yl-phenyl)-ethyl]-benzonitrile was used without further purification.

Ex-53B: In a 50 mL round-bottom flask 4-[2-hydroxy-2-(2-methoxy-5-thien-2-yl-phenyl)-ethyl]-benzonitrile obtained from Ex-53A (2.30 g, 6.87 mmol) was dissolved in CH₂Cl₂ (25 mL) followed by the addition of Celite (5 g). Pyridinium chlorochromate (1.5 g, 6.96 mmol) was added and the reaction was stirred at room temperature for 7 h. The reaction mixture was then passed through a plug of Celite which was washed with additional CH₂Cl₂. The combined organic was concentrated to a solid. Purification by silica gel chromatography (10% EtOAc in hexanes) followed by crystallization with EtOAc and hexanes provided 600 mg (26%) of 4-[2-(2-methoxy-5-thien-2-yl-phenyl)-2-oxo-ethyl]-benzonitrile. ¹H-NMR (DMSO-d₆) δ 7.80 (dd, J=9.0, 2.5 Hz, 1H), 7.73-7.76 (m, 3H), 7.46 (d, J=5.2 Hz, 1H), 7.39-7.42 (m, 3H), 7.21 (d, J=9.0 Hz, 1H), 7.08 (dd, J=5.2, 3.8 Hz, 1H), 4.40 (s, 2H), 3.90 (s, 3H).

Ex-53C: The title comound was prepared from 4-[2-(2-methoxy-5-thien-2-yl-phenyl)-2-oxo-ethyl]-benzonitrile obtained from Ex-53B in a similar manner as described in Ex-51E. The crude material was purified by silica gel chromatography (25% EtOAc in hexanes) followed by slurrying with ether to provide 55 mg (38%) of product, mp 86-88° C. ¹H-NMR (CDCl₃) δ 7.61-7.70 (m, 4H), 7.51 (d, J=7.8 Hz, 2H), 7.23 (m, 2H), 7.05 (dd, J=5.3, 3.7 Hz, 1H), 6.89 (d, J=8.3 Hz, 1H), 6.07 (s, 1H), 5.94 (s, 1H), 3.70 (s, 3H). HRMS (EI⁺) m/z: calc. 345.0824, found 345.0829.

### Example 54

### 4-[1-(2-Methoxy-5-thien-2-yl-benzoyl)-vinyl]-benzoic acid

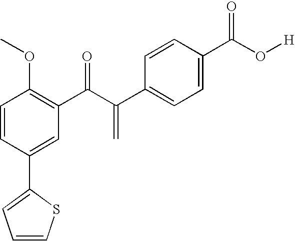

Ex-54A: In a 25 mL round-bottom flask 4-[2-(2-methoxy-5-thien-2-yl-phenyl)-2-oxo-ethyl]-benzonitrile obtained from Ex-53B (440 mg, 1.32 mmol) was combined with EtOH (8 mL) and KOH (45 wt % in H₂₀, 1 mL, 11.7 mmol). The solution was stirred at 75° C. for 1 h and then 10 mL H2O was added. After 5 h additional KOH (45 wt % in H₂O, 2 mL, 23.4 mmol) was added. The reaction was heated for an additional 5 h and then allowed to cool to room temperature overnight. EtOAc and H₂O were added and the layers were cut. The aqueous layer was acidified with conc. HCl and extracted with EtOAc. The resulting organic was extracted with saturated NaHCO₃ and the aqueous layer was acidified with conc. HCl and extracted with fresh EtOAc. The organic cut was concentrated to dryness. The crude material was purified by silica gel chromatography (10% MeOH in hexanes) to provide 28 mg (6%) of 4-[2-(2-methoxy-5-thien-2-yl-phenyl)-2-oxo-ethyl]-benzoic acid. ¹H-NMR (CDCl₃) δ 8.02 (d, J=8.1 Hz, 2H), 7.91 (d, J=1.9 Hz, 1H), 7.67 (dd, J=8.4, 2.6 Hz, 1H), 7.32 (d, J=8.1 Hz, 2H), 7.21-7.24 (m, 2H), 7.01-7.04 (m, 1H), 6.96 (d, J=9.1 Hz, 1H), 4.37 (s, 2H), 3.93 (s, 3H).

Ex-54B: The title compound was prepared from 4-[2-(2-methoxy-5-thien-2-yl-phenyl)-2-oxo-ethyl]-benzoic acid obtained from Ex-54A in a similar manner as described in Ex-49B. The crude material was purified by silica gel chromatography (10% MeOH in CH₂Cl₂) followed by crystallization from EtOAc and hexanes to provide 6.6 mg (31%) of pure product. ¹H-NMR (DMSO-d) 67.89 (d, J=8.3 Hz, 2H), 7.75 (dd, J=9.2, 2.1 Hz, 1H), 7.67 (d, J=2.1 Hz, 1H), 7.44-7.48 (m, 4H), 7.07-7.12 (m, 2H), 6.19 (s, 1H), 5.81 (s, 1H), 3.66 (s, 3H). HRMS (EI⁺) m/z: calc. 364.0769, found 364.0775.

### Example 55

### 4-[2-(2,4-Dimethoxy-5-thien-2-yl-phenyl)-acryloyl]-benzoic acid

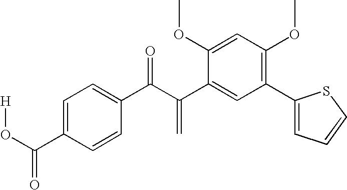

Ex-55A: In a 250 mL round-bottom flask (methoxymethyl) triphenylphosphonium chloride (14 g, 40.8 mmol) was combined with THF (150 mL). The reaction was cooled to −5° C. and nBuLi (1.6 M in hexanes, 26 mL, 41.6 mmol) was added over 10 min. The resulting solution was aged for 30 min at 5° C. and then 5-bromo-2,4-dimethoxy-benzaldehyde (5.0 g, 20.4 mmol) was added in 1 portion. The resulting solution was aged for 2 h. The solution was diluted with 1 N HCl and then extracted with EtOAc. The organic phase was washed with H₂O, dried over MgSO₄, filtered and concentrated to dryness. The crude 1-bromo-2,4-dimethoxy-5-(2-methoxy-vinyl)-benzene was used in the hydrolysis without additional purification.

Ex-55B: In a 200 mL round-bottom flask 1-bromo-2,4-dimethoxy-5-(2-methoxy-vinyl)-benzene obtained from Ex-55A (5.57 g, 20.4 mmol) was combined with THF (40 mL), H₂O (20 mL), and concentrated HCl (10 mL). The resulting solution was heated at 65° C. and aged for 1 h. The reaction was cooled, diluted with H₂O and extracted with EtOAc. The organic phase was concentrated onto silica gel. The crude material was purified by silica gel chromatography (10% EtOAc in hexanes) to provide 2.95 g (2 steps, 56%) of (5-bromo-2,4-dimethoxy-phenyl)-acetaldehyde. ¹H-NMR (CDCl₃) δ 9.61 (t, J=2.0 Hz, 1H), 7.25 (s, 1H), 6.47 (s, 1H), 3.89 (s, 3H), 3.80 (s, 3H), 3.52 (d, J=2.0 Hz, 2H).

Ex-55C: In a 100 mL round-bottom flask (5-bromo-2,4-dimethoxy-phenyl)-acetaldehyde obtained from Ex-55B (2.85 g, 11.0 mmol) was combined with thiophene 2-boronic acid (1.5 g, 11.7 mmol), THF (30 mL) and H₂O (200 μL). The resulting solution was purged subsurface with nitrogen for 10 min and then KF (2.0 g, 34.4 mmol), tri-t-butylphosphine (10 wt % in hexanes, 270 mg, 0.13 mmol) and tris(dibenzylideneacetone) dipalladium (0) (50 mg, 0.05 mmol). The solution was heated to reflux for 1.25 h followed by additional thiophene 2-boronic acid (0.5 g, 3.9 mmol) and H₂O (200 μL). After 15 min additional tri-t-butylphosphine (10 wt % in hexanes, 270 mg, 0.13 mmol), tris(dibenzylideneacetone) dipalladium (0) (50 mg, 0.05 mmol) and thiophene 2-boronic acid (1.5 g, 11.7 mmol) was added as a solution in THF (5 mL). The reaction was charged with 10 mL 2 N Na₂CO₃ and tetrakis (triphenylphospine) palladium (0) (25 mg, 0.02 mmol). The reaction was diluted with H₂O and EtOAc and the organic layer was washed with 1 N HCl dried over MgSO₄, filtered and concentrated to dryness. The crude material was purified by slurrying (20% MTBE in hexanes) to provide 800 mg (28%) (2,4-dimethoxy-5-thien-2-yl-phenyl)-acetaldehyde. The product and sm co-elude on the HPLC and TLC therefore the additional reagent charges were probably not neccessary. ¹H-NMR (CDCl₃) δ 9.69 (t, J=2.2 Hz, 1H), 7.37-7.39 (m, 2H), 7.27 (d, J=5.1 Hz, 1H), 7.05-7.08 (m, 1H), 6.57 (s, 1H), 3.95 (s, 3H), 3.87 (s, 3H), 3.62 (d, J=2.2 Hz, 2H).

Ex-55D: In a 25 mL round-bottom flask 4-iodo-benzoic acid methyl ester (875 mg, 3.34 mmol) was combined with THF (12 mL) and the resulting solution was cooled to −40° C. Isopropyl magnesium chloride (2M in THF, 1.67 mL, 3.34 mmol) was added over 5 min and the reaction was aged for 40 min. The resulting 4-cyano-phenylmagnesium chloride was added slowly into a THF (5 mL) solution of (2-methoxy-5-thien-2-yl-phenyl)-acetaldehyde obtained from Ex-55C (710 mg, 2.71 mmol) precooled to −40° C. After 30 min, the reaction was quenched with saturated NH₄Cl and extracted with EtOAc. The organic phase was concentrated to dryness and the resulting oil was put through a plug of silica gel (30% EtOAc in hexanes). Concentrating to dryness afforded 600 mg (56%) of semi-pure 4-[2-(2,4-dimethoxy-5-thien-2-yl-phenyl)-1-hydroxy-ethyl]-benzoic acid methyl ester which was used without further purification.

Ex-55E: 4-[2-(2,4-Dimethoxy-5-thien-2-yl-phenyl)-acetyl]-benzoic acid methyl ester was prepared from 4-[2-(2,4-dimethoxy-5-thien-2-yl-phenyl)-1-hydroxy-ethyl]-benzoic acid methyl ester obtained from Ex-55D in a similar manner as described in Ex-53B. The crude material was purified by silica gel chromatography (10% EtOAc in hexanes) followed by slurrying with ether to provide 84 mg (14%) of pure product. 1H-NMR (CDCl₃) δ 8.09 (d, J=8.4 Hz, 2H), 8.05 (d, J=8.4 Hz, 2H), 7.39 (s, 1H), 7.32 (d, J=2.9 Hz, 1H), 7.21-7.24 (m, 1H), 7.01 (dd, J=5.4, 3.9 Hz, 1H), 6.52 (s, 1H), 4.23 (s, 2H), 3.92 (s, 3H), 3.91 (s, 3H), 3.80 (s, 3H).

Ex-55F: 4-[2-(2,4-Dimethoxy-5-thien-2-yl-phenyl)-acetyl]-benzoic acid was prepared from 4-[2-(2,4-dimethoxy-5-thien-2-yl-phenyl)-acetyl]-benzoic acid methyl ester obtained from Ex-55E in a similar manner as described in Ex-49A to provide 53 mg (77%) of pure product. ¹H-NMR (CDCl₃) δ 8.16 (d, J=8.1 Hz, 2H), 8.08 (d, J=8.1 Hz, 2H), 7.39 (s, 1H), 7.33 (d, J=3.0 Hz, 1H), 7.21-7.25 (m, 1H), 7.00-7.03 (m, 1H), 6.52 (s, 1H), 4.24 (s, 2H), 3.91 (s, 3H), 3.81 (s, 3H).

Ex-55G: The title compound was prepared from 4-[2-(2,4-dimethoxy-5-thien-2-yl-phenyl)-acetyl]-benzoic acid obtained from Ex-55F in a similar manner as described in Ex-1H. The crude material was purified by crystallization from EtOAc and hexanes to provide 23 mg (45%) of pure product, mp 196-198° C. ¹H-NMR (DMSO-d₆) δ 7.97 (d, J=8.3 Hz, 2H), 7.80 (d, J=8.3 Hz, 2H), 7.70 (s, 1H), 7.53 (d, J=3.4 Hz, 1H), 7.45 (d, J=5.4 Hz, 1H), 7.05-7.08 (m, 1H), 6.65 (s, 1H), 6.07 (s, 1H), 5.64 (s, 1H), 3.88 (s, 3H), 3.50 (s, 3H). HRMS (EI⁺) m/z: calc. 394.0875, found 394.0885.

### Example 56

### 3-[2-(2-Methoxy-5-thien-2-yl-phenyl)-acryloyl]-benzoic acid

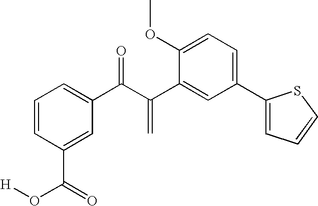

Ex-56A: In a 25 mL 2-neck round-bottom flask, 2-(3-bromomethyl-4-methoxy-phenyl)-thiophene obtained from Ex-7D (250 mg, 0.88 mmol), K₂CO₃ (370 mg, 2.68 mmol), 3-ethoxycarbonylphenylboronic acid (190 mg, 0.98 mmol) and bis(triphenylphospine) palladium (II) chloride (19 mg, 0.027 mmol) were combined and the round bottom was flushed with carbon monoxide for 5 min. Anisole (5 mL) was added and the resulting solution was heated to 80° C. under carbon monoxide. After 5 h the reaction was cooled to room temperature, diluted with MTBE and H₂O and the layers were cut. The organic layer was concentrated to dryness and the crude was purified by silica gel chromatography (15% EtOAc in hexanes) to afford 100 mg (30% yield) of desired 3-[2-(2-methoxy-5-thien-2-yl-phenyl)-acetyl]-benzoic acid ethyl ester. ¹H-NMR

MHz, CDCl₃): δ 8.72-8.74 (m, 1H), 8.21-8.25 (m, 2H), 7.56 (d, J=8.3 Hz, 1H), 7.44-7.52 (m, 2H), 7.17-7.21 (m, 2H), 7.01-7.04 (m, 1H), 6.90 (d, J=7.9 Hz, 1H), 4.42 (q, J=7.1 Hz, 2H), 4.34 (s, 2H), 3.82 (s, 3H). 1.41 (t, J=7.1 Hz, 3H).

Ex-56B: 3-[2-(2-Methoxy-5-thien-2-yl-phenyl)-acetyl]-benzoic acid was prepared from 3-[2-(2-methoxy-5-thien-2-yl-phenyl)-acetyl]-benzoic acid ethyl ester obtained from Ex-56A in a similar manner as described in Ex-49A to provide 55 mg (80%) of pure product. ¹H-NMR (DMSO-d₆) δ 13.21 (s, 1H), 8.51 (s, 1H), 8.25 (d, J=8.6 Hz, 1H), 8.15 (d, J=8.6 Hz, 1H), 7.65 (m, 1H), 7.47-7.55 (m, 2H), 7.41 (d, J=5.2 Hz, 1H), 7.31 (d, J=3.5 Hz, 1H), 7.05 (dd, J=5.2, 3.5 Hz, 1H), 6.99 (d, J=8.8 Hz, 1H), 4.38 (s, 2H), 3.69 (s, 3H).

Ex-56C: The title compound was prepared from 3-[2-(2-methoxy-5-thien-2-yl-phenyl)-acetyl]-benzoic acid obtained from Ex-56B in a similar manner as described in Ex-1H. The crude material was purified by crystallization from EtOAc and hexanes to provide 20 mg (39%) of pure product, mp 186-188° C. ¹H-NMR (DMSO-d₆) δ 13.21 (s, 1H), 8.31 (s, 1H), 8.11 (d, J=7.8 Hz, 1H), 7.97 (d, J=7.8 Hz, 1H), 7.66 (d, J=2.6 Hz, 1H), 7.57-7.62 (m, 2H), 7.44-7.47 (m, 2H), 7.07-7.11 (m, 1H), 6.97 (d, J=8.9 Hz, 1H), 6.17 (s, 1H), 5.73 (s, 1H), 3.47 (s, 3H). HRMS (EI⁺) m/z: calc. 364.0769, found 364.0763.

### Example 57

### 1-(4-tert-Butyl-phenyl)-2-(2-methoxy-5-thien-2-yl-phenyl)-propenone

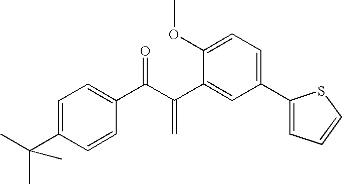

Ex-57A: 1-(4-tert-Butyl-phenyl)-2-(2-methoxy-5-thien-2-yl-phenyl)-ethanone was prepared from 2-(3-bromomethyl-4-methoxy-phenyl)-thiophene obtained from Ex-7D and 4-tert-butylphenyl boronic acid in a similar manner as described in Ex-56A. The crude material was purified by silica gel chrmotography with 10% EtOAc in hexanes to provide 115 mg (36%) of pure product. ¹H-NMR (CDCl₃) δ 8.00 (d, J=7.7 Hz, 2H), 7.47-7.51 (m, 3H), 7.42 (d, J=2.2 Hz, 1H), 7.17-7.21 (m, 2H), 7.03 (dd, J=5.1, 3.4 Hz, 1H), 6.90 (d, J=9.3 Hz, 1H), 4.29 (s, 2H), 3.82 (s, 3H), 1.35 (s, 9H).

Ex-57B: The title compound was prepared from 1-(4-tert-Butyl-phenyl)-2-(2-methoxy-5-thien-2-yl-phenyl)-ethanone obtained from Ex-57A in a similar manner as described in Ex-51E. The crude material was purified by crystallization from EtOAc and hexanes to provide 50 mg (50%) of pure product, mp 130-132° C. ¹H-NMR (DMSO-d₆) δ 7.02 (d, J=8.6 Hz, 2H), 7.56-7.62 (m, 2H), 7.44-7.50 (m, 4H), 7.07-7.10 (m, 1H), 6.97 (d, J=9.3 Hz, 1H), 6.09 (s, 1H), 5.65 (s, 1H), 3.49 (s, 3H), 1.26 (s, 9H). HRMS (EI⁺) m/z: calc. 376.1497, found 376.1495.

### Example 58

### 2-(2-Methoxy-5-thien-2-yl-phenyl)-1-(4-trifluoromethyl-phenyl)-propenone

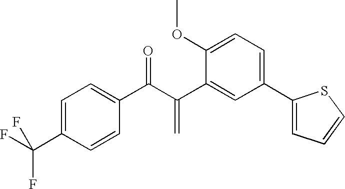

Ex-58A: 2-(2-Methoxy-5-thien-2-yl-phenyl)-1-(4-trifluoromethyl-phenyl)-ethanone was prepared from 2-(3-bromomethyl-4-methoxy-phenyl)-thiophene obtained from Ex-7D and 4-trifluoromethyl-phenyl boronic acid in a similar manner as described in Ex-56A. The crude material was purified by silica gel chrmotography with 10% EtOAc in hexanes to provide 168 mg (51%) of pure product. ¹H-NMR (CDCl₃) δ 8.15 (d, J=8.1 Hz, 2H), 7.73 (d, J=8.1 Hz, 2H), 7.51 (dd, J=7.9, 2.1 Hz, 1H), 7.42 (d, J=2.1 Hz, 1H), 7.18-7.22 (m, 2H), 7.02-7.05 (m, 1H), 6.90 (d, J=9.0 Hz, 1H), 4.31 (s, 2H), 3.81 (s, 3H).

Ex-58B: The title compound was prepared from 2-(2-methoxy-5-thien-2-yl-phenyl)-1-(4-trifluoromethyl-phenyl)-ethanone obtained from Ex-58A in a similar manner as described in Ex-51E. The crude material was purified by crystallization from EtOAc and hexanes to provide 90 mg (60%) of pure product, mp 131-133° C. ¹H-NMR (CDCl₃) δ 7.92 (d, J=8.1 Hz, 2H), 7.83 (d, J=8.1 Hz, 2H), 7.67 (d, J=2.3 Hz, 1H), 7.58 (dd, J=9.0, 2.3 Hz, 1H), 7.44-7.48 (m, 2H), 7.08 (dd, J=5.3, 3.6 Hz, 1H), 6.96 (d, J=9.0 Hz, 1H), 6.19 (s, 1H), 5.77 (s, 1H), 3.47 (s, 3H). HRMS (EI⁺) m/z: calc. 388.0745, found 388.0753.

### Example 59

### 4-[2-(2-Isopropoxy-5-thien-2-yl-phenyl)-acryloyl]-benzoic acid

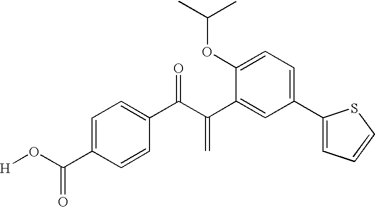

Ex-59A: 5-Bromo-2-hydroxy-benzaldehyde (10.0 g, 49.7 mmol) and thiophene-2-boronic acid (6.99 g, 54.63 mmol) were dissolved in THF (15 mL). Nitrogen was bubbled into the solution for 10 min followed by the sequential addition of KF (8.70 g, 149.7 mmol, spray-dried) and bis(tri-t-butylphosphine)palladium (0) (0.38 g, 0.74 mmol). The solution was immediately heated to 60° C. and aged for 30 min. The reaction was diluted with water and extracted with ethyl acetate. The organic layer was washed with 1 N HCl, dried over MgSO₄ and concentrated to dryness. Crystallization from THF and hexanes gave 7.9 g (78%) of 2-hydroxy-5-thien-2-yl-benzaldehyde. ¹H-NMR (CDCl₃) δ 11.00 (s, 1H), 9.95 (s, 1H), 7.75-7.79 (m, 2H), 7.24-7.29 (m, 2H), 7.09 (dd, J=5.5, 3.9 Hz, 1H), 7.03 (d, J=9.1 Hz, 1H).

Ex-59B: 2-Hydroxy-5-thien-2-yl-benzaldehyde obtained from Ex-59A (1.0 g, 4.9 mmol) was dissolved in isopropyl alcohol (10 mL) and potassium carbonate (2.2 g, 15.9 mmol) was added. Isopropyl iodide (500 μL, 5.0 mmol) was added and the solution was aged at 80° C. for 1.75 h. DMF (5 mL) was then added and the reaction was aged for 1.5 h. Additional isopropyl iodide (250 μL, 2.5 mmol) was added to the reaction mixture and the resulting solution was aged at 80° C. overnight. The crude reaction was diluted with EtOAc and H₂O and the layers were cut. The organic layer was washed with 1 N HCl and concentrated on the rotavap. The crude material was purified by silica gel chromatography (10% EtOAc in hexanes) to provide 1.14 g (94%) of 2-isopropoxy-5-thien-2-yl-benzaldehyde, mp 186-187° C. ¹H-NMR (CDCl₃) δ 10.50 (s, 1H), 8.05 (d, J=3.0 Hz, 1H), 7.75 (dd, J=8.7, 2.9 Hz, 1H), 7.23-7.26 (m, 2H), 7.05 (dd, J=5.0, 3.6 Hz, 1H), 7.00 (d, J=9.0 Hz, 1H), 4.63-4.75 (m, 1H), 1.24 (d, J=6.4 Hz, 6H).

Ex-59C: 4-[2-(2-Isopropoxy-5-thien-2-yl-phenyl)-acetyl]-benzoic acid methyl ester was prepared from 2-isopropoxy-5-thien-2-yl-benzaldehyde obtained from Ex-59B and 4-[(diphenoxy-phosphoryl)-phenylamino-methyl]-benzoic acid methyl ester obtained from Ex-2A in a similar manner as described in Ex-2C. The crude material was purified by silica gel chromatography (20% EtOAc in hexanes) to provide 477 mg (59%) of pure product. ¹H-NMR (CDCl₃) δ 8.11 (m, 4H), 7.45-7.48 (m, 2H), 7.17-7.21 (m, 2H), 7.01-7.05 (m, 11H), 6.86 (d, J=9.7 Hz, 1H), 4.52-4.60 (m, 1H), 4.27 (s, 2H), 3.95 (s, 3H), 1.24 (d, J=5.8 Hz, 6H).

Ex-59D: 4-[2-(2-Isopropoxy-5-thien-2-yl-phenyl)-acetyl]-benzoic acid was prepared from 4-[2-(2-isopropoxy-5-thien-2-yl-phenyl)-acetyl]-benzoic acid methyl ester obtained from Ex-59C in a similar manner as described in Ex-49A. The crude material was purified by slurrying in EtOAc and hexanes to provide 331 mg (76%) of pure product. ¹H-NMR (DMSO-d₆) δ 13.3 (bs, 1H), 8.09 (d, J=9.0 Hz, 2H), 8.03 (d, J=9.0 Hz, 2H), 7.44-7.49 (m, 2H), 7.41 (d, J=5.3 Hz, 1H), 7.31 (dd, J=5.1, 1.6 Hz, 1H), 7.05 (dd, J=5.2, 3.5 Hz, 1H), 6.96 (d, J=7.5 Hz, 1H), 4.49-4.57 (m, 1H), 4.28 (s, 2H), 1.05 (d, J=5.9 Hz, 6H).

Ex-59E: The title compound was prepared from 4-[2-(2-isopropoxy-5-thien-2-yl-phenyl)-acetyl]-benzoic acid obtained from Ex-59D in a similar manner as described in Ex-49B. The crude material was purified by crystallization from EtOAc and hexanes to provide 90 mg (60%) of product. ¹H-NMR (CDCl₃) δ 13.3 (bs, 1H), 8.05 (d, J=8.1 Hz, 2H), 7.96 (d, J=8.1 Hz, 2H), 7.71 (d, J=2.0 Hz, 1H), 7.60 (dd, J=9.1, 2.0 Hz, 1H), 7.48-7.51 (m, 2H), 7.08-7.15 (m, 1H), 7.00 (d, J=9.1 Hz, 1H), 6.17 (s, 1H), 5.69 (s, 1H), 4.49-4.57 (m, 1H), 0.85 (d, J=6.6 Hz, 6H). HRMS (EI⁺) m/z: calc. 392.1082, found 392.1084.

### Example 60

### 4-{2-[2-(2-Oxo-2-piperidin-1-yl-ethoxy)-5-thien-2-yl-phenyl]-acryloyl}-benzoic acid

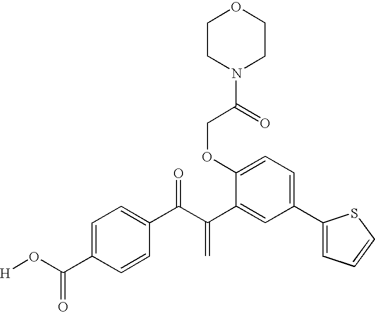

Ex-60A: 2-Hydroxy-5-thien-2-yl-benzaldehyde obtained from Ex-59A (1.0 g, 4.9 mmol) was dissolved in DMF (10 mL) and potassium carbonate (2.2 g, 15.9 mmol) was added. 2-Chloro-1-piperidin-1-yl-ethanone (885 mg, 5.41 mmol) was added and the solution was aged at 80° C. overnight. The crude reaction was diluted with EtOAc and H₂O and the layers were cut. The organic layer was washed with 1 N HCl, dried with Na₂SO₄ and concentrated on the rotavap. The crude material was purified by slurrying in EtOAc and hexanes to provide 1.38 g (85%) of 2-(2-oxo-2-piperidin-1-yl-ethoxy)-5-thien-2-yl-benzaldehyde. ¹H-NMR (DMSO-d₆) δ 10.42 (s, 1H), 7.89 (dd, J=8.2, 2.1 Hz, 1H), 7.83 (d, J=1.7 Hz, 1H), 7.50 (d, J=5.6 Hz, 1H), 7.45-7.46 (m, 1H), 7.19 (d, J=8.7 Hz, 1H), 7.09 (dd, J=5.6, 3.8 Hz, 1H), 5.10 (s, 2H), 3.52-3.59 (m, 4H), 3.43 (m, 4H).

Ex-60B: 4-{2-[2-(2-oxo-2-piperidin-1-yl-ethoxy)-5-thien-2-yl-phenyl]-acetyl}-benzoic acid methyl ester was prepared from 2-(2-oxo-2-piperidin-1-yl-ethoxy)-5-thien-2-yl-benzaldehyde obtained from Ex-60A and 4-[(diphenoxy-phosphoryl)-phenylamino-methyl]-benzoic acid methyl ester obtained from Ex-2A in a similar manner as described in Ex-2C. The crude material was purified by silica gel chromatography (80% EtOAc in hexanes) to provide 992 mg (68%) of pure product. ¹H-NMR (DMSO-d₆) δ 8.15 (d, J=7.9 Hz, 2H), 8.06 (d, J=7.9 Hz, 2H), 7.47-7.49 (m, 2H), 7.41 (d, J=4.7 Hz, 1H), 7.32 (d, J=3.1 Hz, 1H), 7.19-7.24 (m, 1H), 7.04-7.12 (m, 1H), 6.93-6.97 (m, 1H), 4.78 (s, 2H), 4.43 (s, 2H), 3.86 (s, 3H), 3.46 (m, 4H), 3.33-3.34 (m, 4H).

Ex-60C: 4-{2-[2-(2-Oxo-2-piperidin-1-yl-ethoxy)-5-thien-2-yl-phenyl]-acetyl}-benzoic acid was prepared from 4-{2-[2-(2-oxo-2-piperidin-1-yl-ethoxy)-5-thien-2-yl-phenyl]-acetyl}-benzoic acid methyl ester obtained from Ex-60B in a similar manner as described in Ex-49A. The crude material was purified by crystallization from EtOAc and hexanes to provide 426 mg (46%) of pure product. ¹H-NMR (DMSO-d₆) δ 13.29 (bs, 1H), 8.12 (d, J=8.2 Hz, 2H), 8.04 (d, J=8.2 Hz, 2H), 7.47-7.50 (d, 2H), 7.42 (d, J=5.2 Hz, 1H), 7.32 (d, J=3.4 Hz, 1H), 7.06 (dd, J=5.2, 3.4 Hz, 1H), 6.95 (d, J=9.6 Hz, 1H), 4.78 (s, 2H), 4.43 (s, 2H), 3.46 (m, 4H), 3.28-3.34 (m, 4H).

Ex-60D: The title compound was prepared from 4-{2-[2-(2-oxo 2-piperidin-1-yl-ethoxy)-5-thien-2-yl-phenyl]-acetyl}-benzoic acid obtained from Ex-60C in a similar manner as described in Ex-49B. The crude material was purified by crystallization from EtOAc and hexanes to provide 359 mg (96%) of product, mp 206-207° C. ¹H-NMR (DMSO-d₆) δ 13.23 (bs, 1H), 8.00 (d, J=8.6 Hz, 2H), 7.86 (d, J=8.6 Hz, 2H), 7.61 (d, J=2.2 Hz, 1H), 7.56 (dd, J=7.9, 2.1 Hz, 1H), 7.43-7.47 (m, 2H), 7.09 (dd, J=4.9, 3.6 Hz, 1H), 6.91 (d, J=8.5 Hz, 1H), 6.21 (s, 1H), 5.75 (s, 1H), 4.57 (s, 2H), 3.42 (m, 4H), 3.20-3.24 (m, 4H). HRMS (EI⁺) m/z: calc. 477.1246, found 477.1238.

### Example 61

### 4-{2-[2-(2-Piperidin-1-yl-ethoxy)-5-thien-2-yl-phenyl]-acryloyl}-benzoic acid; hydrochloride salt

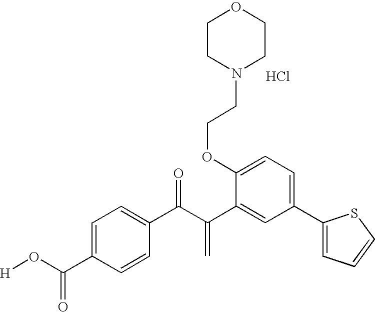

Ex-61A: 2-Hydroxy-5-thien-2-yl-benzaldehyde obtained from Ex-59A (1.0 g, 4.9 mmol) was dissolved in DMF (10 mL) and potassium carbonate (3.3 g, 23.9 mmol) was added. 1-(2-Chloro-ethyl)-piperidine hydrochloride salt (1.0 g, 5.37 mmol) was added and the solution was aged at 80° C. overnight. The crude reaction was diluted with EtOAc and H₂O and the layers were cut. The organic layer was washed with brine, dried with Na₂SO₄ and concentrated on the rotavap. The crude material was purified by slurrying with EtOAc and hexanes to provide 1.32 g (85%) of 2-(2-piperidin-1-yl-ethoxy)-5-thien-2-yl-benzaldehyde. ¹H-NMR (DMSO-d₆) δ 10.34 (s, 1H), 7.89 (dd, J=8.2, 2.8 Hz, 1H), 7.81 (d, J=1.9 Hz, 1H), 7.49 (d, J=5.2 Hz, 1H), 7.44 (d, J=3.6 Hz, 1H), 7.27 (d, J=8.9 Hz, 1H), 7.08 (dd, J=5.2, 3.6 Hz, 1H), 4.25 (t, J=5.2 Hz, 2H), 3.51 (t, J=4.4 Hz, 4H), 2.73 (t, J=5.2 Hz, 2H), 2.45 (t, J=4.4 Hz, 4H).

Ex-61B: 4-{2-[2-(2-Piperidin-1-yl-ethoxy)-5-thien-2-yl-phenyl]-acetyl}-benzoic acid methyl ester was prepared from 2-(2-piperidin-1-yl-ethoxy)-5-thien-2-yl-benzaldehyde obtained from Ex-61A and 4-[(diphenoxy-phosphoryl)-phenylamino-methyl]-benzoic acid methyl ester obtained from Ex-2A in a similar manner as described in Ex-2C. The crude material was purified by silica gel chromatography (10% MeOH in CH₂Cl₂) followed by crystallization from EtOAc and hexanes to provide 0.79 g (54%) of pure product. ¹H-NMR (DMSO-d₆) δ 8.13 (d, J=8.9 Hz, 2H), 8.06 (d, J=8.9 Hz, 2H), 7.47-7.51 (m, 2H), 7.41 (d, J=5.1 Hz, 1H), 7.32 (dd, J=3.7, 1.6 Hz, 1H), 7.06 (dd, J=4.0, 3.0 Hz, 1H), 6.99 (d, J=9.2 Hz, 1H), 4.37 (m, 2H), 4.00 (t, J=5.9 Hz, 2H), 3.86 (s, 3H), 3.33-3.36 (m, 4H), 2.46 (m, 2H), 2.20-2.23 (m, 4H).

Ex-61C: 4-{2-[2-(2-Piperidin-1-yl-ethoxy)-5-thien-2-yl-phenyl]-acetyl}-benzoic acid; hydrochloride salt was prepared from 4-{2-[2-(2-piperidin-1-yl-ethoxy)-5-thien-2-yl-phenyl]-acetyl}-benzoic acid methyl ester obtained from Ex-61B in a similar manner as described in Ex-49A. The crude material was purified by slurrying with EtOAc and hexanes to provide 590 mg (71%) of product. ¹H-NMR (DMSO-d₆) δ 13.36 (bs, 1H), 11.70 (bs, 1H), 8.19 (d, J=8.3 Hz, 2H), 8.10 (d, J=8.3 Hz, 2H), 7.46-7.60 (m, 3H), 7.38 (d, J=3.6 Hz, 1H), 7.07-7.12 (m, 2H), 4.55 (s, 2H), 4.45 (m, 2H), 3.76 (m, 4H), 3.30-3.41 (m, 4H), 3.01 (m, 2H).

Ex-61D: The title compound was prepared from 4-{2-[2-(2-piperidin-1-yl-ethoxy)-5-thien-2-yl-phenyl]-acetyl}-benzoic acid; hydrochloride salt obtained from Ex-61C in a similar manner as described in Ex-49B. DMF was added to the reaction to improve solubility. Standard workup afforded 23 mg (6%) of product. ¹H-NMR (DMSO-d₆) δ 13.30 (bs, 1H), 11.50 (bs, 1H), 8.02 (d, J=8.6 Hz, 2H), 7.90-7.93 (m, 2H), 7.67 (d, J=2.1 Hz, 1H), 7.62 (m, 1H), 7.47-7.49 (m, 2H), 7.05-7.12 (m, 2H), 6.26 (s, 1H), 5.84 (s, 1H), 4.28 (m, 2H), 3.64-3.76 (m, 4H), 3.32 (m, 2H), 3.17 (m, 4H). HRMS (EI⁺-Cl) m/z: calc. 464.1532, found 464.1537.

### Example 62

### 4-[2-(2-Methoxy-5-thien-3-yl-phenyl)-acryloyl]-benzoic acid

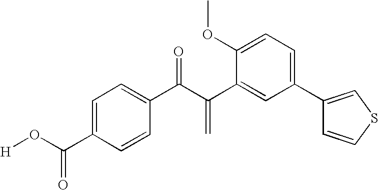

Ex-62A: 2-Methoxy-5-thien-3-yl-benzaldehyde was prepared from 5-bromo-2-methoxybenzaldehyde and thiophene 3-boronic acid in a similar manner as described in Ex-7B. The crude material was purified by silica gel chromatography (25% EtOAc in hexanes) followed by crystallization from EtOAc and hexanes to provide 1.8 g (74%) of product which was used without further purification

Ex-62B: 4-[2-(2-Methoxy-5-thien-3-yl-phenyl)-acetyl]-benzoic acid methyl ester was prepared from 2-methoxy-5-thien-3-yl-benzaldehyde obtained from Ex-62A and 4-[(diphenoxy-phosphoryl)-phenylamino-methyl]-benzoic acid methyl ester obtained from Ex-2A in a similar manner as described in Ex-2C. The crude material was purified by silica gel chromatography (10% EtOAc in hexanes) followed by slurrying with EtOAc and hexanes to provide 0.86 g (34%) of pure product. ¹H-NMR (DMSO-d₆) δ 8.12 (d, J=8.4 Hz, 2H), 8.5 (d, J=8.4 Hz, 2H), 7.65-7.66 (m, 1H), 7.55-7.59 (m, 3H), 7.43-7.45 (m, 1H), 6.97 (d, J=8.6 Hz, 1H), 4.36 (s, 2H), 3.86 (s, 3H), 3.67 (s, 3H).

Ex-62C: 4-[2-(2-Methoxy-5-thien-3-yl-phenyl)-acetyl]-benzoic acid was prepared from 4-[2-(2-methoxy-5-thien-3-yl-phenyl)-acetyl]-benzoic acid methyl ester obtained from Ex-62B in a similar manner as described in Ex-49A. The crude material was purified by slurrying with EtOAc and hexanes to provide 620 mg (89%) of pure product. ¹H-NMR (DMSO-d₆) δ 13.2 (bs, 1H), 8.10 (d, J=8.9 Hz, 2H), 8.04 (d, J=8.9 Hz, 2H), 7.66 (dd, J=2.7, 1.3 Hz, 1H), 7.53-7.58 (m, 3H), 7.45 (dd, J=5.0, 1.5 Hz, 1H), 6.98 (d, J=9.0 Hz, 1H), 4.35 (s, 2H), 3.68 (s, 3H).

Ex-62D: The title compound was prepared from 4-[2-(2-methoxy-5-thien-3-yl-phenyl)-acetyl]-benzoic acid obtained from Ex-62C in a similar manner as described in Ex-49B. The addition of DMF to improve solubility was necessary providing 23 mg (6%) of product, mp 192-194° C. ¹H-NMR (DMSO-d₆) δ 13.2 (bs, 1H), 7.96-8.00 (m, 2H), 7.81-7.83 (m, 3H), 7.76 (d, J=2.3 Hz, 1H), 7.64-7.67 (m, 1H), 7.55-7.62 (m, 2H), 6.93 (d, J=8.8 Hz, 1H), 6.17 (s, 1H), 5.73 (s, 1H), 3.45 (s, 3H). HRMS (EI⁺) m/z: calc. 364.0769, found 364.0758.

### Example 63

### ({4-[1-(2,6-Dimethoxy-3-thien-2-yl-benzoyl)-vinyl]-benzenesulfonyl}-methyl-amino)-acetic acid

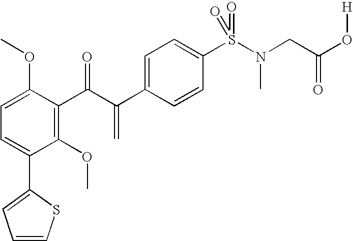

Ex-63A: 4-Bromomethyl-benzenesulfonyl chloride (2.5 g, 9.3 mmol), methylamino-acetic acid methyl ester hydrochloride salt (1.3 g, 9.3 mmol) was dissolved in Et₂O (10 mL) and THF (5 mL) and triethylamine (2.7 mL, 19.4 mmol) was added. After 1.5 h the reaction was diluted with EtOAc and 1 N HCl and the layers were cut. The organic layer was concentrated onto silica gel. The crude material was purified by silica gel chromatography (20% EtOAc in hexanes) to provide 0.91 g (29%) of [(4-bromomethyl-benzenesulfonyl)-methyl-amino]-acetic acid methyl ester. ¹H-NMR (CDCl₃) δ 7.78 (d, J=8.6 Hz, 2H), 7.53 (d, J=8.6 Hz, 2H), 4.50 (s, 2H), 4.03 (s, 2H), 3.64 (s, 3H), 2.91 (s, 3H).

Ex-63B: ({4-[2-(3-Bromo-2,6-dimethoxy-phenyl)-2-(tert-butyl-dimethyl-silanyloxy)-2-cyano-ethyl]-benzenesulfonyl}-methyl-amino)-acetic acid methyl ester was prepared from [(4-bromomethyl-benzenesulfonyl)-methyl-amino]-acetic acid methyl ester obtained from Ex-63A and (3-bromo-2,6-dimethoxy-phenyl)-(tert-butyl-dimethyl-silanyloxy)-acetonitrile obtained from Ex-30B in a similar manner as described in Ex-30C. The crude material was purified via silica gel chromatographpy (10% EtOAc in hexanes) to provide 740 mg (45%) of semi-product which was used without further purification.

Ex-63C: ({4-[2-(3-Bromo-2,6-dimethoxy-phenyl)-2-oxo-ethyl]-benzenesulfonyl}-methyl-amino)-acetic acid methyl ester was prepared from ({4-[2-(3-bromo-2,6-dimethoxy-phenyl)-2-(tert-butyl-dimethyl-silanyloxy)-2-cyano-ethyl]-benzenesulfonyl}-methyl-amino)-acetic acid methyl ester obtained from Ex-63B in a similar manner as described in Ex-7F. The crude material was purified by silica gel chromatography (10% EtOAc in hexanes) to provide 0.40 g (69%) of pure product. ¹H-NMR (CDCl₃) δ 7.76 (d, J=8.7 Hz, 2H), 7.50 (d, J=8.8 Hz, 1H), 7.38 (d, J=8.7 Hz, 2H), 6.61 (d, J=8.8 Hz, 1H), 4.13 (s, 2H), 3.98 (s, 2H), 3.80 (s, 3H), 3.78 (s, 3H), 3.65 (s, 3H), 2.89 (s, 3H).

Ex-63D: ({4-[2-(2,6-Dimethoxy-3-thien-2-yl-phenyl)-2-oxo-ethyl]-benzenesulfonyl}-methyl-amino)-acetic acid methyl ester was prepared from ({4-[2-(3-bromo-2,6-dimethoxy-phenyl)-2-oxo-ethyl]-benzenesulfonyl}-methyl-amino)-acetic acid methyl ester obtained from Ex-63C and thiophene 2-boronic acid in a similar manner as described in Ex-30E. The addition of 2 drops of H₂O was necessary to drive the reaction to completion. The crude material was purified by silica gel chromatography (15% EtOAc in hexanes) to provide 325 mg (81%) of pure product. ¹H-NMR (CDCl₃) δ 7.77 (d, J=8.4 Hz, 2H), 7.56 (d, J=8.3 Hz, 1H), 7.42 (d, J=8.4 Hz, 2H), 7.32-7.35 (m, 2H), 7.08 (dd, J=5.3, 3.5 Hz, 1H), 6.73 (d, J=8.6 Hz, 1H), 4.20 (s, 2H), 3.97 (s, 2H), 3.82 (s, 3H), 3.66 (s, 3H), 3.59 (s, 3H), 2.89 (s, 3H).

Ex-63E: ({4-[2-(2,6-Dimethoxy-3-thien-2-yl-phenyl)-2-oxo-ethyl]-benzenesulfonyl}-methyl-amino)-acetic acid was prepared from ({4-[2-(2,6-dimethoxy-3-thien-2-yl-phenyl)-2-oxo-ethyl]-benzenesulfonyl}-methyl-amino)-acetic acid methyl ester obtained from Ex-63D in a similar manner as described in Ex-49A to provide 270 mg (93%) of pure product. ¹H-NMR (CDCl₃) δ 7.77 (d, J=9.0 Hz, 2H), 7.56 (d, J=8.8 Hz, 1H), 7.42 (d, J=9.0 Hz, 2H), 7.33 (d, J=4.7 Hz, 2H), 7.07-7.10 (m, 1H), 6.74 (d, J=8.8 Hz, 1H), 4.20 (s, 2H), 3.99 (s, 2H), 3.82 (s, 3H), 3.59 (s, 3H), 2.89 (s, 3H).

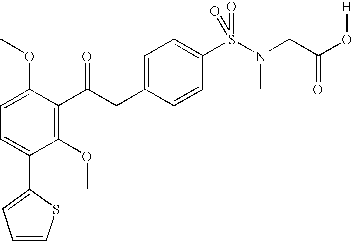

### ([4-[2-(2,6-Dimethoxy-3-thien-2-yl-phenyl)-2-oxo-ethyl]-benzenesulfonyl]-methyl-amino)-acetic acid

Ex-63F: The title compound was prepared from ({4-[2-(2,6-dimethoxy-3-thien-2-yl-phenyl)-2-oxo-ethyl]-benzenesulfonyl}-methyl-amino)-acetic acid obtained from Ex-63E in a similar manner as described in Ex-49B. Purification was accomplished by crystallization from hot EtOH to provide 120 mg (44%) of pure product, mp 164-166° C.

¹H-NMR (DMSO-d₆) δ 12.84 (bs, 1H), 7.80 (d, J=8.4 Hz, 2H), 7.72 (d, J=8.8 Hz, 1H), 7.61 (d, J=8.4 Hz, 2H), 7.54 (d, J=4.8 Hz, 1H), 7.45 (d, J=3.5 Hz, 1H), 7.10 (dd, J=4.8, 3.5 Hz, 1H), 6.99 (d, J=8.8 Hz, 1H), 6.45 (s, 1H), 5.97 (s, 1H), 3.88 (s, 2H), 3.76 (s, 3H), 3.51 (s, 3H), 2.78 (s, 3H). HRMS (EI⁺) m/z: calc. 501.0916, found 501.0902.

### Example 64

### 1-(2,6-Dimethoxy-3-thien-2-yl-phenyl)-2-(2-fluoro-6-trifluoromethyl-phenyl)-propenone

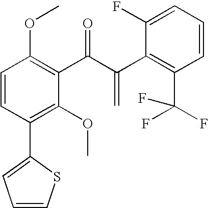

Ex-64A: 2-(3-bromo-2,6-dimethoxy-phenyl)-2-(tert-butyl-dimethyl-silanyloxy)-3-(2-fluoro-6-trifluoromethyl-phenyl)-propionitrile was prepared from (3-bromo-2,6-dimethoxy-phenyl)-(tert-butyl-dimethyl-silanyloxy)-acetonitrile obtained from Ex-30B and 2-bromomethyl-1-fluoro-3-trifluoromethyl-benzene in a similar manner as described in Ex-30C. Silica gel chromatography (5% EtOAc in hexanes) provided 650 mg (30%) of semi-pure product which was used without further purification.

Ex-64B: 1-(3-Bromo-2,6-dimethoxy-phenyl)-2-(2-fluoro-6-trifluoromethyl-phenyl)-ethanone was prepared from 2-(3-bromo-2,6-dimethoxy-phenyl)-2-(tert-butyl-dimethyl-silanyloxy)-3-(2-fluoro-6-trifluoromethyl-phenyl)-propionitrile obtained from Ex-64A in a similar manner as described in Ex-7F. The crude material was purified by silica gel chromatography (10% EtOAc in hexanes) to provide 280 g (58%) of pure product. ¹H-NMR (CDCl₃) δ 7.7.50-7.54 (m, 2H), 7.37-7.44 (m, 1H), 7.27-7.33 (m, 1H), 6.64 (d, J=8.9 Hz, 1H), 4.38 (s, 2H), 3.84 (s, 6H).

Ex-64C: 1-(2,6-Dimethoxy-3-thien-2-yl-phenyl)-2-(2-fluoro-6-trifluoromethyl-phenyl)-ethanone was prepared from 1-(3-bromo-2,6-dimethoxy-phenyl)-2-(2-fluoro-6-trifluoromethyl-phenyl)-ethanone obtained from Ex-64B and thiophene 2-boronic acid in a similar manner as described in Ex-30E. The addition of 2 drops of H₂O was necessary to help drive the reaction to completion. The crude material was purified by silica gel chromatography (5% EtOAc in hexanes) to provide 240 mg (95%) of pure product. 1H-NMR (CDCl₃) δ 7.58 (d, J=9.0 Hz, 1H), 7.51-7.54 (m, 1H), 7.40-7.44 (m, 2H), 7.30-7.37 (m, 2H), 7.09 (dd, J=5.4, 1.7 Hz, 1H), 6.77 (d, J=9.0 Hz, 1H), 4.45 (s, 2H), 3.87 (s, 3H), 3.64 (s, 3H).

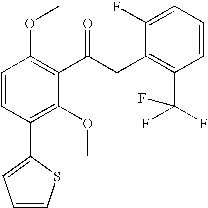

### 1-(2,6-Dimethoxy-3-thien-2-yl-phenyl)-2-(2-fluoro-6-trifluoromethyl-phenyl)-ethanone

Ex-64D: The title compound was prepared from 1-(2,6-dimethoxy-3-thien-2-yl-phenyl)-2-(2-fluoro-6-trifluoromethyl-phenyl)-ethanone obtained from Ex-64C in a similar manner as described in Ex-51E to provide 117 mg (53%) of pure product, mp 76-78° C. ¹H-NMR (CDCl₃) δ 7.61 (d, J=8.6 Hz, 1H), 7.55-7.57 (m, 1H), 7.44-7.51 (m, 1H), 7.40-7.41 (m, 1H), 7.28-7.33 (m, 2H), 7.09 (dd, J=5.2, 3.6 Hz, 1H), 6.78 (d, J=9.1 Hz, 1H), 6.35 (s, 1H), 6.28 (s, 1H), 3.83 (s, 3H), 3.63 (s, 3H). HRMS (EI⁺) m/z: calc. 437.0835, found 437.0849.

### Example 65

### 1-(2,6-Dimethoxy-phenyl)-2-(2-methoxy-5-thien-2-yl-phenyl)-propenone

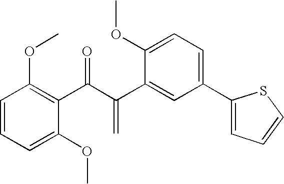

Ex-65A: 2-(tert-Butyl-dimethyl-silanyloxy)-2-(2,6-dimethoxy-phenyl)-3-(2-methoxy-5-thien-2-yl-phenyl)-propionitrile was prepared from 2-(3-bromomethyl-4-methoxy-phenyl)-thiophene and (tert-butyl-dimethyl-silanyloxy)-(2,6-dimethoxy-phenyl)-acetonitrile obtained from Ex-36A in a similar manner as described in Ex-30C. Tris(2-aminoethyl)amine polymer-bound resin was added to aid in the removal of the unreacted bromide prior to workup. The crude material was semi-purified by silica gel chromatography (10% EtOAc in hexanes) to provide 460 mg (41%) of semi-pure product which was used without further purification.

Ex-65B: 1-(2,6-Dimethoxy-phenyl)-2-(2-methoxy-5-thien-2-yl-phenyl)-ethanone was prepared from 2-(tert-butyl-dimethyl-silanyloxy)-2-(2,6-dimethoxy-phenyl)-3-(2-methoxy-5-thien-2-yl-phenyl)-propionitrile obtained from Ex-65A in a similar manner as described in Ex-7F. The crude material was purified by silica gel chromatography (10% EtOAc in hexanes) to provide 125 g (54%) of pure product. ¹H-NMR (CDCl₃) δ 7.41-7.45 (m, 2H), 7.14-7.24 (m, 3H), 7.02-7.05 (dd, J=4.9, 3.6 Hz, 1H), 6.79-6.82 (m, 1H), 6.49 (d, J=8.2 Hz, 2H), 4.10 (s, 2H), 3.75 (m, 9H).

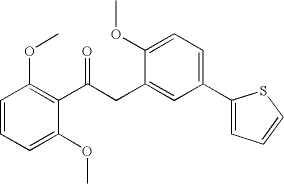

### 1-(2,6-Dimethoxy-phenyl)-2-(2-methoxy-5-thien-2-yl-phenyl)-ethanone

Ex-65C: The title compound was prepared from 1-(2,6-dimethoxy-phenyl)-2-(2-methoxy-5-thien-2-yl-phenyl)-ethanone obtained from Ex-65B in a similar manner as described in Ex-49B. The crude material was purified by silica gel chromatography (5% EtOAc in hexanes) to provide 30 mg (24%) of pure product, mp 162-163° C. ¹H-NMR (DMSO-d₆) δ 7.52 (dd, J=8.7, 2.7 Hz, 1H), 7.42 (d, J=4.6 Hz, 1H), 7.32-7.34 (m, 2H), 7.22-7.28 (m, 1H), 7.05-7.08 (m, 1H), 6.92 (d, J=8.1 Hz, 1H), 6.60 (d, J=9.0 Hz, 1H), 5.92 (d, J=11.9 Hz, 1H), 3.67 (s, 6H), 3.62 (s, 3H). HRMS (EI⁺) m/z: calc. 380.1082, found 380.1080.

### Example 66

### 2-{2-[1-(2,6-Dimethoxy-benzoyl)-vinyl]-4-thien-2-yl-phenoxy}-2-methyl-propionic acid

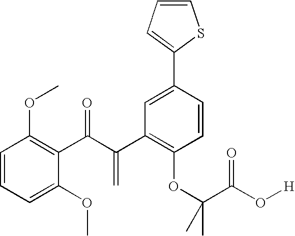

Ex-66A: 2-Hydroxy-5-thien-2-yl-benzaldehyde obtained from Ex-59A (2.0 g, 9.8 mmol) was dissolved in DMF (15 mL) and potassium carbonate (4.4 g, 31.8 mmol) was added. 2-Bromo-2-methyl-propionic acid ethyl ester (4.2 mL, 30.0 mmol) was added in 3 portions over 1.5 h at 80° C. and the resulting solution was aged overnight. The crude reaction was cooled to room temperature, diluted with EtOAc and H₂O and the layers were cut. The organic layer was washed with 1N HCl and then brine, dried with MgSO₄ and concentrated on the rotavap. The crude material was purified by silica gel chromtagraphy (20% EtOAc in hexanes) to provide 2.91 g (92%) of 2-(2-formyl-4-thien-2-yl-phenoxy)-2-methyl-propionic acid ethyl ester. ¹H-NMR (CDCl₃) δ 10.52 (s, 1H), 8.07 (d, J=2.5 Hz, 1H), 7.68 (dd, J=8.4, 2.5 Hz, 1H), 7.25-7.28 (m, 2H), 7.06 (dd, J=4.7, 3.4 Hz, 1H), 6.81 (d, J=9.0 Hz, 1H), 4.25 (q, J=6.9 Hz, 2H), 1.70 (s, 6H), 1.25 (t, J=6.9 Hz, 3H).

Ex-66B: 2-(2-Hydroxymethyl-4-thien-2-yl-phenoxy)-2-methyl-propionic acid ethyl ester was prepared from 2-(2-formyl-4-thien-2-yl-phenoxy)-2-methyl-propionic acid ethyl ester obtained from Ex-66A in a similar manner as described in Ex-1C. The crude product was used without further purification

Ex-66C: 2-(2-Bromomethyl-4-thien-2-yl-phenoxy)-2-methyl-propionic acid ethyl ester was prepared from 2-(2-hydroxymethyl-4-thien-2-yl-phenoxy)-2-methyl-propionic acid ethyl ester obtained from Ex-66B in a similar manner as described in Ex-1D. The crude material was purified by silica gel chromatography (10% EtOAc in hexanes) to provide 1.9 g (79%) of pure product. ¹H-NMR (CDCl₃) δ 7.57 (d, J=3.0 Hz, 1H), 7.42 (dd, J=8.1, 3.1 Hz, 1H), 7.20-7.24 (m, 2H), 7.05 (dd, J=4.9, 3.6 Hz, 1H), 6.68 (d, J=8.1 Hz, 1H), 4.59 (s, 2H), 4.27 (q, J=7.4 Hz, 2H), 1.70 (s, 6H), 1.27 (t, J=7.4 Hz, 3H).

Ex-66D: 2-{2-[2-(tert-Butyl-dimethyl-silanyloxy)-2-cyano-2-(2,6-dimethoxy-phenyl)-ethyl]-4-thien-2-yl-phenoxy}-2-methyl-propionic acid ethyl ester was prepared from 2-(2-bromomethyl-4-thien-2-yl-phenoxy)-2-methyl-propionic acid ethyl ester obtained from Ex-66C and (tert-butyl-dimethyl-silanyloxy)-(2,6-dimethoxy-phenyl)-acetonitrile obtained from Ex-36A in a similar manner as described in Ex-1E. The crude material was purified by silica gel chromatography (10% EtOAc in hexanes) to provide 0.95 g (52%) of semi-pure product. ¹H-NMR (CDCl₃) δ 7.29 (m, 5H), 6.96-6.99 (m, 2H), 6.49-6.58 (d, 2H), 4.21 (q, J=6.6 Hz, 2H), 3.88 (s, 1H), 3.75-3.78 (m, 1H), 3.69 (s, 6H), 1.66 (s, 3H), 1.65 (s, 3H), 1.42 (s, 6H), 1.22 (t, J=6.6 Hz, 3H), 0.90 (s, 9H), 0.17 (s, 3H), −0.02 (s, 3H).

Ex-66E: 2-{2-[2-(2,6-Dimethoxy-phenyl)-2-oxo-ethyl]-4-thien-2-yl-phenoxy}-2-methyl-propionic acid ethyl ester was prepared from 2-{2-[2-(tert-butyl-dimethyl-silanyloxy)-2-cyano-2-(2,6-dimethoxy-phenyl)-ethyl]-4-thien-2-yl-phenoxy}-2-methyl-propionic acid ethyl ester obtained from Ex-66D in a similar manner as described in Ex-1F. The crude material was purified by silica gel chromatography (15% EtOAc in hexanes) to provide 0.45 g (63%) of pure product. ¹H-NMR (DMSO-d₆) δ 7.39-7.44 (m, 2H), 7.29-7.33 (m, 3H), 7.06 (dd, J=5.2, 3.4 Hz, 1H), 6.66 (d, J=8.6 Hz, 2H), 6.59 (d, J=8.6 Hz, 1H), 4.14 (q, J=6.9 Hz, 2H), 3.99 (s, 2H), 3.69 (s, 6H), 1.42 (s, 6H), 1.14 (t, J=6.9 Hz, 3H).

Ex-66F: 2-{2-[2-(2,6-Dimethoxy-phenyl)-2-oxo-ethyl]-4-thien-2-yl-phenoxy}-2-methyl-propionic acid was prepared from 2-{2-[2-(2,6-dimethoxy-phenyl)-2-oxo-ethyl]-4-thien-2-yl-phenoxy}-2-methyl-propionic acid ethyl ester obtained from Ex-66E in a similar manner as described in Ex-35G. The crude material was purified by silica gel chromatography (15% EtOAc in hexanes) to provide 0.12 g (28%) of pure product. ¹H-NMR (CDCl₃) δ 7.37-7.42 (m, 2H), 7.26-7.31 (m, 1H), 7.21-7.23 (m, 1H), 7.16-7.17 (m, 1H), 7.04 (dd, J=5.3, 3.7 Hz, 1H), 6.87 (d, J=7.5 Hz, 1H), 6.56 (d, J=7.5 Hz, 2H), 4.17 (s, 2H), 3.78 (s, 6H), 1.67 (s, 6H).

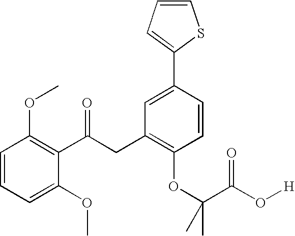

### 2-{2-[2-(2,6-Dimethoxy-phenyl)-2-oxo-ethyl]-4-thien-2-yl-phenoxy}-2-methyl-propionic acid

Ex-66G: The title compound was prepared from 2-{2-[2-(2,6-dimethoxy-phenyl)-2-oxo-ethyl]-4-thien-2-yl-phenoxy}-2-methyl-propionic acid obtained from Ex-66F in a similar manner as described in Ex-49B. The crude material was purified by silica gel chromatography (15% EtOAc in hexanes) to provide 52 mg (46%) of product as an amorphous solid, mp 72-84° C. ¹H-NMR (DMSO-d₆) δ 7.48-7.51 (m, 2H), 7.28-7.32 (m, 3H), 7.06-7.07 (m, 1H), 6.77 (d, J=7.5 Hz, 1H), 6.61-6.70 (m, 2H), 6.11 (s, 1H), 5.91 (s, 1H), 3.70 (s, 6H), 1.43 (s, 6H). HRMS (EI⁺) m/z: calc. 452.1294, found 452.1286.

Stereoisomerism and Polymorphism

It is appreciated that compounds of the present invention having a chiral center may exist in and be isolated in optically active and racemic forms. Some compounds may exhibit polymorphism. It is to be understood that the present invention encompasses any racemic, optically-active, diastereomeric, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase).

Examples of methods to obtain optically active materials are known in the art, and include at least the following.
i) physical separation of crystals—a technique whereby macroscopic crystals of the individual enantiomers are manually separated. This technique can be used if crystals of the separate enantiomers exist, i.e., the material is a conglomerate, and the crystals are visually distinct;
ii) simultaneous crystallization—a technique whereby the individual enantiomers are separately crystallized from a solution of the racemate, possible only if the latter is a conglomerate in the solid state;
iii) enzymatic resolutions—a technique whereby partial or complete separation of a racemate by virtue of differing rates of reaction for the enantiomers with an enzyme;
iv) enzymatic asymmetric synthesis—a synthetic technique whereby at least one step of the synthesis uses an enzymatic reaction to obtain an enantiomerically pure or enriched synthetic precursor of the desired enantiomer;
v) chemical asymmetric synthesis—a synthetic technique whereby the desired enantiomer is synthesized from an achiral precursor under conditions that produce asymmetry (i.e., chirality) in the product, which may be achieved using chiral catalysts or chiral auxiliaries;
vi) diastereomer separations—a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;
vii) first- and second-order asymmetric transformations—a technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer;
viii) kinetic resolutions—this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;
ix) enantiospecific synthesis from non-racemic precursors—a synthetic technique whereby the desired enantiomer is obtained from non-chiral starting materials and where the stereochemical integrity is not or is only minimally compromised over the course of the synthesis;
x) chiral liquid chromatography—a technique whereby the enantiomers of a racemate are separated in a liquid mobile phase by virtue of their differing interactions with a stationary phase. The stationary phase can be made of chiral material or the mobile phase can contain an additional chiral material to provoke the differing interactions;
xi) chiral gas chromatography—a technique whereby the racemate is volatilized and enantiomers are separated by virtue of their differing interactions in the gaseous mobile phase with a column containing a fixed non-racemic chiral adsorbent phase;
xii) extraction with chiral solvents—a technique whereby the enantiomers are separated by virtue of preferential dissolution of one enantiomer into a particular chiral solvent;
xiii) transport across chiral membranes—a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane which allows only one enantiomer of the racemate to pass through.
   Pharmaceutically Acceptable Salt Formulations

In cases where compounds are sufficiently basic or acidic to form stable nontoxic acid or base salts, administration of the compound as a pharmaceutically acceptable salt may be appropriate. The term “pharmaceutically acceptable salts” or “complexes” refers to salts or complexes that retain the desired biological activity of the compounds of the present invention and exhibit minimal undesired toxicological effects.

Examples of pharmaceutically acceptable salts are organic acid addition salts formed with acids, which form a physiological acceptable anion, for example, tosylate, methanesulfonate, acetate, citrate, malonate, tartarate, succinate, benzoate, ascorbate, α-ketoglutarate and α-glycerophosphate. Suitable inorganic salts may also be formed, including, sulfate, nitrate, bicarbonate and carbonate salts. Alternatively, the pharmaceutically acceptable salts may be made with sufficiently basic compounds such as an amine with a suitable acid affording a physiologically acceptable anion. Alkali metal (for example, sodium, potassium or lithium) or alkaline earth metal (for example calcium) salts of carboxylic acids can also be made.

Nonlimiting examples of such salts are (a) acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalcturonic acid; (b) base addition salts formed with metal cations such as zinc, calcium, bismuth, barium, magnesium, aluminum, copper, cobalt, nickel, cadmium, sodium, potassium, and the like, or with a cation formed from ammonia, N,N-dibenzylethylenediamine, D-glucosamine, tetraethylammonium, or ethylenediamine; or (c) combinations of (a) and (b); e.g., a zinc tannate salt or the like. Also included in this definition are pharmaceutically acceptable quaternary salts known by those skilled in the art, which specifically include the quaternary ammonium salt of the formula —NR⁺A⁻, wherein R is as defined above and A is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

Particular FDA-approved salts can be conveniently divided between anions and cations (Approved Drug Products with Therapeutic Equivalence Evaluations (1994) U.S. Department of Health and Human Services, Public Health Service, FDA, Center for Drug Evaluation and Research, Rockville, Md.; L. D. Bighley, S. M. Berge and D.C. Monkhouse, Salt Forms of Drugs and Absorption, Encyclopedia of Pharmaceutical Technology, Vol. 13, J. Swarbridk and J. Boylan, eds., Marcel Dekker, NY (1996)). Among the approved anions include aceglumate, acephyllinate, acetamidobenzoate, acetate, acetylasparaginate, acetylaspartate, adipate, aminosalicylate, anhydromethylenecitrate, ascorbate, aspartate, benzoate, besylate, bicarbonate, bisulfate, bitartrate, borate, bromide, camphorate, camsylate, carbonate, chloride, chlorophenoxyacetate, citrate, closylate, cromesilate, cyclamate, dehydrocholate, dihydrochloride, dimalonate, edentate, edisylate, estolate, esylate, ethylbromide, ethylsulfate, fendizoate, fosfatex, fumarate, gluceptate, gluconate, glucuronate, glutamate, glycerophosphate, glysinate, glycollylarsinilate, glycyrrhizate, hippurate, hemisulfate, hexylresorcinate, hybenzate, hydrobromide, hydrochloride, hydroiodid, hydroxybenzenesulfonate, hydroxybenzoate, hydroxynaphthoate, hyclate, iodide, isethionate, lactate, lactobionate, lysine, malate, maleate, mesylate, methylbromide, methyliodide, methylnitrate, methylsulfate, monophosadenine, mucate, napadisylate, napsylate, nicotinate, nitrate, oleate, orotate, oxalate, oxoglurate, pamoate, pantothenate, pectinate, phenylethylbarbiturate, phosphate, pacrate, plicrilix, polistirex, polygalacturonate, propionate, pyridoxylphosphate, saccharinate, salicylate, stearate, succinate, stearylsulfate, subacetate, succinate, sulfate, sulfosalicylate, tannate, tartrate, teprosilate, terephthalate, teoclate, thiocyante, tidiacicate, timonacicate, tosylate, triethiodide, triethiodide, undecanoate, and xinafoate. The approved cations include ammonium, benethamine, benzathine, betaine, calcium, camitine, clemizole, chlorcyclizine, choline, dibenylamine, diethanolamine, diethylamine, diethylammonium diolamine, eglumine, erbumine, ethylenediamine, heptaminol, hydrabamine, hydroxyethylpyrrolidone, imadazole, meglumine, olamine, piperazine, 4-phenylcyclohexylamine, procaine, pyridoxine, triethanolamine, and tromethamine. Metallic cations include, aluminum, bismuth, calcium lithium, magnesium, neodymium, potassium, rubidium, sodium, strontium and zinc.

A particular class of salts can be classified as organic amine salts. The organic amines used to form these salts can be primary amines, secondary amines or tertiary amines, and the substituents on the amine can be straight, branched or cyclic groups, including ringed structures formed by attachment of two or more of the amine substituents. Of particular interest are organic amines that are substituted by one or more hydroxyalkyl groups, including alditol or carbohydrate moieties. These hydroxy substituted organic amines can be cyclic or acyclic, both classes of which can be primary amines, secondary amines or tertiary amines. A common class of cyclic hydroxy substituted amines are the amino sugars.

Carbohydrate moieties that can comprise one or more substituents in the amine salt include those made from substituted and unsubstituted monosaccharides, disaccharides, oligosaccharides, and polysaccharides. The saccharide can be an aldose or ketose, and may comprise 3, 4, 5, 6, or 7 carbons. In one embodiment the carbohydrates are monosaccharides. In another embodiment the carbohydrates are pyranose and furanose sugars. Non limiting examples of pyranose and furanose moieties that can be part of the organic amine salt include threose, ribulose, ketose, gentiobiose, aldose, aldotetrose, aldopentose, aldohexose, ketohexose, ketotetrose, ketopentose, erythrose, threose, ribose, deoxyribose, arabinose, xylose, lyxose, allose, altrose, glucose, mannose, gulose, idose, glactose, talose, erythrulose, ribulose, xylulose, psicose, fructose, sorbose, tagatose, dextrose, maltose, lactose, sucrose, cellulose, aldose, amylose, palatinose, trehalose, turanose, cellobiose, amylopectin, glucosamine, mannosamine, fucose, phamnose, glucuronate, gluconate, glucono-lactone, muramic acid, abequose, rhamnose, gluconic acid, glucuronic acid, and galactosamine. The carbohydrate moiety can optionally be deoxygenated at any corresponding C-position, and/or substituted with one or more moieties such as hydrogen, halo, haloalkyl, carboxyl, acyl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, thioester, thioether, oxime, hydrazine, carbamate, phosphonic acid, phosphonate, or any other viable functional group that does not inhibit the pharmacological activity of this compound. Exemplary substituents include amine and halo, particularly fluorine. The substituent or carbohydrate can be either unprotected, or protected as necessary, as known to those skilled in the art, for example, as taught in Greene, et al., Protective Groups in Organic Synthesis, John Wiley and Sons, Second Edition, 1991, hereby incorporated by reference. In one embodiment the monosaccharide is a furanose such as (L or D)-ribose.

Of particular interest among the acyclic organic amines are a class represented by the formula 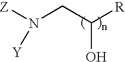
wherein Y and Z are independently hydrogen or lower alkyl or, may be taken together to form a ring, R is hydrogen, alkyl or hydroxyloweralkyl, and n is 1, 2, 3, 4, or 5. Among these hydroxylamines are a particular class characterized when n is 4. A representative of this group is meglumine, represented when Y is hydrogen, Z is methyl and R is methoxy. Meglumine is also known in the art as N-methylglucamine, N-MG, and 1-deoxy-1-(methylamino)-D-glucitol.

The invention also includes pharmaceutically acceptable prodrugs of the compounds. Pharmaceutically acceptable prodrugs refer to a compound that is metabolized, for example hydrolyzed or oxidized, in the host to form the compound of the present invention. Typical examples of prodrugs include compounds that have biologically labile protecting groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, dephosphorylated to produce the active compound.

Any of the compounds described herein can be administered as a prodrug to increase the activity, bioavailability, stability or otherwise alter the properties of the compound. A number of prodrug ligands are known. In general, alkylation, acylation or other lipophilic modification of the compound will increase the stability of the chalcone. Examples of substituent groups that can replace one or more hydrogens on the compound are alkyl, aryl, steroids, carbohydrates, including sugars, 1,2-diacylglycerol and alcohols. Many are described in R. Jones and N. Bischofberger, Antiviral Research, 27 (1995) 1-17. Any of these can be used in combination with the disclosed compounds to achieve a desired effect.

The compounds can be used to treat inflammatory disorders that involve VCAM-1 including, but not limited to arthritis, asthma, dermatitis, psoriasis, cystic fibrosis, post transplantation late and chronic solid organ rejection, multiple sclerosis, systemic lupus erythematosis, inflammatory bowel diseases, autoimmune diabetes, diabetic retinopathy, diabetic nephropathy, diabetic vasculopathy, rhinitis, ischemia-reperfusion injury, post-angioplasty restenosis, chronic obstructive pulmonary disease (COPD), glomerulonephritis, Graves disease, gastrointestinal allergies, conjunctivitis, atherosclerosis, coronary artery disease, angina and small artery disease.

The compounds disclosed herein can be used in the treatment of inflammatory skin diseases that involve VCAM-1, and in particular, human endothelial disorders that involve VCAM-1, which include, but are not limited to, psoriasis, dermatitis, including eczematous dermatitis, and Kaposi's sarcoma, as well as proliferative disorders of smooth muscle cells.

In yet another embodiment, the compounds disclosed herein can be selected to treat anti-inflammatory conditions that are mediated by mononuclear leucocytes.

In yet another embodiment, the compounds of the present invention can be selected for the prevention or treatment of tissue or organ transplant rejection. Treatment and prevention of organ or tissue transplant rejection includes, but are not limited to treatment of recipients of heart, lung, combined heart-lung, liver, kidney, pancreatic, skin, spleen, small bowel, or corneal transplants. They are also indicated for the prevention or treatment of graft-versus-host disease, which sometimes occurs following bone marrow transplantation.

In an alternative embodiment, the compounds described herein are useful in both the primary and adjunctive medical treatment of cardiovascular disease. The compounds are used in primary treatment of, for example, coronary disease states including atherosclerosis, post-angioplasty restenosis, coronary artery diseases and angina. The compounds can be administered to treat small vessel disease that is not treatable by surgery or angioplasty, or other vessel disease in which surgery is not an option. The compounds can also be used to stabilize patients prior to revascularization therapy.

In another aspect the invention provides pharmaceutical compositions for the treatment of diseases or disorders involving VCAM-1 wherein such compositions comprise a VCAM-1 inhibiting amount of a chalcone derivatives of the invention or a pharmaceutically acceptable salt thereof and/or a pharmaceutically acceptable carrier.

In another aspect the invention provides a method for treating a disease or disorder involving VCAM-1 comprising administering to a patient a VCAM-1 inhibiting effective amount of a compound of the invention or a pharmaceutically acceptable salt thereof.

In another aspect the invention provides a method for treating cardiovascular and inflammatory disorders in a patient in need thereof comprising administering to said patient an VCAM-1 inhibiting effective amount of a compound of the invention or a pharmaceutically acceptable salt thereof.

In another aspect the invention provides a method and composition for treating asthma or arthritis in a patient in need thereof comprising administering to said patient an effective amount of a compound of the invention or a pharmaceutically acceptable salt thereof.

The compounds of the present invention can be used to treat any disorder that involves VCAM-1. VCAM-1 is upregulated in a wide variety of disease states, including but not limited to arthritis, asthma, dermatitis, psoriasis, cystic fibrosis, post transplantation late and chronic solid organ rejection, multiple sclerosis, systemic lupus erythematosis, inflammatory bowel diseases, autoimmune diabetes, diabetic retinopathy, diabetic nephropathy, diabetic vasculopathy, rhinitis, ischemia-reperfusion injury, post-angioplasty restenosis, chronic obstructive pulmonary disease (COPD), glomerulonephritis, Graves disease, gastrointestinal allergies, atherosclerosis, coronary artery disease, angina, small artery disease, and conjunctivitis.

Nonlimiting examples of arthritis include rheumatoid (such as soft-tissue rheumatism and non-articular rheumatism, fibromyalgia, fibrositis, muscular rheumatism, myofascil pain, humeral epicondylitis, frozen shoulder, Tietze's syndrome, fascitis, tendinitis, tenosynovitis, bursitis), juvenile chronic, spondyloarthropaties (ankylosing spondylitis), osteoarthritis, hyperuricemia and arthritis associated with acute gout, chronic gout and systemic lupus erythematosus.

Human endothelial disorders involving VCAM-1 include psoriasis, eczematous dermatitis, Kaposi's sarcoma, as well as proliferative disorders of smooth muscle cells.

In yet another embodiment, the compounds disclosed herein can be selected to treat anti-inflammatory conditions that are mediated by mononuclear leucocytes.

In one embodiment, the compounds of the present invention are selected for the prevention or treatment of tissue or organ transplant rejection. Treatment and prevention of organ or tissue transplant rejection includes, but are not limited to treatment of recipients of heart, lung, combined heart-lung, liver, kidney, pancreatic, skin, spleen, small bowel, or corneal transplants. The compounds can also be used in the prevention or treatment of graft-versus-host disease, such as sometimes occurs following bone marrow transplantation.

In an alternative embodiment, the compounds described herein are useful in both the primary and adjunctive medical treatment of cardiovascular disease. The compounds are used in primary treatment of, for example, coronary disease states including atherosclerosis, post-angioplasty restenosis, coronary artery diseases and angina. The compounds can be administered to treat small vessel disease that is not treatable by surgery or angioplasty, or other vessel disease in which surgery is not an option. The compounds can also be used to stabilize patients prior to revascularization therapy.

Combination and Alternation Therapy

Any of the compounds disclosed herein can be administered in combination or alternation with a second biologically active agent to increase its effectiveness against the target disorder.

In combination therapy, effective dosages of two or more agents are administered together, whereas during alternation therapy an effective dosage of each agent is administered serially. The dosages will depend on absorption, inactivation and excretion rates of the drug as well as other factors known to those of skill in the art. It is to be noted that dosage values will also vary with the severity of the condition to be alleviated. It is to be further understood that for any particular subject, specific dosage regimens and schedules should be adjusted over time according to the individual need and the professional judgment of the person administering or supervising the administration of the compositions.

The efficacy of a drug can be prolonged, augmented, or restored by administering the compound in combination or alternation with a second, and perhaps third, agent that induces a different biological pathway from that caused by the principle drug. Alternatively, the pharmacokinetics, biodistribution or other parameter of the drug can be altered by such combination or alternation therapy. In general, combination therapy is typically preferred over alternation therapy because it induces multiple simultaneous stresses on the condition.

Any method of alternation can be used that provides treatment to the patient. Nonlimiting examples of alternation patterns include 1-6 weeks of administration of an effective amount of one agent followed by 1-6 weeks of administration of an effective amount of a second agent. The alternation schedule can include periods of no treatment. Combination therapy generally includes the simultaneous administration of an effective ratio of dosages of two or more active agents.

Illustrative examples of specific agents that can be used in combination or alternation with the compounds of the present invention are described below in regard to asthma and arthritis. The agents set out below or others can alternatively be used to treat a host suffering from any of the other disorders listed above or that involve VCAM-1. Illustrative second biologically active agents for the treatment of cardiovascular disease are also provided below.

Asthma

In one embodiment, the compounds of the present invention are administered in combination or alternation with heparin, frusemide, ranitidine, an agent that effects respiratory function, such as DNAase, or immunosuppressive agents, IV gamma globulin, troleandomycin, cyclosporin (Neoral), methotrexate, FK-506, gold compounds such as Myochrysine (gold sodium thiomalate), platelet activating factor (PAF) antagonists such as thromboxane inhibitors, leukotriene-D₄-receptor antagonists such as Accolate (zafirlukast), Ziflo (zileuton), leukotriene C₁ or C₂ antagonists and inhibitors of leukotriene synthesis such as zileuton for the treatment of asthma, or an inducible nitric oxide synthase inhibitor.

In another embodiment, the active compound is administered in combination or alternation with one or more other prophylactic agent(s). Examples of prophylactic agents that can be used in alternation or combination therapy include but are not limited to sodium cromoglycate, Intal (cromolyn sodium, Nasalcrom, Opticrom, Crolom, Ophthalmic Crolom), Tilade (nedocromil, nedocromil sodium) and ketotifen.

In another embodiment, the active compound is administered in combination or alternation with one or more other β₂-adrenergic agonist(s) (β agonists). Examples of β₂-adrenergicagonists (β agonists) that can be used in alternation or combination therapy include but are not limited to albuterol (salbutamol, Proventil, Ventolin), terbutaline, Maxair (pirbuterol), Serevent (salmeterol), epinephrine, metaproterenol (Alupent, Metaprel), Brethine (Bricanyl, Brethaire, terbutaline sulfate), Tornalate (bitolterol), isoprenaline, ipratropium bromide, bambuterol hydrochloride, bitolterol meslyate, broxaterol, carbuterol hydrochloride, clenbuterol hydrochloride, clorprenaline hydrochloride, efirmoterol fumarate, ephedra (source of alkaloids), ephedrine (ephedrine hydrochloride, ephedrine sulfate), etafedrine hydrochloride, ethylnoradrenaline hydrochloride, fenoterol hydrochloride, hexoprenaline hydrochloride, isoetharine hydrochloride, isoprenaline, mabuterol, methoxyphenamine hydrochloride, methylephedrine hydrochloride, orciprenaline sulphate, phenylephrine acid tartrate, phenylpropanolamine (phenylpropanolamine polistirex, phenylpropanolamine sulphate), pirbuterol acetate, procaterol hydrochloride, protokylol hydrochloride, psuedoephedrine (psuedoephedrine polixtirex, psuedoephedrine tannate, psuedoephedrine hydrochloride, psuedoephedrine sulphate), reproterol hydrochloride, rimiterol hydrobromide, ritodrine hydrochloride, salmeterol xinafoate, terbutaline sulphate, tretoquinol hydrate and tulobuterol hydrochloride.

In another embodiment, the active compound is administered in combination or alternation with one or more other corticosteriod(s). Examples of corticosteriods that can be used in alternation or combination therapy include but are not limited to glucocorticoids (GC), Aerobid (Aerobid-M, flunisolide), Azmacort (triamcinolone acetonide), Beclovet (Vanceril, beclomethasone dipropionate), Flovent (fluticasone), Pulmicort (budesonide), prednisolone, hydrocortisone, adrenaline, Alclometasone Dipropionate, Aldosterone, Amcinonide, Beclomethasone Dipropionate, Bendacort, Betamethasone (Betamethasone Acetate, Betamethasone Benzoate, Betamethasone Dipropionate, Betamethasone Sodium Phosphate, Betamethasone Valerate), Budesonide, Ciclomethasone, Ciprocinonide, Clobetasol Propionate, Clobetasone Butyrate, Clocortolone Pivalate, Cloprednol, Cortisone Acetate, Cortivazol, Deflazacort, Deoxycortone Acetate (Deoxycortone Pivalate), Deprodone, Desonide, Desoxymethasone, Dexamethasone (Dexamethasone Acetate, Dexamethasone Isonicotinate, Dexamethasone Phosphate, Dexamethasone Sodium Metasulphobenzoate, Dexamethasone Sodium Phosphate), Dichlorisone Acetate, Diflorasone Diacetate, Diflucortolone Valerate, Difluprednate, Domoprednate, Endrysone, Fluazacort, Fluclorolone Acetonide, Fludrocortisone Acetate, Flumethasone (Flumethasone Pivalate), Flunisolide, Fluocinolone Acetonide, Fluocinonide, Fluocortin Butyl, Fluocortolone (Fluocortolone Hexanoate, Fluocortolone Pivalate), Fluorometholone (Fluorometholone Acetate), Fluprednidene Acetate, Fluprednisolone, Flurandrenolone, Fluticasone Propionate, Formocortal, Halcinonide, Halobetasol Propionate, Halometasone, Hydrocortamate Hydrochloride, Hydrocortisone (Hydrocortisone Acetate, Hydrocortisone Butyrate, Hydrocortisone Cypionate, Hydrocortisone Hemisuccinate, Hydrocortisone Sodium Phosphate, Hydrocortisone Sodium Succinate, Hydrocortisone Valerate), Medrysone, Meprednisone, Methylprednisolone (Methylprednisolone Acetate, Methylprednisolone, Hemisuccinate, Methylprednisolone Sodium Succinate), Mometasone Furoate, Paramethasone Acetate, Prednicarbate, Prednisolamate Hydrochloride, Prednisolone (Prednisolone Acetate, Prednisolone Hemisuccinate, Prednisolone Hexanoate, Prednisolone Pivalate, Prednisolone Sodium Metasulphobenzoate, Prednisolone Sodium Phosphate, Prednisolone Sodium Succinate, Prednisolone Steaglate, Prednisolone Tebutate), Prednisone (Prednisone Acetate), Prednylidene, Procinonide, Rimexolone, Suprarenal Cortex, Tixocortol Pivalate, Triamcinolone (Triamcinolone Acetonide, Triamcinolone Diacetate and Triamcinolone Hexacetonide).

In another embodiment, the active compound is administered in combination or alternation with one or more other antihistimine(s) (H₁ receptor antagonists). Examples of antihistimines (H₁ receptor antagonists) that can be used in alternation or combination therapy include alkylamines, ethanolamines ethylenediamines, piperazines, piperidines or phenothiazines. Some non-limiting examples of antihistimes are Chlortrimeton (Teldrin, chlorpheniramine), Atrohist (brompheniramine, Bromarest, Bromfed, Dimetane), Actidil (triprolidine), Dexchlor (Poladex, Polaramine, dexchlorpheniramine), Benadryl (diphen-hydramine), Tavist (clemastine), Dimetabs (dimenhydrinate, Dramamine, Marmine), PBZ (tripelennamine), pyrilamine, Marezine (cyclizine), Zyrtec (cetirizine), hydroxyzine, Antivert (meclizine, Bonine), Allegra (fexofenadine), Hismanal (astemizole), Claritin (loratadine), Seldane (terfenadine), Periactin (cyproheptadine), Nolamine (phenindamine, Nolahist), Phenameth (promethazine, Phenergan), Tacaryl (methdilazine) and Temaril (trimeprazine).

Alternatively, the compound of the present invention is administered in combination or alternation with
(a) xanthines and methylxanthines, such as Theo-24 (theophylline, Slo-Phylline, Uniphyllin, Slobid, Theo-Dur), Choledyl (oxitriphylline), aminophylline;
(b) anticholinergic agents (antimuscarinic agents) such as belladonna alkaloids, Atrovent (ipratropium bromide), atropine, oxitropium bromide;
(c) phosphodiesterase inhibitors such as zardaverine;
(d) calcium antagonists such as nifedipine; or
(e) potassium activators such as cromakalim for the treatment of asthma.
   Arthritic Disorders

In one embodiment, the compound of the present invention can also be administered in combination or alternation with apazone, amitriptyline, chymopapain, collegenase, cyclobenzaprine, diazepam, fluoxetine, pyridoxine, ademetionine, diacerein, glucosamine, hylan (hyaluronate), misoprostol, paracetamol, superoxide dismutase mimics, TNFα receptor antagonists, TNFα antibodies, P38 Kinase inhibitors, tricyclic antidepressents, cJun kinase inhibitors or immunosuppressive agents, IV gamma globulin, troleandomycin, cyclosporin (Neoral), methotrexate, FK-506, gold compounds such as Myochrysine (gold sodium thiomalate), platelet activating factor (PAF) antagonists such as thromboxane inhibitors, and inducible nitric oxide sythase inhibitors.

In another embodiment, the active compound is administered in combination or alternation with one or more other corticosteriod(s). Examples of corticosteriods that can be used in alternation or combination therapy include but are not limited to glucocorticoids (GC), Aerobid (Aerobid-M, flunisolide), Azmacort (triamcinolone acetonide), Beclovet (Vanceril, beclomethasone dipropionate), Flovent (fluticasone), Pulmicort (budesonide), prednisolone, hydrocortisone, adrenaline, Alclometasone Dipropionate, Aldosterone, Amcinonide, Beclomethasone Dipropionate, Bendacort, Betamethasone (Betamethasone Acetate, Betamethasone Benzoate, Betamethasone Dipropionate, Betamethasone Sodium Phosphate, Betamethasone Valerate), Budesonide, Ciclomethasone, Ciprocinonide, Clobetasol Propionate, Clobetasone Butyrate, Clocortolone Pivalate, Cloprednol, Cortisone Acetate, Cortivazol, Deflazacort, Deoxycortone Acetate (Deoxycortone Pivalate), Deprodone, Desonide, Desoxymethasone, Dexamethasone (Dexamethasone Acetate, Dexamethasone Isonicotinate, Dexamethasone Phosphate, Dexamethasone Sodium Metasulphobenzoate, Dexamethasone Sodium Phosphate), Dichlorisone Acetate, Diflorasone Diacetate, Diflucortolone Valerate, Difluprednate, Domoprednate, Endrysone, Fluazacort, Fluclorolone Acetonide, Fludrocortisone Acetate, Flumethasone (Flumethasone Pivalate), Flunisolide, Fluocinolone Acetonide, Fluocinonide, Fluocortin Butyl, Fluocortolone (Fluocortolone Hexanoate, Fluocortolone Pivalate), Fluorometholone (Fluorometholone Acetate), Fluprednidene Acetate, Fluprednisolone, Flurandrenolone, Fluticasone Propionate, Formocortal, Halcinonide, Halobetasol Propionate, Halometasone, Hydrocortamate Hydrochloride, Hydrocortisone (Hydrocortisone Acetate, Hydrocortisone Butyrate, Hydrocortisone Cypionate, Hydrocortisone Hemisuccinate, Hydrocortisone Sodium Phosphate, Hydrocortisone Sodium Succinate, Hydrocortisone Valerate), Medrysone, Meprednisone, Methylprednisolone (Methylprednisolone Acetate, Methylprednisolone, Hemisuccinate, Methylprednisolone Sodium Succinate), Mometasone Furoate, Paramethasone Acetate, Prednicarbate, Prednisolamate Hydrochloride, Prednisolone (Prednisolone Acetate, Prednisolone Hemisuccinate, Prednisolone Hexanoate, Prednisolone Pivalate, Prednisolone Sodium Metasulphobenzoate, Prednisolone Sodium Phosphate, Prednisolone Sodium Succinate, Prednisolone Steaglate, Prednisolone Tebutate), Prednisone (Prednisone Acetate), Prednylidene, Procinonide, Rimexolone, Suprarenal Cortex, Tixocortol Pivalate, Triamcinolone (Triamcinolone Acetonide, Triamcinolone Diacetate and Triamcinolone Hexacetonide).

In another embodiment, the active compound is administered in combination or alternation with one or more other non-steroidal anti-inflammatory drug(s) (NSAIDS). Examples of NSAIDS that can be used in alternation or combination therapy are carboxylic acids, propionic acids, fenamates, acetic acids, pyrazolones, oxicans, alkanones, gold compounds and others that inhibit prostaglandin synthesis, preferably by selectively inhibiting cylcooxygenase-2 (COX-2). Some nonlimiting examples of COX-2 inhibitors are Celebrex (celecoxib), Bextra (valdecoxib), Dynastat (parecoxib sodium) and Vioxx (rofacoxib). Some non-limiting examples of NSAIDS are aspirin (acetylsalicylic acid), Dolobid (diflunisal), Disalcid (salsalate, salicylsalicylate), Trisilate (choline magnesium trisalicylate), sodium salicylate, Cuprimine (penicillamine), Tolectin (tolmetin), ibuprofen (Motrin, Advil, Nuprin Rufen), Naprosyn (naproxen, Anaprox, naproxen sodium), Nalfon (fenoprofen), Orudis (ketoprofen), Ansaid (flurbiprofen), Daypro (oxaprozin), meclofenamate (meclofanamic acid, Meclomen), mefenamic acid, Indocin (indomethacin), Clinoril (sulindac), tolmetin, Voltaren (diclofenac), Lodine (etodolac), ketorolac, Butazolidin (phenylbutazone), Tandearil (oxyphenbutazone), piroxicam (Feldene), Relafen (nabumetone), Myochrysine (gold sodium thiomalate), Ridaura (auranofin), Solganal (aurothioglucose), acetaminophen, colchicine, Zyloprim (allopurinol), Benemid (probenecid), Anturane (sufinpyrizone), Plaquenil (hydroxychloroquine), Aceclofenac, Acemetacin, Acetanilide, Actarit, Alclofenac, Alminoprofen, Aloxiprin, Aluminium Aspirin, Amfenac Sodium, Amidopyrine, Aminopropylone, Ammonium Salicylate, Ampiroxicam, Amyl Salicylate, Anirolac, Aspirin, Auranofin, Aurothioglucose, Aurotioprol, Azapropazone, Bendazac (Bendazac Lysine), Benorylate, Benoxaprofen, Benzpiperylone, Benzydamine, Hydrochloride, Bornyl Salicylate, Bromfenac Sodium, Bufexamac, Bumadizone Calcium, Butibufen Sodium, Capsaicin, Carbaspirin Calcium, Carprofen, Chlorthenoxazin, Choline Magnesium Trisalicylate, Choline Salicylate, Cinmetacin, Clofexamide, Clofezone, Clometacin, Clonixin, Cloracetadol, Cymene, Diacerein, Diclofenac (Diclofenac Diethylammonium Salt, Diclofenac Potassium, Diclofenac Sodium), Diethylamine Salicylate, Diethylsalicylamide, Difenpiramide, Diflunisal, Dipyrone, Droxicam, Epirizole, Etenzamide, Etersalate, Ethyl Salicylate, Etodolac, Etofenamate, Felbinac, Fenbufen, Fenclofenac, Fenoprofen Calcium, Fentiazac, Fepradinol, Feprazone, Floctafenine, Flufenamic, Flunoxaprofen, Flurbiprofen (Flurbiprofen Sodium), Fosfosal, Furprofen, Glafenine, Glucametacin, Glycol Salicylate, Gold Keratinate, Harpagophytum Procumbens, Ibufenac, Ibuprofen, Ibuproxam, Imidazole Salicylate, Indomethacin (Indomethacin Sodium), Indoprofen, Isamifazone, Isonixin, Isoxicam, Kebuzone, Ketoprofen, Ketorolac Trometamol, Lithium Salicylate, Lonazolac Calcium, Lomoxicam, Loxoprofen Sodium, Lysine Aspirin, Magnesium Salicylate, Meclofenamae Sodium, Mefenamic Acid, Meloxicam, Methyl Butetisalicylate, Methyl Gentisate, Methyl Salicylate, Metiazinic Acid, Metifenazone, Mofebutazone, Mofezolac, Morazone Hydrochloride, Morniflumate, Morpholine Salicylate, Nabumetone, Naproxen (Naproxen Sodium), Nifenazone, Niflumic Acid, Nimesulide, Oxametacin, Oxaprozin, Oxindanac, Oxyphenbutazone, Parsalmide, Phenybutazone, Phenyramidol Hydrochloride, Picenadol Hydrochloride, Picolamine Salicylate, Piketoprofen, Pirazolac, Piroxicam, Pirprofen, Pranoprofen, Pranosal, Proglumetacin Maleate, Proquazone, Protizinic Acid, Ramifenazone, Salacetamide, Salamidacetic Acid, Salicylamide, Salix, Salol, Salsalate, Sodium Aurothiomalate, Sodium Gentisate, Sodium Salicylate, Sodium Thiosalicylate, Sulindac, Superoxide Dismutase (Orgotein, Pegorgotein, Sudismase), Suprofen, Suxibuzone, Tenidap Sodium, Tenoxicam, Tetrydamine, Thurfyl Salicylate, Tiaprofenic, Tiaramide Hydrochloride, Tinoridine Hydrochloride, Tolfenamic Acid, Tometin Sodium, Triethanolamine Salicylate, Ufenamate, Zaltoprofen, Zidometacin and Zomepirac Sodium.

Cardiovascular Disease

Compounds useful for combining with the compounds of the present invention for the treatment of cardiovascular disease encompass a wide range of therapeutic compounds.

Ileal bile acid transporter (IBAT) inhibitors, for example, are useful in the present invention, and are disclosed in patent application no. PCT/US95/10863, herein incorporated by reference. More IBAT inhibitors are described in PCT/US97/04076, herein incorporated by reference. Still further IBAT inhibitors useful in the present invention are described in U.S. application Ser. No. 08/816,065, herein incorporated by reference. More IBAT inhibitor compounds useful in the present invention are described in WO 98/40375, and WO 00/38725, herein incorporated by reference. Additional IBAT inhibitor compounds useful in the present invention are described in U.S. application Ser. No. 08/816,065, herein incorporated by reference.

In another aspect, the second biologically active agent is a statin. Statins lower cholesterol by inhibiting of 3-hydroxy-3-methylglutaryl coenzyme A (HMG CoA) reductase, a key enzyme in the cholesterol biosynthetic pathway. The statins decrease liver cholesterol biosynthesis, which increases the production of LDL receptors thereby decreasing plasma total and LDL cholesterol (Grundy, S. M. New Engl. J. Med. 319, 24 (1988); Endo, A. J. Lipid Res. 33, 1569 (1992)). Depending on the agent and the dose used, statins may decrease plasma triglyceride levels and may increase HDLc. Currently the statins on the market are lovastatin (Merck), simvastatin (Merck), pravastatin (Sankyo and Squibb) and fluvastatin (Sandoz). A fifth statin, atorvastatin (Parke-Davis/Pfizer), is the most recent entrant into the statin market. Any of these statins or other statins known in the art can be used in combination with the chalcones of the present invention.

Microsomal triglyceride transfer protein (MTP) inhibitor compounds useful in the combinations and methods of the present invention comprise a wide variety of structures and functionalities. Some of the MTP inhibitor compounds of particular interest for use in the present invention are disclosed in WO 00/38725, the disclosure from which is incorporated by reference. Descriptions of these therapeutic compounds can be found in the article by Wetterau, et al., Science, 282, 23 Oct. 1998, pp. 751-754, herein incorporated by reference, as well in U.S. Pat. Nos. 5,739,135, 5,712,279, and 5,760,246.

Cholesterol absorption antagonist compounds useful in the combinations and methods of the present invention comprise a wide variety of structures and functionalities. Some of the cholesterol absorption antagonist compounds of particular interest for use in the present invention are described in U.S. Pat. No. 5,767,115, herein incorporated by reference. Further cholesterol absorption antagonist compounds of particular interest for use in the present invention, and methods for making such cholesterol absorption antagonist compounds are described in U.S. Pat. No. 5,631,365, herein incorporated by reference.

A number of phytosterols suitable for the combination therapies of the present invention are described by Ling and Jones in “Dietary Phytosterols: A Review of Metabolism, Benefits and Side Effects,” Life Sciences 57 (3), 195-206 (1995). Without limitation, some phytosterols of particular use in the combination of the present invention are Clofibrate, Fenofibrate, Ciprofibrate, Bezafibrate, Gemfibrozil, as well as beta-sitosterol, brassicasterol, delta-7-stigmasterol, delta-7-avenasterol, campesterol and stigmasterol. The structures of several of the foregoing compounds can be found in WO 00/38725.

Phytosterols are also referred to generally by Nes (Physiology and Biochemistry of Sterols, American Oil Chemists' Society, Champaign, Ill., 1991, Table 7-2). Especially preferred among the phytosterols for use in the combinations of the present invention are saturated phytosterols or stanols. Additional stanols are also described by Nes (Id.) and are useful in the combination of the present invention. In the combination of the present invention, the phytosterol preferably comprises a stanol. In one preferred embodiment the stanol is campestanol. In another preferred embodiment the stanol is cholestanol. In another preferred embodiment the stanol is clionastanol. In another preferred embodiment the stanol is coprostanol. In another preferred embodiment the stanol is 22,23-dihydrobrassicastanol. In another embodiment the stanol is epicholestanol. In another preferred embodiment the stanol is fucostanol. In another preferred embodiment the stanol is stigmastanol.

Another embodiment the present invention encompasses a therapeutic combination of a compound of the present invention and an HDLc elevating agent. In one aspect, the second HDLc elevating agent can be a cholesteryl ester transfer protein (CETP) inhibitor. Individual CETP inhibitor compounds useful in the present invention are separately described in WO 00/38725, the disclosure of which is herein incorporated by reference. Other individual CETP inhibitor compounds useful in the present invention are separately described in WO 99/14174, EP818448, WO 99/15504, WO 99/14215, WO 98/04528, and WO 00/17166, the disclosures of which are herein incorporated by reference. The chemical CETP inhibitor JTT-705 (S-[2-([[1-(2-ethylbutyl)cyclohexyl]carbonyl]amino)phenyl]2-methylpropanethioate; Japan Tobacco Inc., Tokyo, Japan), as described by Huang, et al. in Clinical Science (London) 103 (6), 587-594 (2002), Okamato et al. in Nature, 406, 203-207 (2000), Okamato, et al. in European Journal of Pharmacology, 466 (1-2), 147-154 (2003), and more recently by Linders in Current Opinion in Investigational Drugs, 5(3), 339-343 (2004), is also envisioned to be useful in the present invention. Still other individual CETP inhibitor compounds useful in the present invention are separately described in WO 00/18724, WO 00/18723, and WO 00/18721, the disclosures of which are herein incorporated by reference. Other individual CETP inhibitor compounds useful in the present invention are separately described in WO 98/35937 as well as U.S. Pat. Nos. 6,313,142, 6,310,075, 6,197,786, 6,147,090, 6,147,089, 6,140,343, and 6,140,343, the disclosures of which is herein incorporated by reference.

In another aspect, the second biologically active agent can be a fibric acid derivative. Fibric acid derivatives useful in the combinations and methods of the present invention comprise a wide variety of structures and functionalities which have been reported and published in the art, such as clofibrate, gemfibrozil, and bezafibrate.

In another embodiment the present invention encompasses a therapeutic combination of a compound of the present invention and an antihypertensive agent. Hypertension is defined as persistently high blood pressure. In another embodiment, the chalcone is administered in combination with an ACE inhibitor, a beta andrenergic blocker, alpha andrenergic blocker, angiotensin II receptor antagonist, vasodilator and diuretic.

Pharmaceutical Compositions

Any host organism, including a pateint, mammal, and specifically a human, suffering from any of the above-described conditions can be treated by the administration of a composition comprising an effective amount of the compound of the invention or a pharmaceutically acceptable salt thereof, optionally in a pharmaceutically acceptable carrier or diluent.

The composition can be administered in any desired manner, including oral, topical, parenteral, intravenous, intradermal, intra-articular, intra-synovial, intrathecal, intra-arterial, intracardiac, intramuscular, subcutaneous, intraorbital, intracapsular, intraspinal, intrasternal, topical, transdermal patch, via rectal, vaginal or urethral suppository, peritoneal, percutaneous, nasal spray, surgical implant, internal surgical paint, infusion pump, or via catheter. In one embodiment, the agent and carrier are administered in a slow release formulation such as an implant, bolus, microparticle, microsphere, nanoparticle or nanosphere. For standard information on pharmaceutical formulations, see Ansel, et al., Pharmaceutical Dosage Forms and Drug Delivery Systems, Sixth Edition, Williams & Wilkins (1995).

An effective dose for any of the herein described conditions can be readily determined by the use of conventional techniques and by observing results obtained under analogous circumstances. In determining the effective dose, a number of factors are considered including, but not limited to: the species of patient; its size, age, and general health; the specific disease involved; the degree of involvement or the severity of the disease; the response of the individual patient; the particular compound administered; the mode of administration; the bioavailability characteristics of the preparation administered; the dose regimen selected; and the use of concomitant medication. Typical systemic dosages for all of the herein described conditions are those ranging from 0.1 mg/kg to 500 mg/kg of body weight per day as a single daily dose or divided daily doses. Preferred dosages for the described conditions range from 5-1500 mg per day. A more particularly preferred dosage for the desired conditions ranges from 25-750 mg per day. Typical dosages for topical application are those ranging from 0.001 to 100% by weight of the active compound.

The compound is administered for a sufficient time period to alleviate the undesired symptoms and the clinical signs associated with the condition being treated.

The active compound is included in the pharmaceutically acceptable carrier or diluent in an amount sufficient to deliver to a patient a therapeutic amount of compound in vivo in the absence of serious toxic effects.

The concentration of active compound in the drug composition will depend on absorption, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the dosage ranges set forth herein are exemplary only and are not intended to limit the scope or practice of the claimed composition. The active ingredient may be administered at once, or may be divided into a number of smaller doses to be administered at varying intervals of time.

A preferred mode of administration of the active compound for systemic delivery is oral. Oral compositions will generally include an inert diluent or an edible carrier. They may be enclosed in gelatin capsules or compressed into tablets. For the purpose of oral therapeutic administration, the active compound can be incorporated with excipients and used in the form of tablets, troches or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or other enteric agents.

The compound or its salts can be administered as a component of an elixir, suspension, syrup, wafer, chewing gum or the like. A syrup may contain, in addition to the active compounds, sucrose as a sweetening agent and certain preservatives, dyes and colorings and flavors.

The compound can also be mixed with other active materials that do not impair the desired action, or with materials that supplement the desired action. The compounds can also be administered in combination with nonsteroidal antiinflammatories such as ibuprofen, indomethacin, fenoprofen, mefenamic acid, flufenamic acid, sulindac. The compound can also be administered with corticosteriods.

Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

If administered intravenously, preferred carriers are physiological saline, bacteriostatic water, Cremophor EL™ (BASF, Parsippany, N.J.) or phosphate buffered saline (PBS).

In a preferred embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) are also preferred as pharmaceutically acceptable carriers. These may be prepared according to methods known to those skilled in the art, for example, as described in U.S. Pat. No. 4,522,811 (which is incorporated herein by reference in its entirety). For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the compound is then introduced into the container. The container is then swirled by hand to free lipid material from the sides of the container and to disperse lipid aggregates, thereby forming the liposomal suspension.

Suitable vehicles or carriers for topical application can be prepared by conventional techniques, such as lotions, suspensions, ointments, creams, gels, tinctures, sprays, powders, pastes, slow-release transdermal patches, suppositories for application to rectal, vaginal, nasal or oral mucosa. In addition to the other materials listed above for systemic administration, thickening agents, emollients and stabilizers can be used to prepare topical compositions. Examples of thickening agents include petrolatum, beeswax, xanthan gum, or polyethylene, humectants such as sorbitol, emollients such as mineral oil, lanolin and its derivatives, or squalene.

Any of the compounds described herein for combination or alternation therapy can be administered as any derivative that upon administration to the recipient, is capable of providing directly or indirectly, the parent compound, or that exhibits activity itself. Nonlimiting examples are the pharmaceutically acceptable salts (alternatively referred to as “physiologically acceptable salts”), and a compound which has been alkylated or acylated at an appropriate position. The modifications can affect the biological activity of the compound, in some cases increasing the activity over the parent compound. This can easily be assessed by preparing the derivative and testing its anti-inflammatory activity according to known methods.

In another embodiment, the invention is a pharmaceutical composition comprising any of the above embodiments or any of the specific Examples together with one or more pharmaceutically acceptable carriers.

Another embodiment includes the embodiments above or any of the Examples as a means to treat or prophylactically treat an inflammatory disorder including arthritis, rheumatoid arthritis, asthma, diabetic retinopathy, diabetic nephropathy, diabetic vasculopathy, multiple sclerosis, allergic rhinitis, chronic obstructive pulmonary disease, systemic lupus erthematosus, atherosclerosis, and restinosis.

A further embodiment includes the intermediates used to make the final compounds of the invention. Said intermediates are useful as starting materials for making the compounds of the invention as well as having pharmaceutical activity alone.

The invention also includes the process for making the intermediates as well as the final compounds.

Biological Activity of Active Compounds

The ability of a compound described herein to inhibit the expression of VCAM-1 or in the treatment of diseases in a host can be assessed using any known method, including that described in detail below.

In Vitro VCAM-1 Assay

Cell Culture and compound dosing: Cultured primary human aortic (HAEC) or pulmonary (HPAEC) endothelial cells were obtained from Clonetics, Inc., and were used below passage 9. Cells were seeded in 96 well plates such that they would reach 90-95% confluency by the following day. On the following day the cells were stimulated with TNF-α (1 ng/ml) in the presence or absence of compounds dissolved in DMSO such that the final concentration of DMSO is 0.25% or less. To establish a dose curve for each compound, four concentrations in 2- to 5-fold increments were used. Cells were exposed to TNF-α and compounds for approximately 16 hours. The next day the cells were examined under microscope to score for visual signs of toxicity or cell stress.

Following 16 hr exposure to TNF-α and compound the media was discarded and the cells were washed once with Hanks Balanced Salt Solution (HBSS)/Phosphate buffered saline (PBS) (1:1). Primary antibodies against VCAM-1 (0.25 μg/ml in HBSS/PBS+5% FBS) were added and incubated for 30-60 minutes at 37° C. Cells were washed with HBSS/PBS three times, and secondary antibody Horse Radish Peroxidase (HRP)-conjugated goat anti-mouse IgG (1:500 in HBSS/PBS+5% FBS) were added and incubated for 30 minutes at 37° C. Cells were washed with HBSS/PBS four time and TMB substrate were added and incubated at room temperature in the dark until there was adequate development of blue color. The length of time of incubation was typically 5-15 minutes. 2N sulfuric acid was added to stop the color development and the data was collected by reading the absorbance on a BioRad ELISA plate reader at OD 450 nm. The results are expressed as IC₅₀ values (the concentration (micromolar) of compound required to inhibit 50% of the maximal response of the control sample stimulated by TNF-α only). Compounds exhibiting IC₅₀'s of less than 10 micromolar are tabulated in Biological Table 1.

BIOLOGICAL TABLE 1

| Example | VCAM-1 |
|---|---|
| Number | IC₅₀ (μM) |
| 1 | <3 |
| 2 | <1 |
| 3 | <3 |
| 4 | <1 |
| 5 | <1 |
| 6 | <1 |
| 7 | <1 |
| 8 | <1 |
| 9 | <1 |
| 10 | <1 |
| 11 | <1 |
| 12 | <3 |
| 13 | <3 |
| 14 | <3 |
| 15 | <3 |
| 16 | <1 |
| 17 | <1 |
| 18 | <3 |
| 19 | <1 |
| 20 | <1 |
| 21 | <1 |
| 22 | <1 |
| 23 | <3 |
| 24 | <1 |
| 25 | <1 |
| 26 | <3 |
| 27 | <1 |
| 28 | <1 |
| 29 | <1 |
| 30 | <1 |
| 31 | <1 |
| 32 | <1 |
| 33 | <3 |
| 34 | >10 |
| 35 | <3 |
| 36 | <3 |
| 37 | <6 |
| 38 | <10 |
| 39 | <6 |
| 40 | <1 |
| 41 | <1 |
| 42 | <1 |
| 43 | <3 |
| 44 | <3 |
| 45 | <1 |
| 46 | <3 |
| 47 | <3 |
| 48 | <10 |
| 49 | <6 |
| 50 | <3 |
| 51 | <3 |
| 52 | <3 |
| 53 | <3 |
| 54 | <1 |
| 55 | <3 |
| 56 | <3 |
| 57 | <3 |
| 58 | <3 |
| 59 | <1 |
| 60 | >10 |
| 61 | <1 |
| 62 | <1 |
| 63 | <10 |
| 64 | >10 |
| 65 | <1 |
| 66 | <3 |

Rheumatoid Arthritis Protocol

Male Lewis rats (150-175 g) from Charles River Laboratories were anesthetized on day 0 with 3-5% isoflurane anesthesia while the tail base was shaved and adjuvant mixture was injected. 50 μL of adjuvant (10 mg/ml M. butyricum in mineral oil) was injected subcutaneously into two sites at the tail base. Paw swelling was monitored using a plethysmometer (UGO Basile), after shaving each leg to the level of the Achilles tendon to mark the level of immersion. A baseline paw measurement for both hindpaws was taken between 4 and a second measurement was taken on day 8. Onset of paw swelling occurred rapidly between days 8-11 and daily measurements were performed every weekday between day 8 and day 14. Compounds of the invention and vehicles were dosed therapeutically (d8-13) after swelling was confirmed. Solutions were injected subcutaneously or given orally by gavage 1-2 times per day. From day 0, rats were weighed on days 0, 8, 11, and 14 and overall health was monitored. Plasma drug levels, if desired, were measured in tail-vein derived blood samples taken on day 13. On day 14, blood samples were obtained by cardiac puncture, rats were euthanized with CO₂, selected organs were removed and both hindpaws were amputated and placed in 10% buffered formalin for histopathological analysis. See Biological Table 2.

BIOLOGICAL TABLE 2

| Compound | % Inhibition 20 mg/kg |
|---|---|
| Example Number | sq, bid, d8-13 |
| 8 | 44 |
| 30 | 77 |

Asthma Protocol

Balb/C mice (6-8 weeks old) are sensitized to ovalbumin (ova) (8 ug ova absorbed in 3.3 mg Alum inject) on days 0 and 5. On day 12, the mice were aerosol challenged with 0.5% ovalbumin dissolved in sterile saline for 1 h in the AM, and then again in the PM (at least 4 h apart). On day 14, the mice were anesthetized with ketamine/xylazine/acepromazine cocktail, exsanguinated, and then euthanized. Following blood collection, bronchoaveolar lavage was performed on each animal. Total cell counts were conducted on the lavage fluid. The lavage fluid was processed and slides were made by spinning the samples with a cytospin centrifuge. Slides were airdried and stained using Diff-Quik® stain. Cell differentials of the lavage fluid were completed at the conclusion of the study. Statistical analysis involved ANOVA and Tukey-Kramer post hoc tests. Compounds were administered by subcutaneous injection twice daily from day 11-13. The formulations used contained various mixtures of the following excipients (pharmasolve, cremophor RH 40, tween 80, PEG 300). See Biological Table 3

BIOLOGICAL TABLE 3

| Compound | % Inhibition 6 mg/kg |
|---|---|
| Example Number | sq, bid, d11-13 |
| 8 | 50 |
| 30 | 45 |
| 37 | 27 |

All of the compositions, methods and/or processes disclosed and claimed herein can be made and executed without undue experimentation in light of the present disclosure. While the compositions and methods of this invention have been described in terms of preferred embodiments, it will be apparent to those of skill in the art that variations may be applied to the compositions, methods and/or processes and in the steps or in the sequence of steps of the methods described herein without departing from the concept and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the scope and concept of the invention.

What is claimed is:

1. A compound of Formula I 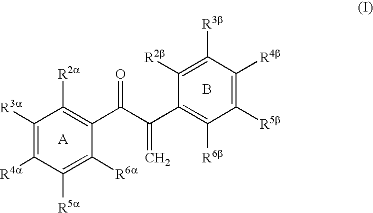
or its pharmaceutically acceptable salt or ester, wherein:
R^{2α, R}^{3α}, R^{4α}, R^{5α}, R^{6α}, R^{2β}, R^{3β}, R^{4β}, R^{5β} and R^{6β} are independently selected from the group consisting of hydrogen, halogen, nitro, alkyl, lower alkyl, alkenyl, alkynyl, carbocycle, cycloalkyl, cycloalkylalkyl, haloalkyl, aryl, arylalkyl, heteroaryl, heteroaryl lower alkyl, heterocyclic, heterocyclic lower alkyl, alkylthioalkyl, cycloalkylthioalkyl, arylthio lower alkyl, aralkyl lower thioalkyl, heteroarylthio lower alkyl, heteroaralkyl lower thioalkyl, heterocyclicthio lower alkyl, heterocyclicalkyl lower thioalkyl, lower alkyl S(O)-lower alkyl, lower alkyl-S(O)₂-lower alkyl, arylsulfinyl lower alkyl, arylsulfonyl lower alkyl, —C(O)R², R²C(O)alkyl, aminoalkyl, cycloalkylaminoalkyl, arylamino lower alkyl, heteroarylamino lower alkyl, heterocyclicamino lower alkyl, hydroxyl, hydroxyalkyl, alditol, carbohydrate, polyol alkyl, alkoxy, lower alkoxy, —(O(CH₂)₂)₁₋₃—O-lower alkyl, polyoxyalkylene, cycloalkyloxy, cycloalkylalkoxy, haloalkoxy, aryloxy, arylalkoxy, heteroaryloxy, heteroarylalkoxy, heteroaryl lower alkoxy, heterocyclicoxy, heterocyclicalkoxy, heterocyclic lower alkoxy, —OC(R¹)₂C(O)OH, —OC(R¹)₂C(O)OR², —OC(R¹)₂C(O)NH₂, —OC(R¹)₂C(O)NHR², —OC(R¹)₂C(O)N(R²)₂, —OC(R¹)₂C(O)NR⁷R⁸, amino, alkylamino, acylamino, dialkylamino, cycloalkylamino, arylamino, aralkylamino, heteroarylamino, heteroaralkylamino, heterocyclicamino, heterocyclicalkylamino, —NHR², N(R²)₂, —NR⁷R⁸, —NHC(R¹)₂C(O)OH, —NHC(R¹)₂C(O)OR², —NHC(O)R², —N(R²)C(O)R², —NHC(O)OR², —NHC(O)SR², —NHSO₂NHR², —NHSO₂R², —NHSO₂NR⁷R⁸, —N(C(O)NHR²)₂, —NR²SO₂R², —NHC(O)NHR², —NHC(O)NR⁷R⁸, —NHC(O)N(R²)₂, thiol, alkylthio, cycloalkylthio, cycloalkylalkylthio, haloalkylthio, arylthio, aralkylthio, heteroarylthio, heteroaralkylthio, heterocyclicthio, heterocyclicalkylthio, alkylsulfonyl, arylsulfonyl, haloalkylsulfonyl, —SC(R¹)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR⁷R⁸, —SO₂NHC(O)R², —SR², —SO₂NHC(O)NHR², —SO₂NHC(O)N(R²) 2, —SO₂NHC(O)NR⁷R⁸, sulfonic acid, sulfonate, sulfate, sulfinic acid, sulfenic acid, cyano, tetrazol-5-yl, carboxy, —C(O)OR², —C(O)NH₂, —C(O)NHR², —C(O)N(R²)₂, —C(O)NR⁷R⁸, —C(O)NHC(O)R², —C(O)NHC(O)NHR², —C(O)NHC(O)N(R²)₂, —C(O)NHC(O)NR⁷R⁸, —C(O)NHSO₂R², —C(O)NHSO₂NHR², —C(O)NHSO₂N(R²), —C(O)NHSO₂NR⁷R⁸—C(CH₃)₂C(O)OH, —(CH₂)_{y}C(O)OH, wherein y is 1, 2, 3, 4, 5, or 6, —PO₂H₂, —PO₃H₂, —P(R²)O₂H, and phosphate, all of which can be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R¹ is independently selected from the group consisting of hydrogen, lower alkyl, carbocycle, cycloalkyl, aryl, heteroaryl, heterocyclic, arylalkyl, heteroarylalkyl, and heterocyclicalkyl, wherein all may be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R² is independently selected from the group consisting of alkyl, lower alkyl, alkenyl, alkynyl, carbocycle, cycloalkyl, aryl, heteroaryl, heterocyclic, arylalkyl, heteroarylalkyl, and heterocyclicalkyl, wherein all may be substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R⁷ and R⁸ are independently selected from the group consisting of alkyl, alkenyl, heteroaryl and aryl and linked together forming a 4- to 12-membered monocyclic, bicylic, tricyclic or benzofused ring;
wherein one of R^{2β}, R^{3β}, R^{4β}, R^{5β} or R^{6β}, or one of R^{2α}, R^{3α}, R^{4α}, R^{5α} or R^{6α} must be a carbon-carbon linked heterocyclic or heteroaryl; or
R^{2α} and R^{3α} taken together or R^{3α} and R^{4α} taken together or R^{4α} and R^{5α} taken together, or R^{2β} and R^{3α} taken together or R^{3α} and R^{4α} taken together or R^{4α} and R^{5α} taken together form a heterocyclic or heteroaryl optionally substituted by one or more alkoxycarbonylalkyl, carboxyalkyl, hydroxyalkyl or aminoalkyl and optionally substituted where possible with one or more selected from the group consisting of hydroxy, alkyl, carboxy, hydroxyalkyl, carboxyalkyl, amino, cyano, alkoxy, alkoxycarbonyl, acyl, oxo, —NR⁷R⁸, and halo; or
R^{2α} and R^{3α} taken together or R^{3α} and R^{4α} taken together or R^{4α} and R^{5α} taken together or R^{2β} and R^{3β} taken together or R^{3β} and R^{4β} taken together or R^{4β} and R^{5α} taken together form a 5- or 6-membered ring containing one nitrogen, which may optionally be substituted where possible with one or more selected from the group consisting of halo, alkyl, lower alkyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
wherein all R¹, R², R⁷ and R⁸ substituents can be optionally substituted where possible with one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂.

2. The compound of claim 1 or its pharmaceutically acceptable salt or ester, wherein:
R^{2α}, R^{3α}, R^{4α}, R^{5α}, R^{6α}, R^{2β}, R^{3β}, R^{4β}, R^{5β} and R^{6β} are independently selected from the group consisting of hydrogen, halogen, nitro, alkyl, lower alkyl, alkenyl, alkynyl, carbocycle, cycloalkyl, cycloalkylalkyl, haloalkyl, aryl, arylalkyl, heteroaryl, heteroaryl lower alkyl, heterocyclic, heterocyclic lower alkyl, alkylthioalkyl, cycloalkylthioalkyl, arylthio lower alkyl, aralkyl lower thioalkyl, heteroarylthio lower alkyl, heteroaralkyl lower thioalkyl, heterocyclicthio lower alkyl, heterocyclicalkyl lower thioalkyl, lower alkyl S(O)-lower alkyl, lower alkyl-S(O)₂-lower alkyl, arylsulfinyl lower alkyl, arylsulfonyl lower alkyl, —C(O)R², R²C(O)alkyl, aminoalkyl, cycloalkylaminoalkyl, arylamino lower alkyl, heteroarylamino lower alkyl, heterocyclicamino lower alkyl, hydroxyl, hydroxyalkyl, alkoxy, lower alkoxy, —(O(CH₂)₂)₁₋₃—O-lower alkyl, cycloalkyloxy, cycloalkylalkoxy, haloalkoxy, aryloxy, arylalkoxy, heteroaryloxy, heteroarylalkoxy, heteroaryl lower alkoxy, heterocyclicoxy, heterocyclicalkoxy, heterocyclic lower alkoxy, —OC(R¹)₂C(O)OH, —OC(R¹)₂C(O)OR², —OC(R¹)₂C(O)NH₂, —OC(R¹)₂C(O)NHR², —OC(R¹)₂C(O)N(R²)₂, —OC(R¹)₂C(O)NR⁷R⁸, amino, alkylamino, acylamino, dialkylamino, cycloalkylamino, arylamino, aralkylamino, heteroarylamino, heteroaralkylamino, heterocyclicamino, heterocyclicalkylamino, —NHR², N(R²)₂, —NR⁷R⁸, —NHC(R¹)₂C(O)OH, —NHC(R¹)₂C(O)OR², —NHC(O)R², —N(R²)C(O)R², —NHC(O)OR², —NHC(O)SR², —NHSO₂NHR², —NHSO₂R², —NHSO₂NR R, —N(C(O)NHR²)₂, —NR²SO₂R², —NHC(O)NHR², —NHC(O)NR⁷R², —NHC(O)N(R²)₂, thiol, alkylthio, cycloalkylthio, cycloalkylalkylthio, haloalkylthio, arylthio, aralkylthio, heteroarylthio, heteroaralkylthio, heterocyclicthio, heterocyclicalkylthio, alkylsulfonyl, arylsulfonyl, haloalkylsulfonyl, —SC(R¹)₂C(O)OH, —SC(R¹)₂C(O)OR², —SCH₂C(O)OH, SCF₂C(O)OH, —SO₂NH₂, —SO₂NHR², —SO₂N(R²)₂, SO₂NR², —SO₂NHC(O)R², —SR², —SO₂NHC(O)NHR², —SO₂NHC(O)N(R²)₂, —SO₂NHC(O)NR⁷R⁸, sulfonic acid, sulfonate, sulfate, sulfinic acid, sulfenic acid, cyano, tetrazol-5-yl, carboxy, —C(O)OR², —C(O)NH₂, —C(O)NHR², —C(O)N(R²)₂, —C(O)NR⁷R⁸, —C(O)NHC(O)R², —C(O)NHC(O)NHR², —C(O)NHC(O)N(R²)₂, —C(O)NHC(O)NR⁷R⁸, —C(O)NHSO₂R², —C(O)NHSO₂NHR², —C(O)NHSO₂N(R²), —C(O)NHSO₂NR⁷R⁸, —C(CH₃)₂C(O)OH, —(CH₂)_{y}C(O)OH, wherein y is 1, 2, 3, 4, 5, or 6, —PO₂H₂, —PO₃H₂, —P(R²)O₂H, and phosphate, all of which can be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R¹ is independently selected from the group consisting of hydrogen, lower alkyl, carbocycle, cycloalkyl, aryl, heteroaryl, heterocyclic, arylalkyl, heteroarylalkyl, and heterocyclicalkyl, wherein all may be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R¹)₂;
R² is independently selected from the group consisting of alkyl, lower alkyl, alkenyl, alkynyl, carbocycle, cycloalkyl, aryl, heteroaryl, heterocyclic, arylalkyl, heteroarylalkyl, and heterocyclicalkyl, wherein all may be substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R⁷ and R⁸ are independently selected from the group consisting of alkyl, alkenyl, heteroaryl and aryl and linked together forming a 4- to 12-membered monocyclic, bicylic, tricyclic or benzofused ring;
wherein one of R^{2β}, R^{3β}, R^{4β}, R^{5β} or R^{6β}, or one of R^{2α}, R^{3α}, R^{4α}, R^{5α} or R^{6α} must be a carbon-carbon linked heterocyclic or heteroaryl;
wherein all R¹, R², R⁷ and R⁸ substituents can be optionally substituted where possible with one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂.

3. The compound of claim 2 or its pharmaceutically acceptable salt or ester, wherein:
R^{2α}, R^{3α}, R^{4α}, R^{5α}, R^{6α}, R^{2β}, R^{3β}, R^{4β}, R^{5β} and R^{6β} are independently selected from the group consisting of hydrogen, halogen, nitro, alkyl, lower alkyl, alkenyl, alkynyl, carbocycle, cycloalkyl, cycloalkylalkyl, haloalkyl, aryl, arylalkyl, heteroaryl, heteroaryl lower alkyl, heterocyclic, heterocyclic lower alkyl, alkylthioalkyl, cycloalkylthioalkyl, arylthio lower alkyl, aralkyl lower thioalkyl, heteroarylthio lower alkyl, heteroaralkyl lower thioalkyl, heterocyclicthio lower alkyl, heterocyclicalkyl lower thioalkyl, —C(O)R², R²C(O)alkyl, aminoalkyl, cycloalkylaminoalkyl, arylamino lower alkyl, heteroarylamino lower alkyl, heterocyclicamino lower alkyl, hydroxyl, hydroxyalkyl, alkoxy, lower alkoxy, —(O(CH₂)₂)₁₋₃—O-lower alkyl, cycloalkyloxy, cycloalkylalkoxy, haloalkoxy, aryloxy, arylalkoxy, heteroaryloxy, heteroarylalkoxy, heteroaryl lower alkoxy, heterocyclicoxy, heterocyclicalkoxy, heterocyclic lower alkoxy, —OC(R¹)₂C(O)OH, —OC(R¹)₂C(O)OR², —OC(R¹)₂C(O)NH₂, —OC(R¹)₂C(O)NHR², —OC(R¹)₂C(O)N(R²)₂, —OC(R¹)₂C(O)NR⁷R⁸, amino, alkyl amino, acylamino, dialkylamino, cycloalkylamino, arylamino, aralkylamino, heteroarylamino, heteroaralkylamino, heterocyclicamino, heterocyclicalkylamino, —NHR², N(R²)₂, —NR⁷R⁸, —NHC(R¹)₂C(O)OH, —NHC(R¹)₂C(O)OR₂, —NHC(O)R², —N(R²)C(O)R², —NHC(O)OR², —NHC(O)SR², —NHSO₂NHR², —NHSO₂R², —NHSO₂NR⁷R⁸, —N(C(O)NHR²)₂, —NR²SO₂R², —NHC(O)NHR², —NHC(O)NR⁷R⁸, —NHC(O)N(R²)₂, thiol, alkylthio, cycloalkylthio, cycloalkylalkylthio, haloalkylthio, arylthio, aralkylthio, heteroarylthio, heteroaralkylthio, heterocyclicthio, heterocyclicalkylthio, alkylsulfonyl, arylsulfonyl, haloalkylsulfonyl, —SC(R¹)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR⁷R⁸, —SO₂NHC(O)R², —SR², —SO₂NHC(O)NHR², —SO₂NHC(O)N(R²) 2, —SO₂NHC(O)NR⁷R⁸, sulfonic acid, sulfonate, sulfate, sulfinic acid, sulfenic acid, cyano, tetrazol-5-yl, carboxy, —C(O)OR², —C(O)NH₂, —C(O)NHR², —C(O)N(R²)₂, —C(O)NR⁷R⁸, —C(O)NHC(O)R², —C(O)NHC(O)NHR², —C(O)NHC(O)N(R²)₂, —C(O)NHC(O)NR⁷R⁸, —C(O)NHSO₂R², —C(O)NHSO₂NHR², —C(O)NHSO₂N(R²), —C(O)NHSO₂NR⁷R⁸, —C(CH₃)₂C(O)OH, —(CH₂)_{y}C(O)OH, wherein y is 1, 2, 3, 4, 5, or 6, —PO₂H₂, —PO₃H₂, —P(R²)O₂H, and phosphate, all of which can be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R¹ is independently selected from the group consisting of hydrogen, lower alkyl, carbocycle, cycloalkyl, aryl, heteroaryl, heterocyclic, arylalkyl, heteroarylalkyl, and heterocyclicalkyl, wherein all may be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R² is independently selected from the group consisting of alkyl, lower alkyl, alkenyl, alkynyl, carbocycle, cycloalkyl, aryl, heteroaryl, heterocyclic, arylalkyl, heteroarylalkyl, and heterocyclicalkyl, wherein all may be substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R⁷ and R⁸ are independently selected from the group consisting of alkyl, alkenyl, heteroaryl and aryl and linked together forming a 4- to 12-membered monocyclic, bicylic, tricyclic or benzofused ring;
wherein one of R^{2β}, R^{3β}, R^{4β}, R^{5β} or R^{6β}, or one of R^{2α}, R^{3α}, R^{4α}, R^{5α} or R^{6α} must be a carbon-carbon linked heteroaryl;
wherein all R¹, R², R⁷ and R⁸ substituents can be optionally substituted where possible with one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂.

4. The compound of claim 1 or its pharmaceutically acceptable salt or ester, wherein:
R^{3α}, R^{4α}, R^{5α}, R^{2β}, R^{3β}, R^{4β}, R^{5β} and R^{6β} are independently selected from the group consisting of hydrogen, halogen, nitro, alkyl, lower alkyl, alkenyl, alkynyl, carbocycle, cycloalkyl, cycloalkylalkyl, haloalkyl, aryl, arylalkyl, heteroaryl, heteroaryl lower alkyl, heterocyclic, heterocyclic lower alkyl, alkylthioalkyl, cycloalkylthioalkyl, arylthio lower alkyl, aralkyl lower thioalkyl, heteroarylthio lower alkyl, heteroaralkyl lower thioalkyl, heterocyclicthio lower alkyl, heterocyclicalkyl lower thioalkyl, —C(O)R², R²C(O)alkyl, aminoalkyl, cycloalkylaminoalkyl, arylamino lower alkyl, heteroarylamino lower alkyl, heterocyclicamino lower alkyl, hydroxyl, hydroxyalkyl, alkoxy, lower alkoxy, —(O(CH₂)₂)₁₋₃—O-lower alkyl, cycloalkyloxy, cycloalkylalkoxy, haloalkoxy, aryloxy, arylalkoxy, heteroaryloxy, heteroarylalkoxy, heteroaryl lower alkoxy, heterocyclicoxy, heterocyclicalkoxy, heterocyclic lower alkoxy, —OC(R¹)₂C(O)OH, —OC(R¹)₂C(O)OR², —OC(R¹)₂C(O)NH₂, —OC(R¹)₂C(O)NHR², —OC(R¹)₂C(O)N(R²)₂, —OC(R¹)₂C(O)NR⁷R⁸, amino, alkylamino, acylamino, dialkylamino, cycloalkylamino, arylamino, aralkylamino, heteroarylamino, heteroaralkylamino, heterocyclicamino, heterocyclicalkylamino, —NHR², N(R²)₂, —NR R, —NHC(R¹)₂C(O)OH, —NHC(R¹)₂C(O)OR², —NHC(O)R², —N(R²)C(O)R², —NHC(O)OR², —NHC(O)SR², —NHSO₂NHR², —NHSO₂R², —NHSO₂NR⁷R⁸, —N(C(O)NHR²)₂, —NR²SO₂R², —NHC(O)NHR², —NHC(O)NR⁷R⁸, —NHC(O)N(R²)₂, thiol, alkylthio, cycloalkylthio, cycloalkylalkylthio, haloalkylthio, arylthio, aralkylthio, heteroarylthio, heteroaralkylthio, heterocyclicthio, heterocyclicalkylthio, alkylsulfonyl, arylsulfonyl, haloalkylsulfonyl, —SC(R¹)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR⁷R⁸, —SO₂NHC(O)R², —SR², —SO₂NHC(O)NHR², —SO₂NHC(O)N(R²)₂, —SO₂NHC(O)NR⁷R⁸, sulfonic acid, sulfonate, sulfate, sulfinic acid, sulfenic acid, cyano, tetrazol-5-yl, carboxy, —C(O)OR², —C(O)NH₂, —C(O)NHR², —C(O)N(R²)₂, —C(O)NR⁷R⁸, —C(O)NHC(O)R², —C(O)NHC(O)NHR², —C(O)NHC(O)N(R²)₂, —C(O)NHC(O)NR⁷R⁸, —C(O)NHSO₂R², —C(O)NHSO₂NHR², —C(O)NHSO₂N(R²), —C(O)NHSO₂NR⁷R⁸, —C(CH₃)₂C(O)OH, —(CH₂)_{y}C(O)OH, wherein y is 1, 2, 3, 4, 5, or 6, —PO₂H₂, —PO₃H₂, —P(R²)O₂H, and phosphate, all of which can be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R^{2α} and R^{6α} are independently selected from the group consisting of halogen, nitro, alkyl, lower alkyl, alkenyl, alkynyl, carbocycle, cycloalkyl, cycloalkylalkyl, haloalkyl, aryl, arylalkyl, heteroaryl, heteroaryl lower alkyl, heterocyclic, heterocyclic lower alkyl, alkylthioalkyl, cycloalkylthioalkyl, arylthio lower alkyl, aralkyl lower thioalkyl, heteroarylthio lower alkyl, heteroaralkyl lower thioalkyl, heterocyclicthio lower alkyl, heterocyclicalkyl lower thioalkyl, R²C(O)alkyl, aminoalkyl, cycloalkylaminoalkyl, arylamino lower alkyl, heteroarylamino lower alkyl, heterocyclicamino lower alkyl, hydroxyalkyl, alkoxy, lower alkoxy, —(O(CH₂)₂)₁₋₃—O-lower alkyl, cycloalkyloxy, cycloalkylalkoxy, haloalkoxy, aryloxy, arylalkoxy, heteroaryloxy, heteroarylalkoxy, heteroaryl lower alkoxy, heterocyclicoxy, heterocyclicalkoxy, heterocyclic lower alkoxy, —OC(R¹)₂C(O)OH, —OC(R¹)₂C(O)OR², —OC(R¹)₂C(O)NH₂, —OC(R¹)₂C(O)NHR², —OC(R¹)₂C(O)N(R²)₂, —OC(R¹)₂C(O)NR⁷R⁸, —NHSO₂NHR², —NHSO₂R², —NHSO₂NR⁷R⁸, —NR²SO₂R², alkylthio, cycloalkylthio, cycloalkylalkylthio, haloalkylthio, arylthio, aralkylthio, heteroarylthio, heteroaralkylthio, heterocyclicthio, heterocyclicalkylthio, alkylsulfonyl, arylsulfonyl, haloalkylsulfonyl, —SC(R¹)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR⁷R⁸, —SO₂NHC(O)R², —SR², —SO₂NHC(O)NHR², —SO₂NHC(O)N(R²) 2, —SO₂NHC(O)NR⁷R⁸, sulfonic acid, sulfonate, sulfate, sulfinic acid, sulfenic acid, cyano, tetrazol-5-yl, carboxy, —C(O)OR², —C(O)NH₂, —C(O)NHR², —C(O)N(R²)₂, —C(O)NR⁷R⁸, —C(O)NHC(O)R², —C(O)NHC(O)NHR², —C(O)NHC(O)N(R²)₂, —C(O)NHC(O)NR⁷R⁸, —C(O)NHSO₂R², —C(O)NHSO₂NHR², —C(O)NHSO₂N(R²), —C(O)NHSO₂NR⁷R⁸, —C(CH₃)₂C(O)OH, —(CH₂)_{y}C(O)OH, wherein y is 1, 2, 3, 4, 5, or 6, —PO₂H₂, —PO₃H₂, —P(R²)O₂H, and phosphate, all of which can be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R¹ is independently selected from the group consisting of hydrogen, lower alkyl, carbocycle, cycloalkyl, aryl, heteroaryl, heterocyclic, arylalkyl, heteroarylalkyl, and heterocyclicalkyl, wherein all may be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R² is independently selected from the group consisting of alkyl, lower alkyl, alkenyl, alkynyl, carbocycle, cycloalkyl, aryl, heteroaryl, heterocyclic, arylalkyl, heteroarylalkyl, and heterocyclicalkyl, wherein all may be substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R⁷ and R⁸ are independently selected from the group consisting of alkyl, alkenyl, heteroaryl and aryl and linked together forming a 4- to 12-membered monocyclic, bicylic, tricyclic or benzofused ring;
wherein one of R^{2β}, R^{3β}, R^{4β}, R^{5β} or R^{6β}, or one of R^{2α}, R^{3α}, R^{4α}, R^{5α} or R^{6α} must be a carbon-carbon linked heteroaryl;
wherein all R¹, R², R⁷ and R⁸ substituents can be optionally substituted where possible with one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂.

5. The compound of claim 4 or its pharmaceutically acceptable salt or ester, wherein:
R^{3α}, R^{4α}, R^{5α}, R^{2β}, R^{3β}, R^{4β}, R^{5β} and R^{6β} are independently selected from the group consisting of hydrogen, halogen, nitro, alkyl, lower alkyl, alkenyl, alkynyl, carbocycle, cycloalkyl, cycloalkylalkyl, haloalkyl, aryl, arylalkyl, heteroaryl, heteroaryl lower alkyl, heterocyclic, heterocyclic lower alkyl, alkylthioalkyl, cycloalkylthioalkyl, arylthio lower alkyl, aralkyl lower thioalkyl, heteroarylthio lower alkyl, heteroaralkyl lower thioalkyl, heterocyclicthio lower alkyl, heterocyclicalkyl lower thioalkyl, —C(O)R², R²C(O)alkyl, aminoalkyl, cycloalkylaminoalkyl, arylamino lower alkyl, heteroarylamino lower alkyl, heterocyclicamino lower alkyl, hydroxyl, hydroxyalkyl, alkoxy, lower alkoxy, —(O(CH₂)₂)₁₋₃—O-lower alkyl, cycloalkyloxy, cycloalkylalkoxy, haloalkoxy, aryloxy, arylalkoxy, heteroaryloxy, heteroarylalkoxy, heteroaryl lower alkoxy, heterocyclicoxy, heterocyclicalkoxy, heterocyclic lower alkoxy, —OC(R¹)₂C(O)OH, —OC(R¹)₂C(O)OR², —OC(R¹)₂C(O)NH₂, —OC(R¹)₂C(O)NHR², —OC(R¹)₂C(O)N(R²)₂, —OC(R¹)₂C(O)NR⁷R⁸, amino, alkylamino, acylamino, dialkylamino, cycloalkylamino, arylamino, aralkylamino, heteroarylamino, heteroaralkylamino, heterocyclicamino, heterocyclicalkylamino, —NHR², N(R²)₂, —NR⁷R⁸, —NHC(R¹)₂C(O)OH, —NHC(R¹)₂C(O)OR², —NHC(O)R², —N(R²)C(O)R², —NHC(O)OR², —NHC(O)SR², —NHSO₂NHR², —NHSO₂R², —NHSO₂NR⁷R⁸, —N(C(O)NHR²)₂, —NR²SO₂R², —NHC(O)NHR², —NHC(O)NR⁷R⁸, —NHC(O)N(R²)₂, thiol, alkylthio, cycloalkylthio, cycloalkylalkylthio, haloalkylthio, arylthio, aralkylthio, heteroarylthio, heteroaralkylthio, heterocyclicthio, heterocyclicalkylthio, alkylsulfonyl, arylsulfonyl, haloalkylsulfonyl, —SC(R¹)₂C(O)OH, —SC(R¹)₂C(O)OR², —SCH₂C(O)OH, —SCF₂C(O)OH, —SO₂NH₂, SO₂NHR², —SO₂N(R²)₂, SO₂NR⁷R⁸, —SO₂NHC(O)R², —SR², —SO₂NHC(O)NHR², —SO₂NHC(O)N(R²)₂, —SO₂NHC(O)NR⁷R⁸, sulfonic acid, sulfonate, sulfate, sulfinic acid, sulfenic acid, cyano, tetrazol-5-yl, carboxy, —C(O)OR², —C(O)NH₂, —C(O)NHR², —C(O)N(R²)₂, —C(O)NR⁷R⁸, —C(O)NHC(O)R², —C(O)NHC(O)NHR², —C(O)NHC(O)N(R²)₂, —C(O)NHC(O)NR⁷R⁸, —C(O)NHSO₂R², —C(O)NHSO₂NHR², —C(O)NHSO₂N(R), —C(O)NHSO₂NR⁷R⁸, —C(CH₃)₂C(O)OH, —(CH₂)_{y}C(O)OH, wherein y is 1, 2, 3, 4, 5, or 6, —PO₂H₂, —PO₃H₂, —P(R²)O₂H, and phosphate, all of which can be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R^{2α} and R^{6α} are independently selected from the group consisting of halogen, nitro, alkyl, lower alkyl, alkenyl, alkynyl, carbocycle, cycloalkyl, haloalkyl, aryl, arylalkyl, heteroaryl, heteroaryl lower alkyl, heterocyclic, heterocyclic lower alkyl, R²C(O)alkyl, aminoalkyl, cycloalkylaminoalkyl, arylamino lower alkyl, heteroarylamino lower alkyl, heterocyclicamino lower alkyl, hydroxyalkyl, alkoxy, lower alkoxy, cycloalkyloxy, cycloalkylalkoxy, haloalkoxy, aryloxy, arylalkoxy, heteroaryloxy, heteroarylalkoxy, heteroaryl lower alkoxy, heterocyclicoxy, heterocyclicalkoxy, heterocyclic lower alkoxy, —OC(R¹)₂C(O)OH, —OC(R¹)₂C(O)OR², —OC(R¹)₂C(O)NH₂, —OC(R¹)₂C(O)NHR², —OC(R¹)₂C(O)N(R²)₂, —OC(R¹)₂C(O)NR⁷R⁸, —NHSO₂NHR², —NHSO₂R, —NHSO₂NR⁷R⁸, —NR²SO₂R², alkylthio, cycloalkylthio, —SC(R¹)₂C(O)OH, —SC(R¹)₂C(O)OR², —SCH₂C(O)OH, —SCF₂C(O)OH, —SO₂NHR², —SO₂NHR², —SO₂N(R²)₂, SO₂NR⁷R⁸, —SO₂NHC(O)R², —SR², —SO₂NHC(O)NHR², —SO₂NHC(O)N(R²) 2, —SO₂NHC(O)NR⁷R⁸, cyano, tetrazol-5-yl, carboxy, —C(O)OR², —C(O)NH₂, —C(O)NHR², —C(O)N(R²)₂, —C(O)NR⁷R⁸, —C(O)NHC(O)R², —C(O)NHC(O)NHR², —C(O)NHC(O)N(R²)₂, —C(O)NHC(O)NR⁷R⁸, —C(O)NHSO₂R², —C(O)NHSO₂NHR², —C(O)NHSO₂N(R²), —C(O)NHSO₂N⁷R⁸, —C(CH₃)₂C(O)OH, —(CH₂)_{y}C(O)OH, wherein y is 1, 2, 3, 4, 5, or 6, all of which can be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R¹ is independently selected from the group consisting of hydrogen, lower alkyl, carbocycle, cycloalkyl, aryl, heteroaryl, heterocyclic, arylalkyl, heteroarylalkyl, and heterocyclicalkyl, wherein all may be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R² is independently selected from the group consisting of alkyl, lower alkyl, alkenyl, alkynyl, carbocycle, cycloalkyl, aryl, heteroaryl, heterocyclic, arylalkyl, heteroarylalkyl, and heterocyclicalkyl, wherein all may be substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R⁷ and R⁸ are independently selected from the group consisting of alkyl, alkenyl, heteroaryl and aryl and linked together forming a 4- to 12-membered monocyclic, bicylic, tricyclic or benzofused ring;
wherein one of R^{2α}, R^{3α}, R^{4α}, R^{5α} or R^{6α} must be a carbon-carbon linked heteroaryl;
wherein all R¹, R², R⁷ and R⁸ substituents can be optionally substituted where possible with one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂.

6. The compound of claim 5 or its pharmaceutically acceptable salt or ester, wherein:
R^{3α}, R^{4α}, R^{5α}, R^{2β}, R^{3β}, R^{4β}, R^{5β} and R^{6β} are independently selected from the group consisting of hydrogen, halogen, nitro, alkyl, lower alkyl, alkenyl, alkynyl, carbocycle, cycloalkyl, cycloalkylalkyl, haloalkyl, aryl, arylalkyl, heteroaryl, heteroaryl lower alkyl, heterocyclic, heterocyclic lower alkyl, R²C(O)alkyl, aminoalkyl, hydroxyl, hydroxyalkyl, alkoxy, lower alkoxy, cycloalkyloxy, haloalkoxy, aryloxy, arylalkoxy, heteroaryloxy, heteroarylalkoxy, —OC(R¹)₂C(O)OH, —OC(R¹)₂C(O)OR², —OC(R¹)₂C(O)NH₂, —OC(R¹)₂C(O)NHR², —OC(R¹)₂C(O)N(R²)₂, —OC(R¹)₂C(O)NR⁷R⁸, amino, alkylamino, acylamino, dialkylamino, cycloalkylamino, arylamino, aralkylamino, heteroarylamino, heteroaralkylamino, heterocyclicamino, heterocyclicalkylamino, NHR², N(R²)₂, —NR⁷R⁸, —NHC(R¹)₂C(O)OH, —NHC(R¹)₂C(O)OR², —NHC(O)R², —N(R²)C(O)R², —NHC(O)OR², —NHC(O)SR², —NHSO₂NHR², —NHSO₂R², —NHSO₂NR⁷R⁸, —N(C(O)NHR²)₂, —NR²SO₂, —NHC(O)NHR², —NHC(O)NR⁷R⁸, —NHC(O)N(R²)₂, alkylsulfonyl, arylsulfonyl, haloalkylsulfonyl, —SC(R¹)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR⁷R⁸, —SO₂NHC(O)R², —SR², —SO₂NHC(O)NHR², —SO₂NHC(O)N(R²) 2, —SO₂NHC(O)NR⁷R⁸, cyano, tetrazol-5-yl, carboxy, —C(O)OR², —C(O)NH₂, —C(O)NHR², —C(O)N(R²)₂, —C(O)NR⁷R⁸, C(O)NHC(O)R², —C(O)NHC(O)NHR², —C(O)NHC(O)N(R²)₂, —C(O)NHC(O)NR⁷R⁸, —C(O)NHSO₂R², —C(O)NHSO₂NHR², —C(O)NHSO₂N(R²), —C(O)NHSO₂NR⁷R⁸, —C(CH₃)₂C(O)OH, all of which can be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R^{2α} and R^{6α} are independently selected from the group consisting of halogen, nitro, alkyl, lower alkyl, alkenyl, alkynyl, carbocycle, cycloalkyl, haloalkyl, aryl, arylalkyl, heteroaryl, heteroaryl lower alkyl, heterocyclic, heterocyclic lower alkyl, R²C(O)alkyl, aminoalkyl, hydroxyalkyl, alkoxy, lower alkoxy, cycloalkyloxy, cycloalkylalkoxy, haloalkoxy, aryloxy, arylalkoxy, heteroaryloxy, heteroarylalkoxy, heteroaryl lower alkoxy, heterocyclicoxy, heterocyclicalkoxy, heterocyclic lower alkoxy, —OC(R¹)₂C(O)OH, —OC(R¹)₂C(O)OR², —OC(R¹)₂C(O)NH₂, —OC(R¹)₂C(O)NHR², —OC(R¹)₂C(O)N(R²)₂, —OC(R¹)₂C(O)NR⁷R⁸, —SC(R¹)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR⁷R⁸, —SO₂NHC(O)R², —SR², —SO₂NHC(O)NHR², —SO₂NHC(O)N(R²)₂, —SO₂NHC(O)NR⁷R⁸, all of which can be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R¹ is independently selected from the group consisting of hydrogen, lower alkyl, carbocycle, cycloalkyl, aryl, heteroaryl, heterocyclic, arylalkyl, heteroarylalkyl, and heterocyclicalkyl, wherein all may be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R² is independently selected from the group consisting of alkyl, lower alkyl, alkenyl, alkynyl, carbocycle, cycloalkyl, aryl, heteroaryl, heterocyclic, arylalkyl, heteroarylalkyl, and heterocyclicalkyl, wherein all may be substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R⁷ and R⁸ are independently selected from the group consisting of alkyl, alkenyl, heteroaryl and aryl and linked together forming a 4- to 12-membered monocyclic, bicylic, tricyclic or benzofused ring;
wherein one of R^{2α}, R^{3α}, R^{4α}, R^{5α} or R^{6α} must be a carbon-carbon linked heteroaryl;
wherein all R¹, R², R⁷ and R⁸ substituents can be optionally substituted where possible with one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂.

7. The compound of claim 6 or its pharmaceutically acceptable salt or ester, wherein:
R^{3α}, R^{4α}, R^{5α}, R^{2β}, R^{3β}, R^{4β}, R^{5β} and R^{6β} are independently selected from the group consisting of hydrogen, halogen, nitro, alkyl, lower alkyl, alkenyl, alkynyl, carbocycle, cycloalkyl, cycloalkylalkyl, haloalkyl, aryl, arylalkyl, heteroaryl, heteroaryl lower alkyl, heterocyclic, heterocyclic lower alkyl, R²C(O)alkyl, aminoalkyl, hydroxyl, hydroxyalkyl, alkoxy, lower alkoxy, cycloalkyloxy, haloalkoxy, aryloxy, arylalkoxy, heteroaryloxy, heteroarylalkoxy, —OC(R¹)₂C(O)OH, —OC(R¹)₂C(O)OR², —OC(R¹)₂C(O)NH₂, —OC(R¹)₂C(O)NHR², —OC(R¹)₂C(O)N(R²)₂, —OC(R¹)₂C(O)NR⁷R⁸, amino, alkylamino, acylamino, dialkylamino, cycloalkylamino, arylamino, aralkylamino, heteroarylamino, heteroaralkylamino, heterocyclicamino, heterocyclicalkylamino, —NHR², N(R²)₂, —NR⁷R⁸, —NHC(R¹)₂C(O)OH, —NHC(R¹)₂C(O)OR², —NHC(O)R², —N(R²)C(O)R², —NHC(O)OR², —NHC(O)SR², —NHSO₂NHR², —NHSO₂R², —NHSO₂NR⁷R⁸, —N(C(O)NHR²)₂, —NR²SO₂R², —NHC(O)NHR², —NHC(O)NR⁷R⁸, —NHC(O)N(R²)₂, alkylsulfonyl, arylsulfonyl, haloalkylsulfonyl, —SC(R¹)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR⁷R⁸, —SO₂NHC(O)R², —SR², —SO₂NHC(O)NHR², —SO₂NHC(O)N(R²)₂, —SO₂NHC(O)NR⁷R⁸, cyano, tetrazol-5-yl, carboxy, —C(O)OR², —C(O)NH₂, —C(O)NHR², —C(O)N(R²)₂, —C(O)NR⁷R⁸, —C(O)NHC(O)R², —C(O)NHC(O)NHR², —C(O)NHC(O)N(R²)₂, —C(O)NHC(O)NR⁷R⁸, —C(O)NHSO₂R², —C(O)NHSO₂NHR², —C(O)NHSO₂N(R²), —C(O)NHSO₂NR⁷R⁸, —C(CH₃)₂C(O)OH, all of which can be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R^{2α} and R^{6α} are independently selected from the group consisting of halogen, nitro, lower alkyl, haloalkyl, aryl, heteroaryl, aminoalkyl, hydroxyalkyl, alkoxy, lower alkoxy, haloalkoxy, aryloxy, arylalkoxy, heteroaryloxy, heteroarylalkoxy, —OC(R¹)₂C(O)OH, —OC(R¹)₂C(O)OR², —OC(R¹)₂C(O)NH₂, —OC(R¹)₂C(O)NHR², —OC(R¹)₂C(O)N(R²)₂, —OC(R¹)₂C(O)NR⁷R⁸, —SC(R¹)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR⁷R⁸, —SO₂NHC(O)R², —SR², —SO₂NHC(O)NHR², —SO₂NHC(O)N(R²)₂, —SO₂NHC(O)NR⁷R⁸, all of which can be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R¹ is independently selected from the group consisting of hydrogen, lower alkyl, carbocycle, cycloalkyl, aryl, heteroaryl, heterocyclic, arylalkyl, heteroarylalkyl, and heterocyclicalkyl, wherein all may be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R² is independently selected from the group consisting of alkyl, lower alkyl, alkenyl, alkynyl, carbocycle, cycloalkyl, aryl, heteroaryl, heterocyclic, arylalkyl, heteroarylalkyl, and heterocyclicalkyl, wherein all may be substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R⁷ and R⁸ are independently selected from the group consisting of alkyl, alkenyl, heteroaryl and aryl and linked together forming a 4- to 12-membered monocyclic, bicylic, tricyclic or benzofused ring;
wherein one of R^{2α}, R^{3α}, R^{4α}, R^{5α} or R^{6α} must be a carbon-carbon linked heteroaryl;
wherein all R¹, R², R⁷ and R⁸ substituents can be optionally substituted where possible with one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂.

8. The compound of claim 5 or its pharmaceutically acceptable salt or ester, wherein:
R^{3α}, R^{4α}, R^{5α}, R^{2β}, R^{3β}, R^{4β}, R^{5β} and R^{6β} are independently selected from the group consisting of hydrogen, halogen, nitro, alkyl, lower alkyl, haloalkyl, aryl, arylalkyl, heteroaryl, heterocyclic, aminoalkyl, hydroxyl, hydroxyalkyl, alkoxy, lower alkoxy, haloalkoxy, aryloxy, arylalkoxy, heteroaryloxy, heteroarylalkoxy, —OC(R¹)₂C(O)OH, —OC(R¹)₂C(O)OR², amino, alkylamino, acylamino, dialkylamino, cycloalkylamino, arylamino, aralkylamino, heteroarylamino, heteroaralkylamino, heterocyclicamino, heterocyclicalkylamino, —NHR², N(R²)₂, —NR⁷R⁸, —NHC(R¹)₂C(O)OH, —NHC(R¹)₂C(O)OR², —NHC(O)R², —N(R²)C(O)R²—NHC(O)OR, —NHC(O)SR², —NHSO₂NHR², —NHSO₂R², —NHSO₂NR⁷R⁸, —N(C(O)NHR²)₂, —NR²SO₂R², —NHC(O)NHR², —NHC(O)NR⁷R⁸, —NHC(O)N(R²)₂, —SC(R¹)₂C(O)OH, —SC(R¹)₂C(O)OR², —SO₂NH₂, —SO₂NHR², —SO₂N(R²)₂, SO₂NR⁷R⁸, —SO₂NHC(O)R², —SR², —SO₂NHC(O)NHR², —SO₂NHC(O)N(R²) 2, —SO₂NHC(O)NR⁷R⁸, cyano, tetrazol-5-yl, carboxy, —C(O)OR², —C(O)NH₂, —C(O)NHR², —C(O)N(R²)₂, —C(O)NR⁷R⁸, —C(O)NHC(O)R², —C(O)NHC(O)NHR², —C(O)NHC(O)N(R²)₂, —C(O)NHC(O)NR⁷R⁸, —C(O)NHSO₂R², —C(O)NHSO₂NHR², —C(O)NHSO₂N(R²), —C(O)NHSO₂NR⁷R⁸, —C(CH₃)₂C(O)OH, all of which can be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R^{2α} and R^{6α} are independently selected from the group consisting of halogen, lower alkyl, lower alkoxy, aryl, and heteroaryl;
R¹ is independently selected from the group consisting of hydrogen, lower alkyl, carbocycle, cycloalkyl, aryl, heteroaryl, heterocyclic, arylalkyl, heteroarylalkyl, and heterocyclicalkyl, wherein all may be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R² is independently selected from the group consisting of alkyl, lower alkyl, alkenyl, alkynyl, carbocycle, cycloalkyl, aryl, heteroaryl, heterocyclic, arylalkyl, heteroarylalkyl, and heterocyclicalkyl, wherein all may be substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R⁷ and R⁸ are independently selected from the group consisting of alkyl, alkenyl, heteroaryl and aryl and linked together forming a 4- to 12-membered monocyclic, bicylic, tricyclic or benzofused ring;
wherein one of R^{2α}, R^{3α}, R^{4α}, R^{5α} or R^{6α} must be a carbon-carbon linked heteroaryl;
wherein all R¹, R², R⁷ and R⁸ substituents can be optionally substituted where possible with one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂.

9. The compound of claim 5 or its pharmaceutically acceptable salt or ester, wherein:
R^{2β}, R^{3β}, R^{4β}, R^{5β} and R^{6β} are independently selected from the group consisting of hydrogen, halogen, nitro, lower alkyl, haloalkyl, heteroaryl, heterocyclic, hydroxyl, lower alkoxy, —OC(R¹)₂C(O)OH, —OC(R¹)₂C(O)OR², —SO₂NHR², —SO₂N(R²)₂, carboxy, and —C(O)OR²;
R^{2α} and R^{6α} are independently selected from the group consisting of halogen, heteroaryl, aryl, lower alkyl, and lower alkoxy;
R¹ is independently selected from the group consisting of hydrogen and lower alkyl;
R² is lower alkyl, optionally substituted by one or more carboxy groups;
wherein one of R^{2α}, R^{3α}, R^{4α}, R^{5α}, or R^{6α} must be a carbon-carbon linked heteroaryl.

10. The compound of claim 5 or its pharmaceutically acceptable salt or ester, wherein:
R^{2β}, R^{3β}, R^{5β} and R^{6β} are independently selected from the group consisting of hydrogen, nitro, lower alkyl, haloalkyl, heteroaryl, heterocyclic, hydroxyl, lower alkoxy, —OC(R¹)₂C(O)OH, —OC(R¹)₂C(O)OR², —SO₂NHR², —SO₂N(R²)₂, carboxy, and —C(O)OR;
R^{4β} is independently selected from the group consisting of hydrogen, halogen, nitro, lower alkyl, haloalkyl, heteroaryl, heterocyclic, hydroxyl, lower alkoxy, —OC(R¹)₂C(O)OH, —OC(R¹)₂C(O)OR², —SO₂NHR², —SO₂N(R²)₂, carboxy, and C(O)OR²;
R^{2α} and R^{6α} are independently selected from the group consisting of halogen, heteroaryl, aryl, lower alkyl, and lower alkoxy;
R¹ is independently selected from the group consisting of hydrogen and lower alkyl;
R² is lower alkyl, optionally substituted by one or more carboxy groups;
wherein one of R^{2α}, R^{3α}, R^{4α}, R^{5α}, or R^{6α} must be a carbon-carbon linked heteroaryl.

11. The compound of claim 5 or its pharmaceutically acceptable salt or ester, wherein:
R^{2β}, R^{3β}, R^{4β}, R^{5β} and R^{6β} are independently selected from the group consisting of hydrogen, chloro, fluoro, bromo, nitro, methyl, tert-butyl, trifluoromethyl, thienyl, benzothienyl, methoxypyridyl, pyridyl, hydroxyl, methoxy, carboxy, —OCH₂C(O)OH, —OC(CH₃)₂C(O)OH, and —SO₂N(CH₃)CH₂C(O)OH;
R^{2α} and R^{6α} are independently selected from the group consisting of chloro, fluoro, bromo, nitro, methyl, and methoxy;
wherein one of R^{2α}, R^{3α}, R^{4α}, R^{5α}, or R^{6α} must be thienyl, benzothienyl, methoxypyridyl, or pyridyl.

12. The compound of claim 1 selected from the group consisting of:
4-[1-(2,6-Dimethoxy-3-thien-2-yl-benzoyl)-vinyl]-benzoic acid;
1-(2,6-Dimethoxy-3-thien-2-yl-phenyl)-2-(2-methoxy-phenyl)-propenone;
2-(3,5-Di-tert-butyl-4-hydroxy-phenyl)-1-(2,6-dimethoxy-3-thien-2-yl-phenyl)-propenone;
2-{4-[1-(2,6-Dimethoxy-3-thien-2-yl-benzoyl)-vinyl]-phenoxy}-2-methyl-propionic acid;
1-(2,6-Dimethoxy-3-thien-2-yl-phenyl)-2-(4-trifluoromethyl-phenyl)-propenone;
1-(2,6-Dimethoxy-3-thien-2-yl-phenyl)-2-(4-nitro-phenyl)-propenone;
2-(2,6-Dichloro-phenyl)-1-(2,6-dimethoxy-3-thien-2-yl-phenyl)-propenone;
2-{3-[1-(2,6-Dimethoxy-3-thien-2-yl-benzoyl)-vinyl]-phenoxy}-2-methyl-propionic acid;
2-{3-[1-(3-Benzo[b]thien-2-yl-2,6-dimethoxy-benzoyl)-vinyl]-phenoxy}-2-methyl-propionic acid;
({4-[1-(2,6-Dimethoxy-3-thien-2-yl-benzoyl)-vinyl]-benzenesulfonyl}-methyl-amino)-acetic acid; and
1-(2,6-Dimethoxy-3-thien-2-yl-phenyl)-2-(2-fluoro-6-trifluoromethyl-phenyl)-propenone.

13. The compound of claim 4 or its pharmaceutically acceptable salt or ester, wherein:
R^{3α}, R^{4α}, R^{5α}, R^{2β}, R^{3β}, R^{4β}, R^{5β} and R^{6β} are independently selected from the group consisting of hydrogen, halogen, nitro, alkyl, lower alkyl, alkenyl, alkynyl, carbocycle, cycloalkyl, cycloalkylalkyl, haloalkyl, aryl, arylalkyl, heteroaryl, heteroaryl lower alkyl, heterocyclic, heterocyclic lower alkyl, alkylthioalkyl, cycloalkylthioalkyl, arylthio lower alkyl, aralkyl lower thioalkyl, heteroarylthio lower alkyl, heteroaralkyl lower thioalkyl, heterocyclicthio lower alkyl, heterocyclicalkyl lower thioalkyl, —C(O)R², R²C(O)alkyl, aminoalkyl, cycloalkylaminoalkyl, arylamino lower alkyl, heteroarylamino lower alkyl, heterocyclicamino lower alkyl, hydroxyl, hydroxyalkyl, alkoxy, lower alkoxy, —(O(CH₂)₂)₁₋₃—O-lower alkyl, cycloalkyloxy, cycloalkylalkoxy, haloalkoxy, aryloxy, arylalkoxy, heteroaryloxy, heteroarylalkoxy, heteroaryl lower alkoxy, heterocyclicoxy, heterocyclicalkoxy, heterocyclic lower alkoxy, —OC(R¹)₂C(O)OH, —OC(R¹)₂C(O)OR², —OC(R¹)₂C(O)NH₂, —OC(R¹)₂C(O)NHR², —OC(R¹)₂C(O)N(R²)₂, —OC(R¹)₂C(O)NR⁷R⁸, amino, alkylamino, acylamino, dialkylamino, cycloalkylamino, arylamino, aralkylamino, heteroarylamino, heteroaralkylamino, heterocyclicamino, heterocyclicalkylamino, —NHR², N(R²)₂, —NR⁷R⁸, —NHC(R¹)₂C(O)OH, —NHC(R¹)₂C(O)OR², —NHC(O)R², —N(R²)C(O)R², —NHC(O)OR², —NHC(O)SR², —NHSO₂NHR², —NHSO₂R², —NHSO₂NR⁷R⁸, —N(C(O)NHR²)₂, —NR²SO₂R², —NHC(O)NHR², —NHC(O)NR⁷R⁸, —NHC(O)N(R²)₂, thiol, alkylthio, cycloalkylthio, cycloalkylalkylthio, haloalkylthio, arylthio, aralkylthio, heteroarylthio, heteroaralkylthio, heterocyclicthio, heterocyclicalkylthio, alkylsulfonyl, arylsulfonyl, haloalkylsulfonyl, —SC(R¹)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR⁷R⁸, —SO₂NHC(O)R², —SR², —SO₂NHC(O)NHR², —SO₂NHC(O)N(R²)₂, —SO₂NHC(O)NR⁷R⁸, sulfonic acid, sulfonate, sulfate, sulfinic acid, sulfenic acid, cyano, tetrazol-5-yl, carboxy, —C(O)OR², —C(O)NH₂, —C(O)NHR², —C(O)N(R²)₂, —C(O)NR⁷R⁸, —C(O)NHC(O)R², —C(O)NHC(O)NHR², —C(O)NHC(O)N(R²)₂, —C(O)NHC(O)NR⁷R⁸, —C(O)NHSO₂R², —C(O)NHSO₂NHR², —C(O)NHSO₂N(R²), —C(O)NHSO₂NR⁷R⁸, —C(CH₃)₂C(O)OH, —(CH₂)_{y}C(O)OH, wherein y is 1, 2, 3, 4, 5, or 6, —PO₂H₂, —PO₃H₂, —P(R²)O₂H, and phosphate, all of which can be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)N⁷R⁸, and —C(O)N(R²)₂;
R^{2α} and R^{6α} are independently selected from the group consisting of halogen, nitro, alkyl, lower alkyl, alkenyl, alkynyl, carbocycle, cycloalkyl, haloalkyl, aryl, arylalkyl, heteroaryl, heteroaryl lower alkyl, heterocyclic, heterocyclic lower alkyl, R²C(O) alkyl, aminoalkyl, cycloalkylaminoalkyl, arylamino lower alkyl, heteroarylamino lower alkyl, heterocyclicamino lower alkyl, hydroxyalkyl, alkoxy, lower alkoxy, cycloalkyloxy, cycloalkylalkoxy, haloalkoxy, aryloxy, arylalkoxy, heteroaryloxy, heteroarylalkoxy, heteroaryl lower alkoxy, heterocyclicoxy, heterocyclicalkoxy, heterocyclic lower alkoxy, —OC(R)₂C(O)OH, —OC(R¹)₂C(O)OR², —OC(R¹)₂C(O)NH₂, —OC(R¹)₂C(O)NHR², —OC(R¹)₂C(O)N(R²)₂, —OC(R¹)₂C(O)NR⁷R⁸, —NHSO₂NHR², —NHSO₂R², —NHSO₂NR⁷R⁸, —NR²SO₂R², alkylthio, cycloalkylthio, —SC(R¹)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR⁷R⁸, —SO₂NHC(O)R², —SR², —SO₂NHC(O)NHR², —SO₂NHC(O)N(R²) 2, —SO₂NHC(O)NR⁷R⁸, cyano, tetrazol-5-yl, carboxy, —C(O)OR², —C(O)NH₂, —C(O)NHR², —C(O)N(R²)₂, —C(O)NR⁷R⁸, C(O)NHC(O)R², —C(O)NHC(O)NHR², —C(O)NHC(O)N(R²)₂, —C(O)NHC(O)NR⁷R⁸, —C(O)NHSO₂R², —C(O)NHSO₂NHR², —C(O)NHSO₂N(R²), —C(O)NHSO₂NR⁷R⁸, —C(CH₃)₂C(O)OH, —(CH₂)_{y}C(O)OH, wherein y is 1, 2, 3, 4, 5, or 6, all of which can be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)N⁷R⁸, and —C(O)N(R²)₂;
R¹ is independently selected from the group consisting of hydrogen, lower alkyl, carbocycle, cycloalkyl, aryl, heteroaryl, heterocyclic, arylalkyl, heteroarylalkyl, and heterocyclicalkyl, wherein all may be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R² is independently selected from the group consisting of alkyl, lower alkyl, alkenyl, alkynyl, carbocycle, cycloalkyl, aryl, heteroaryl, heterocyclic, arylalkyl, heteroarylalkyl, and heterocyclicalkyl, wherein all may be substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R⁷ and R⁸ are independently selected from the group consisting of alkyl, alkenyl, heteroaryl and aryl and linked together forming a 4- to 12-membered monocyclic, bicylic, tricyclic or benzofused ring;
wherein one of R^{2β}, R^{3β}, R^{4β}, R^{5β} or R^{6β} must be a carbon-carbon linked heteroaryl;
wherein all R¹, R², R⁷ and R⁸ substituents can be optionally substituted where possible with one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂.

14. The compound of claim 13 or its pharmaceutically acceptable salt or ester, wherein:
R^{3α}, R^{4α}, R^{5α}, R^{2β}, R^{3β}, R^{4β}, R^{5β} and R^{6β} are independently selected from the group consisting of hydrogen, halogen, nitro, alkyl, lower alkyl, alkenyl, alkynyl, carbocycle, cycloalkyl, cycloalkylalkyl, haloalkyl, aryl, arylalkyl, heteroaryl, heteroaryl lower alkyl, heterocyclic, heterocyclic lower alkyl, R²C(O)alkyl, aminoalkyl, hydroxyl, hydroxyalkyl, alkoxy, lower alkoxy, cycloalkyloxy, haloalkoxy, aryloxy, arylalkoxy, heteroaryloxy, heteroarylalkoxy, —OC(R¹)₂C(O)OH, —OC(R¹)₂C(O)OR², —OC(R¹)₂C(O)NH₂, —OC(R¹)₂C(O)NHR², —OC(R¹)₂C(O)N(R²)₂, —OC(R¹)₂C(O)NR⁷R⁸, amino, alkylamino, acylamino, dialkylamino, cycloalkylamino, arylamino, aralkylamino, heteroarylamino, heteroaralkylamino, heterocyclicamino, heterocyclicalkylamino, —NHR², N(R²)₂, —NR⁷R⁸, —NHC(R¹)₂C(O)OH, —NHC(R¹)₂C(O)OR², —NHC(O)R², —N(R²)C(O)R², —NHC(O)OR², —NHC(O)SR², —NHSO₂NHR², —NHSO₂R², —NHSO₂NR⁷R⁸, —N(C(O)NHR²)₂, —NR²SO₂R², —NHC(O)NHR², —NHC(O)NR⁷R⁸, —NHC(O)N(R²)₂, alkylsulfonyl, arylsulfonyl, haloalkylsulfonyl, —SC(R¹)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR⁷R⁸, —SO₂NHC(O)R², —SR², —SO₂NHC(O)NHR², —SO₂NHC(O)N(R²) 2, —SO₂NHC(O)NR⁷R⁸, cyano, tetrazol-5-yl, carboxy, —C(O)OR², —C(O)NH₂, —C(O)NHR², —C(O)N(R²)₂, —C(O)NR⁷R⁸, —C(O)NHC(O)R², —C(O)NHC(O)NHR², —C(O)NHC(O)N(R²)₂, —C(O)NHC(O)NR⁷R⁸, —C(O)NHSO₂R², —C(O)NHSO₂NHR², —C(O)NHSO₂N(R²), —C(O)NHSO₂NR⁷R⁸, —C(CH₃)₂C(O)OH, all of which can be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R^{2α} and R^{6α} are independently selected from the group consisting of halogen, nitro, alkyl, lower alkyl, alkenyl, alkynyl, carbocycle, cycloalkyl, haloalkyl, aryl, arylalkyl, heteroaryl, heteroaryl lower alkyl, heterocyclic, heterocyclic lower alkyl, R²C(O)alkyl, aminoalkyl, hydroxyalkyl, alkoxy, lower alkoxy, cycloalkyloxy, cycloalkylalkoxy, haloalkoxy, aryloxy, arylalkoxy, heteroaryloxy, heteroarylalkoxy, heteroaryl lower alkoxy, heterocyclicoxy, heterocyclicalkoxy, heterocyclic lower alkoxy, —OC(R¹)₂C(O)OH, —OC(R¹)₂C(O)OR², —OC(R¹)₂C(O)NH₂, —OC(R¹)₂C(O)NHR², —OC(R¹)₂C(O)N(R²)₂, —OC(R¹)₂C(O)NR⁷R⁸, —SC(R)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR⁷R⁸, —SO₂NHC(O)R², —SR², —SO₂NHC(O)NHR², —SO₂NHC(O)N(R²) 2, —SO₂NHC(O)NR⁷R⁸, all of which can be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R¹ is independently selected from the group consisting of hydrogen, lower alkyl, carbocycle, cycloalkyl, aryl, heteroaryl, heterocyclic, arylalkyl, heteroarylalkyl, and heterocyclicalkyl, wherein all may be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R² is independently selected from the group consisting of alkyl, lower alkyl, alkenyl, alkynyl, carbocycle, cycloalkyl, aryl, heteroaryl, heterocyclic, arylalkyl, heteroarylalkyl, and heterocyclicalkyl, wherein all may be substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R⁷ and R⁸ are independently selected from the group consisting of alkyl, alkenyl, heteroaryl and aryl and linked together forming a 4- to 12-membered monocyclic, bicylic, tricyclic or benzofused ring;
wherein one of R^{2β}, R^{3β}, R^{4β}, R^{5β} or R^{6β} must be a carbon-carbon linked heteroaryl;
wherein all R¹, R², R⁷ and R⁸ substituents can be optionally substituted where possible with one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂.

15. The compound of claim 14 or its pharmaceutically acceptable salt or ester, wherein:
R^{3α}, R^{4α}, R^{5α}, R^{2β}, R^{3β}, R^{4β}, R^{5β} and R^{6β} are independently selected from the group consisting of hydrogen, halogen, nitro, alkyl, lower alkyl, alkenyl, alkynyl, carbocycle, cycloalkyl, cycloalkylalkyl, haloalkyl, aryl, arylalkyl, heteroaryl, heteroaryl lower alkyl, heterocyclic, heterocyclic lower alkyl, R²C(O)alkyl, aminoalkyl, hydroxyl, hydroxyalkyl, alkoxy, lower alkoxy, cycloalkyloxy, haloalkoxy, aryloxy, arylalkoxy, heteroaryloxy, heteroarylalkoxy, —OC(R¹)₂C(O)OH, —OC(R¹)₂C(O)OR², —OC(R¹)₂C(O)NH₂, —OC(R¹)₂C(O)NHR², —OC(R¹)₂C(O)N(R²)₂, —OC(R¹)₂C(O)NR⁷R⁸, amino, alkylamino, acylamino, dialkylamino, cycloalkylamino, arylamino, aralkylamino, heteroarylamino, heteroaralkylamino, heterocyclicamino, heterocyclicalkylamino, —NHR, N(R²)₂, —NR⁷R⁸, —NHC(R¹)₂C(O)OH, —NHC(R¹)₂C(O)OR², —NHC(O)R², —N(R²)C(O)R², —NHC(O)OR², —NHC(O)SR², —NHSO₂NHR², —NHSO₂R², —NHSO₂NR⁷R⁸, —N(C(O)NHR²)₂, —NR²SO₂R², —NHC(O)NHR², —NHC(O)NR⁷R⁸, —NHC(O)N(R²)₂, alkylsulfonyl, arylsulfonyl, haloalkylsulfonyl, —SC(R¹)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR⁷R⁸, —SO₂NHC(O)R², —SR², —SO₂NHC(O)NHR², —SO₂NHC(O)N(R²) 2, —SO₂NHC(O)NR⁷R⁸, cyano, tetrazol-5-yl, carboxy, —C(O)OR², —C(O)NH₂, —C(O)NHR², —C(O)N(R²)₂, —C(O)NR⁷R⁷, —C(O)NHC(O)R², —C(O)NHC(O)NHR², —C(O)NHC(O)N(R²)₂, —C(O)NHC(O)NR⁷R⁸, —C(O)NHSO₂R², —C(O)NHSO₂NHR², —C(O)NHSO₂N(R²), —C(O)NHSO₂NR⁷R⁸, —C(CH₃)₂C(O)OH, all of which can be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R^{2α} and R^{6α} are independently selected from the group consisting of halogen, nitro, lower alkyl, haloalkyl, aryl, heteroaryl, aminoalkyl, hydroxyalkyl, alkoxy, lower alkoxy, haloalkoxy, aryloxy, arylalkoxy, heteroaryloxy, heteroarylalkoxy, —OC(R¹)₂C(O)OH, —OC(R¹)₂C(O)OR², —OC(R¹)₂C(O)NH₂, —OC(R¹)₂C(O)NHR², —OC(R¹)₂C(O)N(R²)₂, —OC(R¹)₂C(O)NR⁷R⁸, —SC(R¹)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR⁷R⁸, —SO₂NHC(O)R², —SR², —SO₂NHC(O)NHR², —SO₂NHC(O)N(R²) 2, —SO₂NHC(O)NR⁷R⁸, all of which can be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R¹ is independently selected from the group consisting of hydrogen, lower alkyl, carbocycle, cycloalkyl, aryl, heteroaryl, heterocyclic, arylalkyl, heteroarylalkyl, and heterocyclicalkyl, wherein all may be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R² is independently selected from the group consisting of alkyl, lower alkyl, alkenyl, alkynyl, carbocycle, cycloalkyl, aryl, heteroaryl, heterocyclic, arylalkyl, heteroarylalkyl, and heterocyclicalkyl, wherein all may be substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R⁷ and R⁸ are independently selected from the group consisting of alkyl, alkenyl, heteroaryl and aryl and linked together forming a 4- to 12-membered monocyclic, bicylic, tricyclic or benzofused ring;
wherein one of R^{2β}, R^{3β}, R^{4β}, R^{5β} or R^{6β} must be a carbon-carbon linked heteroaryl;
wherein all R¹, R², R⁷ and R⁸ substituents can be optionally substituted where possible with one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂.

16. The compound of claim 13 or its pharmaceutically acceptable salt or ester, wherein:
R^{3α}, R^{4α}, R^{5α}, R^{2β}, R^{3β}, R^{4β}, R^{5β} and R^{6β} are independently selected from the group consisting of hydrogen, halogen, nitro, alkyl, haloalkyl, aryl, arylalkyl, heteroaryl, heterocyclic, aminoalkyl, hydroxyl, hydroxyalkyl, alkoxy, haloalkoxy, aryloxy, arylalkoxy, heteroaryloxy, heteroarylalkoxy, —OC(R¹)₂C(O)OH, —OC(R¹)₂C(O)OR², amino, alkylamino, acylamino, dialkylamino, cycloalkylamino, arylamino, aralkylamino, heteroarylamino, heteroaralkylamino, heterocyclicamino, heterocyclicalkylamino, —NHR², N(R²)₂, —NR⁷R⁸, —NHC(R¹)₂C(O)OH, —NHC(R¹)₂C(O)OR², —NHC(O)R², —N(R²)C(O)R², —NHC(O)OR², —NHC(O)SR², —NHSO₂NHR², —NHSO₂R², —NHSO₂NR⁷R⁸, —N(C(O)NHR²)₂, —NR²SO₂R², —NHC(O)NHR², —NHC(O)NR⁷R⁸, —NHC(O)N(R²)₂, —SC(R¹)₂C(O)OH, —SC(R¹)₂C(O)OR², —SO₂NH₂, —SO₂NHR², —SO₂N(R²)₂, SO₂NR⁷R⁸, —SO₂NHC(O)R², —SR², —SO₂NHC(O)NHR², —SO₂NHC(O)N(R²) 2, —SO₂NHC(O)NR⁷R⁸, cyano, tetrazol-5-yl, carboxy, —C(O)OR², —C(O)NH₂, —C(O)NHR², —C(O)N(R²)₂, —C(O)NR⁷R⁸, —C(O)NHC(O)R², —C(O)NHC(O)NHR², —C(O)NHC(O)N(R²)₂, —C(O)NHC(O)NR⁷R⁸, —C(O)NHSO₂R², —C(O)NHSO₂NHR², —C(O)NHSO₂N(R²), —C(O)NHSO₂NR⁷R⁸, —C(CH₃)₂C(O)OH, all of which can be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R^{2α} and R^{6α} are independently selected from the group consisting of F, Cl, Br, —CH₃, —OCH₃, aryl, and heteroaryl;
R¹ is independently selected from the group consisting of hydrogen, lower alkyl, carbocycle, cycloalkyl, aryl, heteroaryl, heterocyclic, arylalkyl, heteroarylalkyl, and heterocyclicalkyl, wherein all may be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R¹ is independently selected from the group consisting of alkyl, lower alkyl, alkenyl, alkynyl, carbocycle, cycloalkyl, aryl, heteroaryl, heterocyclic, arylalkyl, heteroarylalkyl, and heterocyclicalkyl, wherein all may be substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R⁷ and R⁸ are independently selected from the group consisting of alkyl, alkenyl, heteroaryl and aryl and linked together forming a 4- to 12-membered monocyclic, bicylic, tricyclic or benzofused ring;
wherein one of R^{2β}, R^{3β}, R^{4β}, R^{5β} or R^{6β} must be a carbon-carbon linked heteroaryl;
wherein all R¹, R², R⁷ and R⁸ substituents can be optionally substituted where possible with one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂.

17. The compound of claim 13 or its pharmaceutically acceptable salt or ester, wherein:
R^{2β}, R^{3β}, R^{4β}, R^{5β} and R^{6β} are independently selected from the group consisting of hydrogen, halogen, lower alkyl, haloalkyl, heteroaryl, heterocyclic, lower alkoxy, —OC(R¹)₂C(O)OH, —OC(R¹)₂C(O)OR², —SO₂NHR², —SO₂N(R²)₂, carboxy, and C(O)OR²;
R^{2α} and R^{6α} are independently selected from the group consisting of halogen, aryl, heteroaryl, lower alkyl, and lower alkoxy;
R¹ is independently selected from the group consisting of hydrogen and lower alkyl;
R² is lower alkyl, optionally substituted by one or more carboxy groups;
wherein one of R^{2β}, R^{3β}, R^{4β}, R^{5β} or R^{6β} must be a carbon-carbon linked heteroaryl.

18. The compound of claim 13 or its pharmaceutically acceptable salt or ester, wherein:
R^{2β}, R^{3β}, R^{4β}, R^{5β} and R^{6β} are independently selected from the group consisting of hydrogen, lower alkyl, haloalkyl, heteroaryl, heterocyclic, lower alkoxy, OC(R¹)₂C(O)OH, —OC(R¹)₂C(O)OR², —SO₂NHR², —SO₂N(R²)₂, carboxy, and —C(O)OR²;
R^{4β} is selected from the group consisting of hydrogen, halogen, lower alkyl, haloalkyl, heteroaryl, heterocyclic, lower alkoxy, —OC(R¹)₂C(O)OH, —OC(R¹)₂C(O)OR², —SO₂NHR², —SO₂N(R²)₂, carboxy, and —C(O)OR²;
R^{2β} and R^{6α} are independently selected from the group consisting of halogen, aryl, heteroaryl, lower alkyl, and lower alkoxy;
R¹ is independently selected from the group consisting of hydrogen and lower alkyl;
R² is lower alkyl, optionally substituted by one or more carboxy groups;
wherein one of R^{2β}, R^{3β}, R^{4β}, R^{5β} or R^{6β} must be a carbon-carbon linked heteroaryl.

19. The compound of claim 13 or its pharmaceutically acceptable salt or ester, wherein:
R^{2β}, R^{3β}, R^{4β}, R^{5β} and R^{6β} are independently selected from the group consisting of hydrogen, chloro, fluoro, bromo, nitro, methyl, tert-butyl, trifluoromethyl, thienyl, benzothienyl, methoxypyridyl, pyridyl, hydroxyl, methoxy, carboxy, —OCH₂C(O)OH, —OC(CH₃)₂C(O)OH, and —SO₂N(CH₃)CH₂C(O)OH;
R^{2α} and R^{6α} are independently selected from the group consisting of chloro, fluoro, bromo, nitro, methyl, and methoxy;
wherein one of R^{2β}, R^{3β}, R^{4β}, R^{5β} or R^{6β} must be thienyl, benzothienyl, methoxypyridyl, or pyridyl.

20. The compound of claim 1 selected from the group consisting of:
1-(2,6-Dichloro-phenyl)-2-(2-methoxy-5-thien-2-yl-phenyl)-propenone;
1-(2,6-Dimethyl-phenyl)-2-(2-methoxy-5-thien-2-yl-phenyl)-propenone;
4-[1-(2-Chloro-6-methyl-benzoyl)-vinyl]-3-thien-2-yl-benzoic acid;
4-[1-(2,6-Dimethyl-benzoyl)-vinyl]-3-thien-2-yl-benzoic acid;
4-[1-(2,6-Dimethoxy-benzoyl)-vinyl]-3-thien-2-yl-benzoic acid;
2-{4-[1-(2,6-Dimethoxy-benzoyl)-vinyl]-2-thien-2-yl-phenoxy}-2-methyl-propionic acid;
1-(2,6-Dimethyl-phenyl)-2-(2-thien-2-yl-phenyl)-propenone;
1-(2,6-Dimethyl-phenyl)-2-[2-(2-methoxy-pyridin-3-yl)-phenyl]-propenone;
1-(2,6-Dimethoxy-phenyl)-2-(2-thien-2-yl-phenyl)-propenone;
1-(2,6-Dimethoxy-phenyl)-2-(2-methoxy-5-thien-2-yl-phenyl)-propenone; and
2-{2-[1-(2,6-Dimethoxy-benzoyl)-vinyl]-4-thien-2-yl-phenoxy}-2-methyl-propionic acid.

21. The compound of claim 1 or its pharmaceutically acceptable salt or ester, wherein:
R^{2α}, R^{3α}, R^{4α}, R^{5α}, R^{6α}, and R^{4β} are independently selected from the group consisting of hydrogen, halogen, nitro, alkyl, lower alkyl, alkenyl, alkynyl, carbocycle, cycloalkyl, cycloalkylalkyl, haloalkyl, aryl, arylalkyl, heteroaryl, heteroaryl lower alkyl, heterocyclic, heterocyclic lower alkyl, alkylthioalkyl, cycloalkylthioalkyl, arylthio lower alkyl, aralkyl lower thioalkyl, heteroarylthio lower alkyl, heteroaralkyl lower thioalkyl, heterocyclicthio lower alkyl, heterocyclicalkyl lower thioalkyl, —C(O)R², R²C(O)alkyl, aminoalkyl, cycloalkylaminoalkyl, arylamino lower alkyl, heteroarylamino lower alkyl, heterocyclicamino lower alkyl, hydroxyl, hydroxyalkyl, alkoxy, lower alkoxy, —(O(CH₂)₂)₁₋₃—O-lower alkyl, cycloalkyloxy, cycloalkylalkoxy, haloalkoxy, aryloxy, arylalkoxy, heteroaryloxy, heteroarylalkoxy, heteroaryl lower alkoxy, heterocyclicoxy, heterocyclicalkoxy, heterocyclic lower alkoxy, —OC(R¹)₂C(O)OH, —OC(R¹)₂C(O)OR², —OC(R¹)₂C(O)NH₂, —OC(R¹)₂C(O)NHR², —OC(R¹)₂C(O)N(R²)₂, —OC(R¹)₂C(O)NR⁷R⁸, amino, alkylamino, acylamino, dialkylamino, cycloalkylamino, arylamino, aralkylamino, heteroarylamino, heteroaralkylamino, heterocyclicamino, heterocyclicalkylamino, —NHR², N(R²)₂, —NR⁷R⁸, —NHC(R¹)₂C(O)OH, —NHC(R¹)₂C(O)OR², —NHC(O)R², —N(R²)C(O)R², —NHC(O)OR², —NHC(O)SR², —NHSO₂NHR², —NHSO₂R², —NHSO₂NR⁷R⁸, —N(C(O)NHR²)₂, —NR²SO₂R², —NHC(O)NHR², —NHC(O)NR⁷R⁸, —NHC(O)N(R²)₂, thiol, alkylthio, cycloalkylthio, cycloalkylalkylthio, haloalkylthio, arylthio, aralkylthio, heteroarylthio, heteroaralkylthio, heterocyclicthio, heterocyclicalkylthio, alkylsulfonyl, arylsulfonyl, haloalkylsulfonyl, —SC(R¹)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR⁷R⁸, —SO₂NHC(O)R², —SR², —SO₂NHC(O)NHR², —SO₂NHC(O)N(R²)₂, —SO₂NHC(O)NR⁷R⁸, sulfonic acid, sulfonate, sulfate, sulfinic acid, sulfenic acid, cyano, tetrazol-5-yl, carboxy, —C(O)OR², —C(O)NH₂, —C(O)NHR², —C(O)N(R²)₂, —C(O)NR⁷R⁸, —C(O)NHC(O)R², —C(O)NHC(O)NHR², —C(O)NHC(O)N(R²)₂, —C(O)NHC(O)NR⁷R⁸, —C(O)NHSO₂R², —C(O)NHSO₂NHR², —C(O)NHSO₂N(R²), —C(O)NHSO₂NR⁷R⁸, —C(CH₃)₂C(O)OH, —(CH₂)_{y}C(O)OH, wherein y is 1, 2, 3, 4, 5, or 6, —PO₂H₂, —PO₃H₂, —P(R²)O₂H, and phosphate, all of which can be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R^{2β}, R^{3β}, R^{5β} and R^{6β} are independently selected from the group consisting of hydrogen, nitro, alkyl, lower alkyl, alkenyl, alkynyl, carbocycle, cycloalkyl, cycloalkylalkyl, haloalkyl, aryl, arylalkyl, heteroaryl, heteroaryl lower alkyl, heterocyclic, heterocyclic lower alkyl, alkylthioalkyl, cycloalkylthioalkyl, arylthio lower alkyl, aralkyl lower thioalkyl, heteroarylthio lower alkyl, heteroaralkyl lower thioalkyl, heterocyclicthio lower alkyl, heterocyclicalkyl lower thioalkyl, —C(O)R², R²C(O)alkyl, aminoalkyl, cycloalkylaminoalkyl, arylamino lower alkyl, heteroarylamino lower alkyl, heterocyclicamino lower alkyl, hydroxyl, hydroxyalkyl, alkoxy, lower alkoxy, —(O(CH₂)₂)₁₋₃—O-lower alkyl, cycloalkyloxy, cycloalkylalkoxy, haloalkoxy, aryloxy, arylalkoxy, heteroaryloxy, heteroarylalkoxy, heteroaryl lower alkoxy, heterocyclicoxy, heterocyclicalkoxy, heterocyclic lower alkoxy, —OC(R¹)₂C(O)OH, —OC(R¹)₂C(O)OR², —OC(R¹)₂C(O)NH₂, —OC(R¹)₂C(O)NHR², —OC(R¹)₂C(O)N(R²)₂, —OC(R¹)₂C(O)NR⁷R⁸, amino, alkylamino, acylamino, dialkylamino, cycloalkylamino, arylamino, aralkylamino, heteroarylamino, heteroaralkylamino, heterocyclicamino, heterocyclicalkylamino, —NHR², N(R²)₂, —NR⁷R⁸, —NHC(R¹)₂C(O)OH, —NHC(R¹)₂C(O)OR², —NHC(O)R², —N(R²)C(O)R², —NHC(O)OR², —NHC(O)SR², —NHSO₂NHR², —NHSO₂R², —NHSO₂NR⁷R⁸, —N(C(O)NHR²)₂, —NR²SO₂R², —NHC(O)NHR², —NHC(O)NR⁷R⁸, —NHC(O)N(R²)₂, thiol, alkylthio, cycloalkylthio, cycloalkylalkylthio, haloalkylthio, arylthio, aralkylthio, heteroarylthio, heteroaralkylthio, heterocyclicthio, heterocyclicalkylthio, alkylsulfonyl, arylsulfonyl, haloalkylsulfonyl, —SC(R¹)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR⁷R⁸, —SO₂NHC(O)R², —SR², —SO₂NHC(O)NHR², —SO₂NHC(O)N(R²) 2, —SO₂NHC(O)NR⁷R⁸, sulfonic acid, sulfonate, sulfate, sulfinic acid, sulfenic acid, cyano, tetrazol-5-yl, carboxy, —C(O)OR², —C(O)NH₂, —C(O)NHR², —C(O)N(R²)₂, —C(O)NR⁷R⁸, —C(O)NHC(O)R², —C(O)NHC(O)NHR², —C(O)NHC(O)N(R²)₂, —C(O)NHC(O)NR⁷R⁸, —C(O)NHSO₂R², —C(O)NHSO₂NHR², —C(O)NHSO₂N(R²), —C(O)NHSO₂NR⁷R⁸, —C(CH₃)₂C(O)OH, —(CH₂)_{y}C(O)OH, wherein y is 1, 2, 3, 4, 5, or 6, —PO₂H₂, —PO₃H₂, —P(R²)O₂H, and phosphate, all of which can be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R¹ is independently selected from the group consisting of hydrogen, lower alkyl, carbocycle, cycloalkyl, aryl, heteroaryl, heterocyclic, arylalkyl, heteroarylalkyl, and heterocyclicalkyl, wherein all may be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R² is independently selected from the group consisting of alkyl, lower alkyl, alkenyl, alkynyl, carbocycle, cycloalkyl, aryl, heteroaryl, heterocyclic, arylalkyl, heteroarylalkyl, and heterocyclicalkyl, wherein all may be substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R⁷ and R⁸ are independently selected from the group consisting of alkyl, alkenyl, heteroaryl and aryl and linked together forming a 4- to 12-membered monocyclic, bicylic, tricyclic or benzofused ring;
wherein one of R^{2α}, R^{3α}, R^{4α}, R^{5α} or R^{6α} must be a carbon-carbon linked heteroaryl;
wherein all R¹, R², R⁷ and R⁸ substituents can be optionally substituted where possible with one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂.

22. The compound of claim 21 or its pharmaceutically acceptable salt or ester, wherein:
R^{2α}, R^{3α}, R^{4α}, R^{5α}, R^{6α} and R^{4β}, are independently selected from the group consisting of hydrogen, halogen, nitro, alkyl, haloalkyl, heteroaryl, heterocyclic, hydroxyl, alkoxy, haloalkoxy, heteroaryloxy, heteroarylalkoxy, heterocyclicoxy, heterocyclicalkoxy, —OC(R¹)₂C(O)OH, —OC(R¹)₂C(O)OR², —OC(R¹)₂C(O)NH₂, —OC(R¹)₂C(O)NHR², —OC(R¹)₂C(O)N(R²)₂, —OC(R¹)₂C(O)NR⁷R⁸, heteroarylamino, heterocyclicamino, —NHR², N(R²)₂, —NR⁷R⁸, —NHC(O)R², —N(R²)C(O)R², —NHC(O)OR², —NHSO₂R², —NR²SO₂R², cyano, tetrazol-5-yl, carboxy, —C(O)OR², —C(O)NH₂, —C(O)NHR², —C(O)N(R²)₂, —C(O)NR⁷R⁸, all of which can be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R^{2β}, R^{3β}, R^{5β} and R^{6β} are independently selected from the group consisting of hydrogen, heteroaryl, heterocyclic, alkyl, alkoxy, and lower alkoxy, all of which can be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R¹ is independently selected from the group consisting of hydrogen, and lower alkyl, optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R¹ is independently selected from the group consisting of alkyl and lower alkyl, optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R⁷ and R⁸ are independently selected from the group consisting of alkyl, alkenyl, heteroaryl and aryl and linked together forming a 4- to 12-membered monocyclic, bicylic, tricyclic or benzofused ring, optionally substituted where possible with one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
wherein one of R^{2α}, R^{3α}, R^{4α}, R^{5α} or R^{6α} must be a carbon-carbon linked heteroaryl.

23. The compound of claim 1 selected from the group consisting of:
4-[1-(5-Benzo[b]thien-2-yl-2,4-dimethoxy-benzoyl)-vinyl]-benzoic acid methyl ester;
4-[1-(4-Methoxy-3-thien-2-yl-benzoyl)-vinyl]-benzoic acid;
4-[1-(3,4-Dimethoxy-5-thien-2-yl-benzoyl)-vinyl]-benzoic acid;
1-(5-Benzo[b]thien-2-yl-2,4-dimethoxy-phenyl)-2-(4-methoxy-phenyl)-propenone;
1-(5-Benzo[b]thien-2-yl-2,4-dimethoxy-phenyl)-2-(4-fluoro-phenyl)-propenone;
1-(2-Methoxy-5-thien-2-yl-phenyl)-2-(4-nitro-phenyl)-propenone;
4-[1-(2-Methoxy-5-thien-2-yl-benzoyl)-vinyl]-benzonitrile; and
4-[1-(2-Methoxy-5-thien-2-yl-benzoyl)-vinyl]-benzoic acid.

24. The compound of claim 1 or its pharmaceutically acceptable salt or ester, wherein:
R^{2α}, R^{3α}, R^{4α}, R^{5α}, R^{6α}, and R^{4β} are independently selected from the group consisting of hydrogen, halogen, nitro, alkyl, lower alkyl, alkenyl, alkynyl, carbocycle, cycloalkyl, cycloalkylalkyl, haloalkyl, aryl, arylalkyl, heteroaryl, heteroaryl lower alkyl, heterocyclic, heterocyclic lower alkyl, alkylthioalkyl, cycloalkylthioalkyl, arylthio lower alkyl, aralkyl lower thioalkyl, heteroarylthio lower alkyl, heteroaralkyl lower thioalkyl, heterocyclicthio lower alkyl, heterocyclicalkyl lower thioalkyl, —C(O)R², R²C(O)alkyl, aminoalkyl, cycloalkylaminoalkyl, arylamino lower alkyl, heteroarylamino lower alkyl, heterocyclicamino lower alkyl, hydroxyl, hydroxyalkyl, alkoxy, lower alkoxy, —(O(CH₂)₂)₁₋₃—O-lower alkyl, cycloalkyloxy, cycloalkylalkoxy, haloalkoxy, aryloxy, arylalkoxy, heteroaryloxy, heteroarylalkoxy, heteroaryl lower alkoxy, heterocyclicoxy, heterocyclicalkoxy, heterocyclic lower alkoxy, —OC(R¹)₂C(O)OH, —OC(R¹)₂C(O)OR², —OC(R¹)₂C(O)NH₂, —OC(R¹)₂C(O)NHR², —OC(R²)₂C(O)N(R²)₂, —OC(R¹)₂C(O)NR⁷R⁸, amino, alkylamino, acylamino, dialkylamino, cycloalkylamino, arylamino, aralkylamino, heteroarylamino, heteroaralkylamino, heterocyclicamino, heterocyclicalkylamino, —NHR², N(R²)₂, —NR⁷R⁸, —NHC(R¹)₂C(O)OH, —NHC(R¹)₂C(O)OR², —NHC(O)R², —N(R²)C(O)R², —NHC(O)OR², —NHC(O)SR², —NHSO₂NHR², —NHSO₂R², —NHSO₂NR⁷R⁸, —N(C(O)NHR²)₂, —NR²SO₂R², —NHC(O)NHR², —NHC(O)NR⁷R⁸, —NHC(O)N(R²)₂, thiol, alkylthio, cycloalkylthio, cycloalkylalkylthio, haloalkylthio, arylthio, aralkylthio, heteroarylthio, heteroaralkylthio, heterocyclicthio, heterocyclicalkylthio, alkylsulfonyl, arylsulfonyl, haloalkylsulfonyl, —SC(R¹)₂C(O)OH, —SC(R¹)₂C(O)OR², —SCH₂C(O)OH, —SCF₂C(O)OH, —SO₂NH₂, SO₂NHR², —SO₂N(R²)₂, SO₂NR⁷R⁸, —SO₂NHC(O)R², —SR², —SO₂NHC(O)NHR², SO₂NHC(O)N(R²) 2, —SO₂NHC(O)NR⁷R⁸, sulfonic acid, sulfonate, sulfate, sulfinic acid, sulfenic acid, cyano, tetrazol-5-yl, carboxy, —C(O)OR², —C(O)NHR₂, —C(O)NHR², —C(O)N(R²)₂, —C(O)NR⁷R⁸, —C(O)NHC(O)R², —C(O)NHC(O)NHR², —C(O)NHC(O)N(R²)₂, —C(O)NHC(O)NR⁷R⁸, —C(O)NHSO₂R², —C(O)NHSO₂NHR², —C(O)NHSO₂N(R²), —C(O)NHSO₂NR⁷R⁸, —C(CH₃)₂C(O)OH, —(CH₂)_{y}C(O)OH, wherein y is 1, 2, 3, 4, 5, or 6, —PO₂H₂, —PO₃H₂, —P(R²)O₂H, and phosphate, all of which can be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R^{2β}, R^{3β}, R^{5β} and R^{6β} are independently selected from the group consisting of hydrogen, nitro, alkyl, lower alkyl, alkenyl, alkynyl, carbocycle, cycloalkyl, cycloalkylalkyl, haloalkyl, aryl, arylalkyl, heteroaryl, heteroaryl lower alkyl, heterocyclic, heterocyclic lower alkyl, alkylthioalkyl, cycloalkylthioalkyl, arylthio lower alkyl, aralkyl lower thioalkyl, heteroarylthio lower alkyl, heteroaralkyl lower thioalkyl, heterocyclicthio lower alkyl, heterocyclicalkyl lower thioalkyl, —C(O)R², R²C(O)alkyl, aminoalkyl, cycloalkylaminoalkyl, arylamino lower alkyl, heteroarylamino lower alkyl, heterocyclicamino lower alkyl, hydroxyl, hydroxyalkyl, alkoxy, lower alkoxy, —(O(CH₂)₂)₁₋₃—O-lower alkyl, cycloalkyloxy, cycloalkylalkoxy, haloalkoxy, aryloxy, arylalkoxy, heteroaryloxy, heteroarylalkoxy, heteroaryl lower alkoxy, heterocyclicoxy, heterocyclicalkoxy, heterocyclic lower alkoxy, —OC(R¹)₂C(O)OH, —OC(R¹)₂C(O)OR², —OC(R¹)₂C(O)NH₂, —OC(R¹)₂C(O)NHR², —OC(R¹)₂C(O)N(R²)₂, —OC(R¹)₂C(O)NR⁷R⁸, amino, alkylamino, acylamino, dialkylamino, cycloalkylamino, arylamino, aralkylamino, heteroarylamino, heteroaralkylamino, heterocyclicamino, heterocyclicalkylamino, —NHR², N(R²)₂, —NR⁷R⁸, —NHC(R¹)₂C(O)OH, —NHC(R¹)₂C(O)OR², —NHC(O)R², —N(R²)C(O)R², —NHC(O)OR², —NHC(O)SR², —NHSO₂NHR², —NHSO₂R², —NHSO₂NR⁷R⁸, —N(C(O)NHR²)₂, —NR²SO₂R², —NHC(O)NHR², —NHC(O)NR⁷R⁸, —NHC(O)N(R²)₂, thiol, alkylthio, cycloalkylthio, cycloalkylalkylthio, haloalkylthio, arylthio, aralkylthio, heteroarylthio, heteroaralkylthio, heterocyclicthio, heterocyclicalkylthio, alkylsulfonyl, arylsulfonyl, haloalkylsulfonyl, —SC(R¹)₂C(O)OH, —SC(R¹)₂C(O)OR², —SCH₂C(O)OH, —SCF₂C(O)OH, —SO₂NH₂, —SO₂NHR², —SO₂N(R²)₂, SO₂NR⁷R⁸, —SO₂NHC(O)R², —SR₂, —SO₂NHC(O)NHR², —SO₂NHC(O)N(R²) 2, SO₂NHC(O)NR⁷R⁸, sulfonic acid, sulfonate, sulfate, sulfinic acid, sulfenic acid, cyano, tetrazol-5-yl, carboxy, —C(O)OR², —C(O)NH₂, —C(O)NHR², —C(O)N(R²)₂, —C(O)NR⁷R⁸, —C(O)NHC(O)R², —C(O)NHC(O)NHR², —C(O)NHC(O)N(R²)₂, —C(O)NHC(O)NR⁷R⁸, —C(O)NHSO₂R², —C(O)NHSO₂NHR², —C(O)NHSO₂N(R²), —C(O)NHSO₂NR⁷R⁸, —C(CH₃)₂C(O)OH, —(CH₂)_{y}C(O)OH, wherein y is 1, 2, 3, 4, 5, or 6, —PO₂H₂, —PO₃H₂, —P(R²)O₂H, and phosphate, all of which can be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R¹ is independently selected from the group consisting of hydrogen, lower alkyl, carbocycle, cycloalkyl, aryl, heteroaryl, heterocyclic, arylalkyl, heteroarylalkyl, and heterocyclicalkyl, wherein all may be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R¹)₂;
R² is independently selected from the group consisting of alkyl, lower alkyl, alkenyl, alkynyl, carbocycle, cycloalkyl, aryl, heteroaryl, heterocyclic, arylalkyl, heteroarylalkyl, and heterocyclicalkyl, wherein all may be substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R⁷ and R⁸ are independently selected from the group consisting of alkyl, alkenyl, heteroaryl and aryl and linked together forming a 4- to 12-membered monocyclic, bicylic, tricyclic or benzofused ring;
wherein one of R^{2β}, R^{3β}, R^{4β}, R^{5β} or R^{6β} must be a carbon-carbon linked heteroaryl;
wherein all R¹, R², R⁷ and R⁸ substituents can be optionally substituted where possible with one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂.

25. The compound of claim 24 or its pharmaceutically acceptable salt or ester, wherein:
R^{2α}, R^{3α}, R^{4α}, R^{5α}, R^{6α} and R^{4β}, are independently selected from the group consisting of hydrogen, halogen, nitro, alkyl, haloalkyl, heteroaryl, heterocyclic, hydroxyl, alkoxy, haloalkoxy, heteroaryloxy, heteroarylalkoxy, heterocyclicoxy, heterocyclicalkoxy, —OC(R¹)₂C(O)OH, —OC(R¹)₂C(O)OR², —OC(R¹)₂C(O)NH₂, —OC(R)₂C(O)NHR², —OC(R¹)₂C(O)N(R²)₂, —OC(R¹)₂C(O)NR⁷R⁸, heteroarylamino, heterocyclicamino, —NHR², N(R²)₂, —NR⁷R⁸, —NHC(O)R², —N(R²)C(O)R², —NHC(O)OR², —NHSO₂R², —NR²SO₂R², cyano, tetrazol-5-yl, carboxy, —C(O)OR², —C(O)NH₂, —C(O)NHR², —C(O)N(R²)₂, —C(O)NR⁷R⁸, all of which can be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R^{2β}, R^{3β}, R^{5β} and R^{6β} are independently selected from the group consisting of hydrogen, heteroaryl, heterocyclic, alkyl, alkoxy, and lower alkoxy, all of which can be optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R¹ is independently selected from the group consisting of hydrogen, and lower alkyl, optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R² is independently selected from the group consisting of alkyl and lower alkyl, optionally substituted where possible by one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
R⁷ and R⁸ are independently selected from the group consisting of alkyl, alkenyl, heteroaryl and aryl and linked together forming a 4- to 12-membered monocyclic, bicylic, tricyclic or benzofused ring, optionally substituted where possible with one or more selected from the group consisting of halo, alkyl, lower alkyl, alkenyl, cycloalkyl, acyl, hydroxy, hydroxyalkyl, heterocyclic, amino, aminoalkyl, —NR⁷R⁸, alkoxy, oxo, cyano, carboxy, carboxyalkyl, alkoxycarbonyl, —C(O)NR⁷R⁸, and —C(O)N(R²)₂;
wherein one of R^{2β}, R^{3β}, R^{4β}, R^{5β} or R^{6β} must be a carbon-carbon linked heteroaryl.

26. The compound of claim 1 selected from the group consisting of:
4-[2-(4-Methoxy-3-thien-2-yl-phenyl)-acryloyl]-benzoic acid;
4-[2-(2-Methoxy-5-quinolin-3-yl-phenyl)-acryloyl]-benzoic acid;
2-{3-[1-(4-Carboxy-benzoyl)-vinyl]-4-methoxy-phenyl}-pyrrole-1-carboxylic acid tert-butyl ester;
4-[2-(2-Methoxy-5-pyridin-3-yl-phenyl)-acryloyl]-benzoic acid;
4-[2-(5-Benzo[b]thien-2-yl-2-methoxy-phenyl)-acryloyl]-benzoic acid;
4-[2-(2-Methoxy-5-thien-2-yl-phenyl)-acryloyl]-benzoic acid;
4-[2-(2-Methoxy-5-thien-2-yl-phenyl)-acryloyl]-benzoic acid;
4-[2-(2-Methoxy-5-thien-2-yl-phenyl)-acryloyl]-benzoic acid methyl ester;
N-(2-Hydroxy-1,1-bis-hydroxymethyl-ethyl)-4-[2-(2-methoxy-5-thien-2-yl-phenyl)-acryloyl]-benzamide;
1-[4-(4-Hydroxy-piperidine-1-carbonyl)-phenyl]-2-(2-methoxy-5-thien-2-yl-phenyl)-propenone;
1-(4-Fluoro-phenyl)-2-(2-methoxy-5-thien-2-yl-phenyl)-propenone;
2-(2-Methoxy-5-thien-2-yl-phenyl)-1-(4-pyrrolidin-1-yl-phenyl)-propenone;
1-(4-Hydroxy-phenyl)-2-(2-methoxy-5-thien-2-yl-phenyl)-propenone;
2-(2-Methoxy-5-thien-2-yl-phenyl)-1-(4-nitro-phenyl)-propenone;
N-{4-[2-(2-Methoxy-5-thien-2-yl-phenyl)-acryloyl]-phenyl}-methanesulfonamide;
N-{4-[2-(2-Methoxy-5-thien-2-yl-phenyl)-acryloyl]-phenyl}-N-methyl-methane-sulfonamide;
N-{4-[2-(2-Methoxy-5-thien-2-yl-phenyl)-acryloyl]-phenyl}-isobutyramide;
2-(2-Methoxy-5-thien-2-yl-phenyl)-1-[4-(pyrimidin-2-ylamino)-phenyl]-propenone;
2-{4-[2-(2-Methoxy-5-thien-2-yl-phenyl)-acryloyl]-phenylamino}-nicotinic acid ethyl ester;
2-{4-[2-(2-Methoxy-5-thien-2-yl-phenyl)-acryloyl]-phenylamino}-nicotinic acid;
2-(2-Methoxy-5-thien-2-yl-phenyl)-1-[4-(pyrazin-2-ylamino)-phenyl]-propenone;
3,5-Dimethyl-isoxazole-4-sulfonic acid {4-[2-(2-methoxy-5-thien-2-yl-phenyl)-acryloyl]-phenyl}-amide;
Isoxazole-5-carboxylic acid {4-[2-(2-methoxy-5-thien-2-yl-phenyl)-acryloyl]-phenyl}-amide;
1-Methyl-1H-pyrrole-2-carboxylic acid {4-[2-(2-methoxy-5-thien-2-yl-phenyl)-acryloyl]-phenyl}-amide;
4-[2-(3,4-Dimethoxy-5-thien-2-yl-phenyl)-acryloyl]-benzoic acid methyl ester;
4-[2-(3,4-Dimethoxy-5-thien-2-yl-phenyl)-acryloyl]-benzoic acid;
4-[2-(3,4-Dimethoxy-5-thien-2-yl-phenyl)-acryloyl]-N-(2-morpholin-4-yl-ethyl)-benzamide;
2-(5-Benzo[b]thien-2-yl-2,4-dimethoxy-phenyl)-1-(4-fluoro-phenyl)-propenone;
4-[2-(2,4-Dimethoxy-5-thien-2-yl-phenyl)-acryloyl]-benzoic acid;
3-[2-(2-Methoxy-5-thien-2-yl-phenyl)-acryloyl]-benzoic acid;
1-(4-tert-Butyl-phenyl)-2-(2-methoxy-5-thien-2-yl-phenyl)-propenone;
2-(2-Methoxy-5-thien-2-yl-phenyl)-1-(4-trifluoromethyl-phenyl)-propenone;
4-[2-(2-Isopropoxy-5-thien-2-yl-phenyl)-acryloyl]-benzoic acid;
4-{2-[2-(2-Oxo-2-piperidin-1-yl-ethoxy)-5-thien-2-yl-phenyl]-acryloyl}-benzoic acid;
4-{2-[2-(2-Piperidin-1-yl-ethoxy)-5-thien-2-yl-phenyl]-acryloyl}-benzoic acid hydrochloride salt; and
4-[2-(2-Methoxy-5-thien-3-yl-phenyl)-acryloyl]-benzoic acid.

27. A pharmaceutical composition comprising a therapeutically effective amount of a compound of claim 1, 2, 3, 4, 5, 6, 7, 8, 9, 10, 11, 12, 13, 14, 15, 16, 17, 18, 19, 20, 21, 22, 23, 24, 25, or 26, together with one or more pharmaceutically acceptable carrier.

28. A method for the treatment or prophylaxis of an inflammatory disorder, comprising administering an effective amount of a compound of claim 1, 2, 3, 4, 5, 6, 7, 8, 9, 10, 11, 12, 13, 14, 15, 16, 17, 18, 19, 20, 21, 22, 23, 24, 25, or 26.

29. The method of claim 28, wherein the disorder is arthritis.

30. The method of claim 28, wherein the disorder is rheumatoid arthritis.

31. The method of claim 28, wherein the disorder is asthma.

32. The method of claim 28, wherein the treatment is disease modifying for the treatment of rheumatoid arthritis.

33. The method of claim 28, wherein the disorder is allergic rhinitis.

34. The method of claim 28, wherein the disorder is chronic obstructive pulmonary disease.

35. The method of claim 28, wherein the disorder is atherosclerosis.

36. The method of claim 28, wherein the disorder is restinosis.

37. A method for inhibiting the expression of VCAM-1, comprising administering an effective amount of a compound of claim 1, 2, 3, 4, 5, 6, 7, 8, 9, 10, 11, 12, 13, 14, 15, 16, 17, 18, 19, 20, 21, 22, 23, 24, 25, or 26.
